(19) United States
(12) Patent Application Publication
(10) Pub. No.: US 2026/0041640 A1
(43) Pub. Date: Feb. 12, 2026

(54) ADVANTAGEOUS HUMAN DOSAGE REGIMENS FOR ADMINISTRATION OF INX-315

(71) Applicant: INCYCLIX BIO, LLC, Research Triangle Park, NC (US)

(72) Inventors: Jay Copeland Strum, Hillsborough, NC (US); Alexander Smith, Apex, NC (US); Patrick Joseph Roberts, Durham, NC (US); Erica Schlesinger, Sisters, OR (US); Randy J. Wald, Bend, OR (US); Cindy Chung, Bend, OR (US); Wesley K. Tatum, Bend, OR (US)

(73) Assignee: INCYCLIX BIO, LLC, Research Triangle Park, NC (US)

(21) Appl. No.: 19/361,699

(22) Filed: Oct. 17, 2025

Related U.S. Application Data
(63) Continuation of application No. PCT/US2024/025295, filed on Apr. 18, 2024.
(60) Provisional application No. 63/460,201, filed on Apr. 18, 2023.
(60) Provisional application No. 63/470,621, filed on Jun. 2, 2023.
(60) Provisional application No. 63/540,884, filed on Sep. 27, 2023.
(60) Provisional application No. 63/612,840, filed on Dec. 20, 2023.

(30) Foreign Application Priority Data
Jun. 20, 2023 (WO) PCT/US2023/025791

(51) Int. Cl. A61K 9/16; A61K 9/20; A61K 31/635

(52) U.S. Cl. CPC A61K 9/1652; A61K 9/1611; A61K 9/1623; A61K 9/1694; A61K 9/2009; A61K 9/2018; A61K 9/2054; A61K 9/2077; A61K 31/635

(57) ABSTRACT

Dosages and regimens based on the discovery of unexpectedly high and sustained exposure of INX-315 in humans in need thereof which results in increased efficacy per dose and minimizes adverse events during treatment, including by minimizing the ratio of Cₘₐₓ to Cₘᵢₙ, resulting in a decrease in an initial drug spike after administration.

### CROSS-REFERENCE TO RELATED APPLICATIONS

This application is a continuation of International Patent Application No. PCT/US2024/025295, filed in the U.S. Receiving Office on Apr. 18, 2024, which claims the benefit of U.S. Provisional Application No. 63/460,201 filed on Apr. 18, 2023, U.S. Provisional Application No. 63/470,621 filed on Jun. 2, 2023, International Application No. PCT/US2023/025791 filed on Jun. 20, 2023, U.S. Provisional Application No. 63/540,884 filed on Sep. 27, 2023, and U.S. Provisional Application No. 63/612,840 filed on Dec. 20, 2023. The entirety of these applications are hereby incorporated by reference for all purposes.

### FIELD OF THE INVENTION

This invention provides dosages and regimens based on the discovery of unexpectedly high and sustained exposure of INX-315 after administration to humans in need thereof. The surprisingly high and sustained exposure of INX-315 could not have been predicted prior to human administration. This surprising pharmacokinetic behavior results in increased efficacy per dose and can minimize adverse events during treatment, including by minimizing initial spikes during dosing, i.e., the ratio of Cₘₐₓ to Cₘᵢₙ, on administration.

### BACKGROUND

Cell division is regulated by the cell cycle, which is divided into four phases: G1 phase (cell growth and machinery synthesis), S phase (DNA replication to generate two identical sets of chromosomes), G2 phase (cell growth and supplemental machinery synthesis), and M phase (single cell divides into two identical daughter cells). The progression between cell cycle phases is primarily governed by cyclins and cyclin-dependent kinases (CDKs) (Asghar et al. Nat Rev Drug Discov. 14(2):130-46(2015)), which are activated or inhibited in response to a complex system of cell signaling networks that interpret extracellular signals.

Cyclin dependent kinase 2 (CDK2), which is activated following binding with cyclin E, is required to enter S-phase in the cell cycle (Merrick, K. A. et al. Switching Cdk2 on or off with small molecules to reveal requirements in human cell proliferation. Mol Cell. 42:624-636(2011)). The CDK2/cyclin E protein complex, as well as the CDK4/6/cyclin D protein complex, phosphorylates retinoblastoma (Rb), releasing the G1 transcription factor E2F to promote S-phase completion (Sherr, C. J. Mammalian G1 cyclins. Cell. 73(6):1059-65(1993 Jun. 18); Asghar et al. The history and future of targeting cyclin-dependent kinases in cancer therapy. Nat Rev Drug Discov. 14(2):130-146(2015)). The CDK2/cyclin E protein complex, in addition to regulating the G1/S transition, mediates histone biosynthesis and centrosome duplication (Matsumoto, Y K. et al. CDK2 is required for centrosome duplication in mammalian cells. Curr Biol. 9:429-432(1999); Nelson, D. M. et al. Coupling of DNA synthesis and histone synthesis in S phase independent of cyclin/cdk2 activity. Mol Cell Biol. 22(21):7459-7472(2002 November)).

CDK2-mediated hyperphosphorylation leads to complete inactivation of the Rb protein, which is necessary to promote S-phase completion. Thus, retinoblastoma is considered a key protein in the cell cycle system that includes the CDK2/cyclin E protein complex. Evidence suggests that CDK2 fundamentally regulates cellular proliferation and CDK4/6 inhibitor resistance mechanisms, highlighting the potential for targeting CDK2 in cancer (Tadesse, S. et al. Cyclin-Dependent Kinase 2 Inhibitors in Cancer Therapy: An Update. J Med Chem. 62(9):4233-4251(2019 May 9)).

There are several CDK2 inhibitors which are currently undergoing human clinical trials for the treatment of cancer (including, for example, BLU-222, Blueprint Medicines Corporation; PF-07104091, Pfizer; INX-315, Incyclix Bio; INCB123667, Incyte Medical; and AVZO-021, Avenzo Therapeutics).

Administration of either BLU-222 or PF-07104091 to humans exhibit a plasma concentration spike that may contribute to side effects. For example, in an open-label, first-in-human, phase 1/2 clinical trial evaluating CDK2 inhibitor BLU-222 in advanced solid tumors (NCT05252416), several ocular adverse events (AEs) affecting vision were observed in 5 patients (19%). These ocular AEs included transient, reversible instances of light sensitivity, blurred vision, photophobia, and vision changes. Most of the ocular AEs were mild, except for one grade 3 blurred vision/photophobia in a patient who received 600 mg of BLU-222 twice per day (BID). Due to these ocular-related AEs, the U.S. Food and Drug Administration (FDA) placed a temporary clinical hold on patient enrollment in February of 2023 (Blueprint Medicines announces partial clinical hold for phase 1/2 VELA trial of BLU-222. News release (2023 Feb. 10)), which was released after a mitigation plan.

BLU-222 has the structure:

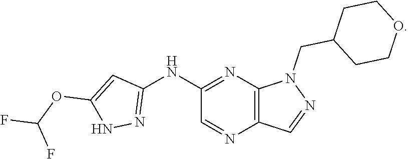 [SMILES: FC(F)Oc1cc(Nc2cnc3cnn(CC4CCOCC4)c3n2)n[nH]1]

The Pfizer CDK2 inhibitor PF-07104091 is currently being evaluated in a Phase 1 study in advanced or metastatic solid tumors (NCT04553133; Yap, T. A. et al. First-in-human phase 1/2a study of a potent and novel CDK2-selective inhibitor PF-07104091 in patients (pts) with advanced solid tumors, enriched for CDK4/6 inhibitor resistant HR+/IER2− breast cancer. 2023 ASCO Annual Meeting I—Developmental Therapeutics—Molecularly Targeted Agents and Tumor Biology. Meeting Abstract 3010. 41(16):suppl(2023)).

PF-07104091 has the structure:

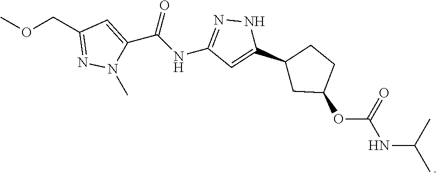 [SMILES: COCc1cc(C(=O)Nc2cc([C@H]3CC[C@@H](OC(=O)NC(C)C)C3)[nH]n2)n(C)n1]

The most common treatment-related AEs noted for PF-07104091 included nausea (77.1%; 14.3% Grade 3), diarrhea (48.6%; 8.6% Grade 3), vomiting (48.6%; 2.9% Grade 3), fatigue (45.7%; 20.0% Grade 3) and anemia (45.7%; 8.6% Grade 3).

Another CDK2 inhibitor that has just entered Phase I clinical trials is INX-315, originally disclosed in WO2021/236650, filed G1 Therapeutics, Inc., and exclusively licensed to Incyclix Bio (see also, WO2023/249974 filed by Incyclix Bio).

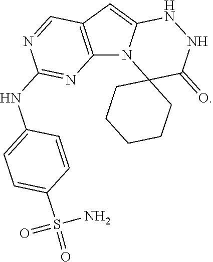 [SMILES: NS(=O)(=O)c1ccc(Nc2ncc3cc4n(c3n2)C2(CCCCC2)C(=O)NN4)cc1]

There is an ongoing need for improved treatment and dosage regimes useful in treating patients with cancers, including CDK4/6-resistant tumors, while minimizing off-target toxicities.

### SUMMARY

The present invention provides dosages and regimens based on the surprising discovery as a result of first time in human dosing of the CDK2 inhibitor of structure:

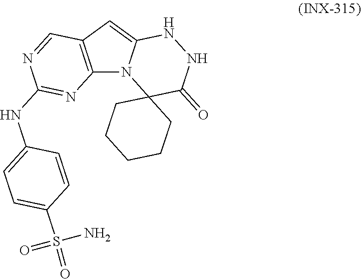 [SMILES: NS(=O)(=O)c1ccc(Nc2ncc3cc4n(c3n2)C2(CCCCC2)C(=O)NN4)cc1]

that this compound has a unique and highly unexpected pharmacokinetic profile, which is significantly inconsistent with predicted mathematical pharmacokinetic models extrapolated from other mammalian species (dogs, rats, and mice).

In one aspect of this invention, advantageous pharmaceutical compositions of INX-315 or its pharmaceutically acceptable salt are provided which, upon administration to humans, result in i) reduced or minimal spikes in plasma concentration of INX-315 over time and ii) sustained release of INX-315 into the plasma that allows for an extended half-life of the drug. While not wanting to be held to one theory, it is proposed that INX-315 binds to human blood cells in vivo to a much greater degree than seen in other tested mammals or as predicted prior to human administration. This increases the amount of drug available in human blood and creates a depot effect for a type of extended availability of the drug as it is released over time from the red blood cells into the plasma and then to the site of therapy.

In another aspect of the invention, solid dosage form pharmaceutical formulations of INX-315 or a pharmaceutically acceptable salt thereof are provided that enhance the bioavailability and release properties of the drug or its pharmaceutically acceptable salt. It has been discovered that when INX-315 is administered in an amorphous spray-dried dispersion (ASD) of INX-315 with a compound that acts as a precipitation preventer, advantageous delivery is achieved. INX-315 is very insoluble and likes to crystallize. The compound tends to self-aggregate and crystallize in the solid form. This can happen during manufacture, during storage or even after human administration. It has been discovered as a result of research on the formulation that the efficacy of the drug is enhanced when a pharmaceutically acceptable precipitation preventer is included in the amorphous spray dried dispersion. Certain suitable precipitation preventer agents include, but are not limited to polyvinylpyrrolidone, polymethacrylate, a pharmaceutically acceptable cellulose derivative such as hydroxypropyl methylcellulose acetate succinate (HPMCAS), and the like, which adequately prevent the precipitation of INX-315, as described further in the Detailed Description. The ASD can be granulated with appropriate excipients. In certain embodiments, the pharmaceutical composition is a tablet that comprises a blend of INX-315 ASD granules that includes an ample amount of a precipitation enhancer and one or more extragranular excipients (i.e., an excipient added after granulation but before compression or tableting). This pharmaceutical composition is well tolerated in human patients and has been successfully administered in exemplary solid dosage forms, for example, tablets of 100, 200, and 300 mg strength.

Surprisingly, it has also been discovered that INX-315 is effective in the treatment of Rb-independent (Rb-negative) cancers in humans based on human data. As indicated in the Background of the Invention, INX-315 is a CDK2 inhibitor which is activated following binding of its binding partner cyclin E. When cyclin E is amplified, then the cell enters into S-phase of the cell cycle, causing in the case of cancer, uncontrolled replication. The CDK2-mediated hyperphosphorylation leads to inactivation of the Rb protein, which is necessary to promote S-phase completion. However, it is now discovered that INX-315 is also able to inhibit the uncontrolled replication of certain cancers that do not go through a cyclinE/retinoblastoma mechanism. It may be that INX-315 can also inhibit cyclin A mediated replication, which occurs downstream of the retinoblastoma pathway. Using this alternative pathway, INX-315 may be used in an effective amount to treat Rb-independent cancers, including but not limited to small cell lung cancer (SCLC) (see, e.g., FIG. 15C-15D). Other tumors include Rb-independent tumors selected from triple negative breast cancer (TNBC), prostate cancer, liver cancer, bladder cancer, ovarian cancer, uterine cancer, cervical cancer, stomach cancer, esophageal cancer, head and neck cancer, glioblastoma, retinoblastoma, osteosarcoma, or lymphoma.

This discovery provides an advance in cancer therapy by providing a CDK2 inhibitor that is useful to treat both Rb-dependent and Rb-independent tumors, which can include heterogeneous tumors of both. In addition, INX-315 can be used in conjunction with a CDK4/6 inhibitor including but not limited to ribociclib (Novartis), abemaciclib (Eli Lilly), palbociclib (Pfizer Inc.), or lerociclib (G1 Therapeutics, Inc.). In some embodiments, INX-315 is administered on a daily basis and the CDK4/6 inhibitor is typically administered for 21 days during a 28-day cycle with a 7 day drug holiday.

It is also highly unexpected that the administration of INX-315 does not result in a spike of drug concentration on initial administration as is often seen with other drugs, such as BLU-222 and PF-07104091. A spike effect is indicated by a large initial Cₘₐₓ to Cₘᵢₙ ratio at the beginning of administration, which tails off after administration (see FIGS. 2 and 3). Conversely, the lack of a spike is indicated when the ratio of Cₘₐₓ to Cₘᵢₙ is small. As a non-limiting example, the Cₘₐₓ to Cₘᵢₙ ratio of INX-315 in the first two humans administered INX-315 at 100 mg once-a-day was measured as about 2.2, whereas the estimated Cₘₐₓ to Cₘᵢₙ ratio based on published human results for BLU-222 (400 mg twice a day) was 9.3 and for BLU-222 (600 mg twice a day) was 22. Likewise, the estimated Cₘₐₓ to Cₘᵢₙ ratio for PF-07104091 was 16-64 based on publicly available human data (see Table 5 and FIGS. 2 and 3). The presence of a spike after dosing, which is a short-term excess amount of drug in the blood, can lead to side effects and is typically disfavored. Based on this human discovery, a method for the treatment of a cancer mediated by CDK2 is provided that includes the administration of an effective amount of INX-315 in a manner wherein the Cₘₐₓ to Cₘᵢₙ ratio on administration is not more than 6, 5, 4 or even 3.

INX-315 exhibits a very high AUC on day 1 that extends through Day 15 of dosing which is much greater than predicted based upon the observed profiles in non-human mammalian models. It was not predictable in advance that the human pharmacokinetic parameters in humans would be better than the pharmacokinetic parameters in other tested mammals, as described more fully in the Detailed Description of the Invention. A non-limiting range of the AUC in humans with only once a day administration of INX-315 in a range of 100-300 mg is from 1,000-40,000 in ng×hr/ml in plasma. The AUC of course increases with dosage and varies somewhat by person based on individual metabolic patterns and individuality.

It was discovered that INX-315 binds to red blood cells at a higher level than in plasma, and that the red blood cell-to-plasma ratio (K_{RBC/P}) may range between approximately 12-30, 15-30 or approximately 12.0-20.0 in humans, which is higher than in other mammals tested (see Example 2, Table 7). Additionally, the whole blood-to-plasma ratio (K_{WB/P}) in humans of INX-315 is approximately 4-10, or 5-10 or 6-10 which is also higher than in other mammals (Table 7).

For example, as described in Example 10, the predicted AUC for INX-315 at 100 mg once-a-day dosing based on the mammal models is about 740 (ng*hr/mL), however, the measured AUC on Day 1 was 2020 (average of five human patients) and on Day 15 was 3290. These results exceeded predictions by factors greater than 2.7 and 4.4, respectively. Likewise, the predicted Cₘₐₓ (ng/mL) for 100 mg at once-a-day dosing is 39.3, whereas the measured Cₘₐₓ was 148 on Day 1 and 211 on Day 15. These results exceeded predictions by factors greater than 3.7 and 5.3, respectively. In addition, the predicted Cₘᵢₙ at the same dosage was 19.1 (ng/mL), whereas the actual Cₘᵢₙ was 103 at Day 1 and 177 at Day 15. The calculated Cₘᵢₙ^{free} (ng/mL) at that dosage was 0.997, whereas the observed was 5.4 on Day 1 and 9.2 on Day 15 (see generally Example 2, Table 2).

As an additional example, as described in Example 10, the predicted AUC for INX-315 at 200 mg once-a-day dosing based on the mammal models is 1380 (ng*hr/mL), however, the measured AUC on Day 1 was 8940 (average of five human patients) and on Day 15 was 12200. These results exceeded predictions by factors greater than 6.4 and 8.8, respectively. Likewise, the predicted Cₘₐₓ (ng/mL) for 200 mg at once-a-day dosing is 72.3, whereas the measured Cₘₐₓ was 590 on Day 1 and 922 on Day 15. These results exceeded predictions by factors greater than 8.1 and 12.7, respectively.

In addition, INX-315 between a range of 0.03 μM to 10 μM exhibits a blood to plasma partitioning ratio of 25-29 (K_{R}BC/P) and the whole blood-to-plasma ratio is 10-16 (K_{WB/P}), both of which average to significantly higher than the mammals tested (Table 8).

In whole blood, the predicted AUC based on mammal models was 5560 (ng*hr/mL), whereas the actual AUC in humans was a surprising 50,550 on Day 1 and 116,350 on Day 15 (Table 9). This exceeded the model by 9× on Day 1 and more than a factor of 20 on Day 15 (Table 9).

As reported herein, the AUC, Cₘₐₓ, Cₘᵢₙ, and Cₘᵢₙ^{free} profiles are all greater than about 2-3 times or more times in plasma and 9 or more times in whole blood, respectively, than predicted from studies in three non-human animal models. This appears to be the case because the drug is disproportionately bound to human red blood cells as compared to those of non-human mammals.

The invention as described herein also includes an amorphous spray-dried dispersion (ASD) formulation of INX-315. As described above and herein, INX-315 is highly insoluble in water (Table 12) and free INX-315 is crystalline (FIG. 5A-6C). It has been discovered that INX-315 can be mixed with a precipitation preventer, for example a polymeric precipitation preventer, in a solvent to create an advantageous amorphous spray-dried dispersion (ASD) formulation. The precipitation preventer minimizes crystallization of the INX-315 in the ASD formulation. In non-limiting embodiments, the mixing solvent may be for example a mixture of THF and water, and as a non-limiting embodiment, 90% THF and 10% water. In some embodiments, the mixing solvent is a mixture of THF and water, for example 92% THF and 8% water. In some embodiments, the THF is stabilized with butylated hydroxytoluene (BHT). As a non-limiting illustration in Example 3, the INX-315 ASD formulation disperses INX-315 across the polymer matrix (e.g., precipitation preventer such as HPMCAS) preventing the crystallization of INX-315 in the ASD formulation, as indicated by high T_{g} and low Tₘ/T_{g} ratios (FIG. 9A-9B).

The invention includes an amorphous spray dried dispersion of INX-315 or a pharmaceutically acceptable salt thereof with a significant amount of a precipitation enhancer, for example, between 20% and 60% of INX-315 by weight of the ASD. In some embodiments, the amorphous INX-315 is included in a range of between for example about 25 and about 60% by weight of the ASD, or for example about 35-45% or even 40% by weight of the ASD (Tables 17 and 20). In some embodiments, INX-315 is spray-dried together with the precipitation preventer, for example but not limited to hydroxy propyl methylcellulose acetate succinate-high substitution (HPMCAS-HG), or other precipitation preventers as described further herein. The precipitation preventer can be, for example included in a range of between about 50% and about 70% by weight of the ASD, or for example between about 55% and about 65% by weight of the ASD, or for example even about 60% by weight of the ASD (Tables 17 and 20).

In an additional aspect, advantageous pharmaceutical compositions are described herein comprising the ASD formulations added together with one or more additional pharmaceutically acceptable excipients. As a non-limiting illustration in Example 4, the INX-315 ASD formulation is dry-granulated, dry-blended, then tableted together with one or more additional pharmaceutically acceptable excipients, for example including but not limited to microcrystalline cellulose, mannitol, croscarmellose sodium, colloidal silicon dioxide, and sodium stearyl fumarate (Tables 21 and 23), to create a pharmaceutical composition to be administered to a subject in need thereof.

In some embodiments, the amorphous INX-315 is included in a range of between about 10% and about 30% by weight of the pharmaceutical composition, or between about 15% and about 35% by weight of the pharmaceutical composition, or for example about 20% by weight of the pharmaceutical composition (Tables 17 and 20). The precipitation preventer can be, for example included in a range of between about 20% and about 50% by weight of the pharmaceutical composition, or for example between about 25% and about 45% by weight of the pharmaceutical composition, or for example about 30% by weight of the pharmaceutical composition (Tables 17 and 20).

Pharmaceutical compositions comprising the INX-315 ASD formulations described herein exhibit advantageous total drug and dissolved drug concentrations over prolonged periods (FIG. 8A-8C), implying that a majority of INX-315 exists in micelles, small colloids, small self-assembled drug aggregates, or molecularly free drug (FIG. 7, FIG. 10A-13B). While not wishing to be bound to one theory, a sustained absorption of INX-315 is promoted by shuttling of the drug across the intestinal mucus boundary layer which is rapidly replaced by free drug as it is absorbed, while the free drug is continually replaced by intermittently dissolving INX-315 drug/polymer colloids. The formation of a variety of INX-315 drug/polymer and micelle structures in vivo lead to sustained INX-315 dissolution and absorption of INX-315, creating a “depot effect” phenomenon, wherein the level of drug is elevated and extended over a greater period of time than what would have been predicted based upon prior preclinical animal studies. In the case of INX-315, the compound advantageously and quickly reaches its Cₘₐₓ without a “spike effect” in humans and maintains a small differential between Cₘₐₓ and Cₘᵢₙ (i.e., a low Cₘₐₓ to Cₘᵢₙ ratio) over many hours as seen for example in FIG. 1.

INX-315 has now been used to treat humans with tumors with intact, functional retinoblastoma protein. Examples of cancers with intact, functional retinoblastoma protein include but are not limited to breast cancer, ovarian cancer, non-small cell lung cancer (NSCLC), colon cancer, prostate cancer, or glioblastoma. In some embodiments, the Rb-positive cancer is breast cancer. In some embodiments, the Rb-positive breast cancer is HR+/HER2− breast cancer. In some embodiments, the Rb-positive breast cancer is ER+/HER2− breast cancer. In some embodiments, the Rb-positive cancer is ovarian cancer. In some embodiments, the Rb-positive ovarian cancer is high-grade serous ovarian cancer (HGSOC).

Unexpectedly, it has also been discovered that INX-315 also effectively treats Rb-independent cancers through Rb-independent mechanisms (see, e.g., FIG. 15C-15D). In some embodiments, the Rb-independent cancer is selected from small cell lung cancer (SCLC), retinoblastoma, triple negative breast cancer (TNBC), human papillomavirus (HPV) positive head and neck cancer, HPV positive cervical cancer, or bladder cancer. In some embodiments, the Rb-independent cancer is SCLC. In some embodiments, the Rb-independent cancer is TNBC. In some embodiments, the Rb-independent cancer is bladder cancer.

In some embodiments, INX-315 or a pharmaceutically acceptable salt in an ASD formulation thereof described herein provides an additive effect to or synergistic effect with the anti-cancer or anti-proliferative activity of an additional therapeutic agent. In some embodiments, the additional therapeutic agent is selected from a CDK4/6 inhibitor, an endocrine therapy, a chemotherapeutic agent, an immune checkpoint inhibitor, or a combination thereof (see, e.g., FIG. 15C-15D and FIG. 16B). This improvement provides a significant advance in the state of the art of cancer treatment. In some embodiments, the additional therapeutic is a CDK4/6 inhibitor. In some embodiments, the CDK4/6 inhibitor is selected from palbociclib, ribociclib, abemaciclib, trilaciclib, lerociclib, or dalpiciclib. In some embodiments, the CDK4/6 inhibitor is palbociclib (Pfizer Inc). In some embodiments, the CDK4/6 inhibitor is ribociclib (Novartis). In some embodiments, the CDK4/6 inhibitor is abemaciclib (Eli Lilly). In some embodiments, the CDK4/6 inhibitor is lerociclib (G1 Therapeutics, Inc.).

INX-315 can be advantageously administered for example to patients with a CDK4/6-resistant tumor or a cyclin E overexpressing tumor as seen for example in FIG. 16B. INX-315 can also be administered in combination with a selective CDK4 inhibitor. In certain embodiments, INX-315 can be administered in combination with a CDK4/6 inhibitor. In some embodiments, the CDK4/6 inhibitor is selected from palbociclib, ribociclib, abemaciclib, trilaciclib, lerociclib, or dalpiciclib. In some embodiments, the CDK4/6 inhibitor is palbociclib (Pfizer Inc). In some embodiments, the CDK4/6 inhibitor is ribociclib (Novartis). In some embodiments, the CDK4/6 inhibitor is abemaciclib (Eli Lilly). In some embodiments, the CDK4/6 inhibitor is lerociclib (G1 Therapeutics, Inc.).

It has been surprisingly discovered that administering INX-315 in certain dosage regimens achieves improved pharmacokinetic parameters compared to predicted values generated using a model based on preclinical mammals. These dosage regimens are sufficient to treat a human patient in need thereof, for example a human having a cancer that is sensitive to a CDK2 inhibitor.

Non-limiting examples of aspects of the present invention include:
1. A pharmaceutical composition comprising an amorphous spray-dried dispersion (ASD) of INX-315 or a pharmaceutically acceptable salt thereof and a pharmaceutically acceptable excipient; wherein the ASD further comprises a precipitation preventer.
2. The pharmaceutical composition of embodiment 1, wherein INX-315 is between about 20% and about 60% by weight of the ASD.
3. The pharmaceutical composition of embodiment 1, wherein INX-315 is between about 30% and about 50% by weight of the ASD.
4. The pharmaceutical composition of embodiment 1, wherein INX-315 is about 40% by weight of the ASD.
5. The pharmaceutical composition of any one of embodiments 1-4, wherein the pharmaceutical composition comprises between about 100 mg and about 500 mg of INX-315.
6. The pharmaceutical composition of any one of embodiments 1-4, wherein the precipitation preventer is selected from a cellulose derivative, polyvinylpyrrolidone (PVP), PVP/VA (vinyl acetate), polymethacrylate, hypromellose, HPMC 2910, hydroxyethyl cellulose (HEC), hydroxypropyl methylcellulose acetate succinate (HPMCAS), HPMCAS-L, HPMCAS-LF, HPMCAS-LG, HPMCAS-M, HPMCAS-MF, HPMCAS-MG, HPMCAS-H, HPMCAS-HF, HPMCAS-HG, HPMCAS-E3, hydroxypropyl methylcellulose phthalate (HPMCP), cellulose acetate phthalate (CAP), sodium carboxymethyl cellulose (Na-CMC), polyacrylic acid, polyethylene glycol, PEG 4000, PEG 6000, PEG 8000, PEG 20000, polyvinylpyrrolidone, PVP K 30, PVP K 25, PVP VA64, or PVP VA37.
7. The pharmaceutical composition of embodiment 6, wherein the precipitation preventer is HPMCAS-FIG.
8. The pharmaceutical composition of any one of embodiments 1-7, wherein the precipitation preventer in the ASD is between about 40% and about 80% by weight of the ASD.
9. The pharmaceutical composition of any one of embodiments 1-7, wherein the precipitation preventer in the ASD is about 60% by weight of the ASD.
10. The pharmaceutical composition of any one of embodiments 1-9, wherein the ASD is between about 40% and about 60% by weight of the pharmaceutical composition.
11. The pharmaceutical composition of any one of embodiments 1-9, wherein the ASD is about 50% by weight of the pharmaceutical composition.
12. The pharmaceutical composition of any one of embodiments 1-11, wherein the pharmaceutical composition comprises an insoluble diluent.
13. The pharmaceutical composition of embodiment 12, wherein the insoluble diluent is microcrystalline cellulose.
14. The pharmaceutical composition of claim 12 or 13, wherein the insoluble diluent is between about 15% and about 25% by weight of the pharmaceutical composition.
15. The pharmaceutical composition of any one of embodiments 1-14, wherein the pharmaceutical composition comprises a soluble diluent.
16. The pharmaceutical composition of embodiment 15, wherein the soluble diluent is mannitol.
17. The pharmaceutical composition of claim 15 or 16, wherein the soluble diluent is between about 15% and about 25% by weight of the pharmaceutical composition.
18. The pharmaceutical composition of any one of embodiments 1-17, wherein the pharmaceutical composition comprises a disintegrant.
19. The pharmaceutical composition of embodiment 18, wherein the disintegrant is croscarmellose sodium.
20. The pharmaceutical composition of embodiment 18 or 19, wherein the disintegrant is between about 1% and about 10% by weight of the pharmaceutical composition.
21. The pharmaceutical composition of any one of embodiments 1-20, wherein the pharmaceutical composition comprises a glidant.
22. The pharmaceutical composition of embodiment 21, wherein the glidant is colloidal silicon dioxide.
23. The pharmaceutical composition of embodiment 21 or 22, wherein the glidant is between about 0.1% and about 5% by weight of the pharmaceutical composition.
24. The pharmaceutical composition of any one of embodiments 1-20, wherein the pharmaceutical composition comprises a lubricant.
25. The pharmaceutical composition of embodiment 24, wherein the lubricant is sodium stearyl fumarate.
26. The pharmaceutical composition of embodiment 24 or 25, wherein the lubricant is between about 0.1% and about 5% by weight of the pharmaceutical composition.
27. A method for the treatment of a human with a heterogenous CDK2-mediated cancer comprising administering an effective amount INX-315 or a pharmaceutically acceptable salt thereof, to the human in need thereof,
   wherein the heterogenous CDK2-mediated cancer comprises a mixture of retinoblastoma (Rb)-dependent cells and Rb-independent cells.
28. The method of embodiment 27, wherein INX-315 or a pharmaceutically acceptable salt thereof is administered as a component of a pharmaceutical composition, wherein the pharmaceutical composition comprises an amorphous spray dried dispersion (ASD) comprising between about 100 mg and about 500 mg of INX-315; and wherein the amount of INX-315 in the ASD is between 20% and 60% by weight.
29. A method for the treatment of a human with a CDK2-mediated cancer comprising administering an effective amount of a pharmaceutical composition comprising INX-315 or a pharmaceutically acceptable salt thereof, to the human in need thereof, which results in a mean plasma area under the curve (AUC) (ng*hr/mL) of between about 1,250 and about 50,000 as measured on day 1 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
30. The method of any one of embodiments 27-29, wherein the mean plasma AUC is between 6,000 and 50,000 as measured on day 1 of dosing.
31. The method of any one of embodiments 27-29, wherein the mean plasma AUC is between 9,000 and 50,000 as measured on day 1 of dosing.
32. A method for the treatment of a human with a CDK2-mediated cancer comprising administering an effective amount of a pharmaceutical composition comprising INX-315 or a pharmaceutically acceptable salt thereof, to the human in need thereof, which results in a mean maximum plasma concentration (Cₘₐₓ) (ng/mL) of between about 75 and about 2,400 as measured on day 1 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
33. The method of any one of embodiments 27-32, wherein the mean plasma Cₘₐₓ is between 350 and 2,400 as measured on day 1 of dosing.
34. The method of any one of embodiments 27-32, wherein the mean plasma Cₘₐₓ is between 525 and 2,400 as measured on day 1 of dosing.
35. A method for the treatment of a human with a CDK2-mediated cancer comprising administering an effective amount of a pharmaceutical composition comprising INX-315 or a pharmaceutically acceptable salt thereof, to the human in need thereof, which results in a mean minimum plasma concentration (Cₘᵢₙ) (ng/mL) of between about 20 and about 1,000 as measured on day 1 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
36. The method of any one of embodiments 27-35, wherein the mean plasma Cₘᵢₙ is between 120 and 1,000 as measured on day 1 of dosing.
37. The method of any one of embodiments 27-35, wherein the mean plasma Cₘᵢₙ is between 180 and 1,000 as measured on day 1 of dosing.
38. A method for the treatment of a human with a CDK2-mediated cancer comprising administering an effective amount of a pharmaceutical composition comprising INX-315 or a pharmaceutically acceptable salt thereof, to the human in need thereof, which results in a mean free minimum plasma concentration (Cₘᵢₙ^{free}) (ng/mL) of between about 4.0 and about 48.0 as measured on day 1 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
39. The method of any one of embodiments 27-38, wherein the mean Cₘᵢₙ^{free} is between 10 and 48 as measured on day 1 of dosing.
40. The method of any one of embodiments 27-38, wherein the mean Cₘᵢₙ^{free} is between 15 and 48 as measured on day 1 of dosing.
41. A method for the treatment of a human with a CDK2-mediated cancer comprising administering an effective amount of a pharmaceutical composition comprising INX-315 or a pharmaceutically acceptable salt thereof, to a human patient in need thereof, which results in a mean plasma area under the curve (AUC) (ng*hr/mL) of between about 1,250 and about 56,000 as measured on day 15 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
42. The method of any one of embodiments 27-41, wherein the mean plasma AUC is between 11,000 and 56,000 as measured on day 15 of dosing.
43. The method of any one of embodiments 27-41, wherein the mean plasma AUC is between 16,500 and 56,000 as measured on day 15 of dosing.
44. A method for the treatment of a human with a CDK2-mediated cancer comprising administering an effective amount of a pharmaceutical composition comprising INX-315 or a pharmaceutically acceptable salt thereof, to the human in need thereof, which results in a mean maximum plasma concentration (Cₘₐₓ) (ng/mL) of between about 90 and about 4,000 as measured on day 15 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
45. The method of any one of embodiments 27-44, wherein the mean plasma Cₘₐₓ is between 700 and 4,000 as measured on day 15 of dosing.
46. The method of any one of embodiments 27-44, wherein the mean plasma Cₘₐₓ is between 1,050 and 4,000 as measured on day 15 of dosing.
47. A method for the treatment of a human with a CDK2-mediated cancer comprising administering an effective amount of a pharmaceutical composition comprising INX-315 or a pharmaceutically acceptable salt thereof, to the human in need thereof, which results in a mean minimum plasma concentration (Cₘᵢₙ) (ng/mL) of between about 100 and about 2,000 as measured on day 15 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
48. The method of any one of embodiments 27-47, wherein the mean plasma Cₘᵢₙ is between 250 and 2,000 as measured on day 15 of dosing.
49. The method of any one of embodiments 27-47, wherein the mean plasma Cₘᵢₙ is between 375 and 2,000 as measured on day 15 of dosing.
50. A method for the treatment of a human with a CDK2-mediated cancer comprising administering an effective amount of a pharmaceutical composition comprising INX-315 or a pharmaceutically acceptable salt thereof, to the human in need thereof, which results in a mean free minimum plasma concentration (Cₘᵢₙ^{free}) (ng/mL) of between about 6.0 and about 80.0 as measured on day 15 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
51. The method of any one of embodiments 27-50, wherein the mean Cₘᵢₙ^{free} is between 14 and 80 as measured on day 15 of dosing.
52. The method of any one of embodiments 27-50, wherein the mean Cₘᵢₙ^{free} is between 21 and 80 as measured on day 15 of dosing.
53. A method for the treatment of a human with a CDK2-mediated cancer comprising administering an effective amount of a pharmaceutical composition comprising INX-315 or a pharmaceutically acceptable salt thereof, to the human in need thereof, which results in a mean whole blood area under the curve (AUC) (ng*hr/mL) of between about 25,000 and about 480,000 as measured on day 1 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
54. The method of any one of embodiments 27-53, wherein the mean whole blood AUC is between 80,000 and 480,000 as measured on day 1 of dosing.
55. The method of any one of embodiments 27-53, wherein the mean whole blood AUC is between 120,000 and 480,000 as measured on day 1 of dosing.
56. A method for the treatment of a human with a CDK2-mediated cancer comprising administering an effective amount of a pharmaceutical composition comprising INX-315 or a pharmaceutically acceptable salt thereof, to the human in need thereof, which results in a mean maximum whole blood concentration (Cₘₐₓ) (ng/mL) of between about 2,000 and about 28,000 as measured on day 1 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
57. The method of any one of embodiments 27-56, wherein the mean whole blood Cₘₐₓ is between 4,500 and 28,000 as measured on day 1 of dosing.
58. The method of any one of embodiments 27-56, wherein the mean whole blood Cₘₐₓ is between 6,750 and 28,000 as measured on day 1 of dosing.
59. A method for the treatment of a human with a CDK2-mediated cancer comprising administering an effective amount of a pharmaceutical composition comprising INX-315 or a pharmaceutically acceptable salt thereof, to the human in need thereof, which results in a mean minimum whole blood concentration (Cₘᵢₙ) (ng/mL) of between about 500 and about 14,400 as measured on day 1 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
60. The method of any one of embodiments 27-59, wherein the mean whole blood Cₘᵢₙ is between 1,800 and 14,400 as measured on day 1 of dosing.
61. The method of any one of embodiments 27-59, wherein the mean whole blood Cₘᵢₙ is between 2,700 and 14,400 as measured on day 1 of dosing.
62. A method for the administration of a pharmaceutical composition comprising INX-315 or a pharmaceutically acceptable salt thereof, to the human in need thereof, which results in a mean whole blood area under the curve (AUC) (ng*hr/mL) of between about 25,000 and about 800,000 as measured on day 15 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
63. The method of any one of embodiments 27-62, wherein the mean whole blood AUC is between 120,000 and 800,000 as measured on day 15 of dosing.
64. The method of any one of embodiments 27-62, wherein the mean whole blood AUC is between 180,000 and 800,000 as measured on day 15 of dosing.
65. A method for the treatment of a human with a CDK2-mediated cancer comprising administering an effective amount of a pharmaceutical composition comprising INX-315 or a pharmaceutically acceptable salt thereof, to the human in need thereof, which results in a mean maximum whole blood concentration (Cₘₐₓ) (ng/mL) of between about 3,000 and about 56,000 as measured on day 15 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
66. The method of any one of embodiments 27-65, wherein the mean whole blood Cₘₐₓ is between 7,000 and 56,000 as measured on day 15 of dosing.
67. The method of any one of embodiments 27-65, wherein the mean whole blood Cₘₐₓ is between 10,500 and 56,000 as measured on day 15 of dosing.
68. A method for the treatment of a human with a CDK2-mediated cancer comprising administering an effective amount of a pharmaceutical composition comprising INX-315 or a pharmaceutically acceptable salt thereof, to the human in need thereof, which results in a mean minimum whole blood concentration (Cₘᵢₙ) (ng/mL) of between about 1,000 and about 24,000 as measured on day 15 of dosing;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
69. The method of any one of embodiments 27-68, wherein the mean whole blood Cₘᵢₙ is between 2,600 and 24,000.
70. The method of any one of embodiments 27-68, wherein the mean whole blood Cₘᵢₙ is between 3,900 and 24,000.
71. The method of any one of embodiments 27-70, which further results in either a) a mean plasma Cₘₐₓ to plasma Cₘᵢₙ ratio of between 1.25 and 5 and/or b) a mean whole blood Cₘₐₓ to whole blood Cₘᵢₙ ratio of between 1.25 and 5.
72. The method of any one of embodiments 27-70, which further results in a mean plasma Cₘₐₓ to plasma Cₘᵢₙ ratio of between 3 and 5.
73. The method of any one of embodiments 27-70, which further results in a mean whole blood Cₘₐₓ to whole blood Cₘᵢₙ ratio of between 3 and 5.
74. A method for the treatment of a human with a CDK2-mediated cancer comprising administering an effective amount of a pharmaceutical composition comprising INX-315 or a pharmaceutically acceptable salt thereof, to the human in need thereof, which results in a mean plasma Cₘₐₓ to Cₘᵢₙ ratio of less than about 5 but greater than about 1.25;
   wherein the pharmaceutical composition comprises an amorphous spray dried dispersion (ASD) comprising between about 100 mg and about 500 mg of INX-315; and
   wherein the amount of INX-315 in the ASD is between 20% and 60% by weight.
75. The method of embodiment 74, wherein the Cₘₐₓ to Cₘᵢₙ ratio is less than 3.
76. The method of embodiment 74, wherein the Cₘₐₓ to Cₘᵢₙ ratio is less than 2.25.
77. The method of any one of embodiments 74-76, wherein the wherein the Cₘₐₓ to Cₘᵢₙ ratio is measured in plasma.
78. The method of any one of embodiments 74-76, wherein the wherein the Cₘₐₓ to Cₘᵢₙ ratio is measured in whole blood.
79. The method of any one of embodiments 27-78, which results in a t_{1/2} of between about 5 hours and about 32 hours.
80. The method of any one of embodiments 27-78, which results in a t_{1/2} of between about 8 hours and about 28 hours.
81. The method of any one of embodiments 27-78, which results in a t_{1/2} of between about 12 hours and about 18 hours.
82. The method of any one of embodiments 79-81, wherein the t_{1/2} is about 14 hours.
83. The method of any one of embodiments 79-81, wherein the t_{1/2} is about 15 hours.
84. The method of any one of embodiments 79-81, wherein the t_{1/2} is about 16 hours.
85. The method of any one of embodiments 27-84, wherein INX-315 is administered once-a-day.
86. The method of any one of embodiments 27-85, wherein INX-315 is orally administered.
87. The method of any one of embodiments 28-86, wherein the amount of INX-315 in the ASD is about 40%.
88. The method of any one of embodiments 28-87, wherein the total amount of INX-315 in the pharmaceutical composition is about 100 mg.
89. The method of any one of embodiments 28-87, wherein the total amount of INX-315 in the pharmaceutical composition is about 125 mg.
90. The method of any one of embodiments 28-87, wherein the total amount of INX-315 in the pharmaceutical composition is about 150 mg.
91. The method of any one of embodiments 28-87, wherein the total amount of INX-315 in the pharmaceutical composition is about 175 mg.
92. The method of any one of embodiments 28-87, wherein the total amount of INX-315 in the pharmaceutical composition is about 200 mg.
93. The method of any one of embodiments 28-87, wherein the total amount of INX-315 in the pharmaceutical composition is about 225 mg.
94. The method of any one of embodiments 28-87, wherein the total amount of INX-315 in the pharmaceutical composition is about 250 mg.
95. The method of any one of embodiments 28-87, wherein the total amount of INX-315 in the pharmaceutical composition is about 275 mg.
96. The method of any one of embodiments 28-87, wherein the total amount of INX-315 in the pharmaceutical composition is about 300 mg.
97. The method of any one of embodiments 28-87, wherein the total amount of INX-315 in the pharmaceutical composition is about 325 mg.
98. The method of any one of embodiments 28-87, wherein the total amount of INX-315 in the pharmaceutical composition is about 350 mg.
99. The method of any one of embodiments 28-87, wherein the total amount of INX-315 in the pharmaceutical composition is about 375 mg.
100. The method of any one of embodiments 28-87, wherein the total amount of INX-315 in the pharmaceutical composition is about 400 mg.
101. The method of any one of embodiments 28-87, wherein the total amount of INX-315 in the pharmaceutical composition is about 425 mg.
102. The method of any one of embodiments 28-87, wherein the total amount of INX-315 in the pharmaceutical composition is about 450 mg.
103. The method of any one of embodiments 28-87, wherein the total amount of INX-315 in the pharmaceutical composition is about 475 mg.
104. The method of any one of embodiments 28-87, wherein the total amount of INX-315 in the pharmaceutical composition is about 500 mg.
105. The method of any one of embodiments 28-104, wherein the pharmaceutical composition further comprises a precipitation preventer.
106. The method of embodiment 105, wherein the amount of the precipitation preventer in the ASD is between 40-70% by weight.
107. The method of embodiment 105 or 106, wherein the amount of the precipitation preventer in the ASD is about 60% by weight.
108. The method of any one of embodiments 105-107, wherein the precipitation preventer is selected from a cellulose derivative, polyvinylpyrrolidone (PVP), PVP/VA (vinyl acetate), polymethacrylate, hypromellose, HPMC 2910, hydroxyethyl cellulose (HEC), hydroxypropyl methylcellulose acetate succinate (HPMCAS), HPMCAS-L, HPMCAS-LF, HPMCAS-LG, HPMCAS-M, HPMCAS-MF, HPMCAS-MG, HPMCAS-H, HPMCAS-HF, HPMCAS-HG, HPMCAS-E3, hydroxypropyl methylcellulose phthalate (HPMCP), cellulose acetate phthalate (CAP), sodium carboxymethyl cellulose (Na-CMC), polyacrylic acid, polyethylene glycol, PEG 4000, PEG 6000, PEG 8000, PEG 20000, polyvinylpyrrolidone, PVP K 30, PVP K 25, PVP VA64, or PVP VA37.
109. The method of embodiment 108, wherein the precipitation preventer is HPMCAS.
110. The method of any one of embodiments 28-109, wherein the pharmaceutical composition further comprises one or more additional pharmaceutically acceptable excipients.
111. The method of any one of embodiments 27-110, wherein the human patient has a tumor.
112. The method of any one of embodiments 29-110, wherein the CDK2-mediated cancer is selected from bladder cancer, breast cancer, cervical cancer, colon cancer, endometrial cancer, esophageal cancer, fallopian tube cancer, glioblastoma multiforme (GBM), head and neck cancer, kidney cancer, liver cancer, lung cancer, ovarian cancer, peritoneal cancer, prostate cancer, sarcoma, skin cancer, stomach cancer, uterine cancer.
113. The method of embodiment 112, wherein the CDK2-mediated cancer is lung cancer.
114. The method of embodiment 113, wherein the lung cancer is small cell lung cancer (SCLC).
115. The method of embodiment 112, wherein the CDK2-mediated cancer is breast cancer.
116. The method of embodiment 115, wherein the breast cancer is hormone receptor-positive (HR+).
117. The method of embodiment 115 or 116, wherein the breast cancer is estrogen receptor-positive (ER+).
118. The method of any one of embodiments 115-117, wherein the breast cancer is progesterone receptor-positive (PR+).
119. The method of any one of embodiments 115-118, wherein the breast cancer is HER2− negative (HER2−).
120. The method of embodiment 115, wherein the breast cancer is triple negative breast cancer.
121. The method of embodiment 112, wherein the CDK2-mediated cancer is bladder cancer.
122. The method of embodiment 112, wherein the CDK2-mediated cancer is ovarian cancer.
123. The method of embodiment 112, wherein the CDK2-mediated cancer is prostate cancer.
124. The method of embodiment 112, wherein the CDK2-mediated cancer is sarcoma.
125. The method of embodiment 112, wherein the CDK2-mediated cancer is uterine cancer.
126. The method of any one of embodiments 29-125, wherein the CDK2-mediated cancer is advanced unresectable and/or metastatic cancer.
127. The method of any one of embodiments 29-126, wherein the CDK2-mediated cancer is cyclin E amplified or overexpressed.
128. The method of embodiment 127, wherein a Next Generation Sequencing (NGS) panel test is used to confirm cyclin E overexpression or amplification status.
129. The method of any one of embodiments 29-128, wherein the CDK2-mediated cancer is Rb-positive cancer.
130. The method of embodiment 129, wherein the Rb-positive cancer is selected from an Rb-positive HR+/IER2− breast cancer, ER+/HER2− breast cancer, high-grade serous ovarian cancer (HGSOC), non-small cell lung cancer (NSCLC), colon cancer, prostate cancer, or glioblastoma.
131. The method of embodiment 130, wherein the Rb-positive cancer is HR+/HER2− breast cancer.
132. The method of embodiment 130, wherein the Rb-positive cancer is ER+/HER2− breast cancer.
133. The method of embodiment 130, wherein the Rb-positive cancer is high-grade serous ovarian cancer (HGSOC).
134. The method of embodiment 130, wherein the Rb-positive cancer is non-small cell lung cancer (NSCLC).
135. The method of any one of embodiments 29-128, wherein the CDK2-mediated cancer is Rb-independent cancer.
136. The method of embodiment 135, wherein the Rb-independent cancer is selected from breast cancer, lung cancer, prostate cancer, liver cancer, bladder cancer, ovarian cancer, uterine cancer, cervical cancer, stomach cancer, esophageal cancer, head and neck cancer, glioblastoma, retinoblastoma, osteosarcoma, or lymphoma.
137. The method of embodiment 135, wherein the Rb-independent cancer is selected from small cell lung cancer (SCLC), retinoblastoma, triple negative breast cancer (TNBC), human papillomavirus (HPV) positive head and neck cancer, HPV positive cervical cancer, or neuroendocrine prostate carcinoma.
138. The method of embodiment 137, wherein the Rb-independent cancer is SCLC.
139. The method of embodiment 137, wherein the Rb-independent cancer is TNBC.
140. The method of any one of embodiments 27-139, wherein the human previously received at least one prior line of chemotherapy.
141. The method of any one of embodiments 27-140, wherein the human previously received at least two prior lines of chemotherapy.
142. The method of any one of embodiments 27-141, wherein the human previously received at least one prior line of CDK4/6 inhibitor therapy.
143. The method of any one of embodiments 27-142, wherein the human previously received at least one prior line of endocrine therapy.
144. The method of any one of embodiments 27-143, wherein the CDK2-mediated cancer is relapsed.
145. The method of any one of embodiments 27-144, wherein the CDK2-mediated cancer is CDK4/6 inhibitor resistant.
146. The method of any one of embodiments 27-145, wherein the CDK2-mediated cancer has progressed following a prior regimen comprising an endocrine therapy.
147. The method of any one of embodiments 27-146, wherein the CDK2-mediated cancer is endocrine therapy resistant.
148. The method of any one of embodiments 27-147, wherein the treatment results in a reduction of incidents of treatment-emergent adverse events in comparison to the predicted number of incidents of treatment-emergent adverse events in subjects receiving CDK2 treatment other than INX-315.
149. The method of any one of embodiments 27-148, wherein the treatment results in a reduction of incidents of laboratory abnormalities in comparison to the predicted number of incidents of laboratory abnormalities in subjects receiving CDK2 treatment other than INX-315.
150. The method of any one of embodiments 27-149, wherein the treatment results in an improved overall survival (OS) in comparison to the predicted overall survival (OS) in subjects receiving CDK2 treatment other than INX-315.
151. The method of any one of embodiments 27-150, wherein the treatment results in an improved overall response rate (ORR) in comparison to the predicted overall response rate (ORR) in subjects receiving CDK2 treatment other than INX-315.
152. The method of any one of embodiments 27-151, wherein the treatment results in an improved disease control rate (DCR) in comparison to the predicted disease control rate (DCR) in subjects receiving CDK2 treatment other than INX-315.
153. The method of any one of embodiments 27-152, wherein the treatment results in an improved progression free survival (PFS) in comparison to the predicted progression free survival (PFS) in subjects receiving CDK2 treatment other than INX-315.
154. The method of any one of embodiments 27-153, wherein the treatment results in an improved duration of response (DOR) in comparison to the predicted duration of response (DOR) in subjects receiving CDK2 treatment other than INX-315.
155. The method of any one of embodiments 27-154, wherein the treatment results in an extension of time to progression (TTP) in comparison to the predicted time to progression (TTP) in subjects receiving CDK2 treatment other than INX-315.
156. The method of any one of embodiments 148-155, wherein the CDK2 treatment other than INX-315 comprises the administration of a CDK2 inhibitor selected from BLU-222 or PF-07104091.
157. The method of embodiment 156, wherein the CDK2 inhibitor is BLU-222.
158. The method of embodiment 156, wherein the CDK2 inhibitor is PF-07104091.
159. The method of any one of embodiments 148-158, wherein the CDK2 inhibitor other than INX-315 is administered in a dosage amount and frequency identical to INX-315.
160. A process for manufacturing the pharmaceutical composition of any one of embodiments 1-26 comprising:
   (i) spray-drying a solution comprising INX-315, or a pharmaceutically acceptable salt thereof, and a precipitation preventer to yield an amorphous spray-dried dispersion (ASD);
   (ii) granulating the ASD together with one or more pharmaceutically acceptable excipients to provide a granulate;
   (iii) dry blending the granulate together with one or more additional pharmaceutically acceptable excipients; and
   (iv) tablet compressing the dry-blended granulate.
161. The process of embodiment 160, wherein the pharmaceutical composition has a nominal tablet strength of between about 100 mg and about 500 mg.
162. The process of embodiment 160, wherein the pharmaceutical composition has a nominal tablet strength of about 100 mg.
163. The process of embodiment 160, wherein the pharmaceutical composition has a nominal tablet strength of about 200 mg.
164. The process of embodiment 160, wherein the pharmaceutical composition has a nominal tablet strength of about 300 mg.
165. The process of embodiment 160, wherein the pharmaceutical composition has a nominal tablet strength of about 400 mg.

### BRIEF DESCRIPTION OF THE DRAWINGS

FIG. 1A is a graph showing the concentration of INX-315 in plasma obtained from oral administration of INX-315 at 100 mg once-a-day (QD) as measured on Days 1 (circles) and 15 (squares). The x-axis is time as measured in hours following administration start and the y-axis is the concentration of INX-315 in plasma as measured in nanograms per milliliter (ng/mL).

FIG. 1B is a graph showing the free concentration of INX-315 unbound from plasma protein obtained from oral administration of INX-315 at 100 mg once-a-day (QD) as measured on Days 1 (circles) and 15 (squares). The x-axis is time as measured in hours following administration start and the y-axis is the free concentration of INX-315 in plasma as measured in nanograms per milliliter (ng/mL).

FIG. 2 is a graph showing the concentration in plasma of INX-315 (circles) dosed at 100 mg once-a-day (QD) versus the concentration of commercial CDK2 inhibitor BLU-222 (triangles) dosed twice a day (BID) at 50 mg, 100 mg, 200 mg, 400 mg, or 600 mg. The x-axis is time as measured in hours following administration start and the y-axis is the concentration of INX-315 or BLU-222 in plasma as measured in nanograms per milliliter (ng/mL).

FIG. 3 is a graph showing the concentration following continuous dosing on day 15 in plasma of INX-315 (circles) dosed at 100 mg once-a-day (QD) versus the concentration of commercial CDK2 inhibitor PF-07104091 (triangles) dosed twice a day (BID) at 75 mg, 150 mg, 225 mg, 300 mg, or 500 mg. The x-axis is time as measured in hours following administration start and the y-axis is the concentration of INX-315 or PF-07104091 in plasma as measured in nanograms per milliliter (ng/mL).

FIG. 4 is a graph showing the concentration of INX-315 in whole blood obtained from oral administration of INX-315 at 100 mg once-a-day (QD) as measured on Days 1 (d1) and 15 (d15). The x-axis is time as measured in hours following administration start and the y-axis is the concentration of INX-315 in whole blood as measured in nanograms per milliliter (ng/mL).

FIG. 5A-5C are SEM images of INX-315 free base as described in Example 3.

FIG. 6A-6C are SEM images of INX-315 HCl salt as described in Example 3.

FIG. 7 represents micelle partitioning of free INX-315 drug as described in Example 3. Volume fraction of micelles (Vm) is represented on the x-axis while the Dt/Df ratio is represented on the y-axis.

FIG. 8A represents the Non-Sink Dissolution method set-up as described in Example 3.

FIG. 8B represents the Non-Sink Dissolution method with samples of free INX-315 and spray-dried INX-315 amorphous solid dispersion formulations microcentrifuged for various time lengths as represented on the x-axis and measured for total INX-315 drug concentration represented on the y-axis as described in Example 3.

FIG. 8C represents the Non-Sink Dissolution method with samples of free INX-315 and spray-dried INX-315 amorphous solid dispersion formulations ultracentrifuged for various time lengths as represented on the x-axis and measured for dissolved INX-315 drug concentration represented on the y-axis as described in Example 3.

FIG. 9A is a differential scanning calorimetry (DSC) thermogram of INX-315 upon first scan (before quench) showing temperature on the x-axis and normalized heat flow (W/g) represented on the y-axis. The DSC thermogram was obtained as described in Example 3.

FIG. 9B is a differential scanning calorimetry (DSC) thermogram of INX-315 upon second scan (after quench) showing normalized reversing heat flow and normalized non-reversing heatflow. Temperature is represented on the x-axis and normalized heat flow (W/g) represented on the y-axis. The DSC thermogram was obtained as described in Example 3.

FIG. 10A-10B are SEM images of 25% INX-315:HPMCAS-H spray-dried amorphous solid dispersion formulation as described in Example 3.

FIG. 11A-11B are SEM images of 40% INX-315:HPMCAS-H spray-dried amorphous solid dispersion formulation as described in Example 3.

FIG. 12A-12B are SEM images of 25% INX-315:HPMCAS-M spray-dried amorphous solid dispersion formulation as described in Example 3.

FIG. 13A-13B are SEM images of 40% INX-315:HPMC-E3 spray-dried amorphous solid dispersion formulation as described in Example 3.

FIG. 14 is a powder X-ray diffraction (PXRD) diffractogram plot of various spray-dried INX-315 amorphous solid dispersion formulations as described in Example 3.

FIG. 15A is a graph showing body weight measurements for six treatment groups in Cohort 1 as described in Example 5. The y-axis is body weight (plus and minus standard error of the mean) as measured in grams (g) over time on the x-axis as measured in study days. The term “mpk” refers to milligrams per kilogram (mg/kg).

FIG. 15B is a graph showing body weight measurements for six treatment groups in Cohort 2 as described in Example 5. The y-axis is body weight (plus and minus standard error of the mean) as measured in grams (g) over time on the x-axis as measured in study days. The term “mpk” refers to milligrams per kilogram (mg/kg).

FIG. 15C is a graph showing tumor volume measurements for six treatment groups in Cohort 1 as described in Example 5. The y-axis is tumor mean volume (plus and minus standard error of the sample) as measured in millimeters cubed (mm³) over time on the x-axis as measured in study days. The term “mpk” refers to milligrams per kilogram (mg/kg).

FIG. 15D is a graph showing tumor volume measurements for six treatment groups in Cohort 2 as described in Example 5. The y-axis is tumor mean volume (plus and minus standard error of the sample) as measured in millimeters cubed (mm³) over time on the x-axis as measured in study days. The term “mpk” refers to milligrams per kilogram (mg/kg).

FIG. 16A is a graph showing body weight measurements for different treatment groups as described in Example 6. The y-axis is body weight (plus or minus standard deviation) as measured in grams (g) over time on the x-axis as measured in study days.

FIG. 16B is a graph showing tumor volume measurements for different treatment groups as described in Example 6. The y-axis is tumor mean volume (plus and minus standard error of the mean) as measured in millimeters cubed (mm³) over time on the x-axis as measured in study days.

FIG. 17A is a graph showing the concentration of INX-315 in whole blood obtained from oral administration of INX-315 at 100 mg once-a-day (QD) as measured on Day 1 in Cohort 1 for six individual subjects as described in Example 7. The x-axis is time as measured in hours following administration start and the y-axis is the concentration of INX-315 in whole blood as measured in nanograms per milliliter (ng/mL).

FIG. 17B is a graph showing the concentration of INX-315 in whole blood obtained from oral administration of INX-315 at 100 mg once-a-day (QD) as measured on Day 15 in Cohort 1 for six individual subjects as described in Example 7. The x-axis is time as measured in hours following administration start and the y-axis is the concentration of INX-315 in whole blood as measured in nanograms per milliliter (ng/mL).

FIG. 17C is a graph showing the concentration of INX-315 in plasma obtained from oral administration of INX-315 at 100 mg once-a-day (QD) as measured on Day 1 in Cohort 1 for six individual subjects as described in Example 7. The x-axis is time as measured in hours following administration start and the y-axis is the concentration of INX-315 in plasma as measured in nanograms per milliliter (ng/mL).

FIG. 17D is a graph showing the concentration of INX-315 in plasma obtained from oral administration of INX-315 at 100 mg once-a-day (QD) as measured on Day 15 in Cohort 1 for six individual subjects as described in Example 7. The x-axis is time as measured in hours following administration start and the y-axis is the concentration of INX-315 in plasma as measured in nanograms per milliliter (ng/mL).

FIG. 17E is a graph showing the concentration of INX-315 in whole blood and plasma obtained from oral administration of INX-315 at 100 mg once-a-day (QD) as measured on Days 1 and 15 in Cohort 1 represented separately for six individual subjects as described in Example 7. The x-axis is time as measured in hours following administration start and the y-axis is the concentration of INX-315 in either whole blood or plasma as measured in nanograms per milliliter (ng/mL).

FIG. 18A is a graph showing the concentration in plasma of INX-315 dosed at 100 mg once-a-day (QD) (gray) versus 200 mg QD (black) as described in Example 8. The x-axis is time as measured in hours following administration start and the y-axis is the concentration of INX-315 in plasma as measured in nanograms per milliliter (ng/mL).

FIG. 18B is a graph showing the concentration in plasma of INX-315 dosed at 100 mg once-a-day (QD) (gray) versus 200 mg QD (black) as described in Example 8. The x-axis represents time points represented by cycle number (C#) and day number (D #) starting with C1D1 and the y-axis is the concentration of INX-315 in plasma as measured in nanograms per milliliter (ng/mL).

FIG. 18C is a graph showing the free concentration in plasma of INX-315 dosed at 100 mg once-a-day (QD) (gray) versus 200 mg QD (black) as described in Example 8. The x-axis represents Day 1 and Day 7 and the y-axis is the concentration of INX-315 in plasma as measured in nanograms per milliliter (ng/mL).

FIG. 19A is a graph illustrating preliminary Plasma INX-315 concentration-time profiles for Cohort 1 (100 mg QD) as described in Example 10. The x-axis represents time in hours (h) after dose administration on Day 1 (black line) and Day 15 (gray line). The y-axis is linear scale and represents the concentration of INX-315 in plasma as measured in nanograms per milliliter (ng/mL). Dose appears in top header row; Subject ID appears in second header row.

FIG. 19B is a graph illustrating preliminary Plasma INX-315 concentration-time profiles for Cohort 1 (100 mg QD) as described in Example 10. The x-axis represents time in hours (h) after dose administration on Day 1 (black line) and Day 15 (gray line). The y-axis is logarithmic scale and represents the concentration of INX-315 in plasma as measured in nanograms per milliliter (ng/mL). Dose appears in top header row; Subject ID appears in second header row.

FIG. 19C is a graph illustrating individual and mean (SD) Plasma INX-315 concentration-time profiles for Cohort 1 (100 mg QD) on Day 1 after dosing as described in Example 10. Gray lines are individual concentration-time profiles and solid black lines represent the mean concentration-time profile of the entire cohort.

FIG. 19D is a graph illustrating individual and mean (SD) Plasma INX-315 concentration-time profiles for Cohort 1 (100 mg QD) on Day 15 after dosing as described in Example 10. Gray lines are individual concentration-time profiles and solid black lines represent the mean±SD concentration-time profile of the entire cohort.

FIG. 20A is a graph illustrating preliminary Plasma INX-315 concentration-time profiles for Cohort 2 (200 mg QD) as described in Example 10. The x-axis represents time in hours (h) after dose administration on Day 1 (black line) and Day 15 (gray line). The y-axis is linear scale and represents the concentration of INX-315 in plasma as measured in nanograms per milliliter (ng/mL). Dose appears in top header row; Subject ID appears in second header row.

FIG. 20B is a graph illustrating preliminary Plasma INX-315 concentration-time profiles for Cohort 2 (200 mg QD) as described in Example 10. The x-axis represents time in hours (h) after dose administration on Day 1 (black line) and Day 15 (gray line). The y-axis is logarithmic scale and represents the concentration of INX-315 in plasma as measured in nanograms per milliliter (ng/mL). Dose appears in top header row; Subject ID appears in second header row.

FIG. 20C is a graph illustrating individual and mean (SD) Plasma INX-315 concentration-time profiles for Cohort 2 (200 mg QD) on Day 1 after dosing as described in Example 10. Gray lines are individual concentration-time profiles and solid black lines represent the mean concentration-time profile of the entire cohort.

FIG. 20D is a graph illustrating individual and mean (SD) Plasma INX-315 concentration-time profiles for Cohort 2 (200 mg QD) on Day 15 after dosing as described in Example 10. Gray lines are individual concentration-time profiles and solid black lines represent the mean concentration-time profile of the entire cohort.

FIG. 21A is a graph illustrating preliminary Plasma INX-315 concentration-time profiles for Cohort 3 (300 mg QD) as described in Example 10. The x-axis represents time in hours (h) after dose administration on Day 1 (black line) and Day 15 (gray line). The y-axis is linear scale and represents the concentration of INX-315 in plasma as measured in nanograms per milliliter (ng/mL). Dose appears in top header row; Subject ID appears in second header row.

FIG. 21B is a graph illustrating preliminary Plasma INX-315 concentration-time profiles for Cohort 3 (300 mg QD) as described in Example 10. The x-axis represents time in hours (h) after dose administration on Day 1 (black line) and Day 15 (gray line). The y-axis is logarithmic scale and represents the concentration of INX-315 in plasma as measured in nanograms per milliliter (ng/mL). Dose appears in top header row; Subject ID appears in second header row.

FIG. 21C is a graph illustrating individual and mean (SD) Plasma INX-315 concentration-time profiles for Cohort 3 (300 mg QD) on Day 1 after dosing as described in Example 10. Gray lines are individual concentration-time profiles and solid black lines represent the mean concentration-time profile of the entire cohort.

FIG. 21D is a graph illustrating individual and mean (SD) Plasma INX-315 concentration-time profiles for Cohort 3 (300 mg QD) on Day 15 after dosing as described in Example 10. Gray lines are individual concentration-time profiles and solid black lines represent the mean concentration-time profile of the entire cohort.

FIG. 22A is a graph illustrating individual and mean (SD) Plasma INX-315 concentration-time profiles for all Cohorts on Day 1 after dosing as described in Example 10. Dotted lines are individual concentration-time profiles. Solid lines are mean±SD concentration-time profiles for Cohort 1 (black line), Cohort 2 (dark gray line), and Cohort 3 (light gray line).

FIG. 22B is a graph illustrating individual and mean (SD) Plasma INX-315 concentration-time profiles for all Cohorts on Day 15 after dosing as described in Example 10. Dotted lines are individual concentration-time profiles. Solid lines are mean±SD concentration-time profiles for Cohort 1 (black line), Cohort 2 (dark gray line) except for Cohort 3 (light gray line) the which is the median concentration-time profile.

FIG. 23 provides the chemical structure of INX-315.

### DETAILED DESCRIPTION

It has been surprisingly discovered as a result of first time in human dosing of INX-315 that this compound has a unique and highly unexpected pharmacokinetic profile, which is significantly inconsistent with predicted mathematical pharmacokinetic models extrapolated from other mammalian species (dogs, rats and mice). While not wanting to be held to one theory, it is proposed that INX-315 binds to human blood cells in vivo to a much greater degree than seen in the other tested mammals and from these other mammalian models, which increases the amount of drug available in the blood and creates a depot effect for a type of extended availability of the drug as it is released over time from the red blood cells into the plasma and then to the site of therapy.

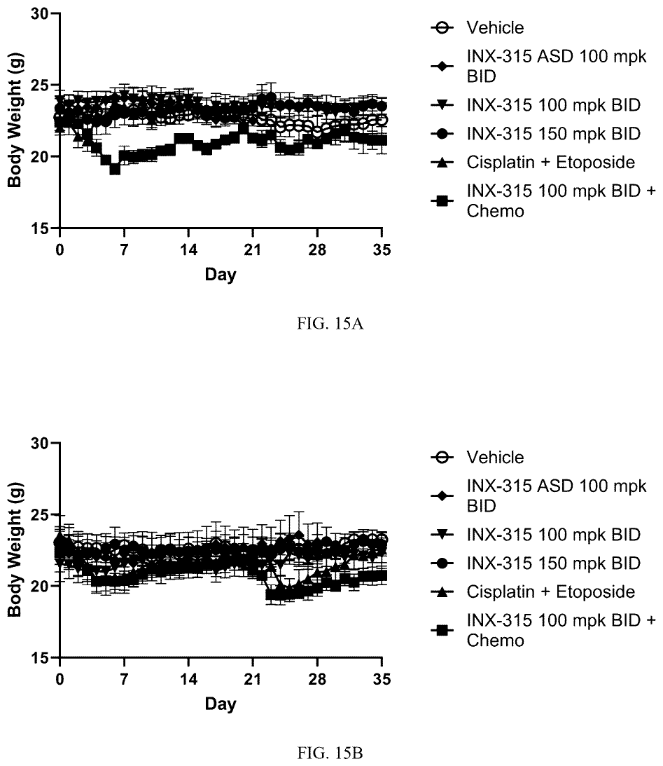

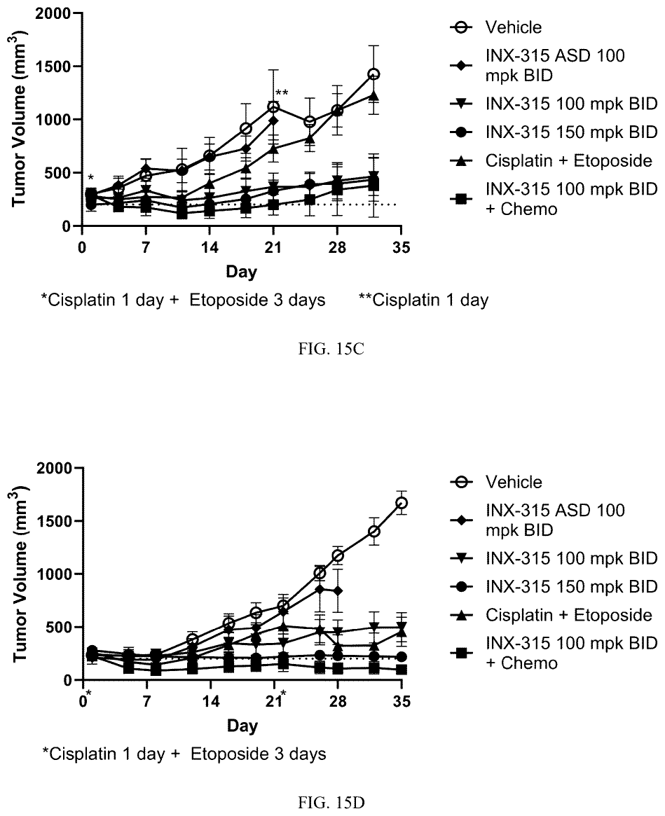

Surprisingly, it has also been discovered that INX-315 is effective in the treatment of Rb-independent (i.e., Rb-negative) cancers. It is shown here that the CDK2 inhibitor INX-315 can be used in an effective amount to treat Rb-independent cancers including but not limited to Rb-independent small cell lung cancer (SCLC) (see, e.g., FIG. 15C-15D). Other Rb-independent tumors include Rb-independent triple negative breast cancer (TNBC), prostate cancer, liver cancer, bladder cancer, ovarian cancer, uterine cancer, cervical cancer, stomach cancer, esophageal cancer, head and neck cancer, glioblastoma, retinoblastoma, osteosarcoma, or lymphoma.

This discovery provides an advance in the art of cancer therapy by providing a CDK2 inhibitor that is useful to treat both Rb-dependent and Rb-independent tumors, which can include heterogeneous tumors of both. In some embodiments, INX-315 is administered in conjunction with a CDK4/6 inhibitor including but not limited to ribociclib (Novartis), abemaciclib (Eli Lilly), palbociclib (Pfizer Inc), or lerociclib (G1 Therapeutics, Inc.). In some embodiments, INX-315 is administered on a daily basis and the CDK4/6 inhibitor is typically administered for 21 days during a 28-day cycle with a 7 day drug holiday. In some embodiments, INX-315 is administered on a daily basis and the CDK4/6 inhibitor is administered continuously.

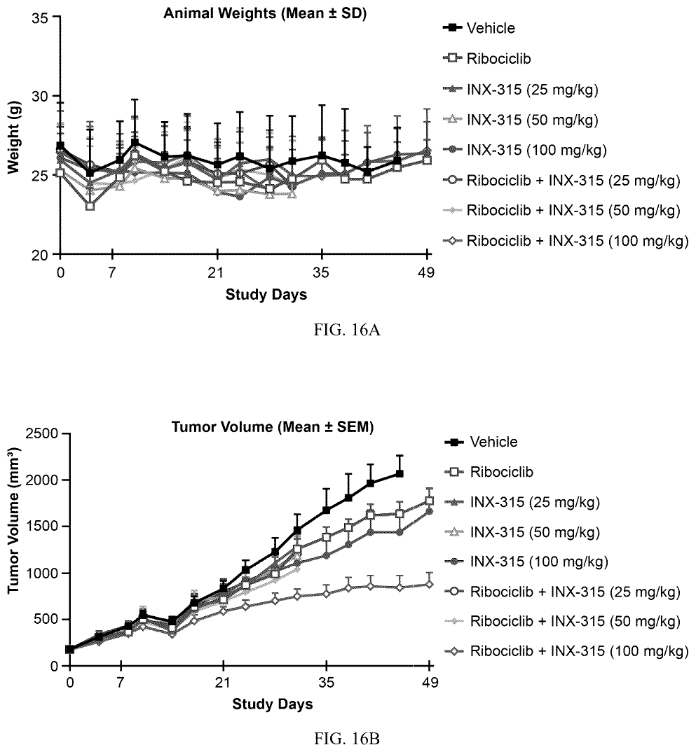

INX-315 can be used according to the present invention to treat tumors with intact, functional retinoblastoma protein (Rb-positive). In some embodiments, the Rb-positive cancer is selected from an Rb-positive HR+/HER2− breast cancer, ER+/HER2− breast cancer, high-grade serous ovarian cancer (HGSOC), non-small cell lung cancer (NSCLC), colon cancer, prostate cancer, or glioblastoma. In some embodiments, the Rb-positive cancer is HR+/IiER2− breast cancer. In some embodiments, the Rb-positive cancer is ER+/HER2− breast cancer. In some embodiments, the Rb-positive cancer is high-grade serous ovarian cancer (HGSOC). In some embodiments, the Rb-positive cancer is non-small cell lung cancer (NSCLC). In some embodiments, INX-315 or a pharmaceutically acceptable salt or ASD formulation thereof described herein provides an additive effect to or synergistic effect with the anti-cancer or anti-proliferative activity of an additional therapeutic agent for the treatment of Rb-positive cancer. In some embodiments, the additional therapeutic agent is selected from a CDK4/6 inhibitor, an endocrine therapy, a chemotherapeutic agent, an immune checkpoint inhibitor, or a combination thereof (see, e.g., FIG. 15C-15D and FIG. 16B). In some embodiments, the additional therapeutic is a CDK4/6 inhibitor. In some embodiments, the CDK4/6 inhibitor is selected from palbociclib, ribociclib, abemaciclib, trilaciclib, lerociclib, or dalpiciclib. In some embodiments, the CDK4/6 inhibitor is palbociclib (Pfizer Inc). In some embodiments, the CDK4/6 inhibitor is ribociclib (Novartis). In some embodiments, the CDK4/6 inhibitor is abemaciclib (Eli Lilly). In some embodiments, the CDK4/6 inhibitor is lerociclib (G1 Therapeutics, Inc.).

Unexpectedly, it has also been discovered that INX-315 can effectively treat Rb-independent cancers (see, e.g., FIG. 15C-15D). In some embodiments, the Rb-independent cancer is selected from small cell lung cancer (SCLC), retinoblastoma, triple negative breast cancer (TNBC), human papillomavirus (HPV) positive head and neck cancer, HPV positive cervical cancer, or bladder cancer. In some embodiments, the Rb-independent cancer is SCLC. In some embodiments, the Rb-independent cancer is TNBC. In some embodiments, the Rb-independent cancer is bladder cancer. In some embodiments, INX-315 or a pharmaceutically acceptable salt or ASD formulation thereof described herein provides an additive effect to or synergistic effect with the anti-cancer or anti-proliferative activity of an additional therapeutic agent for the treatment of Rb-independent cancer. In some embodiments, the additional therapeutic agent is selected from a CDK4/6 inhibitor, an endocrine therapy, a chemotherapeutic agent, an immune checkpoint inhibitor, or a combination thereof (see, e.g., FIG. 15C-15D and FIG. 16B). In some embodiments, the additional therapeutic is a CDK4/6 inhibitor. In some embodiments, the CDK4/6 inhibitor is selected from palbociclib, ribociclib, abemaciclib, trilaciclib, lerociclib, or dalpiciclib. In some embodiments, the CDK4/6 inhibitor is palbociclib (Pfizer Inc). In some embodiments, the CDK4/6 inhibitor is ribociclib (Novartis). In some embodiments, the CDK4/6 inhibitor is abemaciclib (Eli Lilly). In some embodiments, the CDK4/6 inhibitor is lerociclib (G1 Therapeutics, Inc.).

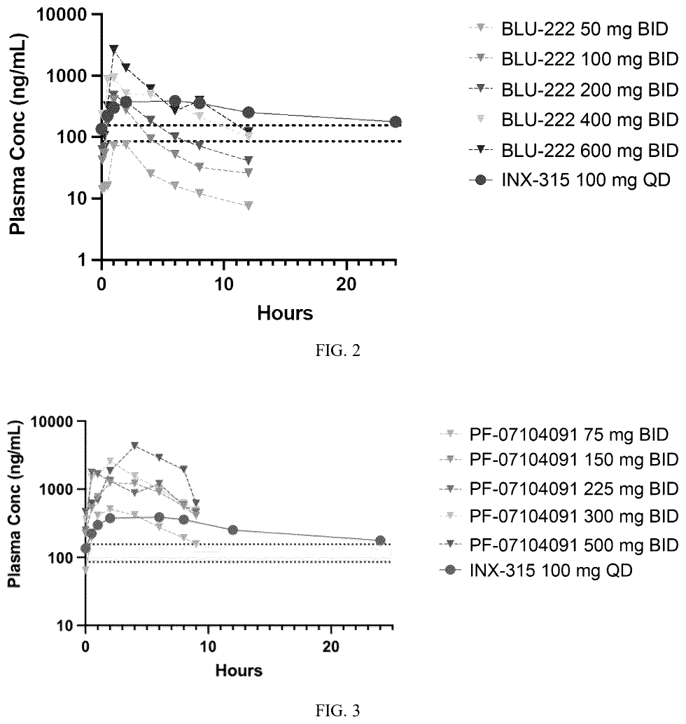

It is also highly unexpected that the administration of INX-315 does not result in a spike of drug concentration on initial administration as often seen with other drugs, such as BLU-222 and PF-07104091. A spike effect is indicated by a large initial Cₘₐₓ to Cₘᵢₙ ratio at the beginning, which tails off after administration (see FIGS. 2 and 3). Conversely, the lack of a spike is indicated when the ratio of Cₘₐₓ to Cₘᵢₙ is small. The Cₘₐₓ to Cₘᵢₙ ratio of INX-315 in the first two humans at 100 mg once-a-day was measured as about 2.2, whereas the estimated Cₘₐₓ to Cₘᵢₙ ratio based on published human results for BLU-222 (400 mg twice a day) was 9.3 and for BLU-222 (600 mg twice a day) was 22. Likewise, the estimated Cₘₐₓ to Cₘᵢₙ ratio for PF-07104091 was 16-64 based on publicly available human data (see Table 5 and FIGS. 2 and 3). The presence of a spike on dosing, which is a short-term excess amount of drug in the blood, can lead to side effects and is typically disfavored.

It has been surprisingly and unexpectedly discovered that the administration of INX-315 or a pharmaceutically acceptable salt thereof provides particularly improved plasma and whole blood pharmacokinetic parameters compared to the values predicted based upon non-human preclinical model studies. In some embodiments, the cancers have amplified or overexpressed cyclin E, are resistant to CDK4/6 inhibitors (see, e.g., FIG. 16B) either through acquired resistance or intrinsic resistance (e.g., SCLC) (see, e.g., FIG. 15C-15D), and/or are resistant to endocrine therapies, for example estrogen receptor degraders. The dosage regimes comprising the administration of INX-315 or a pharmaceutically acceptable salt thereof described herein can be used to treat Rb-positive and Rb-independent cancers such as SCLC (see, e.g., FIG. 15C-15D) alike. The improved dosage regimes described herein can be used to treat difficult-to-treat cyclin E amplified cancers including CCNE1 amplified unresectable solid tumors and CCNE1 amplified platinum-resistant or platinum-refractory cancers. Non-limiting examples of favorable properties exhibited by the dosage regimes comprising administration of INX-315 described herein include enhanced area under the curve (AUC) (ng*hr/mL) values, mean maximum concentration (Cₘₐₓ) (ng/mL) values, enhanced mean minimum concentration (Cₘᵢₙ) (ng/mL) values, enhanced mean free minimum plasma concentration (Cₘᵢₙ^{free}) (ng/mL) values, and enhanced Cₘₐₓ to Cₘᵢₙ ratios than what was predicted based upon non-human mammalian preclinical models. These improved PK profiles provide lengthened duration of concentration above therapeutic threshold, superior exposure profiles, extended half-life (t_{1/2}), and decreased off-target activity compared to clinically developing CDK2 inhibitors. The sustained steady-state levels of INX-315 in the plasma leads to continuous on-target drug activity and a tumor microenvironment that is favorable for re-sensitizing tumors to other therapeutic agents including but not limited to CDK4/6 inhibitors, endocrine therapies such as SERDs, chemotherapeutic agents, immune checkpoint inhibitors, or a combination thereof (see, e.g., FIG. 15C-15D and FIG. 16B). This improvement provides a significant advance in the state of the art of cancer treatment.

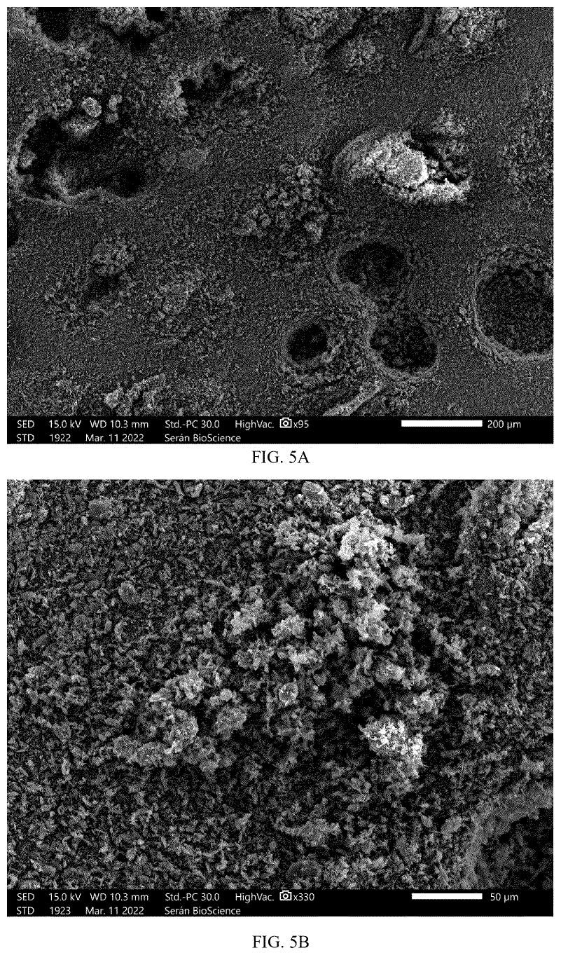

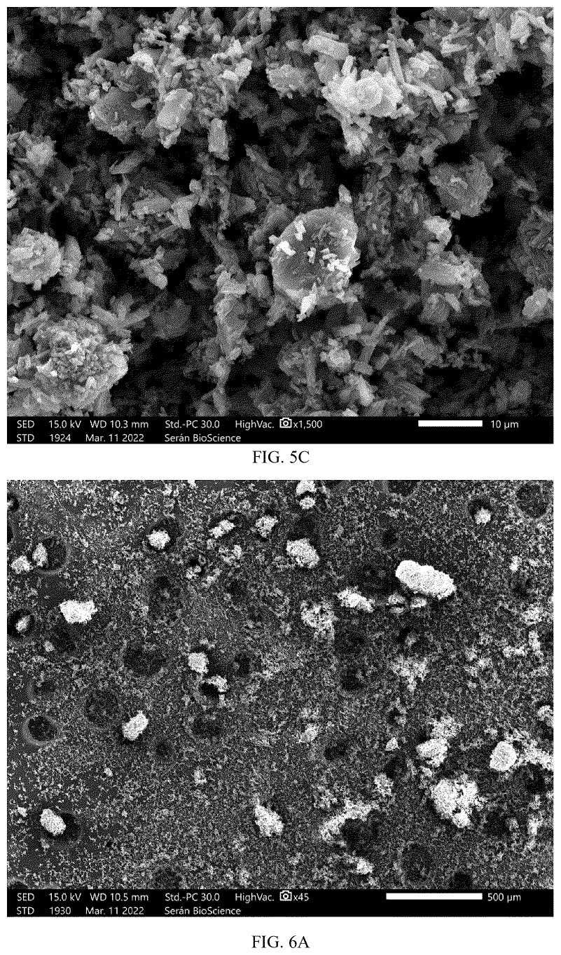

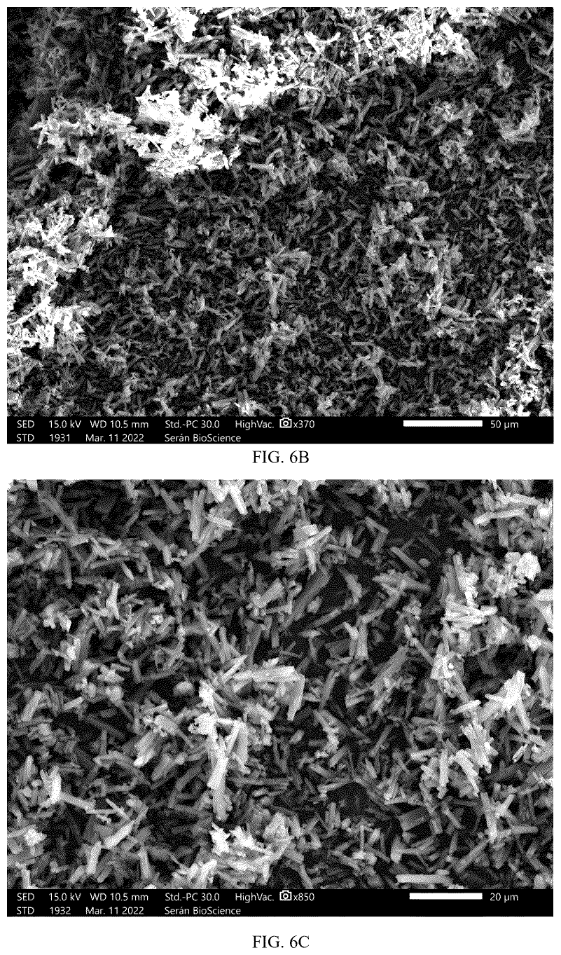

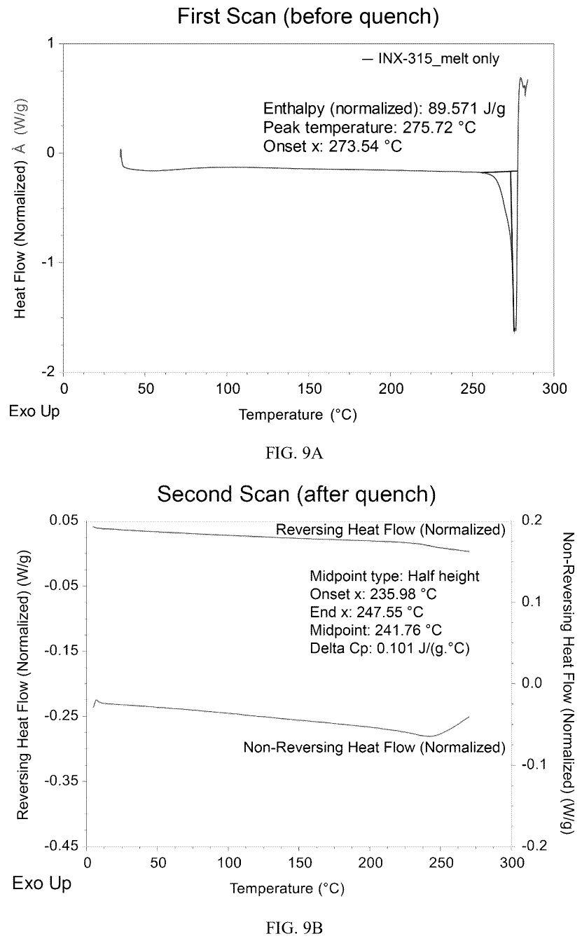

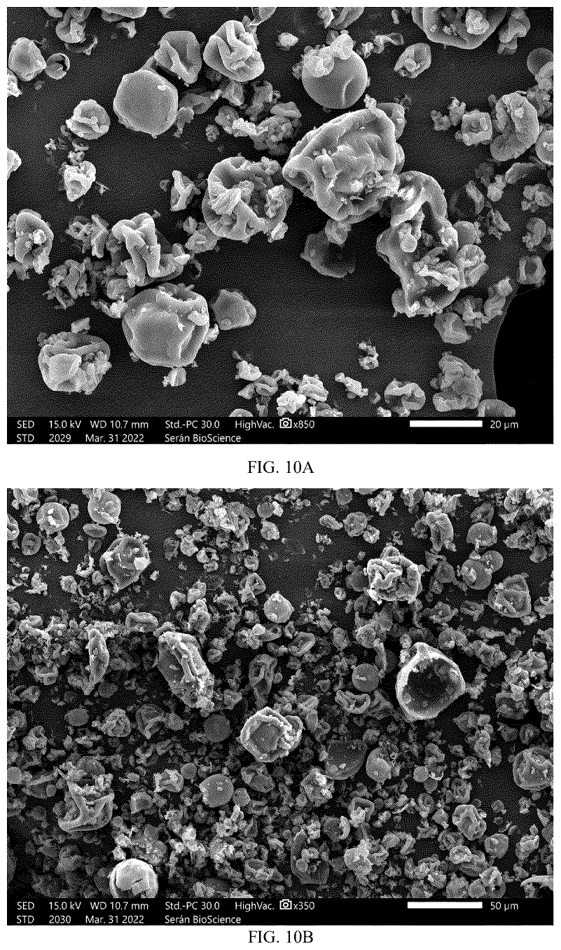

As described herein, INX-315 is highly insoluble in water (Table 12) and free INX-315 is crystalline (FIG. 5A-6C). It has been discovered that INX-315 can be mixed with a polymeric precipitation preventer in a solvent to create an advantageous amorphous spray-dried dispersion (ASD) formulation. The precipitation preventer minimizes crystallization of the INX-315 in the ASD formulation. In non-limiting embodiments, the solvent is a mixture of THF and water, for example 90% THF and 10% water (see e.g., Table 20). In some embodiments, the solvent is a mixture of THF and water, for example 92% THF and 8% water (see, e.g., Table 33). In some embodiments, the THF is stabilized with butylated hydroxytoluene (BHT). As a non-limiting illustration in Example 3, the INX-315 ASD formulation disperses INX-315 across the polymer matrix (e.g., precipitation preventer such as HPMCAS) preventing the crystallization of INX-315 in the ASD formulation, as indicated by high T_{g} and low Tₘ/T_{g} ratios (FIG. 9A-9B).

In some embodiments, the amorphous INX-315 is included in a range of between about 20% and about 50% by weight of the ASD, for example between about 25 and about 45% by weight of the ASD, or for example about 40% by weight of the ASD (Tables 17 and 20). In some embodiments, INX-315 is spray-dried together with a precipitation preventer, for example but not limited to hydroxy propyl methylcellulose acetate succinate-high substitution (HPMCAS-HG), or other precipitation preventers as described further herein. The precipitation preventer can be, for example included in a range of between about 50% and about 70% by weight of the ASD, or for example between about 55% and about 65% by weight of the ASD, or for example about 60% by weight of the ASD (Tables 17 and 20).

In an additional aspect, advantageous pharmaceutical compositions are described herein comprising the ASD formulations added together with one or more additional pharmaceutically acceptable excipients. As a non-limiting illustration in Example 4, the INX-315 ASD formulation is dry-granulated, dry-blended, then tableted together with one or more additional pharmaceutically acceptable excipients, for example including but not limited to microcrystalline cellulose, mannitol, croscarmellose sodium, colloidal silicon dioxide, and sodium stearyl fumarate (Tables 21 and 23), to create a pharmaceutical composition to be administered to a subject in need thereof.

In some embodiments, the amorphous INX-315 is included in a range of between about 10% and about 30% by weight of the pharmaceutical composition, or between about 15% and about 35% by weight of the pharmaceutical composition, or for example about 20% by weight of the pharmaceutical composition (Tables 17 and 20). The precipitation preventer can be, for example included in a range of between about 20% and about 50% by weight of the pharmaceutical composition, or for example between about 25% and about 45% by weight of the pharmaceutical composition, or for example about 30% by weight of the pharmaceutical composition (Tables 17 and 20).

### Terminology

Unless defined otherwise, all technical and scientific terms used herein have the same meaning as is commonly understood by one of skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

In some embodiments, the term “about” means±10%.

The “patient” or “subject” or “participant” treated is typically a human patient, unless otherwise indicated. In alternative embodiments, the methods described herein can be used to treat or in testing of other animals that respond similarly, such as mammals, for example, such as those used in preclinical testing including but not limited to mice, rats, monkeys, dogs, pigs, and rabbits; as well as domesticated swine (pigs and hogs), ruminants, equine, poultry, felines, bovines, murines, canines, and the like.

An “effective amount” as used herein, means an amount which provides a therapeutic benefit.

The term “mean” as used herein is synonymous with average, e.g., the central numerical point among a set of values. In some embodiments, for PK parameters with an assumed skew distribution (e.g., Cₘₐₓ, AUC), mean refers to geometric mean to account for compounding effects.

The term “treatment-emergent adverse events” as used herein, means an adverse event that occurs or worsens after the first dose or during treatment.

The term “overall survival (OS)” as used herein, means the time from the first dosing until death due to any causes.

The term “overall response rate (ORR)” as used herein, means the occurrence of either a confirmed best overall response of CR or PR, as determined by the RECIST v1.1 criteria.

The term “disease control rate (DCR)” as used herein, means the occurrence of a confirmed best overall response of CR, PR, or SD, as determined by the RECIST v1.1 criteria.

The term “progression free survival (PFS)” as used herein, means the duration of time from the first dosing until the first documented disease progression or death of any causes, whichever occurs first.

The term “duration of response (DOR)” as used herein, means the time from when confirmed best overall response of CR or PR was first documented to the first documented disease progression or death of any causes, whichever occurs first.

The term “time to progression (TTP)” as used herein, means the time from the first dosing until the first documented disease progression.

The term “MTD”, as used herein, refers to maximum tolerated dose of a compound, for example INX-315.

The term “AUC”, as used herein, refers to area under the concentration-time curve of a compound in whole blood or plasma.

The term “AUC₀₋ₜ” or “AUCₗₐₛₜ”, as used herein, refers to AUC to time of last measurable concentration of a compound, for example INX-315.

The term “AUC₀₋₂₈”, as used herein, refers to AUC to time infinity of a compound, for example INX-315.

The term “Cl”, as used herein, refers to clearance of a compound, for example INX-315.

The term “Cₘₐₓ”, as used herein, refers to maximum concentration of a compound in whole blood or plasma, for example INX-315.

The term “Cₗₐₛₜ”, as used herein, refers to the last quantifiable concentration of a compound in whole blood or plasma, for example INX-315.

The term “IC₅₀”, as used herein, refers to the half-maximal inhibitory concentration of a compound, for example INX-315. IC₅₀, a measure of the potency of a drug, indicates the concentration of a target compound that inhibits a selected biological process by half compared to a control compound.

The term “t_{1/2}”, as used herein, refers to half-life of a compound, for example INX-315.

The term “tₘₐₓ”, as used herein, refers to time to reach Cₘₐₓ of compound in whole blood or plasma, for example INX-315.

The term “tₗₐₛₜ”, as used herein, refers to time to reach last measurable concentration of a compound, for example INX-315.

The term “precipitation preventer” as used herein refers to a polymer that is used in an amorphous spray-dried dispersion (ASD) formulation to increase the solubility of INX-315 in the ASD formulation and/or prevent crystallization of INX-315 in the ASD formulation. Non-limiting examples of precipitation preventers include but are not limited to hydroxypropylmethylcellulose, for example HPMCAS, or other agents as further described herein.

“Intrinsic resistance,” also known as primary resistance, as used herein, refers to a condition wherein a cancer is not responsive to, or sufficiently responsive to, the inhibitory effects of initial anti-cancer treatment including for example, but not limited to CDK4/6 inhibitor treatment or endocrine therapy treatment. In some embodiments of the methods of treatment described herein, the cancer to be treated is intrinsically resistant to a CDK4/6 inhibitor. Mutations and conditions associated with, for example, CDK4/6 inhibitor intrinsic resistance include, but are not limited to: increased activity of cyclin-dependent kinase 1 (CDK1); increased activity of cyclin-dependent kinase 2 (CDK2); loss, deficiency, or absence of retinoblastoma tumor suppressor protein (Rb) (Rb-null); high levels of pl6Ink4a expression; high levels of MYC expression; increased expression of cyclin E1, cyclin E2, and cyclin A; and combinations thereof. The CDK4/6 inhibitor intrinsically resistant cancer may be characterized by reduced expression of the retinoblastoma tumor suppressor protein or a retinoblastoma family member protein or proteins (such as, but not limited to p107 and p130). In some embodiments, a tumor or cancer that is intrinsically resistant to selective CDK4/6 inhibitor inhibition is a tumor or cancer whose cell population, as a whole, does not experience substantial G1 cell-cycle arrest when exposed to a selective CDK4/6 inhibitor. In some embodiments, a tumor or cancer that is intrinsically resistant to CDK4/6 inhibitor inhibition is a tumor or cancer who has a cell population wherein less than 25%, 20%, 15%, 10%, or 5% of its cells experience G1 cell-cycle arrest when exposed to a selective CDK4/6 inhibitor. In some alternative embodiments of the methods of treatment described herein, the cancer to be treated is intrinsically resistant to an endocrine therapy, for example an estrogen inhibitor therapy such as a SERD. In some alternative embodiments of the methods of treatment described herein, the cancer treated is intrinsically resistant to a therapeutic agent or other anti-cancer therapy.

“Acquired resistance,” as used herein, refers to a condition wherein a cancer that was or is initially sensitive to the inhibitory effects of an anti-cancer therapy becomes non-responsive or less-responsive over time to the effects of the administration of that therapy. In some embodiments of the methods described herein, the cancer to be treated has acquired resistance to a selective CDK4/6 inhibitor. Without wishing to be bound by any one theory, it is believed that acquired resistance to CDK4/6 inhibitors occurs due to one or more additional mutations or genetic alterations in bypass signaling that develops after the onset of CDK4/6 inhibitor treatment regimen. For example, non-limiting exemplary causes of acquired resistance to CDK4/6 inhibitors may be a result of the development of one or more genetic aberrations associated with “intrinsic resistance”. In addition, other non-limiting exemplary causes of acquired resistance to CDK4/6 inhibitors may include an increase in cyclin E expression; CCNE1/2 amplification; E2F amplification; CDK2 amplification; amplification of CDK6; amplification of CDK4; p16 amplification; WEE1 overexpression; MDM2 overexpression; CDK7 overexpression; loss of FZR1; HDAC activation; activation of the FGFR pathway; activation of the PI3K/AKT/mTOR pathway; loss of ER or PR expression; higher transcriptional activity of AP-1; epithelial-mesenchymal transition; Smad 3 suppression; autophagy activation; Rb1-loss or inactivating RB1 mutations; or a combination thereof A general review of CDK4/6-resistant mechanisms can be found, for example, in Pandey et al., Molecular mechanisms of resistance to CDK4/6 inhibitors in breast cancer: A review. Int. J. Cancer:00,1-10 (2019), incorporated herein by reference. In some embodiments, a tumor or cancer that has acquired resistance to selective CDK4/6 inhibitor inhibition is a tumor or cancer whose cell population, as a whole, no longer experiences substantial G1 cell-cycle arrest when exposed to a selective CDK4/6 inhibitor, resulting in disease progression. In some embodiments, a tumor or cancer that has acquired resistance to CDK4/6 inhibitor inhibition is a tumor or cancer who has a cell population wherein less than 50%, 40%, 30% 20%, 15%, 10%, or 5% of its cells experience G1 cell-cycle arrest when exposed to a selective CDK4/6 inhibitor, leading to disease progression. In some embodiments, the cancer has progressed following a prior therapeutic regimen comprising the administration of a CDK4/6 inhibitor. In some alternative embodiments of the methods of treatment described herein, the cancer treated has acquired resistance, for example to an estrogen inhibitor therapy such as a SERD, for example, but not limited to, fulvestrant or elacestrant. In some alternative embodiments of the methods of treatment described herein, the cancer treated has acquired resistance to an additional anti-cancer therapy.

The retinoblastoma susceptibility gene (RB1) was the first molecularly defined tumor suppressed gene. Retinoblastoma (Rb), the protein encoded by RB1, is frequently mutated or deleted in retinoblastoma and osteosarcoma, and is variably mutated or deleted in certain other tumor or cancer types including but not limited to prostate cancer, neuroendocrine prostate carcinoma, breast cancer, triple negative breast cancer (TNBC), lung cancer, small cell lung cancer (SCLC), liver cancer, bladder cancer, ovarian cancer, uterine cancer, cervical cancer, stomach cancer, esophageal cancer, head and neck cancer, glioblastoma, and lymphoma. In some cancers, Rb may be neutralized by a binding partner, for example human papilloma virus-E7 protein in cervical carcinoma (Ishiji, T. J Dermatol. 27:73-86(2000)) or through the dysregulation of the Rb pathway.

The term “Rb pathway” refers to the entire molecular signaling pathway including retinoblastoma protein (Rb) and other proteins and protein families in the pathway including but not limited to CDK2, CDK4, CDK6, E2f, atypical protein kinase C, and Skp2.

The terms “Rb-plus”, “Rb+”, “Rb-proficient”, “Rb-dependent”, or “Rb-positive” refers to several types of cells, tumors, or cancers wherein functional Rb is expressed in detectable amounts. In some embodiments, Rb-positive cells, tumors, or cancers comprise cells, tumors, or cancers which contain a functional RB1 gene. In some embodiments, Rb-positive cells, tumors, or cancers comprise cells, tumors, or cancers which encode a functional Rb protein. In some embodiments, Rb-positive cells, tumors, or cancers comprise cells, tumors, or cancers which express detectable amounts of RB1 or Rb.

The presence or normal functioning of the retinoblastoma (Rb) tumor suppressor protein (Rb-positive) can be determined through any of the standard assays known to one of ordinary skill in the art, including but not limited to Western Blot, ELISA (enzyme linked immunoadsorbent assay), IHC (immunohistochemistry), and FACS (fluorescent activated cell sorting). The selection of the assay will depend upon the tissue, cell line or surrogate tissue sample that is utilized e.g., for example Western Blot and ELISA may be used with any or all types of tissues, cell lines or surrogate tissues, whereas the IHC method would be more appropriate wherein the tissue utilized in the methods of the present invention was a tumor biopsy. FACs analysis would be most applicable to samples that were single cell suspensions such as cell lines and isolated peripheral blood mononuclear cells. See for example, US 20070212736 “Functional Immunohistochemical Cell Cycle Analysis as a Prognostic Indicator for Cancer”. Alternatively, molecular genetic testing may be used for determination of retinoblastoma gene status. Molecular genetic testing for retinoblastoma includes the following as described in Lohmann and Gallie “Retinoblastoma. Gene Reviews” (2010) or Parsam et al. “A comprehensive, sensitive and economical approach for the detection of mutations in the RB1 gene in retinoblastoma” Journal of Genetics, 88(4), 517-527 (2009).

The terms “Rb-independent”, “Rb-negative”, “Rb-deficient”, “Rb-minus”, “Rb-null”, or “Rb-” refers to several types of cells, tumors, or cancers wherein the function of Rb is disrupted and includes, in some cases, cells, tumors, or cancers wherein Rb is deleted or there is no detectable amount of Rb. In some embodiments, Rb-independent cells, tumors, or cancers comprise cells, tumors, or cancers which do not contain a functional RB1 gene. In some embodiments, Rb-independent cells, tumors, or cancers comprise cells, tumors, or cancers which encode a dysfunctional Rb protein. In some embodiments, Rb-independent cells, tumors, or cancers comprise cells, tumors, or cancers which are Rb deficient. In some embodiments, Rb-independent cells, tumors, or cancers comprise cells, tumors, or cancers which are characterized by loss of function mutations in RB1, which may comprise missense mutations (i.e., encoding for an incorrect amino acid) or nonsense mutations (i.e., encoding for a stop codon). In some embodiments, Rb-independent cells, tumors, or cancers comprise cells, tumors, or cancers which are characterized by deletion of all or part of the RB1 gene.

### Dosing Regimes of INX-315

The invention described herein provides methods of treating a human patient in need thereof, for example a human patient having a CDK2-mediated cancer including but not limited to hormone receptor-positive (HR+), HER2⁻ breast cancer, ovarian cancer, or small cell lung cancer (SCLC), comprising administration of INX-315, or a pharmaceutically acceptable salt thereof, which provide plasma and whole blood profile ranges of INX-315 in humans. In another embodiment, the invention includes administering INX-315 to a human patient with a CDK2-mediated cancer that is sensitive to a CDK2 inhibitor. In yet another embodiment, the invention includes administering INX-315 to a human patient with a CDK2-mediated cancer that overexpresses cyclin E.

In one aspect, provided herein is a method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 1,250 up to about 50,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean AUC is at least about 6,000. In some embodiments, the mean AUC is at least about 9,000.

In another aspect, provided herein is a method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 75 up to about 2,400 as measured on day 1 of dosing, wherein the pharmaceutical composition comprises an amorphous spray dried dispersion (ASD) comprising between about 100 mg and about 500 mg of INX-315 and wherein the amount of INX-315 in the ASD is between 20% and 50% by weight. In some embodiments, the mean Cₘₐₓ is at least about 350. In some embodiments, the mean Cₘₐₓ is at least about 525.

In another aspect, provided herein is a method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 20 up to about 1,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean Cₘᵢₙ is at least about 120. In some embodiments, the mean Cₘᵢₙ is at least about 180.

In another aspect, provided herein is a method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean free minimum plasma concentration (Cₘᵢₙ^{free}) (ng/mL) of at least about 4.0 up to about 48.0 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean Cₘᵢₙ^{free} is at least about 10.0. In some embodiments, the mean Cₘᵢₙ^{free} is at least about 15.0.

In another aspect, provided herein is a method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 1,250 up to about 56,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean AUC is at least about 11,000. In some embodiments, the mean AUC is at least about 16,500.

In another aspect, provided herein is a method for the treatment of a human with a CDK2-mediated cancer comprising administering an effective amount of a pharmaceutical composition comprising a compound of structure INX-315, or a pharmaceutically acceptable salt thereof, to the human in need thereof, which results in a mean maximum plasma concentration (Cₘₐₓ) (ng/mL) of at least about 90 up to about 4,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean Cₘₐₓ is at least about 700. In some embodiments, the mean Cₘₐₓ is at least about 1,050.

In another aspect, provided herein is a method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 100 up to about 2,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean Cₘᵢₙ is at least about 250. In some embodiments, the mean Cₘᵢₙ is at least about 375.

In another aspect, provided herein is a method for the treatment of a human with a CDK2-mediated cancer comprising administering an effective amount of a pharmaceutical composition comprising a compound of structure INX-315, or a pharmaceutically acceptable salt thereof, to the human in need thereof, which results in a mean free minimum plasma concentration (Cₘᵢₙ^{free}) (ng/mL) of at least about 6.0 up to about 80.0 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean Cₘᵢₙ^{free} is at least about 14.0. In some embodiments, the mean Cₘᵢₙ^{free} is at least about 21.0.

In another aspect, provided herein is a method for the treatment of a human with a CDK2-mediated cancer comprising administering an effective amount of a pharmaceutical composition comprising a compound of structure INX-315, or a pharmaceutically acceptable salt thereof, to the human in need thereof, which results in a mean whole blood area under the curve (AUC) (ng*hr/mL) of at least about 25,000 up to about 480,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean AUC is at least about 80,000. In some embodiments, the mean AUC is at least about 120,000.

In another aspect, provided herein is a method for the treatment of a human with a CDK2-mediated cancer comprising administering an effective amount of a pharmaceutical composition comprising a compound of structure INX-315, or a pharmaceutically acceptable salt thereof, to the human in need thereof, which results in a mean maximum whole blood concentration (Cₘₐₓ) (ng/mL) of at least about 2,000 up to about 28,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean Cₘₐₓ is at least about 4,500. In some embodiments, the mean Cₘₐₓ is at least about 6,750.

In another aspect, provided herein is a method for the treatment of a human with a CDK2-mediated cancer comprising administering an effective amount of a pharmaceutical composition comprising a compound of structure INX-315, or a pharmaceutically acceptable salt thereof, to the human in need thereof, which results in a mean minimum whole blood concentration (Cₘᵢₙ) (ng/mL) of at least about 500 up to about 14,400 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean Cₘᵢₙ is at least about 1,800. In some embodiments, the mean Cₘᵢₙ is at least about 2,700.

In another aspect, provided herein is a method for the treatment of a human with a CDK2-mediated cancer comprising administering an effective amount of a pharmaceutical composition comprising a compound of structure INX-315, or a pharmaceutically acceptable salt thereof, to the human in need thereof, which results in a mean whole blood area under the curve (AUC) (ng*hr/mL) of at least about 25,000 up to about 800,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean AUC is at least about 120,000. In some embodiments, the mean AUC is at least about 180,000.

In another aspect, provided herein is a method for the treatment of a human with a CDK2-mediated cancer comprising administering an effective amount of a pharmaceutical composition comprising a compound of structure INX-315, or a pharmaceutically acceptable salt thereof, to the human in need thereof, which results in a mean maximum whole blood concentration (Cₘₐₓ) (ng/mL) of at least about 3,000 up to about 56,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean Cₘₐₓ is at least about 7,000. In some embodiments, the mean Cₘₐₓ is at least about 10,500.

In another aspect, provided herein is a method for the treatment of a human with a CDK2-mediated cancer comprising administering an effective amount of a pharmaceutical composition comprising a compound of structure INX-315, or a pharmaceutically acceptable salt thereof, to the human in need thereof, which results in a mean minimum whole blood concentration (Cₘᵢₙ) (ng/mL) of at least about 1,000 up to about 24,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the mean Cₘᵢₙ is at least about 2,600. In some embodiments, the mean Cₘᵢₙ is at least about 3,900.

In another aspect, provided herein is a method for the treatment of a human with a CDK2-mediated cancer comprising administering an effective amount of a pharmaceutical composition comprising a compound of structure INX-315, or a pharmaceutically acceptable salt thereof, to the human in need thereof, which results in a mean plasma Cₘₐₓ to Cₘᵢₙ ratio of less than about 5 but greater than about 1.25, wherein the pharmaceutical composition comprises an amorphous spray dried dispersion (ASD) comprising between about 100 mg and about 500 mg of INX-315, and wherein the amount of INX-315 in the ASD is between 20% and 50% by weight. In some embodiments, the Cₘₐₓ to Cₘᵢₙ ratio is less than 3. In some embodiments, the Cₘₐₓ to Cₘᵢₙ ratio is less than 2.25. In some embodiments, the wherein the Cₘₐₓ to Cₘᵢₙ ratio is measured in plasma. In some embodiments, the wherein the Cₘₐₓ to Cₘᵢₙ ratio is measured in whole blood.

In some embodiments, t_{1/2} is between about 5 hours and about 32 hours. In some embodiments, the t_{1/2} is between about 8 hours and about 28 hours. In some embodiments, the t_{1/2} is between about 12 hours and about 18 hours. In some embodiments, the t_{1/2} is about 14 hours. In some embodiments, the t_{1/2} is about 15 hours. In some embodiments, the t_{1/2} is about 16 hours. In some embodiments, INX-315 is administered once-a-day. In some embodiments, INX-315 is orally administered. In some embodiments, the amount of INX-315 in the ASD is about 40%. In some embodiments, the total amount of INX-315 in the pharmaceutical composition is about 100 mg. In some embodiments, the total amount of INX-315 in the pharmaceutical composition is about 125 mg. In some embodiments, the total amount of INX-315 in the pharmaceutical composition is about 150 mg. In some embodiments, the total amount of INX-315 in the pharmaceutical composition is about 175 mg. In some embodiments, the total amount of INX-315 in the pharmaceutical composition is about 200 mg. In some embodiments, the total amount of INX-315 in the pharmaceutical composition is about 225 mg. In some embodiments, the total amount of INX-315 in the pharmaceutical composition is about 250 mg. In some embodiments, the total amount of INX-315 in the pharmaceutical composition is about 275 mg. In some embodiments, the total amount of INX-315 in the pharmaceutical composition is about 300 mg. In some embodiments, the total amount of INX-315 in the pharmaceutical composition is about 325 mg. In some embodiments, the total amount of INX-315 in the pharmaceutical composition is about 350 mg. In some embodiments, the total amount of INX-315 in the pharmaceutical composition is about 375 mg. In some embodiments, the total amount of INX-315 in the pharmaceutical composition is about 400 mg. In some embodiments, the total amount of INX-315 in the pharmaceutical composition is about 425 mg. In some embodiments, the total amount of INX-315 in the pharmaceutical composition is about 450 mg. In some embodiments, the total amount of INX-315 in the pharmaceutical composition is about 475 mg. In some embodiments, the total amount of INX-315 in the pharmaceutical composition is about 500 mg.

In some embodiments, the pharmaceutical composition further comprises a precipitation preventer. In some embodiments, the amount of the precipitation preventer in the ASD is between 40-70% by weight. In some embodiments, the amount of the precipitation preventer in the ASD is about 60% by weight. In some embodiments, the precipitation preventer comprises a hydroxypropyl methylcellulose acetate succinate (HPMCAS) selected from HPMCAS-L, HPMCAS-LF, HPMCAS-LG, HPMCAS-M, HPMCAS-MF, HPMCAS-MG, HPMCAS-H, HPMCAS-HF, HPMCAS-HG, or HPMCAS-E3. In some embodiments, the HPMCAS is HPMCAS-HG. In some embodiments, the pharmaceutical composition further comprises one or more additional pharmaceutically acceptable excipients.

In some embodiments, the INX-315 is administered at a dose of about 100, about 125, about 150, or about 175. In some embodiments, the INX-315 is administered at a dose of about 200 mg, about 225 mg, about 250 mg, or about 275 mg. In other embodiments, the INX-315 is administered at a dose of about 300 mg, about 325 mg, about 350 mg, or about 375 mg. In some embodiments, the INX-315 is administered at a dose of about 400 mg, about 425 mg, about 450, about 475 mg, or about 500 mg. In some embodiments, the INX-315 is orally administered. In some embodiments, the INX-315 is formulated as an amorphous spray-dried dispersion (ASD).

In some embodiments, the administration of INX-315 or a pharmaceutically acceptable salt thereof in a human patient in need thereof results in a mean Cₘₐₓ to Cₘᵢₙ ratio of less than about 5 but greater than about 1.25. In some embodiments, the administration of INX-315 or a pharmaceutically acceptable salt thereof in a human patient in need thereof results in a mean Cₘₐₓ to Cₘᵢₙ ratio of less than about 4.75 but greater than about 1.5. In some embodiments, the administration of INX-315 or a pharmaceutically acceptable salt thereof in a human patient in need thereof results in a mean Cₘₐₓ to Cₘᵢₙ ratio of less than about 4.5 but greater than about 1.75. In some embodiments, the administration of INX-315 or a pharmaceutically acceptable salt thereof in a human patient in need thereof results in a mean Cₘₐₓ to Cₘᵢₙ ratio of less than about 4.25 but greater than about 2. In some embodiments, the administration of INX-315 or a pharmaceutically acceptable salt thereof in a human patient in need thereof results in a mean Cₘₐₓ to Cₘᵢₙ ratio of about 2.2. In some embodiments, the INX-315 is administered once-a-day. In some embodiments, the INX-315 is administered at a dose of between about 100 mg and about 500 mg. In some embodiments, the INX-315 is administered at a dose of about 100 mg, about 125 mg, or about 150 mg. In some embodiments, the INX-315 is administered at a dose of about 200 mg, about 225 mg, or about 250 mg. In some embodiments, the INX-315 is administered at a dose of about 300 mg, about 325 mg, or about 350 mg. In some embodiments, the INX-315 is administered at a dose of about 400 mg, about 425 mg, or about 450 mg. In some embodiments, the INX-315 is orally administered. In some embodiments, the INX-315 is formulated as a spray dried dispersion. In some embodiments, the Cₘₐₓ to Cₘᵢₙ ratio is measured in plasma. In some embodiments, the Cₘₐₓ to Cₘᵢₙ ratio is measured in whole blood.

In some embodiments, the administration of INX-315 or a pharmaceutically acceptable salt thereof in a human patient in need thereof results in a t_{1/2} of between about 12 hours and about 24 hours. In some embodiments, the administration of INX-315 or a pharmaceutically acceptable salt thereof in a human patient in need thereof results in a t_{1/2} of between about 12 hours and about 18 hours. In some embodiments, the administration of INX-315 or a pharmaceutically acceptable salt thereof in a human patient in need thereof results in a t_{1/2} of between about 12.5 hours and about 17.5 hours. In some embodiments, the administration of INX-315 or a pharmaceutically acceptable salt thereof in a human patient in need thereof results in a t_{1/2} of between about 13 hours and about 17 hours. In some embodiments, the administration of INX-315 or a pharmaceutically acceptable salt thereof in a human patient in need thereof results in a t_{1/2} of between about 13.5 hours and about 16.5 hours. In some embodiments, the administration of INX-315 or a pharmaceutically acceptable salt thereof in a human patient in need thereof results in a t_{1/2} of between about 14 hours and about 16 hours. In some embodiments, the administration of INX-315 or a pharmaceutically acceptable salt thereof in a human patient in need thereof results in a t_{1/2} of about 14 hours. In some embodiments, the administration of INX-315 or a pharmaceutically acceptable salt thereof in a human patient in need thereof results in a t_{1/2} of about 15 hours. In some embodiments, the administration of INX-315 or a pharmaceutically acceptable salt thereof in a human patient in need thereof results in a t_{1/2} of about 16 hours. In some embodiments, the t₁/2 of INX-315 is measured for plasma INX-315. In some embodiments, the t₁/2 of INX-315 is measured for whole blood INX-315.

In different embodiments, the administration of INX-315 or a pharmaceutically acceptable salt thereof in a human patient in need thereof results in a t_{1/2} of between about 8 hours and about 28 hours. In different embodiments, the administration of INX-315 or a pharmaceutically acceptable salt thereof in a human patient in need thereof results in a t_{1/2} of between about 10 hours and about 26 hours. In different embodiments, the administration of INX-315 or a pharmaceutically acceptable salt thereof in a human patient in need thereof results in a t_{1/2} of between about 12 hours and about 26 hours. In different embodiments, the administration of INX-315 or a pharmaceutically acceptable salt thereof in a human patient in need thereof results in a t_{1/2} of between about 13 hours and about 25 hours. In different embodiments, the administration of INX-315 or a pharmaceutically acceptable salt thereof in a human patient in need thereof results in a t_{1/2} of between about 14 hours and about 24 hours. In different embodiments, the administration of INX-315 or a pharmaceutically acceptable salt thereof in a human patient in need thereof results in a t_{1/2} of between about 15 hours and about 23 hours. In different embodiments, the administration of INX-315 or a pharmaceutically acceptable salt thereof in a human patient in need thereof results in a t_{1/2} of about 14 hours. In different embodiments, the administration of INX-315 or a pharmaceutically acceptable salt thereof in a human patient in need thereof results in a t_{1/2} of about 15 hours. In different embodiments, the administration of INX-315 or a pharmaceutically acceptable salt thereof in a human patient in need thereof results in a t_{1/2} of about 16 hours. In different embodiments, the administration of INX-315 or a pharmaceutically acceptable salt thereof in a human patient in need thereof results in a t_{1/2} of about 17 hours. In different embodiments, the administration of INX-315 or a pharmaceutically acceptable salt thereof in a human patient in need thereof results in a t_{1/2} of about 18 hours. In different embodiments, the administration of INX-315 or a pharmaceutically acceptable salt thereof in a human patient in need thereof results in a t_{1/2} of about 19 hours. In different embodiments, the administration of INX-315 or a pharmaceutically acceptable salt thereof in a human patient in need thereof results in a t_{1/2} of about 20 hours. In different embodiments, the administration of INX-315 or a pharmaceutically acceptable salt thereof in a human patient in need thereof results in a t_{1/2} of about 21 hours. In different embodiments, the administration of INX-315 or a pharmaceutically acceptable salt thereof in a human patient in need thereof results in a t_{1/2} of about 22 hours. In some embodiments, the t_{1/2} of INX-315 is measured for plasma INX-315. In some embodiments, the t_{1/2} of INX-315 is measured for whole blood INX-315.

In different embodiments, the administration of INX-315 or a pharmaceutically acceptable salt thereof in a human patient in need thereof results in a t_{1/2} of between about 5 hours and about 32 hours. In different embodiments, the administration of INX-315 or a pharmaceutically acceptable salt thereof in a human patient in need thereof results in a t_{1/2} of between about 8 hours and about 28 hours. In different embodiments, the administration of INX-315 or a pharmaceutically acceptable salt thereof in a human patient in need thereof results in a t_{1/2} of between about 12 hours and about 24 hours. In some embodiments, the t_{1/2} of INX-315 is measured for plasma INX-315. In some embodiments, the t_{1/2} of INX-315 is measured for whole blood INX-315.

In some embodiments, the method of treating provides a red blood cell-to-plasma ratio (K_{RBCP}) of greater than about 12 but less than about 50. In some embodiments, the K_{RBC/P} is between about 15 and about 45. In some embodiments, the K_{RBC/P} is between about 20 and about 40. In some embodiments, the method of treating provides a K_{RBC/P} of greater than about 12 but less than 40. In some embodiments, the method of treating provides a K_{RBC/P} of greater than about 15 but less than 40. In some embodiments, the method of treating provides a K_{RBC/P} of greater than about 20 but less than 40. In some embodiments, the method of treating provides a K_{RBC/P} of greater than about 25 but less than 40.

In some embodiments, the method of treating provides a whole blood-to-plasma ratio (K_{WB/P}) of greater than about 4 and less than about 20. In some embodiments, the K_{WB/P} is between about 6 and about 18. In some embodiments, the K_{WB/P} is between about 10 and about 14. In some embodiments, the method of treating provides a K_{WB/P} of greater than about 4 but less than 14. In some embodiments, the method of treating provides a K_{WB/P} of greater than about 6 but less than 14. In some embodiments, the method of treating provides a K_{WB/P} of greater than about 8 but less than 14. In some embodiments, the method of treating provides a K_{WB/P} of greater than about 10 but less than 14.

Importantly, these PK/PD parameters indicate that sufficiently effective drug levels of INX-315 can be achieved to treat CDK2 inhibitor susceptible cancers with daily oral dosing without a significant drug spike on human administration, which minimizes adverse and/or off-target events as seen with other CDK2 inhibitors currently in development. The INX-315 dosage regimes have been identified in a Phase I human clinical trial in the United States under the supervision of the U.S. Food and Drug Administration (FDA) for the treatment of humans with recurrent advanced/metastatic cancer, including hormone receptor positive (HR+)/Human Epidermal Growth Factor Receptor 2 negative (HER2−) breast cancer who progressed on a prior cyclin-dependent kinase 4/6 inhibitor (CDK4/6i) regimen, and solid tumors (with and without CCNE1 amplification) who progressed on standard of care treatment. INX-315 can also be used to treat other cancers that can be treated with a CDK2 inhibitor, and especially those cancers that show an overexpression of cyclin E or that are otherwise resistant to a CDK4/6 inhibitor (see, e.g., FIG. 16B).

The results described here are surprising because two other selective CDK2 inhibitors presently being developed in the clinic, BLU-222 and PF-07104091, have significantly higher maximum drug plasma concentration (Cₘₐₓ) values and cannot achieve the pharmacokinetic parameters described herein for INX-315, and most notably during a once-a-day administration dosage regime. The present methods provide surprising treatment against cancers while significantly reducing therapy-limiting off-target side effects, such as diarrhea and vomiting, which are associated with other CDK2 inhibitors. These dosage regimes for administration in a human are particularly useful in therapeutic regimens for the long-term treatment of, for example, HR+/IER2− breast cancer, ovarian cancer, and solid tumors (with and without CCNE1 amplification) who progressed on CDK4/6i regimens and/or prior standard of care treatments. These improved dosage regimes are facilitated by a long half-life (t_{1/2}) of INX-315 relative to other CDK2 inhibitors (due to the red blood cell depot effect) without significant accumulation, allowing for continuous dosing INX-315 for extended periods of time while benefiting from the low Cₘₐₓ to Cₘᵢₙ ratio.

Because of the low Cₘₐₓ to Cₘᵢₙ ratio, INX-315 can be administered over an extended period, for example, 2, 3, 4, 5, 6 or more months, or even indefinitely as authorized by the health care provider, until the cancer or other disorder is successfully treated or other therapy is used. In some embodiments, INX-315 can be given daily for an extended period as prescribed by a health care practitioner, for example, once daily, which improved patient compliance. In some embodiments, INX-315 or its pharmaceutically acceptable salt thereof, is orally administered once daily (QD) at a dose of between, for example, about 100 mg and 400 or 500 mg (for example, 100, 125, 150, 175, 200, 250, 300 or 400 mg/day) enhancing patient compliance of the dosing regimens described herein.

In one aspect, the administration of INX-315 or a pharmaceutically acceptable salt thereof in a human patient in need thereof results in a mean plasma area under the curve (AUC) (ng*hr/mL) of between about 2750 ng*hr/mL and about 28000 ng*hr/mL as measured on day 1 of dosing. In a different aspect, the administration of INX-315 or a pharmaceutically acceptable salt thereof in a human patient in need thereof results in a mean plasma area under the curve (AUC) (ng*hr/mL) of between about 1250 ng*hr/mL and about 20000 ng*hr/mL as measured on day 1 of dosing. In a different aspect, the administration of INX-315 or a pharmaceutically acceptable salt thereof in a human patient in need thereof results in a mean plasma area under the curve (AUC) (ng*hr/mL) of between about 5000 ng*hr/mL and about 50000 ng*hr/mL as measured on day 1 of dosing. In some embodiments, the mean plasma AUC is about or greater than 2750 ng*hr/mL, about or greater than 3000 ng*hr/mL, or about or greater than 3250 ng*hr/mL but less than about 7000 ng*hr/mL. In some embodiments, the mean AUC is about or greater than 2750 ng*hr/mL but less than about 7000 ng*hr/mL. In some embodiments, the mean AUC is about or greater than 3000 ng*hr/mL but less than about 7000 ng*hr/mL. In some embodiments, the mean AUC is about or greater than 3250 ng*hr/mL but less than about 7000 ng*hr/mL. In different embodiments, the mean plasma AUC is about or greater than 1250 ng*hr/mL, about or greater than 1500 ng*hr/mL, or about or greater than 1750 ng*hr/mL but less than about 5000 ng*hr/mL. In different embodiments, the mean AUC is about or greater than 1250 ng*hr/mL but less than about 5000 ng*hr/mL. In different embodiments, the mean AUC is about or greater than 1500 ng*hr/mL but less than about 5000 ng*hr/mL. In different embodiments, the mean AUC is about or greater than 1750 ng*hr/mL but less than about 5000 ng*hr/mL. In different embodiments, the mean plasma AUC is about or greater than 5000 ng*hr/mL, about or greater than 6000 ng*hr/mL, or about or greater than 7000 ng*hr/mL but less than about 12500 ng*hr/mL. In different embodiments, the mean AUC is about or greater than 5000 ng*hr/mL but less than about 12500 ng*hr/mL. In different embodiments, the mean AUC is about or greater than 6000 ng*hr/mL but less than about 12500 ng*hr/mL. In different embodiments, the mean AUC is about or greater than 7000 ng*hr/mL but less than about 12500 ng*hr/mL. In some embodiments, the mean plasma AUC is about or greater than 5500 ng*hr/mL, about or greater than 6000 ng*hr/mL, or about or greater than 6500 ng*hr/mL but less than about 14000 ng*hr/mL. In some embodiments, the mean AUC is about or greater than 5500 ng*hr/mL but less than about 14000 ng*hr/mL. In some embodiments, the mean AUC is about or greater than 6000 ng*hr/mL but less than about 14000 ng*hr/mL. In some embodiments, the mean AUC is about or greater than 6500 ng*hr/mL but less than about 14000 ng*hr/mL. In different embodiments, the mean plasma AUC is about or greater than 2500 ng*hr/mL, about or greater than 3000 ng*hr/mL, or about or greater than 3500 ng*hr/mL but less than about 10000 ng*hr/mL. In different embodiments, the mean AUC is about or greater than 2500 ng*hr/mL but less than about 10000 ng*hr/mL. In different embodiments, the mean AUC is about or greater than 3000 ng*hr/mL but less than about 10000 ng*hr/mL. In different embodiments, the mean AUC is about or greater than 3500 ng*hr/mL but less than about 10000 ng*hr/mL. In different embodiments, the mean plasma AUC is about or greater than 10000 ng*hr/mL, about or greater than 12000 ng*hr/mL, or about or greater than 14000 ng*hr/mL but less than about 25000 ng*hr/mL. In different embodiments, the mean AUC is about or greater than 10000 ng*hr/mL but less than about 25000 ng*hr/mL. In different embodiments, the mean AUC is about or greater than 12000 ng*hr/mL but less than about 25000 ng*hr/mL. In different embodiments, the mean AUC is about or greater than 14000 ng*hr/mL but less than about 25000 ng*hr/mL. In some embodiments, the mean plasma AUC is about or greater than 8250 ng*hr/mL, about or greater than 9000 ng*hr/mL, or about or greater than 9750 ng*hr/mL but less than about 21000 ng*hr/mL. In some embodiments, the mean AUC is about or greater than 8250 ng*hr/mL but less than about 21000 ng*hr/mL. In some embodiments, the mean AUC is about or greater than 9000 ng*hr/mL but less than about 21000 ng*hr/mL. In some embodiments, the mean AUC is about or greater than 9750 ng*hr/mL but less than about 21000 ng*hr/mL. In different embodiments, the mean plasma AUC is about or greater than 3750 ng*hr/mL, about or greater than 4500 ng*hr/mL, or about or greater than 5250 ng*hr/mL but less than about 15000 ng*hr/mL. In different embodiments, the mean AUC is about or greater than 3750 ng*hr/mL but less than about 15000 ng*hr/mL. In different embodiments, the mean AUC is about or greater than 4500 ng*hr/mL but less than about 15000 ng*hr/mL. In different embodiments, the mean AUC is about or greater than 5250 ng*hr/mL but less than about 15000 ng*hr/mL. In different embodiments, the mean plasma AUC is about or greater than 15000 ng*hr/mL, about or greater than 18000 ng*hr/mL, or about or greater than 21000 ng*hr/mL but less than about 37500 ng*hr/mL. In different embodiments, the mean AUC is about or greater than 15000 ng*hr/mL but less than about 37500 ng*hr/mL. In different embodiments, the mean AUC is about or greater than 18000 ng*hr/mL but less than about 37500 ng*hr/mL. In different embodiments, the mean AUC is about or greater than 21000 ng*hr/mL but less than about 37500 ng*hr/mL. In some embodiments, the mean plasma AUC is about or greater than 11000 ng*hr/mL, about or greater than 12000 ng*hr/mL, or about or greater than 13000 ng*hr/mL but less than about 28000 ng*hr/mL. In some embodiments, the mean AUC is about or greater than 11000 ng*hr/mL but less than about 28000 ng*hr/mL. In some embodiments, the mean AUC is about or greater than 12000 ng*hr/mL but less than about 28000 ng*hr/mL. In some embodiments, the mean AUC is about or greater than 13000 ng*hr/mL but less than about 28000 ng*hr/mL. Indifferent embodiments, the mean plasma AUC is about or greater than 5000 ng*hr/mL, about or greater than 5750 ng*hr/mL, or about or greater than 6500 ng*hr/mL but less than about 20000 ng*hr/mL. In different embodiments, the mean AUC is about or greater than 5000 ng*hr/mL but less than about 20000 ng*hr/mL. In different embodiments, the mean AUC is about or greater than 5750 ng*hr/mL but less than about 20000 ng*hr/mL. In different embodiments, the mean AUC is about or greater than 6500 ng*hr/mL but less than about 20000 ng*hr/mL. In different embodiments, the mean plasma AUC is about or greater than 20000 ng*hr/mL, about or greater than 24000 ng*hr/mL, or about or greater than 28000 ng*hr/mL but less than about 50000 ng*hr/mL. In different embodiments, the mean AUC is about or greater than 20000 ng*hr/mL but less than about 50000 ng*hr/mL. In different embodiments, the mean AUC is about or greater than 24000 ng*hr/mL but less than about 50000 ng*hr/mL. In different embodiments, the mean AUC is about or greater than 28000 ng*hr/mL but less than about 50000 ng*hr/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean maximum plasma concentration (Cₘₐₓ) (ng/mL) in humans of between about 150 ng/mL and about 2000 ng/mL as measured on day 1 of dosing. In a different aspect, the administration of INX-315 or a pharmaceutically acceptable salt thereof in a human patient in need thereof results in a mean maximum plasma concentration (Cₘₐₓ) (ng/mL) in humans of between about 100 ng/mL and about 1600 ng/mL as measured on day 1 of dosing. In a different aspect, the administration of INX-315 or a pharmaceutically acceptable salt thereof in a human patient in need thereof results in a mean maximum plasma concentration (Cₘₐₓ) (ng/mL) in humans of between about 75 ng/mL and about 2400 ng/mL as measured on day 1 of dosing. In some embodiments, the mean Cₘₐₓ is about or greater than 150 ng/mL, about or greater than 175 ng/mL, or about or greater than 200 ng/mL but less than about 500 ng/mL. In some embodiments, the Cₘₐₓ is about or greater than 150 ng/mL but less than about 500 ng/mL. In some embodiments, the Cₘₐₓ is about or greater than 175 ng/mL but less than about 500 ng/mL. In some embodiments, the Cₘₐₓ is about or greater than 200 ng/mL but less than about 500 ng/mL. In different embodiments, the mean Cₘₐₓ is about or greater than 100 ng/mL, about or greater than 125 ng/mL, or about or greater than 150 ng/mL but less than about 400 ng/mL. In different embodiments, the Cₘₐₓ is about or greater than 100 ng/mL but less than about 400 ng/mL. In different embodiments, the Cₘₐₓ is about or greater than 125 ng/mL but less than about 400 ng/mL. In different embodiments, the Cₘₐₓ is about or greater than 150 ng/mL but less than about 400 ng/mL. In different embodiments, the mean Cₘₐₓ is about or greater than 75 ng/mL, about or greater than 180 ng/mL, or about or greater than 210 ng/mL but less than about 600 ng/mL. In different embodiments, the Cₘₐₓ is about or greater than 75 ng/mL but less than about 600 ng/mL. In different embodiments, the Cₘₐₓ is about or greater than 180 ng/mL but less than about 600 ng/mL. In different embodiments, the Cₘₐₓ is about or greater than 210 ng/mL but less than about 600 ng/mL. In some embodiments, the mean Cₘₐₓ is about or greater than 300 ng/mL, about or greater than 350 ng/mL, or about or greater than 400 ng/mL but less than about 1000 ng/mL. In some embodiments, the Cₘₐₓ is about or greater than 300 ng/mL but less than about 1000 ng/mL. In some embodiments, the Cₘₐₓ is about or greater than 350 ng/mL but less than about 1000 ng/mL. In some embodiments, the Cₘₐₓ is about or greater than 400 ng/mL but less than about 1000 ng/mL. In different embodiments, the mean Cₘₐₓ is about or greater than 200 ng/mL, about or greater than 250 ng/mL, or about or greater than 300 ng/mL but less than about 800 ng/mL. In different embodiments, the Cₘₐₓ is about or greater than 200 ng/mL but less than about 800 ng/mL. In different embodiments, the Cₘₐₓ is about or greater than 250 ng/mL but less than about 800 ng/mL. In different embodiments, the Cₘₐₓ is about or greater than 300 ng/mL but less than about 800 ng/mL. In different embodiments, the mean Cₘₐₓ is about or greater than 150 ng/mL, about or greater than 360 ng/mL, or about or greater than 420 ng/mL but less than about 1200 ng/mL. In different embodiments, the Cₘₐₓ is about or greater than 150 ng/mL but less than about 1200 ng/mL. In different embodiments, the Cₘₐₓ is about or greater than 360 ng/mL but less than about 1200 ng/mL. In different embodiments, the Cₘₐₓ is about or greater than 420 ng/mL but less than about 1200 ng/mL. In some embodiments, the mean Cₘₐₓ is about or greater than 450 ng/mL, about or greater than 525 ng/mL, or about or greater than 600 ng/mL but less than about 1500 ng/mL. In some embodiments, the Cₘₐₓ is about or greater than 450 ng/mL but less than about 1500 ng/mL. In some embodiments, the Cₘₐₓ is about or greater than 525 ng/mL but less than about 1500 ng/mL. In some embodiments, the Cₘₐₓ is about or greater than 600 ng/mL but less than about 1500 ng/mL. In different embodiments, the mean Cₘₐₓ is about or greater than 300 ng/mL, about or greater than 375 ng/mL, or about or greater than 450 ng/mL but less than about 1200 ng/mL. In different embodiments, the Cₘₐₓ is about or greater than 300 ng/mL but less than about 1200 ng/mL. In different embodiments, the Cₘₐₓ is about or greater than 375 ng/mL but less than about 1200 ng/mL. In different embodiments, the Cₘₐₓ is about or greater than 450 ng/mL but less than about 1200 ng/mL. In different embodiments, the mean Cₘₐₓ is about or greater than 225 ng/mL, about or greater than 540 ng/mL, or about or greater than 630 ng/mL but less than about 1800 ng/mL. In different embodiments, the Cₘₐₓ is about or greater than 225 ng/mL but less than about 1800 ng/mL. In different embodiments, the Cₘₐₓ is about or greater than 540 ng/mL but less than about 1800 ng/mL. In different embodiments, the Cₘₐₓ is about or greater than 630 ng/mL but less than about 1800 ng/mL. In some embodiments, the mean Cₘₐₓ is about or greater than 600 ng/mL, about or greater than 700 ng/mL, or about or greater than 800 ng/mL but less than about 2000 ng/mL. In some embodiments, the Cₘₐₓ is about or greater than 600 ng/mL but less than about 2000 ng/mL. In some embodiments, the Cₘₐₓ is about or greater than 700 ng/mL but less than about 2000 ng/mL. In some embodiments, the Cₘₐₓ is about or greater than 800 ng/mL but less than about 2000 ng/mL. In different embodiments, the mean Cₘₐₓ is about or greater than 400 ng/mL, about or greater than 500 ng/mL, or about or greater than 600 ng/mL but less than about 1600 ng/mL. In different embodiments, the Cₘₐₓ is about or greater than 400 ng/mL but less than about 1600 ng/mL. In different embodiments, the Cₘₐₓ is about or greater than 500 ng/mL but less than about 1600 ng/mL. In different embodiments, the Cₘₐₓ is about or greater than 600 ng/mL but less than about 1600 ng/mL. In different embodiments, the mean Cₘₐₓ is about or greater than 300 ng/mL, about or greater than 720 ng/mL, or about or greater than 840 ng/mL but less than about 2400 ng/mL. In different embodiments, the Cₘₐₓ is about or greater than 300 ng/mL but less than about 2400 ng/mL. In different embodiments, the Cₘₐₓ is about or greater than 720 ng/mL but less than about 2400 ng/mL. In different embodiments, the Cₘₐₓ is about or greater than 840 ng/mL but less than about 2400 ng/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean minimum plasma concentration (Cₘᵢₙ) (ng/mL) of between about 50 ng/mL and about 1000 ng/mL as measured on day 1 of dosing. In a different aspect, the administration of INX-315 or a pharmaceutically acceptable salt thereof in a human patient in need thereof results in a mean minimum plasma concentration (Cₘᵢₙ) (ng/mL) of between about 20 ng/mL and about 600 ng/mL as measured on day 1 of dosing. In some embodiments, the mean plasma Cₘᵢₙ is about or greater than 50 ng/mL, about or greater than 60 ng/mL, or about or greater than 75 ng/mL but less than about 250 ng/mL. In some embodiments, the Cₘᵢₙ is about or greater than 50 ng/mL but less than about 250 ng/mL. In some embodiments, the Cₘᵢₙ is about or greater than 60 ng/mL but less than about 250 ng/mL. In some embodiments, the Cₘᵢₙ is about or greater than 75 ng/mL but less than about 250 ng/mL. In different embodiments, the mean plasma Cₘᵢₙ is about or greater than 20 ng/mL, about or greater than 40 ng/mL, or about or greater than 60 ng/mL but less than about 150 ng/mL. In different embodiments, the Cₘᵢₙ is about or greater than 20 ng/mL but less than about 150 ng/mL. In different embodiments, the Cₘᵢₙ is about or greater than 40 ng/mL but less than about 150 ng/mL. In different embodiments, the Cₘᵢₙ is about or greater than 60 ng/mL but less than about 150 ng/mL. In some embodiments, the mean plasma Cₘᵢₙ is about or greater than 100 ng/mL, about or greater than 120 ng/mL, or about or greater than 150 ng/mL but less than about 500 ng/mL. In some embodiments, the Cₘᵢₙ is about or greater than 100 ng/mL but less than about 500 ng/mL. In some embodiments, the Cₘᵢₙ is about or greater than 120 ng/mL but less than about 500 ng/mL. In some embodiments, the Cₘᵢₙ is about or greater than 150 ng/mL but less than about 500 ng/mL. In different embodiments, the mean plasma Cₘᵢₙ is about or greater than 40 ng/mL, about or greater than 80 ng/mL, or about or greater than 120 ng/mL but less than about 300 ng/mL. In different embodiments, the Cₘᵢₙ is about or greater than 40 ng/mL but less than about 300 ng/mL. In different embodiments, the Cₘᵢₙ is about or greater than 80 ng/mL but less than about 300 ng/mL. In different embodiments, the Cₘᵢₙ is about or greater than 120 ng/mL but less than about 300 ng/mL. In some embodiments, the mean plasma Cₘᵢₙ is about or greater than 150 ng/mL, about or greater than 180 ng/mL, or about or greater than 225 ng/mL but less than about 750 ng/mL. In some embodiments, the Cₘᵢₙ is about or greater than 150 ng/mL but less than about 750 ng/mL. In some embodiments, the Cₘᵢₙ is about or greater than 180 ng/mL but less than about 750 ng/mL. In some embodiments, the Cₘᵢₙ is about or greater than 225 ng/mL but less than about 750 ng/mL. In different embodiments, the mean plasma Cₘᵢₙ is about or greater than 60 ng/mL, about or greater than 120 ng/mL, or about or greater than 180 ng/mL but less than about 450 ng/mL. In different embodiments, the Cₘᵢₙ is about or greater than 60 ng/mL but less than about 450 ng/mL. In different embodiments, the Cₘᵢₙ is about or greater than 120 ng/mL but less than about 450 ng/mL. In different embodiments, the Cₘᵢₙ is about or greater than 180 ng/mL but less than about 450 ng/mL. In some embodiments, the mean plasma Cₘᵢₙ is about or greater than 200 ng/mL, about or greater than 240 ng/mL, or about or greater than 300 ng/mL but less than about 1000 ng/mL. In some embodiments, the Cₘᵢₙ is about or greater than 200 ng/mL but less than about 1000 ng/mL. In some embodiments, the Cₘᵢₙ is about or greater than 240 ng/mL but less than about 1000 ng/mL. In some embodiments, the Cₘᵢₙ is about or greater than 300 ng/mL but less than about 1000 ng/mL. In different embodiments, the mean plasma Cₘᵢₙ is about or greater than 80 ng/mL, about or greater than 160 ng/mL, or about or greater than 240 ng/mL but less than about 600 ng/mL. In different embodiments, the Cₘᵢₙ is about or greater than 80 ng/mL but less than about 600 ng/mL. In different embodiments, the Cₘᵢₙ is about or greater than 160 ng/mL but less than about 600 ng/mL. In different embodiments, the Cₘᵢₙ is about or greater than 240 ng/mL but less than about 600 ng/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean free minimum plasma concentration (Cₘᵢₙ^{free}) (ng/mL) of between about 4.0 ng/mL and about 48.0 ng/mL as measured on day 1 of dosing. In some embodiments, the mean Cₘᵢₙ^{free} is about or greater than 4.0 ng/mL, about or greater than 5.0 ng/mL, or about or greater than 6.0 ng/mL but less than about 12.0 ng/mL. In some embodiments, the mean Cₘᵢₙ^{free} is about or greater than 4.0 ng/mL but less than about 12.0 ng/mL. In some embodiments, the mean Cₘᵢₙ^{free} is about or greater than 5.0 ng/mL but less than about 12.0 ng/mL. In some embodiments, the mean Cₘᵢₙ^{free} is about or greater than 6.0 ng/mL but less than about 12.0 ng/mL. In some embodiments, the mean Cₘᵢₙ^{free} is about or greater than 8.0 ng/mL, about or greater than 10.0 ng/mL, or about or greater than 12.0 ng/mL but less than about 24.0 ng/mL. In some embodiments, the mean Cₘᵢₙ^{free} is about or greater than 8.0 ng/mL but less than about 24.0 ng/mL. In some embodiments, the mean Cₘᵢₙ^{free} is about or greater than 10.0 ng/mL but less than about 24.0 ng/mL. In some embodiments, the mean Cₘᵢₙ^{free} is about or greater than 12.0 ng/mL but less than about 24.0 ng/mL. In some embodiments, the mean Cₘᵢₙ^{free} is about or greater than 12.0 ng/mL, about or greater than 15.0 ng/mL, or about or greater than 18.0 ng/mL but less than about 36.0 ng/mL. In some embodiments, the mean Cₘᵢₙ^{free} is about or greater than 12.0 ng/mL but less than about 36.0 ng/mL. In some embodiments, the mean Cₘᵢₙ^{free} is about or greater than 15.0 ng/mL but less than about 36.0 ng/mL. In some embodiments, the mean Cₘᵢₙ^{free} is about or greater than 18.0 ng/mL but less than about 36.0 ng/mL. In some embodiments, the mean Cₘᵢₙ^{free} is about or greater than 16.0 ng/mL, about or greater than 20.0 ng/mL, or about or greater than 24.0 ng/mL but less than about 48.0 ng/mL. In some embodiments, the mean Cₘᵢₙ^{free} is about or greater than 16.0 ng/mL but less than about 48.0 ng/mL. In some embodiments, the mean Cₘᵢₙ^{free} is about or greater than 20.0 ng/mL but less than about 48.0 ng/mL. In some embodiments, the mean Cₘᵢₙ^{free} is about or greater than 24.0 ng/mL but less than about 48.0 ng/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean plasma area under the curve (AUC) (ng*hr/mL) of between about 5000 ng*hr/mL and about 56000 ng*hr/mL as measured on day 15 of dosing. In a different aspect, the administration of INX-315 or a pharmaceutically acceptable salt thereof in a human patient in need thereof results in a mean plasma area under the curve (AUC) (ng*hr/mL) of between about 1250 ng*hr/mL and about 28000 ng*hr/mL as measured on day 15 of dosing. In some embodiments, the mean plasma AUC is about or greater than 5000 ng*hr/mL, about or greater than 5500 ng*hr/mL, or about or greater than 6000 ng*hr/mL but less than about 14000 ng*hr/mL. In some embodiments, the mean AUC is about or greater than 5000 ng*hr/mL but less than about 14000 ng*hr/mL. In some embodiments, the mean AUC is about or greater than 5500 ng*hr/mL but less than about 14000 ng*hr/mL. In some embodiments, the mean AUC is about or greater than 6000 ng*hr/mL but less than about 14000 ng*hr/mL. In different embodiments, the mean plasma AUC is about or greater than 1250 ng*hr/mL, about or greater than 2100 ng*hr/mL, or about or greater than 2450 ng*hr/mL but less than about 7000 ng*hr/mL. In different embodiments, the mean AUC is about or greater than 1250 ng*hr/mL but less than about 7000 ng*hr/mL. In different embodiments, the mean AUC is about or greater than 2100 ng*hr/mL but less than about 7000 ng*hr/mL. In different embodiments, the mean AUC is about or greater than 2450 ng*hr/mL but less than about 7000 ng*hr/mL. In some embodiments, the mean plasma AUC is about or greater than 10000 ng*hr/mL, about or greater than 11000 ng*hr/mL, or about or greater than 12000 ng*hr/mL but less than about 28000 ng*hr/mL. In some embodiments, the mean AUC is about or greater than 10000 ng*hr/mL but less than about 28000 ng*hr/mL. In some embodiments, the mean AUC is about or greater than 11000 ng*hr/mL but less than about 28000 ng*hr/mL. In some embodiments, the mean AUC is about or greater than 12000 ng*hr/mL but less than about 28000 ng*hr/mL. In different embodiments, the mean plasma AUC is about or greater than 2500 ng*hr/mL, about or greater than 4200 ng*hr/mL, or about or greater than 4900 ng*hr/mL but less than about 14000 ng*hr/mL. In different embodiments, the mean AUC is about or greater than 2500 ng*hr/mL but less than about 14000 ng*hr/mL. In different embodiments, the mean AUC is about or greater than 4200 ng*hr/mL but less than about 14000 ng*hr/mL. In different embodiments, the mean AUC is about or greater than 4900 ng*hr/mL but less than about 14000 ng*hr/mL. In some embodiments, the mean plasma AUC is about or greater than 15000 ng*hr/mL, about or greater than 16500 ng*hr/mL, or about or greater than 18000 ng*hr/mL but less than about 42000 ng*hr/mL. In some embodiments, the mean AUC is about or greater than 15000 ng*hr/mL but less than about 42000 ng*hr/mL. In some embodiments, the mean AUC is about or greater than 16500 ng*hr/mL but less than about 42000 ng*hr/mL. In some embodiments, the mean AUC is about or greater than 18000 ng*hr/mL but less than about 42000 ng*hr/mL. In different embodiments, the mean plasma AUC about or greater than 3750 ng*hr/mL, about or greater than 6300 ng*hr/mL, or about or greater than 7350 ng*hr/mL but less than about 21000 ng*hr/mL. In different embodiments, the mean AUC is about or greater than 3750 ng*hr/mL but less than about 21000 ng*hr/mL. In different embodiments, the mean AUC is about or greater than 6300 ng*hr/mL but less than about 21000 ng*hr/mL. In different embodiments, the mean AUC is about or greater than 7350 ng*hr/mL but less than about 21000 ng*hr/mL. In some embodiments, the mean plasma AUC is about or greater than 20000 ng*hr/mL, about or greater than 22000 ng*hr/mL, or about or greater than 24000 ng*hr/mL but less than about 56000 ng*hr/mL. In some embodiments, the mean AUC is about or greater than 20000 ng*hr/mL but less than about 56000 ng*hr/mL. In some embodiments, the mean AUC is about or greater than 22000 ng*hr/mL but less than about 56000 ng*hr/mL. In some embodiments, the mean AUC is about or greater than 24000 ng*hr/mL but less than about 56000 ng*hr/mL. In different embodiments, the mean plasma AUC about or greater than 5000 ng*hr/mL, about or greater than 8400 ng*hr/mL, or about or greater than 9800 ng*hr/mL but less than about 28000 ng*hr/mL. In different embodiments, the mean AUC is about or greater than 5000 ng*hr/mL but less than about 28000 ng*hr/mL. In different embodiments, the mean AUC is about or greater than 8400 ng*hr/mL but less than about 28000 ng*hr/mL. In different embodiments, the mean AUC is about or greater than 9800 ng*hr/mL but less than about 28000 ng*hr/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In an alternative aspect, the administration of INX-315 or a pharmaceutically acceptable salt thereof in a human patient in need thereof results in a mean maximum plasma concentration (Cₘₐₓ) (ng/mL) in humans of between about 300 ng/mL and about 4000 ng/mL as measured on day 15 of dosing. In a different aspect, the administration of INX-315 or a pharmaceutically acceptable salt thereof in a human patient in need thereof results in a mean maximum plasma concentration (Cₘₐₓ) (ng/mL) in humans of between about 90 ng/mL and about 2800 ng/mL as measured on day 15 of dosing. In some embodiments, the mean plasma Cₘₐₓ is about or greater than 300 ng/mL, about or greater than 300 ng/mL, or about or greater than 350 ng/mL but less than about 1000 ng/mL. In some embodiments, the mean Cₘₐₓ is about or greater than 300 ng/mL but less than about 1000 ng/mL. In some embodiments, the mean Cₘₐₓ is about or greater than 350 ng/mL but less than about 1000 ng/mL. In some embodiments, the mean Cₘₐₓ is about or greater than 400 ng/mL but less than about 1000 ng/mL. In different embodiments, the mean plasma Cₘₐₓ is about or greater than 90 ng/mL, about or greater than 210 ng/mL, or about or greater than 245 ng/mL but less than about 700 ng/mL. In different embodiments, the mean Cₘₐₓ is about or greater than 90 ng/mL but less than about 700 ng/mL. In different embodiments, the mean Cₘₐₓ is about or greater than 210 ng/mL but less than about 700 ng/mL. In different embodiments, the mean Cₘₐₓ is about or greater than 245 ng/mL but less than about 700 ng/mL. In some embodiments, the mean plasma Cₘₐₓ is about or greater than 600 ng/mL, about or greater than 700 ng/mL, or about or greater than 800 ng/mL but less than about 2000 ng/mL. In some embodiments, the mean Cₘₐₓ is about or greater than 600 ng/mL mL but less than about 2000 ng/mL. In some embodiments, the mean Cₘₐₓ is about or greater than 700 ng/mL mL but less than about 2000 ng/mL. In some embodiments, the mean Cₘₐₓ is about or greater than 800 ng/mL mL but less than about 2000 ng/mL. In different embodiments, the mean plasma Cₘₐₓ is about or greater than 180 ng/mL, about or greater than 420 ng/mL, or about or greater than 490 ng/mL but less than about 1400 ng/mL. In different embodiments, the mean Cₘₐₓ is about or greater than 180 ng/mL but less than about 1400 ng/mL. In different embodiments, the mean Cₘₐₓ is about or greater than 420 ng/mL but less than about 1400 ng/mL. In different embodiments, the mean Cₘₐₓ is about or greater than 490 ng/mL but less than about 1400 ng/mL. In some embodiments, the mean plasma Cₘₐₓ is about or greater than 900 ng/mL, about or greater than 1050 ng/mL, or about or greater than 1200 ng/mL but less than about 3000 ng/mL. In some embodiments, the mean Cₘₐₓ is about or greater than 900 ng/mL but less than about 3000 ng/mL. In some embodiments, the mean Cₘₐₓ is about or greater than 1050 ng/mL but less than about 3000 ng/mL. In some embodiments, the mean Cₘₐₓ is about or greater than 1200 ng/mL but less than about 3000 ng/mL. In different embodiments, the mean plasma Cₘₐₓ is about or greater than 270 ng/mL, about or greater than 630 ng/mL, or about or greater than 735 ng/mL but less than about 2100 ng/mL. In different embodiments, the mean Cₘₐₓ is about or greater than 270 ng/mL but less than about 2100 ng/mL. In different embodiments, the mean Cₘₐₓ is about or greater than 630 ng/mL but less than about 2100 ng/mL. In different embodiments, the mean Cₘₐₓ is about or greater than 735 ng/mL but less than about 2100 ng/mL. In some embodiments, the mean plasma Cₘₐₓ is about or greater than 1200 ng/mL, about or greater than 1400 ng/mL, or about or greater than 1600 ng/mL but less than about 4000 ng/mL. In some embodiments, the mean Cₘₐₓ is about or greater than 1200 ng/mL but less than about 4000 ng/mL. In some embodiments, the mean Cₘₐₓ is about or greater than 1400 ng/mL but less than about 4000 ng/mL. In some embodiments, the mean Cₘₐₓ is about or greater than 1600 ng/mL but less than about 4000 ng/mL. In different embodiments, the mean plasma Cₘₐₓ is about or greater than 360 ng/mL, about or greater than 840 ng/mL, or about or greater than 980 ng/mL but less than about 2800 ng/mL. In different embodiments, the mean Cₘₐₓ is about or greater than 360 ng/mL but less than about 2800 ng/mL. In different embodiments, the mean Cₘₐₓ is about or greater than 840 ng/mL but less than about 2800 ng/mL. In different embodiments, the mean Cₘₐₓ is about or greater than 980 ng/mL but less than about 2800 ng/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean minimum plasma concentration (Cₘᵢₙ) (ng/mL) of between about 100 ng/mL and about 2000 ng/mL as measured on day 15 of dosing. In some embodiments, the mean Cₘᵢₙ is about or greater than 100 ng/mL, about or greater than 125 ng/mL, or about or greater than 150 ng/mL but less than about 500 ng/mL. In some embodiments, the mean Cₘᵢₙ is about or greater than 100 ng/mL but less than about 500 ng/mL. In some embodiments, the mean Cₘᵢₙ is about or greater than 125 ng/mL but less than about 500 ng/mL. In some embodiments, the mean Cₘᵢₙ is about or greater than 150 ng/mL but less than about 500 ng/mL. In some embodiments, the mean Cₘᵢₙ is about or greater than 200 ng/mL, about or greater than 250 ng/mL, or about or greater than 300 ng/mL but less than about 1000 ng/mL. In some embodiments, the mean Cₘᵢₙ is about or greater than 200 ng/mL but less than about 1000 ng/mL. In some embodiments, the mean Cₘᵢₙ is about or greater than 250 ng/mL but less than about 1000 ng/mL. In some embodiments, the mean Cₘᵢₙ is about or greater than 300 ng/mL but less than about 1000 ng/mL. In some embodiments, the mean Cₘᵢₙ is about or greater than 300 ng/mL, about or greater than 375 ng/mL, or about or greater than 450 ng/mL but less than about 1500 ng/mL. In some embodiments, the mean Cₘᵢₙ is about or greater than 300 ng/mL but less than about 1500 ng/mL. In some embodiments, the mean Cₘᵢₙ is about or greater than 375 ng/mL but less than about 1500 ng/mL. In some embodiments, the mean Cₘᵢₙ is about or greater than 450 ng/mL but less than about 1500 ng/mL. In some embodiments, the mean Cₘᵢₙ is about or greater than 400 ng/mL, about or greater than 500 ng/mL, or about or greater than 600 ng/mL but less than about 2000 ng/mL. In some embodiments, the mean Cₘᵢₙ is about or greater than 400 ng/mL but less than about 2000 ng/mL. In some embodiments, the mean Cₘᵢₙ is about or greater than 500 ng/mL but less than about 2000 ng/mL. In some embodiments, the mean Cₘᵢₙ is about or greater than 600 ng/mL but less than about 2000 ng/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean free minimum plasma concentration (Cₘᵢₙ^{free}) (ng/mL) of between about 6.0 ng/mL and about 80.0 ng/mL as measured on day 15 of dosing. In some embodiments, the mean Cₘᵢₙ^{free} is about or greater than 6.0 ng/mL, about or greater than 7.0 ng/mL, about or greater than 8.0 ng/mL but less than about 20.0 ng/mL. In some embodiments, the mean Cₘᵢₙ^{free} is about or greater than 6.0 ng/mL but less than about 20.0 ng/mL. In some embodiments, the mean Cₘᵢₙ^{free} is about or greater than 7.0 ng/mL but less than about 20.0 ng/mL. In some embodiments, the mean Cₘᵢₙ^{free} is about or greater than 8.0 ng/mL but less than about 20.0 ng/mL. In some embodiments, the mean Cₘᵢₙ^{free} is about or greater than 12.0 ng/mL, about or greater than 14.0 ng/mL, about or greater than 16.0 ng/mL but less than about 40.0 ng/mL. In some embodiments, the mean Cₘᵢₙ^{free} is about or greater than 12.0 ng/mL but less than about 40.0 ng/mL. In some embodiments, the mean Cₘᵢₙ^{free} is about or greater than 14.0 ng/mL but less than about 40.0 ng/mL. In some embodiments, the mean Cₘᵢₙ^{free} is about or greater than 16.0 ng/mL but less than about 40.0 ng/mL. In some embodiments, the mean Cₘᵢₙ^{free} is about or greater than 18.0 ng/mL, about or greater than 21.0 ng/mL, about or greater than 24.0 ng/mL but less than about 60.0 ng/mL. In some embodiments, the mean Cₘᵢₙ^{free} is about or greater than 18.0 ng/mL but less than about 60.0 ng/mL. In some embodiments, the mean Cₘᵢₙ^{free} is about or greater than 21.0 ng/mL but less than about 60.0 ng/mL. In some embodiments, the mean Cₘᵢₙ^{free} is about or greater than 24.0 ng/mL but less than about 60.0 ng/mL. In some embodiments, the mean Cₘᵢₙ^{free} is about or greater than 24.0 ng/mL, about or greater than 28.0 ng/mL, about or greater than 32.0 ng/mL but less than about 80.0 ng/mL. In some embodiments, the mean Cₘᵢₙ^{free} is about or greater than 24.0 ng/mL but less than about 80.0 ng/mL. In some embodiments, the mean Cₘᵢₙ^{free} is about or greater than 28.0 ng/mL but less than about 80.0 ng/mL. In some embodiments, the mean Cₘᵢₙ^{free} is about or greater than 32.0 ng/mL but less than about 80.0 ng/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean whole blood area under the curve (AUC) (ng*hr/mL) of between about 30000 ng*hr/mL and about 480000 ng*hr/mL as measured on day 1 of dosing. In a different aspect, the administration of INX-315 or a pharmaceutically acceptable salt thereof in a human patient in need thereof results in a mean whole blood area under the curve (AUC) (ng*hr/mL) of between about 25000 ng*hr/mL and about 400000 ng*hr/mL as measured on day 1 of dosing. In some embodiments, the mean whole blood AUC is about or greater than 30000 ng*hr/mL, about or greater than 40000 ng*hr/mL, or about or greater than 50000 ng*hr/mL but less than about 120000 ng*hr/mL. In some embodiments, the mean AUC is about or greater than 30000 ng*hr/mL but less than about 120000 ng*hr/mL. In some embodiments, the mean AUC is about or greater than 40000 ng*hr/mL but less than about 120000 ng*hr/mL. In some embodiments, the mean AUC is about or greater than 50000 ng*hr/mL but less than about 120000 ng*hr/mL. In different embodiments, the mean whole blood AUC is about or greater than 25000 ng*hr/mL, about or greater than 27500 ng*hr/mL, or about or greater than 30000 ng*hr/mL but less than about 100000 ng*hr/mL. In different embodiments, the mean AUC is about or greater than 25000 ng*hr/mL but less than about 100000 ng*hr/mL. In different embodiments, the mean AUC is about or greater than 27500 ng*hr/mL but less than about 100000 ng*hr/mL. In different embodiments, the mean AUC is about or greater than 30000 ng*hr/mL but less than about 100000 ng*hr/mL. In some embodiments, the mean whole blood AUC is about or greater than 60000 ng*hr/mL, about or greater than 80000 ng*hr/mL, or about or greater than 100000 ng*hr/mL but less than about 240000 ng*hr/mL. In some embodiments, the mean AUC is about or greater than 60000 ng*hr/mL but less than about 240000 ng*hr/mL. In some embodiments, the mean AUC is about or greater than 80000 ng*hr/mL but less than about 240000 ng*hr/mL. In some embodiments, the mean AUC is about or greater than 100000 ng*hr/mL but less than about 240000 ng*hr/mL. In different embodiments, the mean whole blood AUC is about or greater than 50000 ng*hr/mL, about or greater than 55000 ng*hr/mL, or about or greater than 60000 ng*hr/mL but less than about 200000 ng*hr/mL. In different embodiments, the mean AUC is about or greater than 50000 ng*hr/mL but less than about 200000 ng*hr/mL. In different embodiments, the mean AUC is about or greater than 55000 ng*hr/mL but less than about 200000 ng*hr/mL. In different embodiments, the mean AUC is about or greater than 60000 ng*hr/mL but less than about 200000 ng*hr/mL. In some embodiments, the mean whole blood AUC is about or greater than 90000 ng*hr/mL, about or greater than 120000 ng*hr/mL, or about or greater than 150000 ng*hr/mL but less than about 360000 ng*hr/mL. In some embodiments, the mean AUC is about or greater than 90000 ng*hr/mL but less than about 360000 ng*hr/mL. In some embodiments, the mean AUC is about or greater than 120000 ng*hr/mL but less than about 360000 ng*hr/mL. In some embodiments, the mean AUC is about or greater than 150000 ng*hr/mL but less than about 360000 ng*hr/mL. In different embodiments, the mean whole blood AUC is about or greater than 75000 ng*hr/mL, about or greater than 82500 ng*hr/mL, or about or greater than 90000 ng*hr/mL but less than about 300000 ng*hr/mL. In different embodiments, the mean AUC is about or greater than 75000 ng*hr/mL but less than about 300000 ng*hr/mL. In different embodiments, the mean AUC is about or greater than 82500 ng*hr/mL but less than about 300000 ng*hr/mL. In different embodiments, the mean AUC is about or greater than 90000 ng*hr/mL but less than about 300000 ng*hr/mL. In some embodiments, the mean whole blood AUC is about or greater than 120000 ng*hr/mL, about or greater than 160000 ng*hr/mL, or about or greater than 200000 ng*hr/mL but less than about 480000 ng*hr/mL. In some embodiments, the mean AUC is about or greater than 120000 ng*hr/mL but less than about 480000 ng*hr/mL. In some embodiments, the mean AUC is about or greater than 160000 ng*hr/mL but less than about 480000 ng*hr/mL. In some embodiments, the mean AUC is about or greater than 200000 ng*hr/mL but less than about 480000 ng*hr/mL. In different embodiments, the mean whole blood AUC is about or greater than 100000 ng*hr/mL, about or greater than 110000 ng*hr/mL, or about or greater than 120000 ng*hr/mL but less than about 400000 ng*hr/mL. In different embodiments, the mean AUC is about or greater than 100000 ng*hr/mL but less than about 400000 ng*hr/mL. In different embodiments, the mean AUC is about or greater than 110000 ng*hr/mL but less than about 400000 ng*hr/mL. In different embodiments, the mean AUC is about or greater than 120000 ng*hr/mL but less than about 400000 ng*hr/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean maximum whole blood concentration (Cₘₐₓ) (ng/mL) in humans of between about 2000 ng/mL and about 28000 ng/mL as measured on day 1 of dosing. In some embodiments, the mean whole blood Cₘₐₓ is about or greater than 2000 ng/mL, about or greater than 2250 ng/mL, or about or greater than 2500 ng/mL but less than about 7000 ng/mL. In some embodiments, the mean Cₘₐₓ is about or greater than 2000 ng/mL but less than about 7000 ng/mL. In some embodiments, the mean Cₘₐₓ is about or greater than 2250 ng/mL but less than about 7000 ng/mL. In some embodiments, the mean Cₘₐₓ is about or greater than 2500 ng/mL but less than about 7000 ng/mL. In some embodiments, the mean whole blood Cₘₐₓ is about or greater than 4000 ng/mL, about or greater than 4500 ng/mL, or about or greater than 5000 ng/mL but less than about 14000 ng/mL. In some embodiments, the mean Cₘₐₓ is about or greater than 4000 ng/mL but less than about 14000 ng/mL. In some embodiments, the mean Cₘₐₓ is about or greater than 4500 ng/mL but less than about 14000 ng/mL. In some embodiments, the mean Cₘₐₓ is about or greater than 5000 ng/mL but less than about 14000 ng/mL. In some embodiments, the mean whole blood Cₘₐₓ is about or greater than 6000 ng/mL, about or greater than 6750 ng/mL, or about or greater than 7500 ng/mL but less than about 21000 ng/mL. In some embodiments, the mean Cₘₐₓ is about or greater than 6000 ng/mL but less than about 21000 ng/mL. In some embodiments, the mean Cₘₐₓ is about or greater than 6750 ng/mL but less than about 21000 ng/mL. In some embodiments, the mean Cₘₐₓ is about or greater than 7500 ng/mL but less than about 21000 ng/mL. In some embodiments, the mean whole blood Cₘₐₓ is about or greater than 8000 ng/mL, about or greater than 9000 ng/mL, or about or greater than 10000 ng/mL but less than about 28000 ng/mL. In some embodiments, the mean Cₘₐₓ is about or greater than 8000 ng/mL but less than about 28000 ng/mL. In some embodiments, the mean Cₘₐₓ is about or greater than 9000 ng/mL but less than about 28000 ng/mL. In some embodiments, the mean Cₘₐₓ is about or greater than 10000 ng/mL but less than about 28000 ng/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean minimum whole blood concentration (Cₘᵢₙ) (ng/mL) of between about 800 ng/mL and about 14400 ng/mL as measured on day 1 of dosing. In a different aspect, the administration of INX-315 or a pharmaceutically acceptable salt thereof in a human patient in need thereof results in a mean minimum whole blood concentration (Cₘᵢₙ) (ng/mL) of between about 500 ng/mL and about 8000 ng/mL as measured on day 1 of dosing. In some embodiments, the mean Cₘᵢₙ is about or greater than 800 ng/mL, about or greater than 900 ng/mL, or about or greater than 1000 ng/mL but less than about 3600 ng/mL. In some embodiments, the mean Cₘᵢₙ is about or greater than 800 ng/mL but less than about 3600 ng/mL. In some embodiments, the mean Cₘᵢₙ is about or greater than 900 ng/mL but less than about 3600 ng/mL. In some embodiments, the mean Cₘᵢₙ is about or greater than 1000 ng/mL but less than about 3600 ng/mL. In different embodiments, the mean Cₘᵢₙ is about or greater than 500 ng/mL, about or greater than 575 ng/mL, or about or greater than 650 ng/mL but less than about 2000 ng/mL. In different embodiments, the mean Cₘᵢₙ is about or greater than 500 ng/mL but less than about 2000 ng/mL. In different embodiments, the mean Cₘᵢₙ is about or greater than 575 ng/mL but less than about 2000 ng/mL. In different embodiments, the mean Cₘᵢₙ is about or greater than 650 ng/mL but less than about 2000 ng/mL. In some embodiments, the mean Cₘᵢₙ is about or greater than 1600 ng/mL, about or greater than 1800 ng/mL, or about or greater than 2000 ng/mL but less than about 7200 ng/mL. In some embodiments, the mean Cₘᵢₙ is about or greater than 1600 ng/mL but less than about 7200 ng/mL. In some embodiments, the mean Cₘᵢₙ is about or greater than 1800 ng/mL but less than about 7200 ng/mL. In some embodiments, the mean Cₘᵢₙ is about or greater than 2000 ng/mL but less than about 7200 ng/mL. In different embodiments, the mean Cₘᵢₙ is about or greater than 1000 ng/mL, about or greater than 1150 ng/mL, or about or greater than 1300 ng/mL but less than about 4000 ng/mL. In different embodiments, the mean Cₘᵢₙ is about or greater than 1000 ng/mL but less than about 4000 ng/mL. In different embodiments, the mean Cₘᵢₙ is about or greater than 1150 ng/mL but less than about 4000 ng/mL. In different embodiments, the mean Cₘᵢₙ is about or greater than 1300 ng/mL but less than about 4000 ng/mL. In some embodiments, the mean Cₘᵢₙ is about or greater than 2400 ng/mL, about or greater than 2700 ng/mL, or about or greater than 3000 ng/mL but less than about 10800 ng/mL. In some embodiments, the mean Cₘᵢₙ is about or greater than 2400 ng/mL but less than about 10800 ng/mL. In some embodiments, the mean Cₘᵢₙ is about or greater than 2700 ng/mL but less than about 10800 ng/mL. In some embodiments, the mean Cₘᵢₙ is about or greater than 3000 ng/mL but less than about 10800 ng/mL. In different embodiments, the mean Cₘᵢₙ is about or greater than 1500 ng/mL, about or greater than 1725 ng/mL, or about or greater than 1950 ng/mL but less than about 6000 ng/mL. In different embodiments, the mean Cₘᵢₙ is about or greater than 1500 ng/mL but less than about 6000 ng/mL. In different embodiments, the mean Cₘᵢₙ is about or greater than 1725 ng/mL but less than about 6000 ng/mL. In different embodiments, the mean Cₘᵢₙ is about or greater than 1950 ng/mL but less than about 6000 ng/mL. In some embodiments, the mean Cₘᵢₙ is about or greater than 3200 ng/mL, about or greater than 3600 ng/mL, or about or greater than 4000 ng/mL but less than about 14400 ng/mL. In some embodiments, the mean Cₘᵢₙ is about or greater than 3200 ng/mL but less than about 14400 ng/mL. In some embodiments, the mean Cₘᵢₙ is about or greater than 3600 ng/mL but less than about 14400 ng/mL. In some embodiments, the mean Cₘᵢₙ is about or greater than 4000 ng/mL but less than about 14400 ng/mL. In different embodiments, the mean Cₘᵢₙ is about or greater than 2000 ng/mL, about or greater than 2300 ng/mL, or about or greater than 2600 ng/mL but less than about 8000 ng/mL. In different embodiments, the mean Cₘᵢₙ is about or greater than 2000 ng/mL but less than about 8000 ng/mL. In different embodiments, the mean Cₘᵢₙ is about or greater than 2300 ng/mL but less than about 8000 ng/mL. In different embodiments, the mean Cₘᵢₙ is about or greater than 2600 ng/mL but less than about 8000 ng/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean whole blood area under the curve (AUC) (ng*hr/mL) of between about 50000 ng*hr/mL and about 800000 ng*hr/mL as measured on day 15 of dosing. In a different aspect, the administration of INX-315 or a pharmaceutically acceptable salt thereof in a human patient in need thereof results in a mean whole blood area under the curve (AUC) (ng*hr/mL) of between about 25000 ng*hr/mL and about 600000 ng*hr/mL as measured on day 15 of dosing. In some embodiments, the mean whole blood AUC is about or greater than 50000 ng*hr/mL, about or greater than 60000 ng*hr/mL, or about or greater than 70000 ng*hr/mL, but less than about 200000 ng*hr/mL. In some embodiments, the mean AUC is about or greater than 50000 ng*hr/mL but less than about 200000 ng*hr/mL. In some embodiments, the mean AUC is about or greater than 60000 ng*hr/mL but less than about 200000 ng*hr/mL. In some embodiments, the mean AUC is about or greater than 70000 ng*hr/mL but less than about 200000 ng*hr/mL. In different embodiments, the mean whole blood AUC is about or greater than 25000 ng*hr/mL, about or greater than 45000 ng*hr/mL, or about or greater than 52500 ng*hr/mL, but less than about 150000 ng*hr/mL. In different embodiments, the mean AUC is about or greater than 25000 ng*hr/mL but less than about 150000 ng*hr/mL. In different embodiments, the mean AUC is about or greater than 45000 ng*hr/mL but less than about 150000 ng*hr/mL. In different embodiments, the mean AUC is about or greater than 52500 ng*hr/mL but less than about 150000 ng*hr/mL. In some embodiments, the mean whole blood AUC is about or greater than 100000 ng*hr/mL, about or greater than 120000 ng*hr/mL, or about or greater than 140000 ng*hr/mL, but less than about 400000 ng*hr/mL. In some embodiments, the mean AUC is about or greater than 100000 ng*hr/mL but less than about 400000 ng*hr/mL. In some embodiments, the mean AUC is about or greater than 120000 ng*hr/mL but less than about 400000 ng*hr/mL. In some embodiments, the mean AUC is about or greater than 140000 ng*hr/mL but less than about 400000 ng*hr/mL. In different embodiments, the mean whole blood AUC is about or greater than 50000 ng*hr/mL, about or greater than 90000 ng*hr/mL, or about or greater than 105000 ng*hr/mL, but less than about 300000 ng*hr/mL. In different embodiments, the mean AUC is about or greater than 50000 ng*hr/mL but less than about 300000 ng*hr/mL. In different embodiments, the mean AUC is about or greater than 90000 ng*hr/mL but less than about 300000 ng*hr/mL. In different embodiments, the mean AUC is about or greater than 105000 ng*hr/mL but less than about 300000 ng*hr/mL. In some embodiments, the mean whole blood AUC is about or greater than 150000 ng*hr/mL, about or greater than 120000 ng*hr/mL, or about or greater than 140000 ng*hr/mL, but less than about 600000 ng*hr/mL. In some embodiments, the mean AUC is about or greater than 150000 ng*hr/mL but less than about 600000 ng*hr/mL. In some embodiments, the mean AUC is about or greater than 180000 ng*hr/mL but less than about 600000 ng*hr/mL. In some embodiments, the mean AUC is about or greater than 210000 ng*hr/mL but less than about 600000 ng*hr/mL. In different embodiments, the mean whole blood AUC is about or greater than 75000 ng*hr/mL, about or greater than 135000 ng*hr/mL, or about or greater than 157500 ng*hr/mL, but less than about 450000 ng*hr/mL. In different embodiments, the mean AUC is about or greater than 75000 ng*hr/mL but less than about 450000 ng*hr/mL. In different embodiments, the mean AUC is about or greater than 135000 ng*hr/mL but less than about 450000 ng*hr/mL. In different embodiments, the mean AUC is about or greater than 157500 ng*hr/mL but less than about 450000 ng*hr/mL. In some embodiments, the mean whole blood AUC is about or greater than 200000 ng*hr/mL, about or greater than 240000 ng*hr/mL, or about or greater than 280000 ng*hr/mL, but less than about 800000 ng*hr/mL. In some embodiments, the mean AUC is about or greater than 200000 ng*hr/mL but less than about 800000 ng*hr/mL. In some embodiments, the mean AUC is about or greater than 240000 ng*hr/mL but less than about 800000 ng*hr/mL. In some embodiments, the mean AUC is about or greater than 280000 ng*hr/mL but less than about 800000 ng*hr/mL. In different embodiments, the mean whole blood AUC is about or greater than 100000 ng*hr/mL, about or greater than 180000 ng*hr/mL, or about or greater than 210000 ng*hr/mL, but less than about 600000 ng*hr/mL. In different embodiments, the mean AUC is about or greater than 100000 ng*hr/mL but less than about 600000 ng*hr/mL. In different embodiments, the mean AUC is about or greater than 180000 ng*hr/mL but less than about 600000 ng*hr/mL. In different embodiments, the mean AUC is about or greater than 210000 ng*hr/mL but less than about 600000 ng*hr/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean maximum whole blood concentration (Cₘₐₓ) (ng/mL) in humans of between about 3000 ng/mL and about 56000 ng/mL as measured on day 15 of dosing. In some embodiments, the mean whole blood Cₘₐₓ is about or greater than 3000 ng/mL, about or greater than 3500 ng/mL, or about or greater than 4000 ng/mL but less than about 14000 ng/mL. In some embodiments, the mean Cₘₐₓ is about or greater than 3000 ng/mL but less than about 14000 ng/mL. In some embodiments, the mean Cₘₐₓ is about or greater than 3500 ng/mL but less than about 14000 ng/mL. In some embodiments, the mean Cₘₐₓ is about or greater than 4000 ng/mL but less than about 14000 ng/mL. In some embodiments, the mean whole blood Cₘₐₓ is about or greater than 6000 ng/mL, about or greater than 7000 ng/mL, or about or greater than 8000 ng/mL but less than about 28000 ng/mL. In some embodiments, the mean Cₘₐₓ is about or greater than 6000 ng/mL but less than about 28000 ng/mL. In some embodiments, the mean Cₘₐₓ is about or greater than 7000 ng/mL but less than about 28000 ng/mL. In some embodiments, the mean Cₘₐₓ is about or greater than 8000 ng/mL but less than about 28000 ng/mL. In some embodiments, the mean whole blood Cₘₐₓ is about or greater than 9000 ng/mL, about or greater than 10500 ng/mL, or about or greater than 12000 ng/mL but less than about 42000 ng/mL. In some embodiments, the mean Cₘₐₓ is about or greater than 9000 ng/mL but less than about 42000 ng/mL. In some embodiments, the mean Cₘₐₓ is about or greater than 10500 ng/mL but less than about 42000 ng/mL. In some embodiments, the mean Cₘₐₓ is about or greater than 12000 ng/mL but less than about 42000 ng/mL. In some embodiments, the mean whole blood Cₘₐₓ is about or greater than 12000 ng/mL, about or greater than 14000 ng/mL, or about or greater than 16000 ng/mL but less than about 56000 ng/mL. In some embodiments, the mean Cₘₐₓ is about or greater than 12000 ng/mL but less than about 56000 ng/mL. In some embodiments, the mean Cₘₐₓ is about or greater than 14000 ng/mL but less than about 56000 ng/mL. In some embodiments, the mean Cₘₐₓ is about or greater than 16000 ng/mL but less than about 56000 ng/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In another aspect, the administration of INX-315 or a pharmaceutically acceptable salt thereof in a human patient in need thereof results in a mean minimum whole blood concentration (Cₘᵢₙ) (ng/mL) of between about 1000 ng/mL and about 24000 ng/mL as measured on day 15 of dosing. In some embodiments, the mean whole blood Cₘᵢₙ is about or greater than 1000 ng/mL, about or greater than 1300 ng/mL, or about or greater than 1600 ng/mL but less than about 6000 ng/mL. In some embodiments, the mean Cₘᵢₙ is about or greater than 1000 ng/mL but less than about 6000 ng/mL. In some embodiments, the mean Cₘᵢₙ is about or greater than 1300 ng/mL but less than about 6000 ng/mL. In some embodiments, the mean Cₘᵢₙ is about or greater than 1600 ng/mL but less than about 6000 ng/mL. In some embodiments, the mean whole blood Cₘᵢₙ is about or greater than 2000 ng/mL, about or greater than 2600 ng/mL, or about or greater than 3200 ng/mL but less than about 12000 ng/mL. In some embodiments, the mean Cₘᵢₙ is about or greater than 2000 ng/mL but less than about 12000 ng/mL. In some embodiments, the mean Cₘᵢₙ is about or greater than 2600 ng/mL but less than about 12000 ng/mL. In some embodiments, the mean Cₘᵢₙ is about or greater than 3200 ng/mL but less than about 12000 ng/mL. In some embodiments, the mean whole blood Cₘᵢₙ is about or greater than 3000 ng/mL, about or greater than 3900 ng/mL, or about or greater than 4800 ng/mL but less than about 18000 ng/mL. In some embodiments, the mean Cₘᵢₙ is about or greater than 3000 ng/mL but less than about 18000 ng/mL. In some embodiments, the mean Cₘᵢₙ is about or greater than 3900 ng/mL but less than about 18000 ng/mL. In some embodiments, the mean Cₘᵢₙ is about or greater than 4800 ng/mL but less than about 18000 ng/mL. In some embodiments, the mean whole blood Cₘᵢₙ is about or greater than 4000 ng/mL, about or greater than 5200 ng/mL, or about or greater than 6400 ng/mL but less than about 24000 ng/mL. In some embodiments, the mean Cₘᵢₙ is about or greater than 4000 ng/mL but less than about 24000 ng/mL. In some embodiments, the mean Cₘᵢₙ is about or greater than 5200 ng/mL but less than about 24000 ng/mL. In some embodiments, the mean Cₘᵢₙ is about or greater than 6400 ng/mL but less than about 24000 ng/mL. In some embodiments, INX-315, or its acceptable salt, is administered once-a-day at a dose of between 100 mg and 500 mg. In some embodiments, the administration of INX-315 or a pharmaceutically acceptable salt thereof in a human patient in need thereof results in a mean plasma Cₘₐₓ to Cₘᵢₙ ratio of less than 5 but greater than about 1.25. In some embodiments, the Cₘₐₓ to Cₘᵢₙ ratio is less than 3. In some embodiments, the Cₘₐₓ to Cₘᵢₙ ratio is less than 2.25.

In one aspect, the administration of INX-315 or a pharmaceutically acceptable salt thereof in a human patient in need thereof results in a mean Cₘₐₓ to Cₘᵢₙ ratio of less than 5, less than 4, less than 3, or less than 2.25 but greater than about 1.25. In some embodiments, the mean Cₘₐₓ to Cₘᵢₙ ratio is less than 5 but greater than about 1.25. In some embodiments, the mean Cₘₐₓ to Cₘᵢₙ ratio is less than 4 but greater than about 1.25. In some embodiments, the mean Cₘₐₓ to Cₘᵢₙ ratio is less than 3 but greater than about 1.25. In some embodiments, the mean Cₘₐₓ to Cₘᵢₙ ratio is less than 2.25 but greater than about 1.25. In some embodiments, the Cₘₐₓ to Cₘᵢₙ ratio is about 2.2. In some embodiments, the Cₘₐₓ to Cₘᵢₙ ratio is measured in plasma. In some embodiments, the Cₘₐₓ to Cₘᵢₙ ratio is measured in whole blood. In some embodiments, INX-315, or its acceptable salt, is administered once-a-day at a dose of between 100 mg and 500 mg.

In one aspect, the treatment of a human with a CDK2-mediated cancer comprising administering an effective amount of INX-315 or a pharmaceutically acceptable salt thereof to the human in need thereof results in a whole blood to plasma area under the curve (AUC) ratio of at least about 6 up to about 22 as measured on day 1 of dosing. In some embodiments, the whole blood to plasma AUC ratio is at least about 12 up to about 20 as measured on day 1 of dosing.

In another aspect, the treatment of a human with a CDK2-mediated cancer comprising administering an effective amount of INX-315 or a pharmaceutically acceptable salt thereof to the human in need thereof results in a whole blood to plasma area under the curve (AUC) ratio of at least about 6 up to about 40 as measured on day 15 of dosing. In some embodiments, the whole blood to plasma AUC ratio is at least about 8 up to about 18 as measured on day 15 of dosing.

In a different aspect, the treatment of a human with a CDK2-mediated cancer comprising administering an effective amount of INX-315 or a pharmaceutically acceptable salt thereof to the human in need thereof results in a RBC-to-plasma ratio (K_{RBC/P}) of at least about 10.0 up to about 20.0. In different embodiments, the RBC-to-plasma ratio (K_{RBC/P}) is at least about 20.0 up to about 50.0.

In a different aspect, the treatment of a human with a CDK2-mediated cancer comprising administering an effective amount of INX-315 or a pharmaceutically acceptable salt thereof to the human in need thereof results in a whole blood-to-plasma ratio (K_{WB/P}) at least about 4.0 up to about 10.0. In different embodiments, the whole blood-to-plasma ratio (K_{WB/P}) is at least about 8.0 up to about 20.0.

In another aspect, the method of treating provides a t_{1/2} of INX-315 in humans of between about 12 hours and about 24 hours. In some embodiments, the t_{1/2} is greater than about 12 hours but less than about 18 hours. In some embodiments, the t_{1/2} is about or greater than about 12 hours, about or greater than about 13 hours, about or greater than about 14 hours, about or greater than about 15 hours, about or greater than about 16 hours, about or greater than about 17 hours, or about 18 hours. In some embodiments, the t_{1/2} is about or greater than about 14 hours, greater than about 16 hours, greater than about 16.5 hours, greater than about 17 hours, greater than about 17.5 hours, or greater than about 18 hours. In some embodiments, the t_{1/2} is about or greater than about 14 hours. In some embodiments, the t_{1/2} is about or greater than about 15 hours. In some embodiments, the t_{1/2} is about or greater than about 16 hours. In some embodiments, the t_{1/2} of INX-315 is measured for plasma INX-315. In some embodiments, the t_{1/2} of INX-315 is measured for whole blood INX-315. In some embodiments, INX-315, or its acceptable salt, is administered once-a-day at a dose of between 100 mg and 500 mg.

In a different aspect, the method of treating provides a t_{1/2} of INX-315 in humans of between about 8 hours and about 28 hours. In different embodiments, the t_{1/2} is greater than about 10 hours but less than about 26 hours. In different embodiments, the t_{1/2} is about or greater than about 8 hours, about or greater than about 10 hours, about or greater than about 12 hours, about or greater than about 14 hours, about or greater than about 15 hours, about or greater than about 16 hours, about or greater than about 18 hours, or about 20 hours. In different embodiments, the t_{1/2} is about or greater than about 12 hours, greater than about 14 hours, greater than about 16 hours, greater than about 18 hours, greater than about 20 hours, or greater than about 20 hours. In different embodiments, the t_{1/2} is about or greater than about 9 hours. In some embodiments, the t_{1/2} is about or greater than about 10 hours. In different embodiments, the t_{1/2} is about or greater than about 11 hours. In different embodiments, the t_{1/2} is about or greater than about 12 hours. In different embodiments, the t_{1/2} is about or greater than about 13 hours. In different embodiments, the t_{1/2} is about or greater than about 14 hours. In different embodiments, the t_{1/2} is about or greater than about 15 hours. In different embodiments, the t_{1/2} is about or greater than about 16 hours. In different embodiments, the t_{1/2} is about or greater than about 17 hours. In different embodiments, the t_{1/2} is about or greater than about 18 hours. In different embodiments, the t_{1/2} is about or greater than about 19 hours. In different embodiments, the t_{1/2} is about or greater than about 20 hours. In different embodiments, the t_{1/2} is about or greater than about 21 hours. In different embodiments, the t_{1/2} is about or greater than about 22 hours. In different embodiments, the t_{1/2} is about or greater than about 23 hours. In different embodiments, the t_{1/2} is about or greater than about 24 hours. In different embodiments, the t_{1/2} of INX-315 is measured for plasma INX-315. In different embodiments, the t_{1/2} of INX-315 is measured for whole blood INX-315. In different embodiments, INX-315, or its acceptable salt, is administered once-a-day at a dose of between 100 mg and 500 mg.

In a different aspect, the method of treating provides a t_{1/2} of INX-315 in humans of between about 5 hours and about 32 hours. In different embodiments, the t_{1/2} is greater than about 8 hours but less than about 28 hours. In different embodiments, the t_{1/2} is about or greater than about 5 hours, about or greater than about 8 hours, about or greater than about 10 hours, about or greater than about 12 hours, about or greater than about 14 hours, about or greater than about 15 hours, about or greater than about 16 hours, about or greater than about 18 hours, or about 20 hours. In different embodiments, the t_{1/2} is about or greater than about 8 hours, about or greater than about 12 hours, greater than about 14 hours, greater than about 16 hours, greater than about 18 hours, greater than about 20 hours, or greater than about 20 hours. In different embodiments, the t_{1/2} is about or greater than about 5 hours. In different embodiments, the t_{1/2} is about or greater than about 6 hours. In different embodiments, the t_{1/2} is about or greater than about 7 hours. In different embodiments, the t_{1/2} is about or greater than about 8 hours. In different embodiments, the t_{1/2} is about or greater than about 9 hours. In some embodiments, the t_{1/2} is about or greater than about 10 hours. In different embodiments, the t_{1/2} is about or greater than about 11 hours. In different embodiments, the t_{1/2} is about or greater than about 12 hours. In different embodiments, the t_{1/2} is about or greater than about 13 hours. In different embodiments, the t_{1/2} is about or greater than about 14 hours. In different embodiments, the t_{1/2} is about or greater than about 15 hours. In different embodiments, the t_{1/2} is about or greater than about 16 hours. In different embodiments, the t_{1/2} is about or greater than about 17 hours. In different embodiments, the t_{1/2} is about or greater than about 18 hours. In different embodiments, the t_{1/2} is about or greater than about 19 hours. In different embodiments, the t_{1/2} is about or greater than about 20 hours. In different embodiments, the t_{1/2} is about or greater than about 21 hours. In different embodiments, the t_{1/2} is about or greater than about 22 hours. In different embodiments, the t_{1/2} is about or greater than about 23 hours. In different embodiments, the t_{1/2} is about or greater than about 24 hours. In different embodiments, the t_{1/2} of INX-315 is measured for plasma INX-315. In different embodiments, the t_{1/2} of INX-315 is measured for whole blood INX-315. In different embodiments, INX-315, or its acceptable salt, is administered once-a-day at a dose of between 100 mg and 500 mg.

### Methods of Treatment

As contemplated herein, or treating a patient in need thereof, including a human, comprising administering an effective amount of INX-315 or its pharmaceutically acceptable salt which provides substantially improved blood pharmacokinetic (PK) or pharmacodynamic (PD) profiles that lead to higher than expected and longer than expected blood and plasma levels without a significant spike immediately after dosing.

In one aspect, the patient has a cancer. In some embodiments, the cancer is a breast cancer. In some embodiments, the breast cancer is hormone receptor positive (HR+) breast cancer. In some embodiments, the breast cancer is estrogen receptor-positive (ER+) breast cancer. In some embodiments, the breast cancer is progesterone receptor-positive (PR+) breast cancer. In some embodiments, the breast cancer is human epidermal growth factor receptor 2-negative (HER2−). In some embodiments, the breast cancer is HR+/HER2− breast cancer. In some embodiments, the breast cancer is ER+/HER2− breast cancer. In some embodiments, the breast cancer is triple negative breast cancer (TNBC). In some embodiments, the TNBC is Rb-independent. In some embodiments, the breast cancer has an amplification or overexpression of cyclin E. In some embodiments, the breast cancer is CDK4/6 inhibitor-resistant. In some embodiments, the breast cancer has progressed on a prior CDK4/6 inhibitor regimen. In some embodiments, the breast cancer is endocrine therapy-resistant. In some embodiments, the administration of INX-315 or a pharmaceutically acceptable salt thereof in a human patient in need thereof results in a mean Cₘₐₓ to Cₘᵢₙ ratio of less than 5 but greater than about 1.25. In some embodiments, the administration of INX-315 or a pharmaceutically acceptable salt thereof in a human patient in need thereof results in a t_{1/2} of between about 12 hours and about 18 hours. In some embodiments, the t_{1/2} is about or greater than about 14 hours. In some embodiments, the t_{1/2} is about or greater than about 15 hours. In some embodiments, the t_{1/2} is about or greater than about 16 hours. In some embodiments, the INX-315 is administered at a dose of between about 100 mg and about 500 mg. In some embodiments, the INX-315 is administered at a dose of about 100 mg, about 125 mg, or about 150 mg. In some embodiments, the INX-315 is administered at a dose of about 200 mg, about 225 mg, or about 250 mg. In some embodiments, the INX-315 is administered at a dose of about 300 mg, about 325 mg, or about 350 mg. In some embodiments, the INX-315 is administered at a dose of about 400 mg, about 425 mg, or about 450 mg. In some embodiments, the INX-315 is orally administered. In some embodiments, the INX-315 is formulated as an amorphous spray-dried dispersion (ASD).

In some embodiments, the cancer is ovarian cancer. In some embodiments, the ovarian cancer has an amplification or overexpression of cyclin E. In some embodiments, the ovarian cancer is CDK4/6 inhibitor-resistant. In some embodiments, the ovarian cancer is endocrine therapy-resistant. In some embodiments, the ovarian cancer comprises high-grade serous ovarian cancer (HGSOC). In some embodiments, the administration of INX-315 or a pharmaceutically acceptable salt thereof in a human patient in need thereof results in a mean Cₘₐₓ to Cₘᵢₙ ratio of less than 5 but greater than about 1.25. In some embodiments, the administration of INX-315 or a pharmaceutically acceptable salt thereof in a human patient having lung cancer results in a t_{1/2} of between about 12 hours and about 24 hours. In some embodiments, the administration of INX-315 or a pharmaceutically acceptable salt thereof in a human patient in need thereof results in a t_{1/2} of between about 12 hours and about 18 hours. In some embodiments, the t_{1/2} is about or greater than about 14 hours but less than about 18 hours. In some embodiments, the t_{1/2} is about or greater than about 15 hours but less than about 18 hours. In some embodiments, the t_{1/2} is about or greater than about 16 hours but less than about 18 hours. In some embodiments, the INX-315 is administered at a dose of between about 100 mg and about 500 mg. In some embodiments, the INX-315 is administered at a dose of about 100 mg, about 125 mg, or about 150 mg. In some embodiments, the INX-315 is administered at a dose of about 200 mg, about 225 mg, or about 250 mg. In some embodiments, the INX-315 is administered at a dose of about 300 mg, about 325 mg, or about 350 mg. In some embodiments, the INX-315 is administered at a dose of about 400 mg, about 425 mg, or about 450 mg. In some embodiments, the INX-315 is orally administered. In some embodiments, the INX-315 is formulated as an amorphous spray-dried dispersion (ASD).

In some embodiments, the cancer is lung cancer. In some embodiments, the lung cancer is CDK4/6 inhibitor-resistant. In some embodiments, the lung cancer is small cell lung cancer (SCLC). In some embodiments, the SCLC has an amplification or overexpression of cyclin E. In some embodiments, the SCLC is Rb-independent. In some embodiments, the administration of INX-315 or a pharmaceutically acceptable salt thereof in a human patient having lung cancer results in a mean Cₘₐₓ to Cₘᵢₙ ratio of less than 5 but greater than about 1.25. In some embodiments, the administration of INX-315 or a pharmaceutically acceptable salt thereof in a human patient having lung cancer results in a t_{1/2} of between about 12 hours and about 24 hours. In some embodiments, the administration of INX-315 or a pharmaceutically acceptable salt thereof in a human patient having lung cancer results in a t_{1/2} of between about 12 hours and about 18 hours. In some embodiments, the t_{1/2} is about or greater than about 14 hours but less than about 18 hours. In some embodiments, the t_{1/2} is about or greater than about 15 hours but less than about 18 hours. In some embodiments, the t_{1/2} is about or greater than about 16 hours but less than about 18 hours. In some embodiments, the INX-315 is administered at a dose of between about 100 mg and about 500 mg. In some embodiments, the INX-315 is administered at a dose of about 100 mg, about 125 mg, or about 150 mg. In some embodiments, the INX-315 is administered at a dose of about 200 mg, about 225 mg, or about 250 mg. In some embodiments, the INX-315 is administered at a dose of about 300 mg, about 325 mg, or about 350 mg. In some embodiments, the INX-315 is administered at a dose of about 400 mg, about 425 mg, or about 450 mg. In some embodiments, the INX-315 is orally administered. In some embodiments, the INX-315 is formulated as an amorphous spray-dried dispersion (ASD).

In some embodiments, the cancer is prostate cancer.

In some embodiments, the cancer is bladder cancer.

In some embodiments, the cancer is sarcoma.

In some embodiments, the cancer is uterine cancer. In some embodiments, the uterine cancer is endometrial cancer.

In some embodiments, the methods of treatment described herein are used to treat solid tumors. In some embodiments, the solid tumors have amplification or overexpression of cyclin E. In some embodiments, the solid tumors progressed on standard of care treatment.

In certain aspects, the cancer to be treated using the methods described herein is selected from bladder cancer, breast cancer, cervical cancer, colon cancer, endometrial cancer, esophageal cancer, fallopian tube cancer, glioblastoma multiforme (GBM), head and neck cancer, kidney cancer, liver cancer, lung cancer, ovarian cancer, peritoneal cancer, prostate cancer, sarcoma, skin cancer, stomach cancer, or uterine cancer. In some embodiments, the cancer is lung cancer. In some embodiments, the cancer is small cell lung cancer (SCLC). In some embodiments, the cancer is breast cancer. In some embodiments, the breast cancer is hormone receptor-positive (HR+) and HER2-negative. In some embodiments, the breast cancer is estrogen receptor-positive (ER+). In some embodiments, the breast cancer is progesterone receptor-positive (PR+). In some embodiments, the breast cancer is HER2-negative (HER2−). In some embodiments, the breast cancer is triple negative breast cancer. In some embodiments, the cancer is bladder cancer. In some embodiments, the cancer is ovarian cancer. In some embodiments, the cancer is esophageal cancer. In some embodiments, the esophageal cancer comprises Esophageal Adenocarcinoma. In some embodiments, the ovarian cancer comprises high-grade serous ovarian cancer (HGSOC). In some embodiments, the cancer is prostate cancer. In some embodiments, the cancer is sarcoma. In some embodiments, the cancer is uterine cancer. In some embodiments, the cancer is advanced unresectable and/or metastatic cancer. In some embodiments, the cancer is cyclin E amplified or overexpressed. In some embodiments, a Next Generation Sequencing (NGS) panel test is used to confirm cyclin E overexpression or amplification status. In some embodiments, the human previously received at least one prior line of chemotherapy. In some embodiments, the human previously received at least two prior lines of chemotherapy. In some embodiments, the human previously received at least one prior line of CDK4/6 inhibitor therapy. In some embodiments, the human previously received at least one prior line of endocrine therapy. In some embodiments, the cancer is relapsed. In some embodiments, the cancer has progressed following a prior standard of care therapy. In some embodiments, the cancer has progressed following a prior systemic therapy. In some embodiments, the cancer is intolerant or ineligible for standard or care therapy. In some embodiments, the cancer is refractory. In some embodiments, the cancer has progressed following a prior regimen comprising a platinum analog. In some embodiments, the cancer is platinum-refractory or platinum-resistant. In some embodiments, the cancer has progressed following a prior regimen comprising a CDK4/6 inhibitor. In some embodiments, the cancer is CDK4/6 inhibitor resistant. In some embodiments, the cancer has progressed following a prior regimen comprising an endocrine therapy. In some embodiments, the cancer is endocrine therapy resistant.

In one aspect, the cancer to be treated using the methods described herein is an Rb-positive cancer. In some embodiments, the Rb-positive cancer is selected from an Rb-positive lung cancer, bone cancer, pancreatic cancer, skin cancer, head and neck cancer, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. In some embodiments, the Rb-positive cancer is selected from an Rb-positive HR+/HER2− breast cancer, ER+/HER2− breast cancer, high-grade serous ovarian cancer (HGSOC), non-small cell lung cancer (NSCLC), colon cancer, prostate cancer, or glioblastoma. In some embodiments, the Rb-positive cancer is HR+/IiER2− breast cancer. In some embodiments, the Rb-positive cancer is ER+/HER2− breast cancer. In some embodiments, the Rb-positive cancer is high-grade serous ovarian cancer (HGSOC). In some embodiments, the Rb-positive cancer is non-small cell lung cancer (NSCLC).

In some embodiments, INX-315 or a pharmaceutically acceptable salt or ASD formulation thereof described herein provides an additive effect to or synergistic effect with effect of a CDK4/6 inhibitor for the treatment of an Rb-positive cancer. In some embodiments the CDK4/6 inhibitor is a selective CDK4/6 inhibitor. In some embodiments, the CDK4/6 inhibitor is selected from palbociclib, ribociclib, abemaciclib, trilaciclib, lerociclib, or dalpiciclib. In some embodiments, the CDK4/6 inhibitor is palbociclib. In some embodiments, the CDK4/6 inhibitor is ribociclib. In some embodiments, the CDK4/6 inhibitor is lerociclib. In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the method further comprises administering an effective amount of a selective CDK4 inhibitor. In some embodiments, the selective CDK4 inhibitor is PF-07220060. In some embodiments, the CDK4/6 inhibitor is administered in a standard dosage amount and frequency of administration regimen. In some embodiments, the CDK4/6 inhibitor is administered at least about 80%, at least about 75%, at least about 70%, at least about 66.7%, at least about 65%, at least about 60%, at least about 50%, or about 50% of the standard dosage amount. In some embodiments, the CDK4/6 inhibitor is administered at 75% of the standard dosage amount. In some embodiments, the CDK4/6 inhibitor is administered at 66.7% of the standard dosage amount. In some embodiments, the CDK4/6 inhibitor is administered at 50% of the standard dosage amount. In some embodiments, the method further comprises administering a chemotherapeutic agent. In some embodiments, the chemotherapeutic agent is selected from 5-fluorouracil (5-FU), capecitabine, carboplatin, cisplatin, docetaxel, etoposide, gemcitabine, irinotecan, mitomycin, paclitaxel, vincristine, or a combination thereof. In some embodiments, the chemotherapeutic agent is carboplatin. In some embodiments, the chemotherapeutic agent is cisplatin. In some embodiments, the method further comprises administering an endocrine therapy. In some embodiments, the endocrine therapy comprises a SERD. In some embodiments, the SERD is selected from fulvestrant or elacestrant. In some embodiments, the human previously received at least one prior line of standard of care therapy. In some embodiments, the human previously received at least two prior lines of standard of care therapy. In some embodiments, the human previously received at least one prior line of CDK4/6 inhibitor therapy. In some embodiments, the human previously received at least one prior line of endocrine therapy. In some embodiments, the cancer is relapsed. In some embodiments, the cancer has progressed following a prior standard of care therapy. In some embodiments, the cancer has progressed following a prior systemic therapy. In some embodiments, the cancer is intolerant or ineligible for standard or care therapy. In some embodiments, the cancer is refractory. In some embodiments, the cancer has progressed following a prior regimen comprising a platinum analog. In some embodiments, the cancer is platinum-refractory or platinum-resistant. In some embodiments, the cancer has progressed following a prior regimen comprising a CDK4/6 inhibitor. In some embodiments, the cancer is CDK4/6 inhibitor resistant. In some embodiments, the cancer has progressed following a prior regimen comprising an endocrine therapy. In some embodiments, the cancer is endocrine therapy resistant.

In a different aspect, the cancer to be treated using the methods described herein is an Rb-independent cancer. In some embodiments, the Rb-independent cancer is selected from breast cancer, lung cancer, prostate cancer, liver cancer, bladder cancer, ovarian cancer, uterine cancer, cervical cancer, stomach cancer, esophageal cancer, head and neck cancer, glioblastoma, retinoblastoma, osteosarcoma, or lymphoma. In some embodiments, the Rb-independent cancer is selected from small cell lung cancer (SCLC), retinoblastoma, triple negative breast cancer (TNBC), human papillomavirus (HPV) positive head and neck cancer, HPV positive cervical cancer, or neuroendocrine prostate carcinoma. In some embodiments, the Rb-independent cancer is SCLC. In some embodiments, the Rb-independent cancer is TNBC. In some embodiments, the Rb-independent cancer is bladder cancer.

In some embodiments, INX-315 or a pharmaceutically acceptable salt or ASD formulation thereof described herein provides an additive effect to or synergistic effect with effect of a CDK4/6 inhibitor for the treatment of an Rb-independent cancer. In some embodiments the CDK4/6 inhibitor is a selective CDK4/6 inhibitor. In some embodiments, the CDK4/6 inhibitor is selected from palbociclib, ribociclib, abemaciclib, trilaciclib, lerociclib, or dalpiciclib. In some embodiments, the CDK4/6 inhibitor is palbociclib. In some embodiments, the CDK4/6 inhibitor is ribociclib. In some embodiments, the CDK4/6 inhibitor is lerociclib. In some embodiments, the CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the method further comprises administering an effective amount of a selective CDK4 inhibitor. In some embodiments, the selective CDK4 inhibitor is PF-07220060. In some embodiments, the CDK4/6 inhibitor is administered in a standard dosage amount and frequency of administration regimen. In some embodiments, the CDK4/6 inhibitor is administered at least about 80%, at least about 75%, at least about 70%, at least about 66.7%, at least about 65%, at least about 60%, at least about 50%, or about 50% of the standard dosage amount. In some embodiments, the CDK4/6 inhibitor is administered at 75% of the standard dosage amount. In some embodiments, the CDK4/6 inhibitor is administered at 66.7% of the standard dosage amount. In some embodiments, the CDK4/6 inhibitor is administered at 50% of the standard dosage amount. In some embodiments, the method further comprises administering a chemotherapeutic agent. In some embodiments, the chemotherapeutic agent is selected from 5-fluorouracil (5-FU), capecitabine, carboplatin, cisplatin, docetaxel, etoposide, gemcitabine, irinotecan, mitomycin, paclitaxel, vincristine, or a combination thereof. In some embodiments, the chemotherapeutic agent is carboplatin. In some embodiments, the chemotherapeutic agent is cisplatin. In some embodiments, the method further comprises administering an endocrine therapy. In some embodiments, the endocrine therapy comprises a SERD. In some embodiments, the SERD is selected from fulvestrant or elacestrant. In some embodiments, the human previously received at least one prior line of standard of care therapy. In some embodiments, the human previously received at least two prior lines of standard of care therapy. In some embodiments, the human previously received at least one prior line of CDK4/6 inhibitor therapy. In some embodiments, the human previously received at least one prior line of endocrine therapy. In some embodiments, the cancer is relapsed. In some embodiments, the cancer has progressed following a prior standard of care therapy. In some embodiments, the cancer has progressed following a prior systemic therapy. In some embodiments, the cancer is intolerant or ineligible for standard or care therapy. In some embodiments, the cancer is refractory. In some embodiments, the cancer has progressed following a prior regimen comprising a platinum analog. In some embodiments, the cancer is platinum-refractory or platinum-resistant. In some embodiments, the cancer has progressed following a prior regimen comprising a CDK4/6 inhibitor. In some embodiments, the cancer is CDK4/6 inhibitor resistant. In some embodiments, the cancer has progressed following a prior regimen comprising an endocrine therapy. In some embodiments, the cancer is endocrine therapy resistant.

In some embodiments, INX-315 or a pharmaceutically acceptable salt or ASD formulation thereof described herein in combination with palbociclib for the treatment of a subject having a cancer. In some embodiments, INX-315 or a pharmaceutically acceptable salt or ASD formulation thereof is administered in conjunction with palbociclib administered for 21 days during a 28-day cycle with a 7 day drug holiday. In some embodiments, the cancer is breast cancer. In some embodiments, the breast cancer is hormone receptor-positive (HR+) and HER2-negative. In some embodiments, the breast cancer is estrogen receptor-positive (ER+). In some embodiments, the breast cancer is progesterone receptor-positive (PR+). In some embodiments, the breast cancer is HER2-negative (HER2−). In some embodiments, the cancer is ER+/HER2− breast cancer. In some embodiments, the cancer is lung cancer. In some embodiments, the cancer is bladder cancer. In some embodiments, the cancer is ovarian cancer. In some embodiments, the cancer is endometrial cancer. In some embodiments, the cancer is esophageal cancer. In some embodiments, the esophageal cancer comprises Esophageal Adenocarcinoma. In some embodiments, the ovarian cancer comprises high-grade serous ovarian cancer (HGSOC). In some embodiments, the cancer is prostate cancer. In some embodiments, the cancer is sarcoma. In some embodiments, the cancer is uterine cancer. In some embodiments, the cancer is gastric cancer. In some embodiments, the cancer is advanced unresectable and/or metastatic cancer. In some embodiments, the cancer comprises an Rb-independent cancer. In some embodiments, the Rb-independent cancer is SCLC. In some embodiments, the Rb-independent cancer is TNBC. In some embodiments, the method further comprises administering a chemotherapeutic agent. In some embodiments, the chemotherapeutic agent is selected from 5-fluorouracil (5-FU), capecitabine, carboplatin, cisplatin, docetaxel, etoposide, gemcitabine, irinotecan, mitomycin, paclitaxel, vincristine, or a combination thereof. In some embodiments, the chemotherapeutic agent is carboplatin. In some embodiments, the chemotherapeutic agent is cisplatin. In some embodiments, the method further comprises administering an endocrine therapy. In some embodiments, the endocrine therapy comprises a SERD. In some embodiments, the SERD is selected from fulvestrant or elacestrant. In some embodiments, the human previously received at least one prior line of standard of care therapy. In some embodiments, the human previously received at least two prior lines of standard of care therapy. In some embodiments, the human previously received at least one prior line of CDK4/6 inhibitor therapy. In some embodiments, the human previously received at least one prior line of endocrine therapy. In some embodiments, the cancer is relapsed. In some embodiments, the cancer has progressed following a prior standard of care therapy. In some embodiments, the cancer has progressed following a prior systemic therapy. In some embodiments, the cancer is intolerant or ineligible for standard or care therapy. In some embodiments, the cancer is refractory. In some embodiments, the cancer has progressed following a prior regimen comprising a platinum analog. In some embodiments, the cancer is platinum-refractory or platinum-resistant. In some embodiments, the cancer has progressed following a prior regimen comprising a CDK4/6 inhibitor. In some embodiments, the cancer is CDK4/6 inhibitor resistant. In some embodiments, the cancer has progressed following a prior regimen comprising an endocrine therapy. In some embodiments, the cancer is endocrine therapy resistant.

In some embodiments, INX-315 or a pharmaceutically acceptable salt or ASD formulation thereof described herein in combination with ribociclib for the treatment of a subject having a cancer. In some embodiments, INX-315 or a pharmaceutically acceptable salt or ASD formulation thereof is administered in conjunction with ribociclib administered for 21 days during a 28-day cycle with a 7 day drug holiday. In some embodiments, the cancer is breast cancer. In some embodiments, the breast cancer is hormone receptor-positive (HR+) and HER2-negative. In some embodiments, the breast cancer is estrogen receptor-positive (ER+). In some embodiments, the breast cancer is progesterone receptor-positive (PR+). In some embodiments, the breast cancer is HER2-negative (HER2−). In some embodiments, the cancer is ER+/HER2− breast cancer. In some embodiments, the cancer is lung cancer. In some embodiments, the cancer is bladder cancer. In some embodiments, the cancer is ovarian cancer. In some embodiments, the cancer is endometrial cancer. In some embodiments, the cancer is esophageal cancer. In some embodiments, the esophageal cancer comprises Esophageal Adenocarcinoma. In some embodiments, the ovarian cancer comprises high-grade serous ovarian cancer (HGSOC). In some embodiments, the cancer is prostate cancer. In some embodiments, the cancer is sarcoma. In some embodiments, the cancer is uterine cancer. In some embodiments, the cancer is gastric cancer. In some embodiments, the cancer is advanced unresectable and/or metastatic cancer. In some embodiments, the cancer comprises an Rb-independent cancer. In some embodiments, the Rb-independent cancer is SCLC. In some embodiments, the Rb-independent cancer is TNBC. In some embodiments, the method further comprises administering a chemotherapeutic agent. In some embodiments, the chemotherapeutic agent is selected from 5-fluorouracil (5-FU), capecitabine, carboplatin, cisplatin, docetaxel, etoposide, gemcitabine, irinotecan, mitomycin, paclitaxel, vincristine, or a combination thereof. In some embodiments, the chemotherapeutic agent is carboplatin. In some embodiments, the chemotherapeutic agent is cisplatin. In some embodiments, the method further comprises administering an endocrine therapy. In some embodiments, the endocrine therapy comprises a SERD. In some embodiments, the SERD is selected from fulvestrant or elacestrant. In some embodiments, the human previously received at least one prior line of standard of care therapy. In some embodiments, the human previously received at least two prior lines of standard of care therapy. In some embodiments, the human previously received at least one prior line of CDK4/6 inhibitor therapy. In some embodiments, the human previously received at least one prior line of endocrine therapy. In some embodiments, the cancer is relapsed. In some embodiments, the cancer has progressed following a prior standard of care therapy. In some embodiments, the cancer has progressed following a prior systemic therapy. In some embodiments, the cancer is intolerant or ineligible for standard or care therapy. In some embodiments, the cancer is refractory. In some embodiments, the cancer has progressed following a prior regimen comprising a platinum analog. In some embodiments, the cancer is platinum-refractory or platinum-resistant. In some embodiments, the cancer has progressed following a prior regimen comprising a CDK4/6 inhibitor. In some embodiments, the cancer is CDK4/6 inhibitor resistant. In some embodiments, the cancer has progressed following a prior regimen comprising an endocrine therapy. In some embodiments, the cancer is endocrine therapy resistant.

In some embodiments, INX-315 or a pharmaceutically acceptable salt or ASD formulation thereof described herein in combination with abemaciclib for the treatment of a subject having a cancer. In some embodiments, the cancer is breast cancer. In some embodiments, INX-315 or a pharmaceutically acceptable salt or ASD formulation thereof is administered in conjunction with abemaciclib administered continuously. In some embodiments, the breast cancer is hormone receptor-positive (HR+) and HER2-negative. In some embodiments, the breast cancer is estrogen receptor-positive (ER+). In some embodiments, the breast cancer is progesterone receptor-positive (PR+). In some embodiments, the breast cancer is HER2-negative (HER2−). In some embodiments, the cancer is ER+/HER2− breast cancer. In some embodiments, the cancer is lung cancer. In some embodiments, the cancer is bladder cancer. In some embodiments, the cancer is ovarian cancer. In some embodiments, the cancer is endometrial cancer. In some embodiments, the cancer is esophageal cancer. In some embodiments, the esophageal cancer comprises Esophageal Adenocarcinoma. In some embodiments, the ovarian cancer comprises high-grade serous ovarian cancer (HGSOC). In some embodiments, the cancer is prostate cancer. In some embodiments, the cancer is sarcoma. In some embodiments, the cancer is uterine cancer. In some embodiments, the cancer is gastric cancer. In some embodiments, the cancer is advanced unresectable and/or metastatic cancer. In some embodiments, the cancer comprises an Rb-independent cancer. In some embodiments, the Rb-independent cancer is SCLC. In some embodiments, the Rb-independent cancer is TNBC. In some embodiments, the method further comprises administering a chemotherapeutic agent. In some embodiments, the chemotherapeutic agent is selected from 5-fluorouracil (5-FU), capecitabine, carboplatin, cisplatin, docetaxel, etoposide, gemcitabine, irinotecan, mitomycin, paclitaxel, vincristine, or a combination thereof. In some embodiments, the chemotherapeutic agent is carboplatin. In some embodiments, the chemotherapeutic agent is cisplatin. In some embodiments, the method further comprises administering an endocrine therapy. In some embodiments, the endocrine therapy comprises a SERD. In some embodiments, the SERD is selected from fulvestrant or elacestrant. In some embodiments, the human previously received at least one prior line of standard of care therapy. In some embodiments, the human previously received at least two prior lines of standard of care therapy. In some embodiments, the human previously received at least one prior line of CDK4/6 inhibitor therapy. In some embodiments, the human previously received at least one prior line of endocrine therapy. In some embodiments, the cancer is relapsed. In some embodiments, the cancer has progressed following a prior standard of care therapy. In some embodiments, the cancer has progressed following a prior systemic therapy. In some embodiments, the cancer is intolerant or ineligible for standard or care therapy. In some embodiments, the cancer is refractory. In some embodiments, the cancer has progressed following a prior regimen comprising a platinum analog. In some embodiments, the cancer is platinum-refractory or platinum-resistant. In some embodiments, the cancer has progressed following a prior regimen comprising a CDK4/6 inhibitor. In some embodiments, the cancer is CDK4/6 inhibitor resistant. In some embodiments, the cancer has progressed following a prior regimen comprising an endocrine therapy. In some embodiments, the cancer is endocrine therapy resistant.

In some embodiments, INX-315 or a pharmaceutically acceptable salt or ASD formulation thereof described herein in combination with lerociclib for the treatment of a subject having a cancer. In some embodiments, the cancer is breast cancer. In some embodiments, INX-315 or a pharmaceutically acceptable salt or ASD formulation thereof is administered in conjunction with lerociclib administered continuously. In some embodiments, the breast cancer is hormone receptor-positive (HR+) and HER2-negative. In some embodiments, the breast cancer is estrogen receptor-positive (ER+). In some embodiments, the breast cancer is progesterone receptor-positive (PR+). In some embodiments, the breast cancer is HER2-negative (HER2−). In some embodiments, the cancer is ER+/HER2− breast cancer. In some embodiments, the cancer is lung cancer. In some embodiments, the cancer is bladder cancer. In some embodiments, the cancer is ovarian cancer. In some embodiments, the cancer is endometrial cancer. In some embodiments, the cancer is esophageal cancer. In some embodiments, the esophageal cancer comprises Esophageal Adenocarcinoma. In some embodiments, the ovarian cancer comprises high-grade serous ovarian cancer (HGSOC). In some embodiments, the cancer is prostate cancer. In some embodiments, the cancer is sarcoma. In some embodiments, the cancer is uterine cancer. In some embodiments, the cancer is gastric cancer. In some embodiments, the cancer is advanced unresectable and/or metastatic cancer. In some embodiments, the cancer comprises an Rb-independent cancer. In some embodiments, the Rb-independent cancer is SCLC. In some embodiments, the Rb-independent cancer is TNBC. In some embodiments, the method further comprises administering a chemotherapeutic agent. In some embodiments, the chemotherapeutic agent is selected from 5-fluorouracil (5-FU), capecitabine, carboplatin, cisplatin, docetaxel, etoposide, gemcitabine, irinotecan, mitomycin, paclitaxel, vincristine, or a combination thereof. In some embodiments, the chemotherapeutic agent is carboplatin. In some embodiments, the chemotherapeutic agent is cisplatin. In some embodiments, the method further comprises administering an endocrine therapy. In some embodiments, the endocrine therapy comprises a SERD. In some embodiments, the SERD is selected from fulvestrant or elacestrant. In some embodiments, the human previously received at least one prior line of standard of care therapy. In some embodiments, the human previously received at least two prior lines of standard of care therapy. In some embodiments, the human previously received at least one prior line of CDK4/6 inhibitor therapy. In some embodiments, the human previously received at least one prior line of endocrine therapy. In some embodiments, the cancer is relapsed. In some embodiments, the cancer has progressed following a prior standard of care therapy. In some embodiments, the cancer has progressed following a prior systemic therapy. In some embodiments, the cancer is intolerant or ineligible for standard or care therapy. In some embodiments, the cancer is refractory. In some embodiments, the cancer has progressed following a prior regimen comprising a platinum analog. In some embodiments, the cancer is platinum-refractory or platinum-resistant. In some embodiments, the cancer has progressed following a prior regimen comprising a CDK4/6 inhibitor. In some embodiments, the cancer is CDK4/6 inhibitor resistant. In some embodiments, the cancer has progressed following a prior regimen comprising an endocrine therapy. In some embodiments, the cancer is endocrine therapy resistant.

In some embodiments, INX-315 or a pharmaceutically acceptable salt or ASD formulation thereof described herein provides an additive effect to or synergistic effect with the anti-cancer or anti-proliferative activity of a chemotherapeutic agent for the treatment of an Rb-independent cancer. In some embodiments, the chemotherapeutic agent is selected from 5-fluorouracil (5-FU), capecitabine, carboplatin, cisplatin, docetaxel, etoposide, gemcitabine, irinotecan, mitomycin, paclitaxel, vincristine, or a combination thereof. In some embodiments, the human previously received at least one prior line of chemotherapy. In some embodiments, the human previously received at least two prior lines of chemotherapy. In some embodiments, the human previously received at least one prior line of CDK4/6 inhibitor therapy. In some embodiments, the human previously received at least one prior line of endocrine therapy. In some embodiments, the cancer is relapsed. In some embodiments, the cancer has progressed following a prior standard of care therapy. In some embodiments, the cancer has progressed following a prior systemic therapy. In some embodiments, the cancer is intolerant or ineligible for standard or care therapy. In some embodiments, the cancer is refractory. In some embodiments, the cancer has progressed following a prior regimen comprising a platinum analog. In some embodiments, the cancer is platinum-refractory or platinum-resistant. In some embodiments, the cancer has progressed following a prior regimen comprising a CDK4/6 inhibitor. In some embodiments, the cancer is CDK4/6 inhibitor resistant. In some embodiments, the cancer has progressed following a prior regimen comprising an endocrine therapy. In some embodiments, the cancer is endocrine therapy resistant.

In some embodiments, the administration of INX-315 or a pharmaceutically acceptable salt thereof in a human patient in need thereof results in a mean plasma area under the curve (AUC) (ng*hr/mL) of about or greater than 2750 ng*hr/mL but less than about 28000 ng*hr/mL as measured on day 1 of dosing. In different embodiments, the administration of INX-315 or a pharmaceutically acceptable salt thereof in a human patient in need thereof results in a mean plasma area under the curve (AUC) (ng*hr/mL) of about or greater than 1250 ng*hr/mL but less than about 20000 ng*hr/mL as measured on day 1 of dosing. In different embodiments, the administration of INX-315 or a pharmaceutically acceptable salt thereof in a human patient in need thereof results in a mean plasma area under the curve (AUC) (ng*hr/mL) of about or greater than 5000 ng*hr/mL but less than about 50000 ng*hr/mL as measured on day 1 of dosing. In some embodiments, the administration of INX-315 or a pharmaceutically acceptable salt thereof in a human patient in need thereof results in a mean maximum plasma concentration (Cₘₐₓ) (ng/mL) in humans of about or greater than 150 ng/mL but less than about 2000 ng/mL as measured on day 1 of dosing. In different embodiments, the administration of INX-315 or a pharmaceutically acceptable salt thereof in a human patient in need thereof results in a mean maximum plasma concentration (Cₘₐₓ) (ng/mL) in humans of about or greater than 100 ng/mL but less than about 1600 ng/mL as measured on day 1 of dosing. In different embodiments, the administration of INX-315 or a pharmaceutically acceptable salt thereof in a human patient in need thereof results in a mean maximum plasma concentration (Cₘₐₓ) (ng/mL) in humans of about or greater than 75 ng/mL but less than about 2400 ng/mL as measured on day 1 of dosing. In some embodiments, the administration of INX-315 or a pharmaceutically acceptable salt thereof in a human patient in need thereof results in a mean minimum plasma concentration (Cₘᵢₙ) (ng/mL) of about or greater than 50 ng/mL but less than about 1000 ng/mL as measured on day 1 of dosing. In different embodiments, the administration of INX-315 or a pharmaceutically acceptable salt thereof in a human patient in need thereof results in a mean minimum plasma concentration (Cₘᵢₙ) (ng/mL) of about or greater than 20 ng/mL but less than about 600 ng/mL as measured on day 1 of dosing. In some embodiments, the administration of INX-315 or a pharmaceutically acceptable salt thereof in a human patient in need thereof results in a mean free minimum plasma concentration (Cₘᵢₙ^{free}) (ng/mL) of about or greater than 4.0 ng/mL but less than about 48.0 ng/mL as measured on day 1 of dosing. In some embodiments, the administration of INX-315 or a pharmaceutically acceptable salt thereof in a human patient in need thereof results in a mean plasma area under the curve (AUC) (ng*hr/mL) of about or greater than 5000 ng*hr/mL but less than about 56000 ng*hr/mL as measured on day 15 of dosing. In different embodiments, the administration of INX-315 or a pharmaceutically acceptable salt thereof in a human patient in need thereof results in a mean plasma area under the curve (AUC) (ng*hr/mL) of about or greater than 1250 ng*hr/mL but less than about 28000 ng*hr/mL as measured on day 15 of dosing. In some embodiments, the administration of INX-315 or a pharmaceutically acceptable salt thereof in a human patient in need thereof results in a mean maximum plasma concentration (Cₘₐₓ) (ng/mL) in humans of about or greater than 300 ng/mL but less than about 4000 ng/mL as measured on day 15 of dosing. In different embodiments, the administration of INX-315 or a pharmaceutically acceptable salt thereof in a human patient in need thereof results in a mean maximum plasma concentration (Cₘₐₓ) (ng/mL) in humans of about or greater than 90 ng/mL but less than about 2800 ng/mL as measured on day 15 of dosing. In some embodiments, the administration of INX-315 or a pharmaceutically acceptable salt thereof in a human patient in need thereof results in a mean minimum plasma concentration (Cₘᵢₙ) (ng/mL) of about or greater than 100 ng/mL but less than about 2000 ng/mL as measured on day 15 of dosing. In some embodiments, the administration of INX-315 or a pharmaceutically acceptable salt thereof in a human patient in need thereof results in a mean free minimum plasma concentration (Cₘᵢₙ^{free}) (ng/mL) of about or greater than 6.0 ng/mL but less than about 80.0 ng/mL as measured on day 15 of dosing. In some embodiments, the administration of INX-315 or a pharmaceutically acceptable salt thereof in a human patient in need thereof results in a mean whole blood area under the curve (AUC) (ng*hr/mL) of about or greater than 30000 ng*hr/mL but less than about 480000 ng*hr/mL as measured on day 1 of dosing. In different embodiments, the administration of INX-315 or a pharmaceutically acceptable salt thereof in a human patient in need thereof results in a mean whole blood area under the curve (AUC) (ng*hr/mL) of about or greater than 25000 ng*hr/mL but less than about 400000 ng*hr/mL as measured on day 1 of dosing. In some embodiments, the administration of INX-315 or a pharmaceutically acceptable salt thereof in a human patient in need thereof results in a mean maximum whole blood concentration (Cₘₐₓ) (ng/mL) in humans of about or greater than 2000 ng/mL but less than about 28000 ng/mL as measured on day 1 of dosing. In some embodiments, the administration of INX-315 or a pharmaceutically acceptable salt thereof in a human patient in need thereof results in a mean minimum whole blood concentration (Cₘᵢₙ) (ng/mL) of about or greater than 800 ng/mL but less than about 14400 ng/mL as measured on day 1 of dosing. In different embodiments, the administration of INX-315 or a pharmaceutically acceptable salt thereof in a human patient in need thereof results in a mean minimum whole blood concentration (Cₘᵢₙ) (ng/mL) of about or greater than 500 ng/mL but less than about 8000 ng/mL as measured on day 1 of dosing. In some embodiments, the administration of INX-315 or a pharmaceutically acceptable salt thereof in a human patient in need thereof results in a mean whole blood area under the curve (AUC) (ng*hr/mL) of about or greater than 50000 ng*hr/mL but less than about 800000 ng*hr/mL as measured on day 15 of dosing. In different embodiments, the administration of INX-315 or a pharmaceutically acceptable salt thereof in a human patient in need thereof results in a mean whole blood area under the curve (AUC) (ng*hr/mL) of about or greater than 25000 ng*hr/mL but less than about 600000 ng*hr/mL as measured on day 15 of dosing. In some embodiments, the administration of INX-315 or a pharmaceutically acceptable salt thereof in a human patient in need thereof results in a mean maximum whole blood concentration (Cₘₐₓ) (ng/mL) in humans of about or greater than 3000 ng/mL but less than about 56000 ng/mL as measured on day 15 of dosing. In some embodiments, the administration of INX-315 or a pharmaceutically acceptable salt thereof in a human patient in need thereof results in a mean minimum whole blood concentration (Cₘᵢₙ) (ng/mL) of about or greater than 1000 ng/mL but less than about 24000 ng/mL as measured on day 15 of dosing. In some embodiments, the administration of INX-315 or a pharmaceutically acceptable salt thereof in a human patient in need thereof results in a whole blood to plasma area under the curve (AUC) ratio of about or greater than 6 but less than 22 as measured on day 1 of dosing. In some embodiments, the administration of INX-315 or a pharmaceutically acceptable salt thereof in a human patient in need thereof results in a whole blood to plasma area under the curve (AUC) ratio of about or greater than 6 but less than 40 as measured on day 15 of dosing. In some embodiments, the administration of INX-315 or a pharmaceutically acceptable salt thereof in a human patient in need thereof results in a mean Cₘₐₓ to Cₘᵢₙ ratio of less than 5 but greater than about 1.25. In some embodiments, the administration of INX-315 or a pharmaceutically acceptable salt thereof in a human patient having lung cancer results in a t_{1/2} of between about 12 hours and about 24 hours. In some embodiments, the administration of INX-315 or a pharmaceutically acceptable salt thereof in a human patient in need thereof results in a t_{1/2} of between about 12 hours and about 18 hours. In some embodiments, the t_{1/2} is about or greater than about 14 hours but less than about 18 hours. In some embodiments, the t_{1/2} is about or greater than about 15 hours but less than about 18 hours. In some embodiments, the t_{1/2} is about or greater than about 16 hours but less than about 18 hours. In some embodiments, the INX-315 is administered at a dose of between about 100 mg and about 500 mg. In some embodiments, the INX-315 is administered at a dose of about 100 mg, about 125 mg, or about 150 mg. In some embodiments, the INX-315 is administered at a dose of about 200 mg, about 225 mg, or about 250 mg. In some embodiments, the INX-315 is administered at a dose of about 300 mg, about 325 mg, or about 350 mg. In some embodiments, the INX-315 is administered at a dose of about 400 mg, about 425 mg, or about 450 mg. In some embodiments, the INX-315 is orally administered. In some embodiments, the INX-315 is formulated as an amorphous spray-dried dispersion (ASD).

In certain aspects, disclosed herein are methods for treating a human with a cancer having overexpression and/or activation of cyclin E1 (CCNE1) and/or cyclin E2 (CCNE2) compared with a control sample, wherein overexpression and/or activation of CCNE1 and/or CCNE2 comprise a cyclin E amplified or overexpressed cancer. In some embodiments, cyclin E1 (CCNE1) and/or cyclin E2 (CCNE2) is overexpressed and/or activated in a sample of a subject to be treated by at least 1.5-fold, at least 2.0-fold, at least 2.5-fold, at least 3.0-fold, at least 3.5-fold, at least 4.0-fold, at least 4.5-fold, at least 5.0-fold, or greater than 5.0-fold compared with a control sample. In some embodiments, the method further comprises administering an effective amount of a CDK4/6 inhibitor. In some embodiments the CDK4/6 inhibitor is a selective CDK4/6 inhibitor. In some embodiments, the additional CDK4/6 inhibitor is selected from palbociclib, ribociclib, abemaciclib, trilaciclib, lerociclib, or dalpiciclib. In some embodiments, the additional CDK4/6 inhibitor is selected from BPI-16350, narazaciclib (ON-123300), FLX-925 (AMG-925), UCT-03-008, GLR2007, birociclib (XZP-3287), LY5219, PF-07220060, or ON-123300. In some embodiments, the method further comprises administering an effective amount of a selective CDK4 inhibitor. In some embodiments, the selective CDK4 inhibitor is PF-07220060. In some embodiments, an NGS panel test is used to confirm CCNE1 or CCNE2 overexpression or amplification status. In some embodiments, an NGS panel test to confirm CCNE1 or CCNE2 overexpression or amplification status is selected from Foundation One® CDx, Foundation One® Liquid CDx, Tempus xT (solid tumor), Tempus xF (liquid biopsy), Caris® Life Sciences Molecular Profiling, or OncoHelix Solid Tumor NGS. In some embodiments, the patient sample is selected from tumor tissue, formalin-fixed paraffin embedded (FFPE) tumor tissue, blood, or blood plasma.

In some embodiments, INX-315 is administered at a dose from about 100 mg to about 500 mg. In some embodiments, INX-315, or a pharmaceutically acceptable salt thereof, is administered at a dose of about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In some embodiments, INX-315, or a pharmaceutically acceptable salt thereof, is administered at a dose of about 100 mg. In some embodiments, INX-315, or a pharmaceutically acceptable salt thereof, is administered at a dose of about 200 mg. In some embodiments, INX-315, or a pharmaceutically acceptable salt thereof, is administered at a dose of about 300 mg. In some embodiments, INX-315, or a pharmaceutically acceptable salt thereof, is administered at a dose of about 400 mg. In some embodiments, INX-315, or a pharmaceutically acceptable salt thereof, is administered at a dose of about 500 mg. In some embodiments, the INX-315 is orally administered. In some embodiments, the INX-315 is formulated as an amorphous spray-dried dispersion (ASD).

### Pharmaceutical Compositions and Dosage Forms

INX-315 or its pharmaceutically acceptable salt can be administered in an effective amount according to the methods described herein to a host to treat any of the disorders described herein using any suitable approach which achieves the desired therapeutic result. The amount and timing of INX-315 administration will, of course, be dependent on the host being treated, the instructions of the supervising medical specialist, on the time course of the exposure, on the manner of administration, on the pharmacokinetic properties, and on the judgment of the prescribing physician. Thus, because of host-to-host variability, the dosages given below are a guideline and the physician can titrate doses of the compound to achieve the treatment that the physician considers appropriate for the host. In considering the degree of treatment desired, the physician can balance a variety of factors such as age and weight of the host, presence of preexisting disease, as well as presence of other diseases.

### Amorphous Solid Dispersion Spray Dry Formulation

In some embodiments, INX-315 is co-spray dried with a precipitation preventer in a solvent as an amorphous spray-dried dispersion (ASD) to achieve desirable functionality of the ASD for a final pharmaceutical composition.

Spray drying is the transformation of a feed from a fluid state into a dried particulate form by spraying the feed into a hot drying medium. Spray drying process used in the manufacturing of an ASD typically includes steps of: (i) feed atomization where the feed is atomized into a fine spray; (ii) drying of sprayed droplets of the atomized feed with the hot drying gas to form solid particles; and (iii) separating the dried particles from the gas.

In some embodiments, the spray-drying solution contains INX-315, a precipitation preventer, and a solvent. In some embodiments, the precipitation preventer is selected from cellulose derivative, polyvinylpyrrolidone (PVP) and PVP/VA (vinyl acetate), or polymethacrylate. In some embodiments, the precipitation preventer is selected from hypromellose, such as HPMC 2910, hydroxyethyl cellulose (HEC), hydroxypropyl methylcellulose acetate succinate (HPMCAS), hydroxypropyl methylcellulose phthalate (HPMCP), cellulose acetate phthalate (CAP), sodium carboxymethyl cellulose (Na-CMC), polyacrylic acid, polyethylene glycol, such as PEG 4000, PEG 6000, PEG 8000, and PEG 20000, polyvinylpyrrolidone, such as PVP K 30, PVP K 25, PVP VA64, and PVP VA37. In some embodiments, the HPMCAS is selected from HPMCAS-L, HPMCAS-LF, HPMCAS-LG, HPMCAS-M, HPMCAS-MF, HPMCAS-MG, HPMCAS-H, HPMCAS-HF, HPMCAS-HG, or HPMCAS-E3. In some embodiments, the HPMCAS is HPMCAS-H. In some embodiments, the HPMCAS is HPMCAS-HF. In some embodiments, the HPMCAS is HPMCAS-HG. In some embodiments, the precipitation preventer is selected from erythritol, maltodextrin, mannitol, crospovidone, colloidal silicon dioxide, polysorbate 80, lactose, PVP, PVP-VA, chitosan chlorhydrate, meglumine, copovidone, HPMC, sodium alginate, or sodium carmellose.

In some embodiments, INX-315 is between about 20% and about 50% by weight of the ASD. In some embodiments, INX-315 is between about 25% and about 45% by weight of the ASD. In some embodiments, INX-315 is about 40% by weight of the ASD. In some embodiments, the precipitation preventer precipitation preventer in the ASD is between about 40% and about 70% by weight of the ASD. In some embodiments, the precipitation preventer precipitation preventer in the ASD is between about 45% and about 65% by weight of the ASD. In some embodiments, the precipitation preventer in the ASD is about 60% by weight of the ASD.

The liquid feed used in spray drying can be a suspension, solution, or emulsion. The liquid solution, suspension, or emulsion can be pumped from the feed container to the drying chamber through an atomization nozzle. In some embodiments, water is used as a solvent in the liquid feed. In some embodiments, a suitably nontoxic organic solvent and/or cosolvent or an aqueous suspension that is appropriate for a pharmaceutical formulation is used to prepare the liquid feed. In some embodiments, the solvent is selected from water, ethanol, isopropanol, dioxane, chloroform, THF, THF stabilized with butylated hydroxytoluene (BHT), methanol, EtOAc, acetone, dichloromethane, or a mixture thereof. In some embodiments, the solvent is selected from water, acetic acid, acetone, anisole, 1-butanol, 2-butanol, butylacetate, tert-butylmethylether, 1,1-diethoxypropane, 1,1-dimethoxymethnae, 2,2-dimethoxypropane, dimethylsulfoxide, ethanol, ethylacetate, ethylether, ethylformate, formic acid, heptane, isobutylacetate, isooctane, isopropylether, methylacetate, methylisopropylketone, methyltetrahydrofuran, 3-methyl-1-butanol, methylethylketone, 2-methyl-1-propanol, pentane, 1-pentanol, petroleumether, 1-propanol, 2-propanol, propylacetate, trichloroacetic acid, trifluoroacetic acid, triethylamine, or a mixture thereof. In some embodiments, the solvent comprises or consists of methanol and water. In some embodiments, the solvent comprises or consists of THF and water. In some embodiments, the solvent comprises or consists of 80% THF and 20% water. In some embodiments, the solvent comprises or consists of 90% THF and 10% water. In some embodiments, the solvent comprises or consists of 92% THF and 8% water. Atomization is typically accomplished using either a pressurized nozzle or rotary disk atomizer. In alternative embodiments, ultrasonic nozzles or two-fluid nozzles may be used.

Once the liquid feed is atomized, the fine spray is brought into contact with the hot drying gas. In some embodiments, the hot drying gas is nitrogen. The flow of the feed and gas can be counter-current, co-current, or mixed flow. In the drying step, liquids evaporate almost instantly, creating solid particles. In the spray drying process, the drying rate of the feed is extremely fast, because the feed can be atomized into a fine spray or mist with a huge surface area that facilitates evaporation of the solvent. The solid particles that result from spray drying can be granules, a powder, or agglomerates.

The final step in the spray drying process is to separate the dry particles from the gas. This can be done, for example, using a filter or a cyclone.

In some embodiments, a pharmaceutical composition is manufactured from the ASD formulations as described herein. For example, in some embodiments, the pharmaceutical composition is prepared from an ASD formulation comprising amorphous INX-315 and a precipitation preventer, wherein the ASD formulation is dry-granulated, dry-blended, then tableted with one or more pharmaceutically acceptable excipients. In some embodiments, the use of a pharmaceutical composition comprising an INX-315 ASD formulation as described herein provides improved PK parameters including for example sustained and elevated drug exposure in plasma and whole blood.

In one aspect, provided herein is a pharmaceutical composition comprising an amorphous spray-dried dispersion (ASD) of a compound of structure of INX-315, or a pharmaceutically acceptable salt thereof, and a pharmaceutically acceptable excipient, wherein the ASD further comprises a precipitation preventer. In some embodiments, INX-315 is between about 20% and about 60% by weight of the ASD. In some embodiments, INX-315 is between about 30% and about 50% by weight of the ASD. In some embodiments, INX-315 is about 40% by weight of the ASD. In some embodiments, the pharmaceutical composition comprises between about 100 mg and about 500 mg of INX-315. In some embodiments, the precipitation preventer is hydroxypropyl methylcellulose acetate succinate (HPMCAS). In some embodiments, the precipitation preventer is HPMCAS-HG. In some embodiments, the precipitation preventer in the ASD is between about 40% and about 80% by weight of the ASD. In some embodiments, the precipitation preventer in the ASD is about 60% by weight of the ASD. In some embodiments, the ASD is between about 40% and about 60% by weight of the pharmaceutical composition. In some embodiments, the ASD is about 50% by weight of the pharmaceutical composition. In some embodiments, the pharmaceutical composition comprises an insoluble diluent. In some embodiments, the insoluble diluent is microcrystalline cellulose. In some embodiments, the insoluble diluent is between about 15% and about 25% by weight of the pharmaceutical composition. In some embodiments, the pharmaceutical composition comprises a soluble diluent. In some embodiments, the soluble diluent is mannitol. In some embodiments, the soluble diluent is between about 15% and about 25% by weight of the pharmaceutical composition. In some embodiments, the pharmaceutical composition comprises a disintegrant. In some embodiments, the disintegrant is croscarmellose sodium. In some embodiments, the disintegrant is between about 1% and about 10% by weight of the pharmaceutical composition. In some embodiments, the pharmaceutical composition comprises a glidant. In some embodiments, the glidant is colloidal silicon dioxide. In some embodiments, the glidant is between about 0.1% and about 5% by weight of the pharmaceutical composition. In some embodiments, the pharmaceutical composition comprises a lubricant. In some embodiments, the lubricant is sodium stearyl fumarate. In some embodiments, the lubricant is between about 0.1% and about 5% by weight of the pharmaceutical composition.

The pharmaceutical composition may be formulated as any pharmaceutically useful form, e.g., a liquid oral dosage form, a solid oral dosage form, a semisolid oral dosage form, as an aerosol, a cream, a gel, a pill, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose.

The INX-315 or its pharmaceutically acceptable salt used as described herein may be administered orally, topically, parenterally, by inhalation or spray, sublingually, via implant, including ocular implant, transdermally, via buccal administration, rectally, as an ophthalmic solution, injection, including ocular injection, intramuscular, inhalation, intra-aortal, intracranial, subdermal, intraperitoneal, subcutaneous, transnasal, sublingual, or rectal or by other means, in dosage unit formulations containing conventional pharmaceutically acceptable carriers. For ocular delivery, the compound can be administered, as desired, for example, via intravitreal, intrastromal, intracameral, sub-tenon, sub-retinal, retro-bulbar, peribulbar, suprachorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

In some embodiments, the pharmaceutical composition comprising amorphous INX-315 or a pharmaceutically acceptable salt thereof in an ASD is administered orally. For example, in some embodiments, a pharmaceutical composition comprising amorphous INX-315 or a pharmaceutically acceptable salt thereof and precipitation preventer in the ASD is administered orally.

The therapeutically effective dosage of INX-315 or a pharmaceutically acceptable salt thereof can be determined by the health care practitioner depending on the condition, size and age of the patient as well as the route of delivery. In certain non-limited embodiments, a dosage from about 0.1 to about 200 mg/kg has therapeutic efficacy, with all weights being calculated based upon the weight of the INX-315, including the cases where a salt is employed. In some embodiments, the dosage is about or greater than 0.1, 0.5, 1, 5, 10, or 25 mg/kg.

In some embodiments, the pharmaceutical composition is in a dosage form that contains from about 50 to 500 mg, for example from 100 to 400 or 500 mg, or further for example, 100, 125, 150, 175, 200, 225, 250, 275, 300, 325, 350, 375, 400, 425, 450, 475, or 500 mg of INX-315 in a unit dosage form. In some embodiments, the pharmaceutical composition is in a dosage form that contains about 100 mg or 150 mg of INX-315, or a pharmaceutically acceptable salt thereof. In some embodiments, the pharmaceutical composition is in a dosage form that contains about 200 mg of INX-315, or a pharmaceutically acceptable salt thereof. In some embodiments, the pharmaceutical composition is in a dosage form that contains about 300 mg of INX-315, or a pharmaceutically acceptable salt thereof. In some embodiments, the pharmaceutical composition is in a dosage form that contains about 400 mg of INX-315, or a pharmaceutically acceptable salt thereof. In some embodiments, the pharmaceutical composition is in a dosage form that contains about 500 mg of INX-315, or a pharmaceutically acceptable salt thereof. The pharmaceutical composition may also include a molar ratio of the INX-315 or a pharmaceutically acceptable salt thereof and an additional active agent (such as a selective CDK 4 inhibitor), in a ratio that achieves the desired results.

In some embodiments, INX-315 or a pharmaceutically acceptable salt thereof is administered once-a-day (QD). In some embodiments, INX-315 or a pharmaceutically acceptable salt thereof is administered twice a day (QD). In some embodiments, INX-315 or a pharmaceutically acceptable salt thereof is administered at least once-a-day for at least 21 days, at least 24 days, at least 28 days, at least 35 days, at least 45 days, at least 60 days, at least 75 days, at least 90 days, at least 120 days, at least 180 days, at least 1 year or longer, including indefinitely, or until the healthcare provider decides that the drug is no longer necessary.

In accordance with the presently disclosed methods, an oral administration can be in any desired form such as a solid, gel or liquid, including a solution, suspension, or emulsion. In some embodiments, the INX-315 or salt thereof is administered by inhalation or intramuscularly as a liposomal suspension. When administered through inhalation INX-315 or salt may be in the form of a plurality of solid particles or droplets having any desired particle size, and for example, from about 0.01, 0.1 or 0.5 to about 5, 10, 20 or more microns, and optionally from about 1 to about 2 microns.

The pharmaceutical formulations can comprise INX-315 or a pharmaceutically acceptable salt thereof, in any pharmaceutically acceptable carrier. If a solution is desired, water may sometimes be the carrier of choice for water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and for illustration by filtration through a 0.22-micron filter.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the INX-315 is sufficient to provide a practical quantity of material for administration per unit dose of INX-315.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of INX-315.

Additionally, auxiliary substances, such as wetting or emulsifying agents, biological buffering substances, surfactants, and the like, can be present in such vehicles. A biological buffer can be any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions can be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of INX-315 in combination with a pharmaceutically acceptable carrier and, in addition, can include other pharmaceutical agents, adjuvants, diluents, buffers, and the like.

In certain independent embodiments of the invention, pharmaceutical compositions are provided comprising i) an ASD formulation of the present invention comprising amorphous INX-315, or a pharmaceutically acceptable salt thereof, spray-dried with a precipitation preventer; and ii) optionally additionally includes one or more pharmaceutically acceptable excipients, wherein the ASD formulation is dry-granulated, dry-blended, then tableted with the one or more optional pharmaceutically acceptable excipients. In some embodiments, the precipitation preventer in the ASD comprises hydroxypropyl methylcellulose acetate succinate (HPMCAS) selected from HPMCAS-L, HPMCAS-LF, HPMCAS-LG, HPMCAS-M, HPMCAS-MF, HPMCAS-MG, HPMCAS-H, HPMCAS-HF, HPMCAS-HG, or HPMCAS-E3. In some embodiments, INX-315 is between about 10% and about 30% by weight of the pharmaceutical composition, or between about 15% and about 25% by weight of the pharmaceutical composition. In some embodiments, INX-315 is about 20% by weight of the pharmaceutical composition. In some embodiments, the precipitation preventer precipitation preventer in the ASD is between about 20% and about 50% by weight of the pharmaceutical composition. In some embodiments, the precipitation preventer precipitation preventer in the ASD is between about 25% and about 45% by weight of the pharmaceutical composition. In some embodiments, the precipitation preventer precipitation preventer in the ASD is about 30% by weight of the pharmaceutical composition.

In some embodiments, the amorphous INX-315 or a pharmaceutically acceptable salt thereof and a precipitation preventer are in the following weight/weight percentage ranges in the ASD formulations and the pharmaceutical compositions as described herein:

| | Represen- | | Percent (w/w) of the |
|---|---|---|---|
| | tative | Percent (w/w) of the | Pharmaceutical |
| Class | Components | ASD formulation | Composition |
| Active | INX-315 | between about 20% | between about 10% |
| Pharmaceutical | | to about 50%, or | to about 30%, or |
| Ingredient | | between about 25% | between about 15% |
| (API) | | to about 45%, or | to about 25%, or |
| | | for example about | for example about |
| | | 40% | 20% |
| Precipitation | HPMCAS | between about 40% | between about 20% |
| Preventer | | to about 70%, or | to about 50%, or |
| | | between about 45% | between about 25% |
| | | to about 65%, or | to about 45%, or |
| | | for example about | for example about |
| | | 60% | 30% |

In some embodiments, the pharmaceutical composition comprises an amorphous INX-315 or a pharmaceutically acceptable salt thereof, and a precipitation preventer spray-dried together in an ASD formulation, and following this, one or more pharmaceutically acceptable excipients selected from insoluble diluent, soluble diluent, disintegrant, glidant, and/or lubricant are dry-granulated, dry-blended, then tableted together. Non-limiting examples of the one or more pharmaceutically acceptable excipients include vitamin E (for example, d-alpha-tocopheryl polyethylene glycol succinate), mannitol, cellulose (for example microcrystalline cellulose), croscarmellose sodium, silicon dioxide (for example untreated fumed colloidal), and sodium stearyl fumarate. In some embodiments, a pharmaceutical composition according to the present invention is formulated into a dosage unit form, such as an oral dosage unit form. In some embodiments, a pharmaceutical composition according to the present invention is formulated into a tablet dosage form.

In some embodiments, the pharmaceutical composition comprising amorphous INX-315 or a pharmaceutically acceptable salt thereof comprises one or more of the following pharmaceutically acceptable excipients:

| | | Percent (w/w) of the |
|---|---|---|
| | Representative | Pharmaceutical |
| Class | Components | Composition |
| Insoluble | Cellulose, for example | about 15% to about 25%, |
| diluent | Microcrystalline Cellulose | for example about 20.5% |
| Soluble | Mannitol | about 15% to about 25%, |
| diluent | | for example about 20.5% |
| Disintegrant | Croscarmellose, for example | about 1% to about 10%, |
| | Croscarmellose Sodium | for example about 6% |
| Glidant | Silicon dioxide, for example | about 1% to about 10%, |
| | Colloidal Silicon Dioxide | for example about 6% |
| Lubricant | Sodium Stearyl Fumarate | about 0.1% to about 5%, |
| | | for example about 1.5% |

In some embodiments, a process for manufacturing a pharmaceutical composition comprising INX-315 or a pharmaceutically acceptable salt thereof, and pharmaceutically acceptable excipients, comprises: (i) spray-drying a solution comprising INX-315, or a pharmaceutically acceptable salt thereof, and a precipitation preventer to yield an amorphous spray-dried dispersion (ASD) comprising INX-315, or a pharmaceutically acceptable salt thereof; (ii) granulating the ASD together with one or more pharmaceutically acceptable excipients to provide a granulate; (iii) dry blending the granulate together with one or more pharmaceutically acceptable excipients; and (iv) tablet compressing the dry-blended granulate to provide a pharmaceutical composition comprising amorphous INX-315, or a pharmaceutically acceptable salt thereof, and pharmaceutically acceptable excipients in an oral dosage unit form. In some embodiments, the oral dosage unit form has a nominal tablet strength of between about 100 mg and about 500 mg. In some embodiments, the oral dosage unit form has a nominal tablet strength of between about 100 mg and about 300 mg. In some embodiments, the oral dosage unit form has a nominal tablet strength of about 100 mg. In some embodiments, the oral dosage unit form has a nominal tablet strength of about 150 mg. In some embodiments, the oral dosage unit form has a nominal tablet strength of between about 200 mg and about 400 mg. In some embodiments, the oral dosage unit form has a nominal tablet strength of about 200 mg. In some embodiments, the oral dosage unit form has a nominal tablet strength of about 250 mg. In some embodiments, the oral dosage unit form has a nominal tablet strength of about 300 mg. In some embodiments, the oral dosage unit form has a nominal tablet strength of about 350 mg. In some embodiments, the oral dosage unit form has a nominal tablet strength of about 400 mg.

Thus, the compositions of the disclosure can be administered as pharmaceutical formulations including those suitable for oral (including buccal and sub-lingual), rectal, nasal, topical, pulmonary, vaginal or parenteral (including intramuscular, intrathecal, and subcutaneous) administration or in a form suitable for administration by inhalation or insufflation. The preferred manner of administration is oral using a convenient daily dosage regimen which can be adjusted according to the degree of affliction.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, INX-315 or a pharmaceutically acceptable salt thereof and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or can be apparent, to those skilled in this art; for example, see Remington's Pharmaceutical Sciences, 23rd Edition, A. Adejare, Editor, Academic Press (2020); Handbook of Pharmaceutical Excipients, 6th Edition, R. C. Rowe, P. J. Sheskey, M. E. Quinn Editors, American Pharmaceutical Association, and Pharmaceutical Press (2009); and Handbook of Pharmaceutical Additives, 3rd Edition, compiled by Michael and Irene Ash, Synapse Information Resources (2007).

In yet another embodiment is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition will generally take the form of a tablet, capsule, a softgel capsule or can be an aqueous or nonaqueous solution, suspension or syrup. Tablets and capsules are preferred oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the INX-315 can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

Parenteral formulations can be prepared in conventional forms, either as liquid solutions or suspensions, solid forms suitable for solubilization or suspension in liquid prior to injection, or as emulsions. Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained. Parenteral administration includes intraarticular, intramuscular, intradermal, intraperitoneal, and subcutaneous routes. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

In addition to INX-315 or a pharmaceutically acceptable salt thereof, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. An antimicrobial preservative is typically employed when the formulations is placed in a vial designed for multi-dose use. The pharmaceutical formulations described herein can be lyophilized using techniques well known in the art.

In some embodiments, INX-315 or its pharmaceutically acceptable salt can be provided in the form of a lyophilizate, which is capable of being reconstituted with a suitable pharmaceutically acceptable carrier to form liquid formulation suitable for injection thereof into a host. When INX-315 or a pharmaceutically acceptable salt thereof is substantially water-insoluble, a sufficient amount of emulsifying agent, which is physiologically acceptable, can be employed in sufficient quantity to emulsify INX-315 or the pharmaceutically acceptable salt thereof in an aqueous carrier. Particularly useful emulsifying agents include phosphatidyl cholines and lecithin.

Pharmaceutical formulations also are provided which provide a controlled release of INX-315 or a pharmaceutically acceptable salt thereof described herein, including through the use of a degradable polymer, as known in the art.

The term “pharmaceutically acceptable salts” as used herein refers to those salts which are, within the scope of sound medical judgment, suitable for use in contact with hosts (e.g., human hosts) without undue toxicity, irritation, allergic response, and the like, commensurate with a reasonable benefit/risk ratio, and effective for their intended use, as well as the zwitterionic forms, where possible, of INX-315 as described herein.

Thus, the term “salts” refers to the relatively non-toxic, inorganic and organic acid addition salt of INX-315. These salts can be prepared during the final isolation and purification of the compound or by separately reacting the purified INX-315 in its free base form with a suitable organic or inorganic acid and isolating the salt thus formed. Basic compounds are capable of forming a wide variety of different salts with various inorganic and organic acids. Acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents.

Salts can be prepared from inorganic acids include hydrochloric, sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, hemisulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Preferably, sterile injectable suspensions are formulated according to techniques known in the art using suitable carriers, dispersing or wetting agents and suspending agents. The sterile injectable formulation can also be a sterile injectable solution or a suspension in a nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating INX-315 of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Formulations suitable for rectal administration are typically presented as unit dose suppositories. These may be prepared by admixing INX-315 or a pharmaceutically acceptable salt thereof with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. In some embodiments, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

### Improved Patient Outcomes

The administration of a treatment described herein to the patient subgroups described herein may provide enhanced anti-tumor efficacy in patients. In some embodiments, the administration of a treatment described herein provides improved progression free survival (PFS) and/or overall survival (OS) in comparison to the predicted PFS and/or OS in subjects receiving a CDK2 inhibitor treatment other than INX-315, for example, BLU-222 or PF-07104091.

In some embodiments, the treatment results in a reduction of incidents of treatment-emergent adverse events in comparison to the predicted number of treatment-emergent adverse events in subjects receiving a CDK2 inhibitor treatment other than INX-315, for example, BLU-222 or PF-07104091. In some embodiments, the treatment-emergent adverse events are selected from nausea, vomiting, anemia, diarrhea, fatigue, blurred vision, or a combination thereof. In some embodiments, the treatment results in a reduction of diarrhea incidents in comparison to the predicted number of diarrhea incidents in subjects receiving a CDK2 inhibitor treatment other than INX-315, for example, BLU-222 or PF-07104091. In some embodiments, the treatment results in a reduction of vomiting incidents in comparison to the predicted number of vomiting incidents in subjects receiving a CDK2 inhibitor treatment other than INX-315, for example, BLU-222 or PF-07104091. In some embodiments, the treatment-emergent adverse events are Grade 3 or greater. In some embodiments, the treatment-emergent adverse events are Grade 4 or greater. In some embodiments, the treatment results in a reduction of incidents of laboratory abnormalities in comparison to the predicted number of incidents of laboratory abnormalities in subjects receiving a CDK2 inhibitor treatment other than INX-315, for example, BLU-222 or PF-07104091. In some embodiments, the treatment results in an improved overall survival (OS) in comparison to the predicted overall survival (OS) in subjects receiving a CDK2 inhibitor treatment other than INX-315, for example, BLU-222 or PF-07104091. In some embodiments, the treatment results in an improved overall response rate (ORR) in comparison to the predicted overall response rate (ORR) in subjects receiving a CDK2 inhibitor treatment other than INX-315, for example, BLU-222 or PF-07104091. In some embodiments, the treatment results in an improved disease control rate (DCR) in comparison to the predicted disease control rate (DCR) in subjects receiving a CDK2 inhibitor treatment other than INX-315, for example, BLU-222 or PF-07104091. In some embodiments, the treatment results in an improved progression free survival (PFS) in comparison to the predicted progression free survival (PFS) in subjects receiving a CDK2 inhibitor treatment INX-315, for example, BLU-222 or PF-07104091. In some embodiments, the treatment results in an improved duration of response (DOR) in comparison to the predicted duration of response (DOR) in subjects receiving a CDK2 inhibitor treatment other than INX-315, for example, BLU-222 or PF-07104091. In some embodiments, the treatment results in an extension of time to progression (TTP) in comparison to the predicted time to progression (TTP) in subjects receiving a CDK2 inhibitor treatment other than INX-315, for example, BLU-222 or PF-07104091. In some embodiments, the CDK2 treatment other than INX-315 comprises the administration of a CDK2 inhibitor selected from BLU-222 or PF-07104091. In some embodiments, the CDK inhibitor other than INX-315 is BLU-222. In some embodiments, the CDK inhibitor other than INX-315 is PF-07104091. In some embodiments, the CDK2 inhibitor other than INX-315 is administered in a dosage amount and frequency identical to INX-315.

### Additional Embodiments

1A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 2,750 up to about 7,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
2A. The method of embodiment 1A, wherein the mean AUC is at least about 3,000.
3A. The method of embodiment 1A, wherein the mean AUC is at least about 3,250.
4A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 1,250 up to about 5,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
5A. The method of embodiment 4A, wherein the mean AUC is at least about 1,500.
6A. The method of embodiment 4A, wherein the mean AUC is at least about 1,750.
7A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 150 up to about 500 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
8A. The method of embodiment 7A, wherein the mean Cₘₐₓ is at least about 175.
9A. The method of embodiment 7A, wherein the mean Cₘₐₓ is at least about 200.
10A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 100 up to about 400 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
11A. The method of embodiment 10A, wherein the mean Cₘₐₓ is at least about 125.
12A. The method of embodiment 10A, wherein the mean Cₘₐₓ is at least about 150.
13A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 50 up to about 250 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
14A. The method of embodiment 13A, wherein the mean Cₘᵢₙ is at least about 60.
15A. The method of embodiment 13A, wherein the mean Cₘᵢₙ is at least about 75.
16A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 20 up to about 150 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
17A. The method of embodiment 16A, wherein the mean Cₘᵢₙ is at least about 40.
18A. The method of embodiment 16A, wherein the mean Cₘᵢₙ is at least about 60.
19A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean free minimum plasma concentration (C_{minfree}) (ng/mL) of at least about 4.0 up to about 12.0 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
20A. The method of embodiment 19A, wherein the mean C_{minfree} is at least about 5.0.
21A. The method of embodiment 19A, wherein the mean C_{minfree} is at least about 6.0.
22A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 5,000 up to about 14,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
23A. The method of embodiment 22A, wherein the mean AUC is at least about 5,500.
24A. The method of embodiment 22A, wherein the mean AUC is at least about 6,000.
25A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 300 up to about 1,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
26A. The method of embodiment 25A, wherein the mean Cₘₐₓ is at least about 350.
27A. The method of embodiment 25A, wherein the mean Cₘₐₓ is at least about 400.
28A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 100 up to about 500 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
29A. The method of embodiment 28A, wherein the mean Cₘᵢₙ is at least about 125.
30A. The method of embodiment 28A, wherein the mean Cₘᵢₙ is at least about 150.
31A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean free minimum plasma concentration (C_{minfree}) (ng/mL) of at least about 6.0 up to about 20.0 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
32A. The method of embodiment 31A, wherein the mean C_{minfree} is at least about 7.0.
33A. The method of embodiment 31A, wherein the mean C_{minfree} is at least about 8.0.
34A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean whole blood area under the curve (AUC) (ng*hr/mL) of at least about 30,000 up to about 120,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
35A. The method of embodiment 34A, wherein the mean AUC is at least about 40,000.
36A. The method of embodiment 34A, wherein the mean AUC is at least about 50,000.
37A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum whole blood concentration (Cₘₐₓ) (ng/mL) of at least about 2000 up to about 7000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
38A. The method of embodiment 37A, wherein the mean Cₘₐₓ is at least about 2,250.
39A. The method of embodiment 37A, wherein the mean Cₘₐₓ is at least about 2,500.
40A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 800 up to about 3,600 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
41A. The method of embodiment 40A, wherein the mean Cₘᵢₙ is at least about 900.
42A. The method of embodiment 40A, wherein the mean Cₘᵢₙ is at least about 1,000.
43A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 500 up to about 2,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
44A. The method of embodiment 43A, wherein the mean Cₘᵢₙ is at least about 575.
45A. The method of embodiment 43A, wherein the mean Cₘᵢₙ is at least about 650.
46A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean whole blood area under the curve (AUC) (ng*hr/mL) of at least about 50,000 up to about 200,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
47A. The method of embodiment 46A, wherein the mean AUC is at least about 60,000.
48A. The method of embodiment 46A, wherein the mean AUC is at least about 70,000.
49A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum whole blood concentration (Cₘₐₓ) (ng/mL) of at least about 3,000 up to about 14,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
50A. The method of embodiment 49A, wherein the mean Cₘₐₓ is at least about 3,500.
51A. The method of embodiment 49A, wherein the mean Cₘₐₓ is at least about 4,000.
52A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 1,000 up to about 6,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
53A. The method of embodiment 52A, wherein the mean Cₘᵢₙ is at least about 1,300.
54A. The method of embodiment 52A, wherein the mean Cₘᵢₙ is at least about 1,600.
55A. The method of any one of embodiments 1A-54A, which further results in a mean Cₘₐₓ to Cₘᵢₙ ratio of at least about 1.25 up to about 5.
56A. The method of any one of embodiments 1A-55A, wherein the Cₘₐₓ to Cₘᵢₙ ratio is less than 3.
57A. The method of any one of embodiments 1A-56A, wherein the Cₘₐₓ to Cₘᵢₙ ratio is less than 2.25.
58A. The method of any one of embodiments 1A-57A, wherein INX-315 is administered once-a-day.
59A. The method of any one of embodiments 1A-58A, wherein INX-315 is administered at a dose of about 100 mg, about 125 mg, or about 150 mg.
60A. The method of any one of embodiments 1A-59A, wherein INX-315 is orally administered.
61A. The method of any one of embodiments 1A-60A, wherein the INX-315 is administered in a pharmaceutical composition comprising: i) an amorphous spray-dried dispersion (ASD) formulation comprising amorphous INX-315 spray-dried with one or more precipitation preventers or additives that provide the desired properties for the ASD; and ii) optionally additionally includes one or more pharmaceutically acceptable excipients.
62A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 5,550 up to about 14,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
63A. The method of embodiment 62A, wherein the mean AUC is at least about 6,000.
64A. The method of embodiment 62A, wherein the mean AUC is at least about 6,500.
65A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 2,500 up to about 10,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
66A. The method of embodiment 65A, wherein the mean AUC is at least about 3,000.
67A. The method of embodiment 65A, wherein the mean AUC is at least about 3,500.
68A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 300 up to about 1,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
69A. The method of embodiment 68A, wherein the mean Cₘₐₓ is at least about 350.
70A. The method of embodiment 68A, wherein the mean Cₘₐₓ is at least about 400.
71A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 200 up to about 800 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
72A. The method of embodiment 71A, wherein the mean Cₘₐₓ is at least about 250.
73A. The method of embodiment 71A, wherein the mean Cₘₐₓ is at least about 300.
74A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 100 up to about 500 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
75A. The method of embodiment 74A, wherein the mean Cₘᵢₙ is at least about 120.
76A. The method of embodiment 74A, wherein the mean Cₘᵢₙ is at least about 150.
77A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 40 up to about 300 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
78A. The method of embodiment 77A, wherein the mean Cₘᵢₙ is at least about 80.
79A. The method of embodiment 77A, wherein the mean Cₘᵢₙ is at least about 120.
80A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean free minimum plasma concentration (C_{minfree}) (ng/mL) of at least about 8.0 up to about 24.0 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
81A. The method of embodiment 80A, wherein the mean C_{minfree} is at least about 10.0.
82A. The method of embodiment 80A, wherein the mean C_{minfree} is at least about 12.0.
83A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 10,000 up to about 28,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
84A. The method of embodiment 83A, wherein the mean AUC is at least about 11,000.
85A. The method of embodiment 83A, wherein the mean AUC is at least about 12,000.
86A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 600 up to about 2,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
87A. The method of embodiment 86A, wherein the mean Cₘₐₓ is at least about 700.
88A. The method of embodiment 86A, wherein the mean Cₘₐₓ is at least about 800.
89A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 200 up to about 1,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
90A. The method of embodiment 89A, wherein the mean Cₘᵢₙ is at least about 250.
91A. The method of embodiment 89A, wherein the mean Cₘᵢₙ is at least about 300.
92A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean free minimum plasma concentration (C_{minfree}) (ng/mL) of at least about 12.0 up to about 40.0 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
93A. The method of embodiment 92A, wherein the mean C_{minfree} is at least about 14.0.
94A. The method of embodiment 92A, wherein the mean C_{minfree} is at least about 16.0.
95A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean whole blood area under the curve (AUC) (ng*hr/mL) of at least about 60,000 up to about 240,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
96A. The method of embodiment 95A, wherein the mean AUC is at least about 80,000.
97A. The method of embodiment 95A, wherein the mean AUC is at least about 100,000.
98A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum whole blood concentration (Cₘₐₓ) (ng/mL) of at least about 4,000 up to about 14,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
99A. The method of embodiment 98A, wherein the mean Cₘₐₓ is at least about 4,500.
100A. The method of embodiment 98A, wherein the mean Cₘₐₓ is at least about 5,000.
101A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 1,600 up to about 7,200 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
102A. The method of embodiment 101A, wherein the mean Cₘᵢₙ is at least about 1,800.
103A. The method of embodiment 101A, wherein the mean Cₘᵢₙ is at least about 2,000.
104A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 1,000 up to about 4,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
105A. The method of embodiment 104A, wherein the mean Cₘᵢₙ is at least about 1,150.
106A. The method of embodiment 104A, wherein the mean Cₘᵢₙ is at least about 1,300.
107A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean whole blood area under the curve (AUC) (ng*hr/mL) of at least about 100,000 up to about 400,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
108A. The method of embodiment 107A, wherein the mean AUC is at least about 120,000.
109A. The method of embodiment 107A, wherein the mean AUC is at least about 140,000.
110A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum whole blood concentration (Cₘₐₓ) (ng/mL) of at least about 6,000 up to about 28,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
111A. The method of embodiment 110A, wherein the mean Cₘₐₓ is at least about 7,000.
112A. The method of embodiment 110A, wherein the mean Cₘₐₓ is at least about 8,000.
113A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 2,000 up to about 12,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
114A. The method of embodiment 113A, wherein the mean Cₘᵢₙ is at least about 2,600.
115A. The method of embodiment 113A, wherein the mean Cₘᵢₙ is at least about 3,200.
116A. The method of any one of embodiments 62A-115A, which further results in a mean Cₘₐₓ to Cₘᵢₙ ratio of at least about 1.25 up to about 5.
117A. The method of any one of embodiments 62A-116A, wherein the Cₘₐₓ to Cₘᵢₙ ratio is less than 3.
118A. The method of any one of embodiments 62A-117A, wherein the Cₘₐₓ to Cₘᵢₙ ratio is less than 2.25.
119A. The method of any one of embodiments 62A-118A, wherein INX-315 is administered once-a-day.
120A. The method of any one of embodiments 62A-119A, wherein INX-315 is administered at a dose of about 200 mg, about 225 mg, or about 250 mg.
121A. The method of any one of embodiments 62A-120A, wherein INX-315 is orally administered.
122A. The method of any one of embodiments 62A-121A, wherein the INX-315 is administered in a pharmaceutical composition comprising: i) an amorphous spray-dried dispersion (ASD) formulation comprising amorphous INX-315 spray-dried with one or more precipitation preventers or additives that provide the desired properties for the ASD; and ii) optionally additionally includes one or more pharmaceutically acceptable excipients.
123A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 8,250 up to about 21,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
124A. The method of embodiment 123A, wherein the mean AUC is at least about 9,000.
125A. The method of embodiment 123A, wherein the mean AUC is at least about 9,750.
126A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 3,750 up to about 15,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
127A. The method of embodiment 126A, wherein the mean AUC is at least about 4,500.
128A. The method of embodiment 126A, wherein the mean AUC is at least about 5,250.
129A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 450 up to about 1,500 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
130A. The method of embodiment 129A, wherein the mean Cₘₐₓ is at least about 525.
131A. The method of embodiment 129A, wherein the mean Cₘₐₓ is at least about 600.
132A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 300 up to about 1,200 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
133A. The method of embodiment 132A, wherein the mean Cₘₐₓ is at least about 375.
134A. The method of embodiment 132A, wherein the mean Cₘₐₓ is at least about 450.
135A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 150 up to about 750 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
136A. The method of embodiment 135A, wherein the mean Cₘᵢₙ is at least about 180.
137A. The method of embodiment 135A, wherein the mean Cₘᵢₙ is at least about 225.
138A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 60 up to about 450 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
139A. The method of embodiment 138A, wherein the mean Cₘᵢₙ is at least about 120.
140A. The method of embodiment 138A, wherein the mean Cₘᵢₙ is at least about 180.
141A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean free minimum plasma concentration (C_{minfree}) (ng/mL) of at least about 12.0 up to about 36.0 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
142A. The method of embodiment 141A, wherein the mean C_{minfree} is at least about 15.0.
143A. The method of embodiment 141A, wherein the mean C_{minfree} is at least about 18.0.
144A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 15,000 up to about 42,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
145A. The method of embodiment 144A, wherein the mean AUC is at least about 16,500.
146A. The method of embodiment 144A, wherein the mean AUC is at least about 18,000.
147A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 900 up to about 3,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
148A. The method of embodiment 147A, wherein the mean Cₘₐₓ is at least about 1,050.
149A. The method of embodiment 147A, wherein the mean Cₘₐₓ is at least about 1,200.
150A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 300 up to about 1,500 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
151A. The method of embodiment 150A, wherein the mean Cₘᵢₙ is at least about 375.
152A. The method of embodiment 150A, wherein the mean Cₘᵢₙ is at least about 450.
153A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean free minimum plasma concentration (C_{minfree}) (ng/mL) of at least about 18.0 up to about 60.0 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
154A. The method of embodiment 153A, wherein the mean C_{minfree} is at least about 21.0.
155A. The method of embodiment 153A, wherein the mean C_{minfree} is at least about 24.0.
156A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean whole blood area under the curve (AUC) (ng*hr/mL) of at least about 90,000 up to about 360,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
157A. The method of embodiment 156A, wherein the mean AUC is at least about 120,000.
158A. The method of embodiment 156A, wherein the mean AUC is at least about 150,000.
159A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum whole blood concentration (Cₘₐₓ) (ng/mL) of at least about 6,000 up to about 21,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
160A. The method of embodiment 159A, wherein the mean Cₘₐₓ is at least about 6,750.
161A. The method of embodiment 159A, wherein the mean Cₘₐₓ is at least about 7,500.
162A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 2,400 up to about 10,800 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
163A. The method of embodiment 162A, wherein the mean Cₘᵢₙ is at least about 2,700.
164A. The method of embodiment 162A, wherein the mean Cₘᵢₙ is at least about 3,000.
165A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 1500 up to about 6,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
166A. The method of embodiment 165A, wherein the mean Cₘᵢₙ is at least about 1,725.
167A. The method of embodiment 165A, wherein the mean Cₘᵢₙ is at least about 1,950.
168A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean whole blood area under the curve (AUC) (ng*hr/mL) of at least about 150,000 up to about 600,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
169A. The method of embodiment 168A, wherein the mean AUC is at least about 180,000.
170A. The method of embodiment 168A, wherein the mean AUC is at least about 210,000.
171A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum whole blood concentration (Cₘₐₓ) (ng/mL) of at least about 9,000 up to about 42,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
172A. The method of embodiment 171A, wherein the mean Cₘₐₓ is at least about 10,500.
173A. The method of embodiment 171 wherein the mean Cₘₐₓ is at least about 12,000.
174A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 3,000 up to about 18,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
175A. The method of embodiment 174A, wherein the mean Cₘᵢₙ is at least about 3,900.
176A. The method of embodiment 174A, wherein the mean Cₘᵢₙ is at least about 4,800.
177A. The method of any one of embodiments 123A-176A, which further results in a mean Cₘₐₓ to Cₘᵢₙ ratio of at least about 1.25 up to about 5.
178A. The method of any one of embodiments 123A-177A, wherein the Cₘₐₓ to Cₘᵢₙ ratio is less than 3.
179A. The method of any one of embodiments 123A-178A, wherein the Cₘₐₓ to Cₘᵢₙ ratio is less than 2.25.
180A. The method of any one of embodiments 123A-179A, wherein INX-315 is administered once-a-day.
181A. The method of any one of embodiments 123A-180A, wherein INX-315 is administered at a dose of about 300 mg, about 325 mg, or about 350 mg.
182A. The method of any one of embodiments 123A-181A, wherein INX-315 is orally administered.
183A. The method of any one of embodiments 123A-182A, wherein the INX-315 is administered in a pharmaceutical composition comprising: i) an amorphous spray-dried dispersion (ASD) formulation comprising amorphous INX-315 spray-dried with one or more precipitation preventers or additives that provide the desired properties for the ASD; and ii) optionally additionally includes one or more pharmaceutically acceptable excipients.
184A. A method for the administration of a compound of structure of INX-315, or a pharmaceutically acceptable salt thereof, to the human in need thereof, which results in a mean plasma area under the curve (AUC) (ng*hr/mL) of at least about 11,000 up to about 28,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
185A. The method of embodiment 184A, wherein the mean AUC is at least about 12,000.
186A. The method of embodiment 184A, wherein the mean AUC is at least about 13,000.
187A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 5,000 up to about 20,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
188A. The method of embodiment 187A, wherein the mean AUC is at least about 5,750.
189A. The method of embodiment 187A, wherein the mean AUC is at least about 6,500.
190A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 600 up to about 2,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
191A. The method of embodiment 190A, wherein the mean Cₘₐₓ is at least about 700.
192A. The method of embodiment 190A, wherein the mean Cₘₐₓ is at least about 800.
193A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 400 up to about 1,600 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
194A. The method of embodiment 193A, wherein the mean Cₘₐₓ is at least about 500.
195A. The method of embodiment 193A, wherein the mean Cₘₐₓ is at least about 600.
196A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 200 up to about 1,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
197A. The method of embodiment 196A, wherein the mean Cₘᵢₙ is at least about 240.
198A. The method of embodiment 196A, wherein the mean Cₘᵢₙ is at least about 300.
199A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 80 up to about 600 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
200A. The method of embodiment 199A, wherein the mean Cₘᵢₙ is at least about 160.
201A. The method of embodiment 199A, wherein the mean Cₘᵢₙ is at least about 240.
202A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean free minimum plasma concentration (C_{minfree}) (ng/mL) of at least about 16.0 up to about 48.0 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
203A. The method of embodiment 202A, wherein the mean C_{minfree} is at least about 20.0.
204A. The method of embodiment 202A, wherein the mean C_{minfree} is at least about 24.0.
205A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean plasma area under the curve (AUC) (ng*hr/mL) of at least about 20,000 up to about 56,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
206A. The method of embodiment 205A, wherein the mean AUC is at least about 22,000.
207A. The method of embodiment 205A, wherein the mean AUC is at least about 24,000.
208A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum plasma concentration (Cₘₐₓ) (ng/mL) of at least about 1,200 up to about 4,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
209A. The method of embodiment 208A, wherein the mean Cₘₐₓ is at least about 1,400.
210A. The method of embodiment 208A, wherein the mean Cₘₐₓ is at least about 1,600.
211A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum plasma concentration (Cₘᵢₙ) (ng/mL) of at least about 400 up to about 2000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
212A. The method of embodiment 211A, wherein the mean Cₘᵢₙ is at least about 500.
213A. The method of embodiment 211A, wherein the mean Cₘᵢₙ is at least about 600.
214A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean free minimum plasma concentration (C_{minfree}) (ng/mL) of at least about 24.0 up to about 80.0 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
215A. The method of embodiment 214A, wherein the mean C_{minfree} is at least about 28.0.
216A. The method of embodiment 214A, wherein the mean C_{minfree} is at least about 32.0.
217A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean whole blood area under the curve (AUC) (ng*hr/mL) of at least about 120,000 up to about 480,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
218A. The method of embodiment 217A, wherein the mean AUC is at least about 160,000.
219A. The method of embodiment 217A, wherein the mean AUC is at least about 200,000.
220A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum whole blood concentration (Cₘₐₓ) (ng/mL) of at least about 8,000 up to about 28,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
221A. The method of embodiment 220A, wherein the mean Cₘₐₓ is at least about 9,000.
222A. The method of embodiment 220A, wherein the mean Cₘₐₓ is at least about 10,000.
223A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 3,200 up to about 14,400 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
224A. The method of embodiment 223A, wherein the mean Cₘᵢₙ is at least about 3,600.
225A. The method of embodiment 223A, wherein the mean Cₘᵢₙ is at least about 4,000.
226A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 2,000 up to about 8,000 as measured on day 1 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
227A. The method of embodiment 226A, wherein the mean Cₘᵢₙ is at least about 2,300.
228A. The method of embodiment 226A, wherein the mean Cₘᵢₙ is at least about 2,600.
229A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean whole blood area under the curve (AUC) (ng*hr/mL) of at least about 200,000 up to about 800,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
230A. The method of embodiment 229A, wherein the mean AUC is at least about 240,000.
231A. The method of embodiment 229A, wherein the mean AUC is at least about 280,000.
232A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean maximum whole blood concentration (Cₘₐₓ) (ng/mL) of at least about 12,000 up to about 56,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
233A. The method of embodiment 232A, wherein the mean Cₘₐₓ is at least about 14,000.
234A. The method of embodiment 232A, wherein the mean Cₘₐₓ is at least about 16,000.
235A. A method for the treatment of a human with a CDK2-mediated cancer comprising administering an effective amount of a pharmaceutical composition comprising a compound of structure of INX-315, or a pharmaceutically acceptable salt thereof, to the human in need thereof, which results in a mean minimum whole blood concentration (Cₘᵢₙ) (ng/mL) of at least about 4,000 up to about 24,000 as measured on day 15 of dosing, wherein the pharmaceutical composition comprises an amorphous spray dried dispersion (ASD) comprising between about 100 mg and about 500 mg of INX-315, and wherein the amount of INX-315 in the ASD is between about 20% and about 50% by weight.
236A. The method of embodiment 235A, wherein the mean Cₘᵢₙ is at least about 5,200.
237A. The method of embodiment 235A, wherein the mean Cₘᵢₙ is at least about 6,400.
238A. The method of any one of embodiments 184A-237A, which further results in a mean Cₘₐₓ to Cₘᵢₙ ratio of at least about 1.25 up to about 5.
239A. The method of any one of embodiments 184A-238A, wherein the Cₘₐₓ to Cₘᵢₙ ratio is less than 3.
240A. The method of any one of embodiments 184A-239A, wherein the Cₘₐₓ to Cₘᵢₙ ratio is less than 2.25.
241A. The method of any one of embodiments 184A-240A, wherein INX-315 is administered once-a-day.
242A. The method of any one of embodiments 184A-241A, wherein INX-315 is administered at a dose of about 400 mg, about 425 mg, or about 450 mg.
243A. The method of any one of embodiments 184A-242A, wherein INX-315 is orally administered.
244A. The method of any one of embodiments 184A-243A, wherein the INX-315 is administered in a pharmaceutical composition comprising: i) an amorphous spray-dried dispersion (ASD) formulation comprising amorphous INX-315 spray-dried with one or more precipitation preventers or additives that provide the desired properties for the ASD; and ii) optionally additionally includes one or more pharmaceutically acceptable excipients.
245A. A method for the administration of a compound of structure of INX-315, or a pharmaceutically acceptable salt thereof, to the human in need thereof, which results in a mean plasma Cₘₐₓ to Cₘᵢₙ ratio of less than about 5 but greater than about 1.25.
246A. The method of embodiment 245A, wherein the Cₘₐₓ to Cₘᵢₙ ratio is less than 3.
247A. The method of embodiment 245A, wherein the Cₘₐₓ to Cₘᵢₙ ratio is less than 2.25.
248A. The method of any one of embodiments 245A-247A, wherein the wherein the Cₘₐₓ to Cₘᵢₙ ratio is measured in plasma.
249A. The method of any one of embodiments 245A-247A, wherein the wherein the Cₘₐₓ to Cₘᵢₙ ratio is measured in whole blood.
250A. The method of any one of embodiments 1A-249A, which results in a t_{1/2} of between about 8 hours and about 28 hours.
251A. The method of any one of embodiments 1A-249A, which results in a t_{1/2} of between about 12 hours and about 18 hours.
252A. The method of embodiment 251A, wherein the t_{1/2} is about 14 hours.
253A. The method of embodiment 251A, wherein the t₁/2 is about 15 hours.
254A. The method of embodiment 251A, wherein the t_{1/2} is about 16 hours.
255A. The method of any one of embodiments 1A-254A, wherein the human has a tumor.
256A. The method of embodiment 255A, wherein the CDK2-mediated cancer is selected from bladder cancer, breast cancer, cervical cancer, colon cancer, endometrial cancer, esophageal cancer, fallopian tube cancer, glioblastoma multiforme (GBM), head and neck cancer, kidney cancer, liver cancer, lung cancer, ovarian cancer, peritoneal cancer, prostate cancer, sarcoma, skin cancer, stomach cancer, uterine cancer.
257A. The method of embodiment 256A, wherein the CDK2-mediated cancer is lung cancer.
258A. The method of embodiment 257A, wherein the lung cancer is small cell lung cancer (SCLC).
259A. The method of embodiment 256A, wherein the CDK2-mediated cancer is breast cancer.
260A. The method of embodiment 259A, wherein the breast cancer is hormone receptor-positive (HR+).
261A. The method of embodiment 259A or 260A, wherein the breast cancer is estrogen receptor-positive (ER+).
262A. The method of any one of embodiments 259A-261A, wherein the breast cancer is progesterone receptor-positive (PR+).
263A. The method of any one of embodiments 259A-262A, wherein the breast cancer is HER2A-negative (HER2A−).
264A. The method of embodiment 259A, wherein the breast cancer is triple negative breast cancer.
265A. The method of embodiment 256A, wherein the CDK2-mediated cancer is bladder cancer.
266A. The method of embodiment 256A, wherein the CDK2-mediated cancer is ovarian cancer.
267A. The method of embodiment 256A, wherein the CDK2-mediated cancer is prostate cancer.
268A. The method of embodiment 256A, wherein the CDK2-mediated cancer is sarcoma.
269A. The method of embodiment 256A, wherein the CDK2-mediated cancer is uterine cancer.
270A. The method of any one of embodiments 255A-269A, wherein the CDK2-mediated cancer is advanced unresectable and/or metastatic cancer.
271A. The method of any one of embodiments 255A-270A, wherein the CDK2-mediated cancer is cyclin E amplified or overexpressed.
272A. The method of embodiment 271A, wherein a Next Generation Sequencing (NGS) panel test is used to confirm cyclin E overexpression or amplification status.
273A. The method of any one of embodiments 255A-272A, wherein the CDK2-mediated cancer is Rb-positive cancer.
274A. The method of embodiment 273A, wherein the Rb-positive cancer is selected from an Rb-positive HR+/IER2A− breast cancer, ER+/HER2A− breast cancer, high-grade serous ovarian cancer (HGSOC), non-small cell lung cancer (NSCLC), colon cancer, prostate cancer, or glioblastoma.
275A. The method of embodiment 274A, wherein the Rb-positive cancer is HR+/HER2A− breast cancer.
276A. The method of embodiment 274A, wherein the Rb-positive cancer is ER+/HER2A− breast cancer.
277A. The method of embodiment 274A, wherein the Rb-positive cancer is high-grade serous ovarian cancer (HGSOC).
278A. The method of embodiment 274A, wherein the Rb-positive cancer is non-small cell lung cancer (NSCLC).
279A. The method of any one of embodiments 255A-272A, wherein the CDK2-mediated cancer is Rb-independent cancer.
280A. The method of embodiment 279A, wherein the Rb-independent cancer is selected from breast cancer, lung cancer, prostate cancer, liver cancer, bladder cancer, ovarian cancer, uterine cancer, cervical cancer, stomach cancer, esophageal cancer, head and neck cancer, glioblastoma, retinoblastoma, osteosarcoma, or lymphoma.
281A. The method of embodiment 279A, wherein the Rb-independent cancer is selected from small cell lung cancer (SCLC), retinoblastoma, triple negative breast cancer (TNBC), human papillomavirus (HPV) positive head and neck cancer, HPV positive cervical cancer, or neuroendocrine prostate carcinoma.
282A. The method of embodiment 281A, wherein the Rb-independent cancer is SCLC.
283A. The method of embodiment 281A, wherein the Rb-independent cancer is TNBC.
284A. The method of any one of embodiments 255A-283A, wherein the human previously received at least one prior line of chemotherapy.
285A. The method of any one of embodiments 255A-284A, wherein the human previously received at least two prior lines of chemotherapy.
286A. The method of any one of embodiments 255A-285A, wherein the human previously received at least one prior line of CDK4/6 inhibitor therapy.
287A. The method of any one of embodiments 255A-286A, wherein the human previously received at least one prior line of endocrine therapy.
288A. The method of any one of embodiments 255A-287A, wherein the cancer is relapsed.
289A. The method of any one of embodiments 255A-288A, wherein the CDK2-mediated cancer is CDK4/6 inhibitor resistant.
290A. The method of any one of embodiments 255A-289A, wherein the CDK2-mediated cancer has progressed following a prior regimen comprising an endocrine therapy.
291A. The method of any one of embodiments 255A-290A, wherein the CDK2-mediated cancer is endocrine therapy resistant.
292A. The method of any one of embodiments 1A-291A, wherein the treatment results in a reduction of incidents of treatment-emergent adverse events in comparison to the predicted number of incidents of treatment-emergent adverse events in subjects receiving CDK2 treatment other than INX-315.
293A. The method of any one of embodiments 1A-292A, wherein the treatment results in a reduction of incidents of laboratory abnormalities in comparison to the predicted number of incidents of laboratory abnormalities in subjects receiving CDK2 treatment other than INX-315.
294A. The method of any one of embodiments 1A-293A, wherein the treatment results in an improved overall survival (OS) in comparison to the predicted overall survival (OS) in subjects receiving CDK2 treatment other than INX-315.
295A. The method of any one of embodiments 1A-294A, wherein the treatment results in an improved overall response rate (ORR) in comparison to the predicted overall response rate (ORR) in subjects receiving CDK2 treatment other than INX-315.
296A. The method of any one of embodiments 1A-295A, wherein the treatment results in an improved disease control rate (DCR) in comparison to the predicted disease control rate (DCR) in subjects receiving CDK2 treatment other than INX-315.
297A. The method of any one of embodiments 1A-296A, wherein the treatment results in an improved progression free survival (PFS) in comparison to the predicted progression free survival (PFS) in subjects receiving CDK2 treatment other than INX-315.
298A. The method of any one of embodiments 1A-297A, wherein the treatment results in an improved duration of response (DOR) in comparison to the predicted duration of response (DOR) in subjects receiving CDK2 treatment other than INX-315.
299A. The method of any one of embodiments 1A-298A, wherein the treatment results in an extension of time to progression (TTP) in comparison to the predicted time to progression (TTP) in subjects receiving CDK2 treatment other than INX-315.
300A. The method of any one of embodiments 292A-299A, wherein the CDK2 treatment other than INX-315 comprises the administration of a CDK2 inhibitor selected from BLU-222 or PF-07104091A.
301A. The method of embodiment 300A, wherein the CDK2 inhibitor is BLU-222A.
302A. The method of embodiment 300A, wherein the CDK2 inhibitor is PF-07104091A.
303A. The method of any one of embodiments 292A-302A, wherein the CDK2 inhibitor other than INX-315 is administered in a dosage amount and frequency identical to INX-315.
304A. A pharmaceutical composition comprising an amorphous spray-dried dispersion (ASD) of INX-315, or a pharmaceutically acceptable salt thereof, and a pharmaceutically acceptable excipient.
305A. The pharmaceutical composition of embodiment 304A, wherein the INX-315 is between about 20% and about 50% by weight of the ASD; and wherein the INX-315 is between about 10% and about 30% by weight of the pharmaceutical composition.
306A. The pharmaceutical composition of embodiment 304A or 305A, wherein the INX-315 is about 40% by weight of the pharmaceutical composition; and wherein the INX-315 is about 20% by weight of the pharmaceutical composition.
307A. The pharmaceutical composition of any one of embodiments 304A-306A, wherein the INX-315 is between about 100 mg and about 500 mg, or between about 100 mg and about 300 mg, or between about 200 mg and about 400 mg.
308A. The pharmaceutical composition of embodiment 307A, wherein the INX-315 is about 100 mg.
309A. The pharmaceutical composition of embodiment 307A, wherein the INX-315 is about 200 mg.
310A. The pharmaceutical composition of embodiment 307A, wherein the INX-315 is about 300 mg.
311A. The pharmaceutical composition of embodiment 307A, wherein the INX-315 is about 400 mg.
312A. The pharmaceutical composition of any one of embodiments 304A-311A, wherein the precipitation preventer in the ASD is a hydroxypropyl methylcellulose acetate succinate (HPMCAS) selected from HPMCAS-L, HPMCAS-LF, HPMCAS-LG, HPMCAS-M, HPMCAS-MF, HPMCAS-MG, HPMCAS-H, HPMCAS-HF, HPMCAS-HG, or HPMCAS-E3A.
313A. The pharmaceutical composition of embodiment 312A, wherein the precipitation preventer in the ASD is HPMCAS-HG.
314A. The pharmaceutical composition of any one of embodiments 304A-313A, wherein the precipitation preventer in the ASD is between about 40% and about 70% by weight of the ASD; and wherein the precipitation preventer in the ASD is between about 20% and about 50% by weight of the pharmaceutical composition.
315A. The pharmaceutical composition of any one of embodiments 304A-314A, wherein the precipitation preventer in the ASD is about 60% by weight of the ASD; and wherein the precipitation preventer in the ASD is about 30% by weight of the pharmaceutical composition.
316A. The pharmaceutical composition of any one of embodiments 304A-315A, wherein the one or more excipients are selected from insoluble diluent, soluble diluent, disintegrant, glidant, lubricant, or a combination thereof.
317A. The pharmaceutical composition of embodiment 316A, wherein the one or more excipients comprise an insoluble diluent.
318A. The pharmaceutical composition of embodiment 317A, wherein the insoluble diluent comprises a microcrystalline cellulose.
319A. The pharmaceutical composition of embodiment 318A, wherein the microcrystalline cellulose comprises Avicel PH-101A.
320A. The pharmaceutical composition of any one of embodiments 317A-319A, wherein the insoluble diluent is between about 15% and about 25% by weight of the pharmaceutical composition.
321A. The pharmaceutical composition of any one of embodiments 317A-320A, wherein the insoluble diluent is about 20.5% by weight of the pharmaceutical composition.
322A. The pharmaceutical composition of any one of embodiments 316A-321A, wherein the one or more excipients comprise a soluble diluent.
323A. The pharmaceutical composition of embodiment 322A, wherein the soluble diluent comprises mannitol.
324A. The pharmaceutical composition of embodiment 323A, wherein the mannitol comprises Parteck M 100A.
325A. The pharmaceutical composition of any one of embodiments 322A-324A, wherein the soluble diluent is between about 15% and about 25% by weight of the pharmaceutical composition.
326A. The pharmaceutical composition of any one of embodiments 322A-325A, wherein the soluble diluent is about 20.5% by weight of the pharmaceutical composition.
327A. The pharmaceutical composition of any one of embodiments 316A-326A, wherein the one or more excipients comprise a disintegrant.
328A. The pharmaceutical composition of embodiment 327A, wherein the disintegrant comprises croscarmellose sodium.
329A. The pharmaceutical composition of embodiment 327A or 328A, wherein the disintegrant is between about 1% and about 10% by weight of the pharmaceutical composition.
330A. The pharmaceutical composition of any one of embodiments 327A-329A, wherein the disintegrant is about 6% by weight of the pharmaceutical composition.
331A. The pharmaceutical composition of any one of embodiments 316A-330A, wherein the one or more excipients comprise a glidant.
332A. The pharmaceutical composition of embodiment 331A, wherein the glidant comprises colloidal silicon dioxide.
333A. The pharmaceutical composition of embodiment 331A or 332A, wherein the glidant is between about 0.1% and about 5% by weight of the pharmaceutical composition.
334A. The pharmaceutical composition of any one of embodiments 331A-333A, wherein the glidant is about 1.5% by weight of the pharmaceutical composition.
335A. The pharmaceutical composition of any one of embodiments 316A-334A, wherein the one or more excipients comprise a lubricant.
336A. The pharmaceutical composition of embodiment 335A, wherein the lubricant comprises sodium stearyl fumarate.
337A. The pharmaceutical composition of embodiment 335A or 336A, wherein the lubricant is between about 0.1% and about 5% by weight of the pharmaceutical composition.
338A. The pharmaceutical composition of any one of embodiments 335A-337A, wherein the lubricant is about 1.5% by weight of the pharmaceutical composition.
339A. A process for manufacturing the pharmaceutical composition of any one of embodiments
304A-338A comprising: (i) spray-drying a solution comprising INX-315, or a pharmaceutically acceptable salt thereof, and one or more precipitation preventers or other additives that provide the desired properties to yield an amorphous spray-dried dispersion (ASD) comprising INX-315, or a pharmaceutically acceptable salt thereof, (ii) granulating the ASD together with one or more pharmaceutically acceptable excipients to provide a granulate; (iii) dry blending the granulate together with one or more pharmaceutically acceptable excipients; and (iv) tablet compressing the dry-blended granulate to provide a pharmaceutical composition comprising amorphous INX-315, or a pharmaceutically acceptable salt thereof, and pharmaceutically acceptable excipients in an oral dosage unit form.
340A. The process of embodiment 339A, wherein the oral dosage unit form has a nominal tablet strength of between about 100 mg and about 500 mg.
341A. The process of embodiment 340A, wherein the oral dosage unit form has a nominal tablet strength of about 100 mg.
342A. The process of embodiment 340A, wherein the oral dosage unit form has a nominal tablet strength of about 200 mg.
343A. The process of embodiment 340A, wherein the oral dosage unit form has a nominal tablet strength of about 300 mg.
344A. The process of embodiment 340A, wherein the oral dosage unit form has a nominal tablet strength of about 400 mg.

### EXAMPLES

### Example 1. Human Pharmacokinetic (PK) Parameter Modeling

The purpose of this study was to model predicted pharmacokinetic (PK) parameters of INX-315 in humans based on toxicity and toxicokinetics studies in animal preclinical models. Previously, INX-315 was administered once or twice daily for 28 consecutive days via intravenous and oral gavage administration routes to male and female Sprague Dawley rats (n=160), mice, and beagle dogs (n=40).

Toxicokinetic analyses were performed on composite plasma concentration versus time data for INX-315 using Phoenix WinNonlin (version 8.1) non-compartment analysis function (linear trapezoidal rule for AUC calculations) sparse data analysis function. Nominal dose values and sampling times were used for calculations. Any concentration reported as BLQ (LLOQ<1.0 ng/mL) was set equal to zero. Toxicokinetic analyses included Cₘₐₓ, Tₘₐₓ, AUC₀₋₂₄, t_{1/2}, RA Cₘₐₓ, and RA AUC₀₋₂₄.

Following this, human dose exposure was simulated using a combined whole blood and plasma compartment PK model using allometry and whole blood parameter estimates for the absorption rate constants and bioavailability per animal species. Absolute dose administration was used for modeling. V1, CL, V2, and Q parameters were extrapolated to human using allometric scaling. CL, Q by exponent equaled 0.818. V1, V2 by exponent equaled 1.03. Human body weight was set to 75 kg. Absorption rate constant (KA) was in reasonable ranges in mouse, rat, and dog, with values of 0.44, 0.53, and 0.18 (1/hr), respectively. KA was allometrically scaled from mouse to human by exponent of −0.025 (slower rate in human, KA=0.064521 (1/hr). Bioavailability (F1) was also observed to be within reasonable ranges in mouse, rat, and dog, with values of 0.62, 0.08, and 0.09, respectively. Bioavailability was assumed for human (i.e., F1=50%). Mouse bioavailability (ASD: 62%) often translates better to human than rat or dog (8% and 9%, respectively). The mouse gastrointestinal tract has lower pH than rat and dog and is closer to that of human. Whole blood (wb) to plasma translation was calculated using mean ex vivo ratios of wb:plasma for mouse and human as follows:

$$- {( \frac{wb}{plasma} )}_{human , in vivo} = {( \frac{wb}{plasma} )}_{mouse , in vivo} \cdot ( \frac{mean {( \frac{wb}{plasma} )}_{human , ex . vivo}}{{mean ( \frac{wb}{plasma} )}_{mouse , ex . vivo}} )$$

Concentration unbound human (Cₘᵢₙ^{free}) was calculated using ex vivo plasma protein data from human and mouse.

Predicted human PK values in plasma and whole blood are provided in Table 1.

TABLE 1
Prediction of Human PK Profiles

| | Plasma | | | | Whole Blood | | |
|---|---|---|---|---|---|---|---|
| Dose | AUC | Cₘₐₓ | Cₘᵢₙ | Cₘᵢₙ^{free} | AUC | Cₘₐₓ | Cₘᵢₙ |
| regimen | (ng*hr/ml) | (ng/mL) | (ng/mL) | (ng/mL) | (ng*hr/ml) | (ng/mL) | (ng/mL) |
| 100 mg QD | 741 | 39.3 | 19.1 | 0.997 | 5560 | 301 | 138 |
| 200 mg QD | 1380 | 72.3 | 36.4 | 1.9 | 11100 | 601 | 276 |
| 400 mg QD | 2490 | 130 | 67.2 | 3.51 | 22200 | 1200 | 552 |
| 600 mg QD | 3500 | 183 | 94.8 | 4.95 | 33300 | 1800 | 827 |
| 800 mg QD | 4480 | 235 | 121 | 6.29 | 44400 | 2400 | 1100 |
| 50 mg BID | 743 | 33.3 | 26.8 | 1.4 | 5550 | 251 | 198 |
| 100 mg BID | 1380 | 61.8 | 50.3 | 2.63 | 11100 | 501 | 396 |
| 200 mg BID | 2500 | 111 | 91.3 | 4.77 | 22200 | 1000 | 792 |
| 3000 mg BID | 3510 | 156 | 128 | 6.69 | 33300 | 1500 | 1190 |
| 4000 mg BID | 4480 | 200 | 163 | 8.53 | 44400 | 2000 | 1580 |

### Example 2. Clinical Study Protocol to Evaluate the Safety, Tolerability, Pharmacokinetics, and Efficacy of INX-315 in Patients with Advanced Unresectable and/or Metastatic Cancer

A first-in-human study was designed to evaluate the safety, tolerability, pharmacokinetics (PK), and preliminary antitumor activity of INX-315 in patients with recurrent advanced/metastatic cancer, including hormone receptor positive (HR+)/Human Epidermal Growth Factor Receptor 2 Negative (HER2−) breast cancer who progressed on a prior cyclin-dependent kinase 4/6 inhibitor (CDK4/6i) regimen, and CCNE1-amplified solid tumors who progressed on standard of care treatment.

### Study Drug Administration

In this first-in-human study, patients in Part A were given daily oral doses of INX-315 (in a tablet produced in a spray-dried amorphous solid dispersion (ASD) formulation as described in Examples 3-4 below) at a starting dose of 100 mg once daily (QD) to evaluate safety and the PK profile. All INX-315 tablets were taken at approximately the same time(s) each day. Based on observations from GLP and non-GLP toxicology studies this dose is considered safe with a significant safety margin of 97-fold compared to the human equivalent dose (HED) of the no observed adverse effect level (NOAEL) in rats (which would be 9730 mg/day) and 160-fold for the HED of the highest nonseverely toxic dose (HNSTD) in dogs (which would be 16,216 mg/day).

### Pharmacokinetic (PK) Assessments

Both plasma and whole blood PK samples for the analysis of INX-315 concentrations were collected on Cycle 1 Day 1 and Day 15, before dosing, and 0.5 (±5 min), 1 (±10 min), 2 (±10 min), 6 (±15 min), 8 (+15 min), and 12 (if possible; +15 min) hours post-dose. Samples will also be collected pre-dose on Cycle 1 Days 2, 8, 16, and Day 22, and pre-dose on Day 1 of each subsequent cycle.

### Pharmacokinetic (PK) Analysis

Standard non-compartmental PK parameters were estimated. Pharmacokinetic parameters were calculated, including, but not limited to:
Maximum plasma concentration (Cₘₐₓ)
Time to Cₘₐₓ (Tₘₐₓ)
Area under the plasma concentration time curve from 0 to 24 hours (AUC_{0-24 h})
Area under the plasma concentration-time curve extrapolated to infinity (AUC_{0-∞})
Clearance (CL/F)
Half-life (t_{1/2})

Plasma concentrations of INX-315 will be summarized descriptively using the PK population and will be summarized by dosing cohort. Geometric mean and geometric CV % will be included in the summary statistics. Individual (for each participant) and mean (standard deviation) concentrations over time for INX-315 will be displayed graphically in linear and semi-logarithmic plots. The actual collection time was used for individual plasma and whole blood INX-315 concentration curves, and the nominal time was used for the plots of mean plasma and whole blood INX-315 concentration curves.

The pharmacokinetic parameters were, where possible, determined from the plasma and whole blood concentrations of INX-315 by non-compartmental method using Phoenix WinNonlin software (Version 8.3 or higher, Certara, USA). PK parameters will be summarized descriptively by dosing cohort. AUC and Cₘₐₓ values will also be presented in scatter plots. Dose proportionality will be assessed for AUC and Cₘₐₓ using a power model. The 90% CI of the slope for each PK parameter will be estimated from the model and presented in a summary table.

### Results

### Plasma Testing

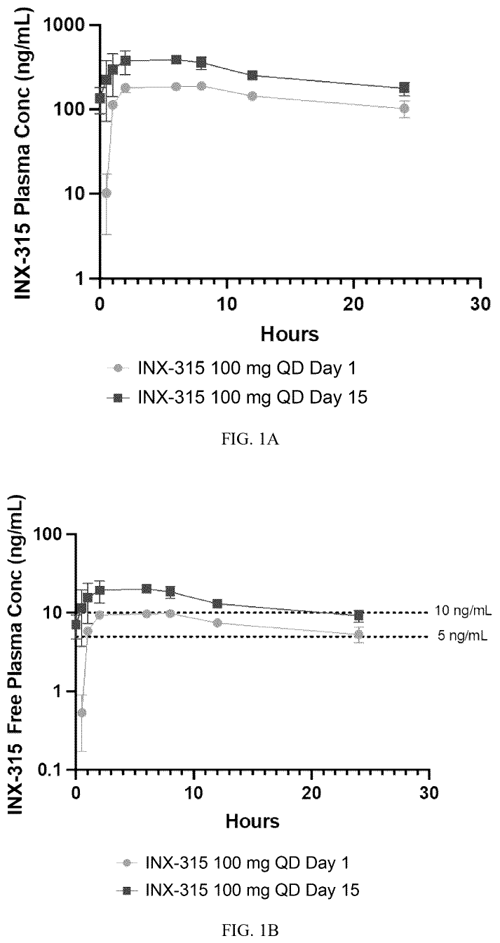

INX-315 plasma concentration versus time showed that the pharmacokinetic (PK) data is very consistent, as evidenced by steady concentration levels until 24 hours post-dose (FIG. 1A-1i).

TABLE 2
Plasma INX-315 Predicted versus Observed

| | | AUC | Cₘₐₓ | Cₘᵢₙ | Cₘᵢₙ^{free} |
|---|---|---|---|---|---|
| Matrix | | (ng*hr/mL) | (ng/mL) | (ng/mL) | (ng/mL) |
| Plasma | Predicted for | 741 | 39.3 | 19.1 | 0.997 |
| | 100 mg QD | | | | |
| | Day 1 | 3450 | 196 | 103 | 5.4 |
| | Observed | (3490, | (194, | (119, | (6.19, |
| | | 3410) | 197) | 86.3) | 4.49) |
| | Day 15 | 6580 | 408 | 177 | 9.2 |
| | Observed | (7240, | (457, | (199, | (10.35, |
| | | 5920) | 358) | 155) | 8.06) |

The Day 15 Cₘᵢₙ^{free} value of 9.2 ng/mL of INX-315 dosed 100 mg QD exceeded all predictions for the entire range of planned dosage regimens (Tables 2-3). INX-315 100 mg QD even exceeded Cₘᵢₙ^{free} predictions for both 800 mg QD and 400 mg BID (Tables 2-3). Furthermore, the Cₘᵢₙ^{free} value at 100 mg QD is well within the predicted efficacious range of 2-14 ng/mL (Table 2; FIG. 1A-1B).

TABLE 3
Human Cₘᵢₙ^{free} (ng/mL) Predictions By Cohort and Possible Dose Levels

| | Predicted | | | | | | | | | |
|---|---|---|---|---|---|---|---|---|---|---|
| | Cohort | 100 | 200 | 100 | 400 | 200 | 600 | 300 | 800 | 400 |
| Cohort | Size | QD | QD | BID | QD | BID | QD | BID | QD | BID |
| 1 | 3 | 0.997 | | | | | | | | |
| 2 | 3 | | 1.9 | 2.63 | | | | | | |
| 3 | 3 | | | | 3.51 | 4.77 | | | | |
| 4 | 3-6 | | | | | | 4.95 | 6.69 | | |
| 5 | 3-9 | | | | | | | | 6.29 | 8.53 |
| 6 | 6-9 | | | | | | | | 6.29 | 8.53 |

A review of published literature (Trub, A. G. et al. INX-315, a potent and selective CDK2 inhibitor, demonstrates robust antitumor activity in CCNE1-amplified cancers. 2023 AACR Annual Meeting. Cyclin-dependent Kinases and Cyclin-Dependent Kinase Inhibitors. Abstract 5994/23 (Poster). (2023 April); Brown, V. et al. BLU-222, an investigational, potent, and selective CDK2 inhibitor, demonstrated robust antitumor activity in CCNE-1 amplified ovarian cancer models. 2022 AACR Annual Meeting. 82(12_Supplement):Abstract 2306(Poster)(2022)) indicates that INX-315 is a more potent CDK2/cyclin E inhibitor in biochemical and intracellular assays compared to current CDK2 inhibitors in development (Table 4).

TABLE 4
Comparison of Selective CDK2 inhibitors

| | INX-315* | PF-07104091 | BLU-222† |
|---|---|---|---|
| Target IC₅₀ (nM) | 0.6 | 2.4 | 2.6 |
| CDK2/cyclin E | | | |
| Nanobret IC₅₀ (nM) | 2 | 32 | 17.7 |
| CDK2/cyclin E | | | |
| Cellular IC₅₀ (nM) | <100 | <100 | >100\hat{ } |
| In Vivo efficacy | 100 mg/kg BID or | 100 mg/kg BID | 100 mg/kg |
| | 200 mg/kg QD | (or greater) | BID |

*Trub, A. G. et al. INX-315, a potent and selective CDK2 inhibitor, demonstrates robust antitumor activity in CCNE1-amplified cancers. 2023 AACR Annual Meeting. Cyclin-dependent Kinases and Cyclin-Dependent Kinase Inhibitors. Abstract 5994/23. (2023 April).
†Brown, V. et al. BLU-222, an investigational, potent, and selective CDK2 inhibitor, demonstrated robust antitumor activity in CCNE-1 amplified ovarian cancer models. 2022 AACR Annual Meeting. 82(12_Supplement): Abstract 2306(Poster)(2022).
\hat{ }Based on Ovcar-3 cell cycle data presented at 2022 AACR meeting suggesting maximum G1 arrest >100 nM.

In agreement with the in vitro potency and in vivo efficacy of INX-315, INX-315 demonstrates a superior exposure profile in comparison to BLU-222 (FIG. 2). Dosing of INX-315 at 100 mg QD achieves plasma concentrations at 24 hours that are greater than BLU-222 600 mg BID at 12 hours (FIG. 2). INX-315, therefore, is more potent than BLU-222.

INX-315 also demonstrates a favorable exposure profile in comparison to PF-07104091 on Day 15 of continuous dosing of each inhibitor, with a substantially lower ratio of plasma Cₘₐₓ to Cₘᵢₙ values (FIG. 3). This is with the caveat that PF-07104091 plasma concentrations were only measured out to 9 hours nominal time.

With 300 mg BID of PF-07104091 being identified as the MTD and the monotherapy recommended dose for expansion (RDE) selection (Cₘₐₓ>2000 ng/mL), and with effective concentrations demonstrated with BLU-222 at 400 mg and 600 mg BID, the pharmacokinetic data of the CDK2 inhibitors at relevant dosage forms and regimens were summarized and compared in Table 5.

TABLE 5
Pharmacokinetic (PK) Comparisons.

| | Dose | Cₘₐₓ | C_{12 hr} | C_{24 hr} | Cₘₐₓ to | |
|---|---|---|---|---|---|---|
| Compound | Regimen | (ng/mL) | (ng/mL) | (ng/mL) | Cₘᵢₙ Ratio | Notes |
| INX-315 | 100 mg QD | 388 | 252 | 177 | 2.2 | First dose |
| | | | | | | level |
| BLU-222 | 400 mg BID | 927* | 100* | NA | 9.3 | Demonstrated |
| | | | | | | Ceff |
| | | | | | | coverage |
| | 600 mg BID | 2639* | 120* | NA | 22 | Demonstrated |
| | | | | | | Ceff |
| | | | | | | coverage |
| PF-07104091 | 300 mg BID | ~2000* | ~31-125* | NA | 16-64.5 | MTD, |
| | | | | | | selected as |
| | | | | | | monotherapy |
| | | | | | | RDE† |

*Estimated from 2023 ASCO presentations. Used computer program to determine data values on BLU-222 poster. Yap, T. A. et al. Journal of Clinical Oncology 41, no. 16_suppl (Jun. 1, 2023) 3010-3010. Published online May 31, 2023.
†PF-07104091 C_{12 hr} using Cₘₐₓ of 2000 ng/mL and t_{1/2} of 2-3 hours. NA, not applicable.

The PF-07104091 and BLU-222 values were partially estimated using a computer program to determine data values from published graphs. For PF-07104091, the Cₘᵢₙ at 12 hours was estimated at approximately 31.25-125 ng/mL based on a Cₘₐₓ of 2000 ng/mL and t_{1/2} of 2-3 hours. The Cₘₐₓ value of INX-315 at 100 mg QD was substantially lower than the estimated values for BLU-222 and PF-07104091. In agreement with this, INX-315 had a significantly lower Cₘₐₓ to Cₘᵢₙ ratio (2.2) than BLU-222 (9.3, 22) and PF-07104091 (16-64.5), as indicated by the relatively ‘flat’ PK curve (see, e.g., FIG. 92-93) compared to the more parabolic PK curves of BLU-222 and PF-07104091. Despite a low Cₘₐₓ to Cₘᵢₙ ratio, INX-315 still remained well above the therapeutic threshold of 5 ng/mL. In contrast, the higher Cₘₐₓ to Cₘᵢₙ ratios observed for PF-07104091 and BLU-222 correlate with a greater chance for off-target toxicity, as dosage amount is frequently increased in response to failure to remain above therapeutic threshold over the course of administration. This is under the assumption that off-target toxicity is driven by Cₘₐₓ and efficacy is driven by time above therapeutic threshold (Table 5).

### Whole Blood Testing

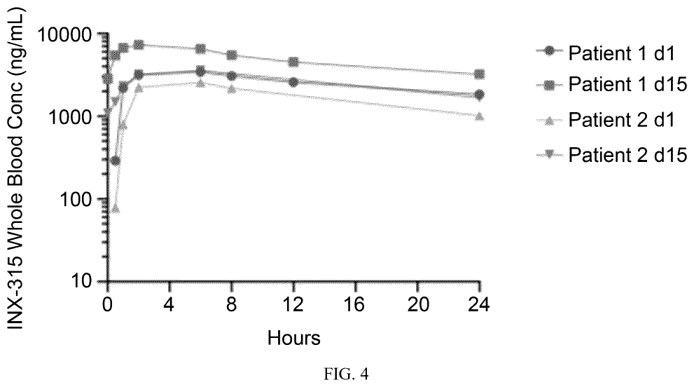

Separately, the PK parameters of Compound was tested in whole blood in individual Patients 1 and 2. INX-315 binds red blood cells (RBCs) at a higher level in whole blood than in plasma in the in vitro binding studies (FIG. 4).

TABLE 6
INX-315 PK Parameters in Whole Blood by Patient

| | Patient 1 d 1 | Patient 1 d 15 | Patient 2 d 1 | Patient 2 d 15 |
|---|---|---|---|---|
| Total Area | 60740 | 118285 | 41732 | 65180 |

INX-315 exhibited decreased human plasma protein binding compared to mouse, rat, and dog plasma (Table 7). The RBC-to-plasma ratio (K_{RBC/P}) of INX-315 is approximately 15.0 in humans (Table 7), which is higher than other species tested (mouse, rat, dog). Additionally, the whole blood-to-plasma ratio (K_{WB/P}) in humans is 6.20, which also exceeds the other species tested (Table 7). These results indicate that INX-315 exhibits enhanced RBC and whole blood partitioning relative to plasma. While not wishing to be bound to any one theory, it is proposed that this effect may be occurring because INX-315 is disproportionately bound to hemoglobin via the sulfonamide group.

TABLE 7
INX-315 protein binding and blood to plasma partitioning

| | | RBC-to-plasma | Whole blood-to-plasma |
|---|---|---|---|
| | Plasma protein | ratio | Ratio |
| Species | binding (%) | (K_{RBC/P}) | (K_{WB/P}) |
| Mouse | 97.7 | 3.45 | 2.09 |
| Rat | 98.2 | 9.70 | 3.40 |
| Dog | 96.0 | 3.60 | 1.86 |
| Human | 94.4 | 15.0 | 6.20 |

RBC, red blood cell.

TABLE 8
INX-315 blood to plasma partitioning

| | RBC-to-plasma ratio | Whole blood-to-plasma Ratio |
|---|---|---|
| Species | (K_{RBC/P}) | (K_{WB/P}) |
| Mouse | 4.02-8.76 | 2.03-3.65 |
| Rat | 9.62-24.6 | 4.02-9.26 |
| Dog | 9.17-24.6 | 3.76-8.99 |
| Human | 25.1-39.1 | 10.6-16.2 |

RBC, red blood cell.
Note:
The partitioning ratios are presented for the range of concentrations, 0.03 μM-10 μM.

INX-315 PK values in whole blood greatly exceeded predicted values estimated by a model based upon preclinical animal studies (Table 9).

TABLE 9
Whole Blood INX-315 Predicted versus Observed

| | | AUC | Cₘₐₓ | Cₘᵢₙ |
|---|---|---|---|---|
| Matrix | | (ng*hr/mL) | (ng/mL) | (ng/mL) |
| Whole | Predicted for | 5560 | 301 | 138 |
| Blood | 100 mg QD | | | |
| | Day 1 | 50550 | 3000 | 1430 |
| | Observed | (40600, 60500) | (2570, 3430) | (1020, 1840) |
| | Day 15 | 116350 | 5435 | 2445 |
| | Observed | (62700, 117000) | (3580, 7290) | (1670, 3220) |

AUC, Cₘₐₓ, and Cₘᵢₙ values in whole blood exceeded the model's prediction by greater than a factor of 9 for each parameter on Day 1, and more than a factor of 17 on day 15.

### Conclusion

INX-315 is the most potent CDK2/E inhibitor in both biochemical and intracellular assays relative to other CDK2 inhibitors currently in clinical development. The longer t_{1/2} observed for INX-315 should allow QD dosing compared to BID regimens for PF-4091 and BLU-222. The PK parameters of INX-315 observed are remarkably consistent in individual human patients (Tables 10 and 11), while greatly exceeding the predicted values based upon preclinical non-human studies.

TABLE 10
INX-315 Pharmacokinetic (PK) Parameters of Patient 1

| | Cₘₐₓ | Tₘₐₓ | Tₗₐₛₜ | Cₗₐₛₜ | AUCₗₐₛₜ | t_{1/2} |
|---|---|---|---|---|---|---|
| Matrix | (ng/mL) | (h) | (h) | (ng/mL) | (ng*h/mL) | (h) |
| | Day 1 | | | | | |
| Plasma | 194 | 8 | 24 | 119 | 3490 | NC |
| Whole Blood | 3430 | 6 | 24 | 1840 | 60500 | 22.4 |
| | Day 15 | | | | | |
| Plasma | 457 | 2 | 24 | 199 | 7240 | NR |
| Whole Blood | 7290 | 2 | 24 | 3220 | 117000 | NR |

NC, not calculated; NR, not reported.

TABLE 11
INX-315 Pharmacokinetic (PK) Parameters of Patient 2

| | Cₘₐₓ | Tₘₐₓ | Tₗₐₛₜ | Cₗₐₛₜ | AUCₗₐₛₜ | t_{1/2} |
|---|---|---|---|---|---|---|
| Matrix | (ng/mL) | (h) | (h) | (ng/mL) | (ng*h/mL) | (h) |
| | Day 1 | | | | | |
| Plasma | 197 | 2 | 24 | 86.3 | 3410 | 15.1 |
| Whole Blood | 2570 | 6 | 24 | 1020 | 40600 | 13.9 |
| | Day 15 | | | | | |
| Plasma | 358 | 6 | 24 | 155 | 5920 | NR |
| Whole Blood | 3580 | 6 | 24 | 1670 | 62700 | NR |

NC, not calculated; NR, not reported.

Preliminary data suggest the t_{1/2} of INX-315 is at least 15 hours (Tables 9-10). The concentration versus time profile at 100 mg QD suggests INX-315 has a low Cₘₐₓ to Cₘᵢₙ ratio (Table 5). The relatively flat concentration versus time profile of INX-315 indicates a lower exposure required to maintain concentrations above therapeutic threshold (FIG. 1A-113). By comparison, PF-07104091 and BLU-222 have significantly higher Cₘₐₓ to Cₘᵢₙ ratios (Table 5). While not wishing to be bound to any one theory, the spray-dried amorphous solid dispersion (ASD) formulation of INX-315 may result in reduced clearance of INX-315 through increased RBC binding and sustained release mechanisms, which could favorably alter the bioavailability, tissue retention, circulation time, and tumor targeting properties of INX-315, and may also limit non-specific, systemic off-target side effects of INX-315. As described more fully below in Table 12 of Example 3, INX-315 is highly insoluble in water and the INX-315 ASD formulation disperses INX-315 between polymer to prevent the crystallization of INX-315 in the ASD formulation, leading to sustained drug dissolution and resulting absorption of INX-315 while the polymer is naturally excreted. The PK profile of INX-315, therefore, could translate to best-in-class safety profile compared to PF-07104091 and BLU-222.

### Example 3. Development and Manufacture of INX-315 Formulations Using Amorphous Spray-Dried Dispersion (ASD)

### Scanning Electron Microscopy (SEM) of INX-315

SEM was used to obtain particle size and morphology information about INX-315 free base and HCl salt.

INX-315 free base formed crystallites comprising large aggregates of small particles comprising platelet or columnar small particles (1-15 um) and large, shapeless particles (15-25 um) (FIG. 5A-5C).

INX-315 HCl salt formed crystallites comprising large aggregates of small particles comprising highly columnar particles (1-25 um) (FIG. 6A-6C). There appeared to be more aggregates than INX-315 free base.

Both the INX-315 free base and the INX-315 HCl salt formed crystallites. The potential of crystallization in vivo from solution formulations would possibly result in suboptimal efficacy in subjects. Amorphous spray-dried dispersion (ASD) formulations were explored to minimize the risk of INX-315 crystal formation in vivo and efficacy loss.

### Solubility of INX-315

INX-315 is a BCS-2 compound (high permeability, low solubility) that was tested for enhanced formulations (Sugano, K. & Terada, K. Rate- and Extent-Limiting Factors of Oral Drug Absorption: Theory and Applications. J Pharm Sci. 104(9):2777-2788(2015 September)), as solubility was observed to be substantially limited at higher doses.

### Aqueous Solubility of JINX-315

Aqueous solubility of INX-315 was measured in 10 solvent systems (Table 12).

TABLE 12
Aqueous Solubility of INX-315

| | Total Drug | Dissolved Drug |
|---|---|---|
| Media | Solubility (μg/mL) | Solubility (μg/mL) |
| 0.01N HCl | 299 ± 11 | 269 ± 92 |
| 50 mM Na Acetate | 19 ± 7 | 3.4 ± 0.4 |
| PBS/Blank FaSSIF | 17 ± 2 | 1 ± 2 |
| 0.224% SIF in PBS | 6.1 ± 0.4 | 5.3 ± 0.4 |
| 0.5% SIF in PBS | 15.0 ± 0.2 | 11 ± 1 |
| 1% SIF in PBS | 16.4 ± 0.1 | 16.3 ± 0.1 |
| 5% TPGS in PBS | 305 ± 10 | 313 ± 21 |
| 0.5% Tween-80 in | 55 ± 1 | 59 ± 8 |
| PBS | | |
| 1% SLS in H₂O | 46 ± 25 | 40 ± 1 |
| H₂O | 61 ± 14 | 0.0 ± 0.0 |

INX-315 was not highly soluble in any solvent system tested, and was exceedingly insoluble in water (Table 12).

### Organic Solubility of JINX-315

The solubility of INX-315 was next assessed by visual inspection in 10 organic solvent systems (Table 13).

TABLE 13
Organic Solubility of INX-315

| | Soluble at | Soluble at | Soluble at | Soluble at | Soluble at | Soluble at |
|---|---|---|---|---|---|---|
| Solvent | 4 wt % | 2 wt % | 1 wt % | 0.5 wt % | 0.4 wt % | 0.25 wt % |
| Acetone | X | X | X | ✓ | ✓ | ✓ |
| Methanol | X | X | X | X | X | ✓ |
| 9:1 | X | X | X | X | ✓ | ✓ |
| Acetone:H₂O | | | | | | |
| 8:2 | X | X | X | X | ✓ | ✓ |
| Acetone:H₂O | | | | | | |
| 3:1 | X | X | X | X | X | X |
| DCM:MeOH | | | | | | |
| 4:1 | ✓ | ✓ | ✓ | ✓ | ✓ | ✓ |
| THF:H₂O | | | | | | |
| 1:9 | X | X | X | X | X | X |
| DMSO:H₂O | | | | | | |
| 9:1 | X | X | X | ✓ | ✓ | ✓ |
| ACN:H₂O | | | | | | |
| 4:1 | X | X | X | X | X | X |
| ACN:H₂O | | | | | | |
| 5:1 | X | X | X | X | X | X |
| DCM:H₂O | | | | | | |

✓, soluble; X, insoluble.

Surprisingly, INX-315 was soluble at all weight percentages only in the 4:1 THF:H₂O solvent (Table 13). INX-315 was only soluble at much lower concentrations of up to 0.5 weight percent (wt %) in acetone or 9:1 ACN: H₂O. INX-315 was largely insoluble in all the remaining solvents. It was concluded that INX-315 would be spray dried in a mixture of THF and H₂O, with reduced H₂O not expected to negatively impact solubility of INX-315.

### Micelle Partitioning of INX-315

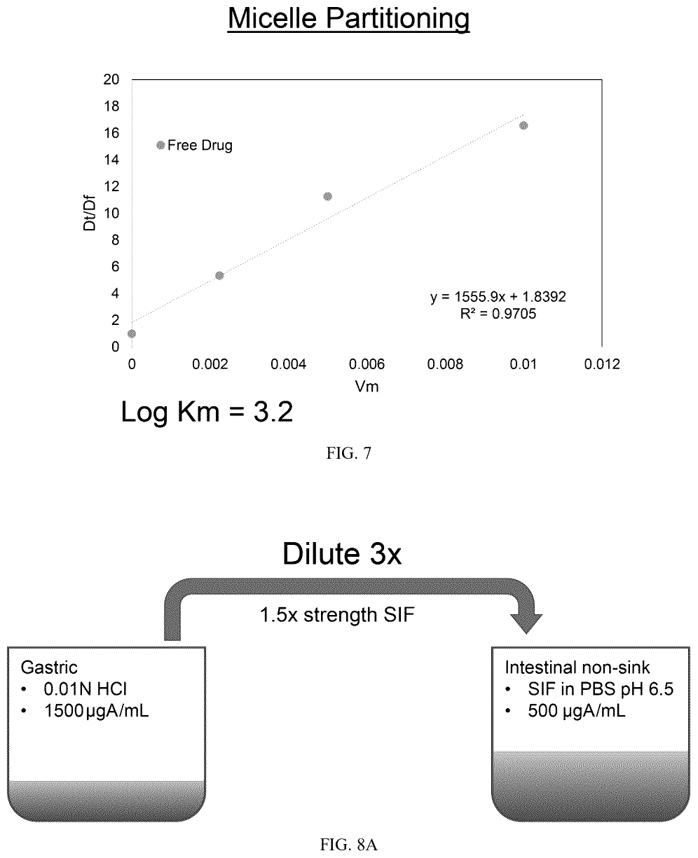

A micelle partitioning coefficient (Kₘ) value of 3.2 indicates some partitioning of INX-315 into bile salt micelles (FIG. 7). Crystalline solubility is significantly enhanced by the presence of micelles, both from bile salts and surfactants.

### Non-Sink Dissolution Method

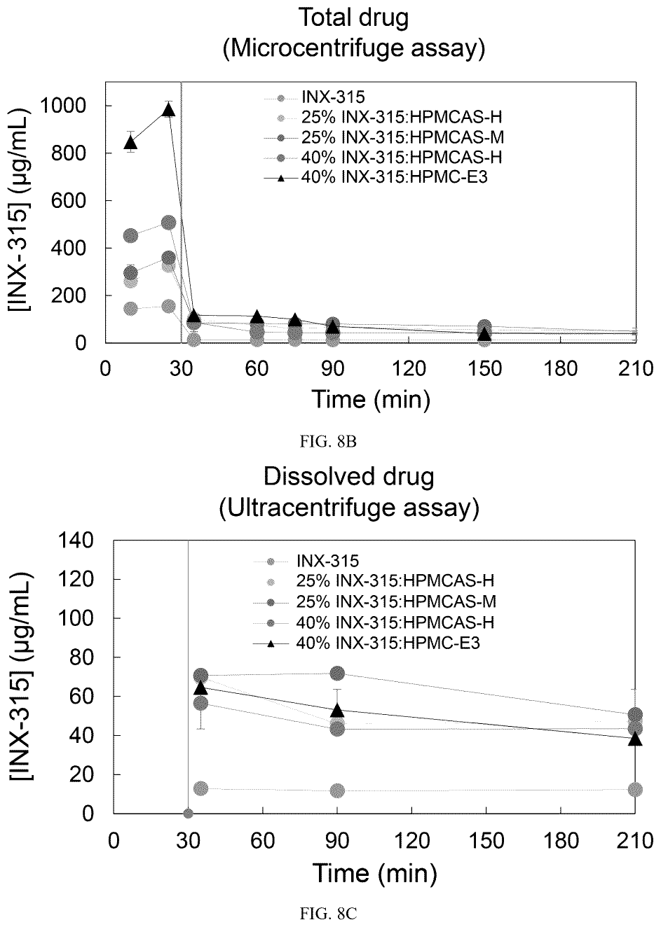

INX-315 was next assayed using the Non-Sink Dissolution Method to observe INX-315 dissolution kinetics changes during gastric to intestinal conditions transit (FIG. 8A).

The Gastric Media comprised 0.01 N HCl (pH 2.0) at a dose of 1500 μgA/mL. Timepoints tested were 10 min and 25 min following microcentrifugation of 100 μL aliquots diluted 5× in MeOH at 18,500 g. The Intestinal Media comprised 0.5 wt % SIF in Phosphate Buffer (pH 6.5) at a dose of 500 μgA/mL. Timepoints tested were 5 min, 30 min, 45 min, 70 min, 180 min, and 24 hr (post-transfer) following microcentrifugation of 100 μL aliquots diluted 5× in MeOH at 18,500 g. Timepoints tested were 5 min, 45 min, and 24 hr (post-transfer) following ultracentrifugation of 100 μL aliquots diluted 5× in MeOH at 470,000 g. The supernatants were assayed by HPLC.

TABLE 14
Non-Sink Dissolution Results

| | Micro | Ultra | Micro | Ultra | Micro | Ultra |
|---|---|---|---|---|---|---|
| | @ | @ | @ | @ | @ | @ |
| Formulation | 5 min | 5 min | 60 min | 60 min | 180 min | 180 min |
| INX-315 | 13 | 13 | 12 | 12 | 13 | 12 |
| 25% INX-315:HPMCAS-H | 103 | 70 | 69 | 46 | 51 | 47 |
| 25% INX-315:HPMCAS-M | 84 | 71 | 81 | 72 | 49 | 51 |
| 40% INX-315:HPMCAS-H | 86 | 57 | 43 | 43 | 42 | 43 |
| 40% INX-315:HPMC-E3 | 117 | 65 | 69 | 53 | 38 | 38 |

All INX-315 ASD formulations outperformed INX-315 alone (FIG. 8B-8C, Table 14). The 25% INX-315:HPMCAS-M formulation exhibited the best sustainment and highest concentration of small species. The 25% INX-315:HPMCAS-H formulation performed similarly to 40% INX-315:HPMCAS-H but with lower gastric solubility. There were negligible differences between total drug and dissolved drug (especially at 3 hours). While not wanting to be bound by any one theory, this implies that a majority of INX-315 exists in micelles, small colloids, small self-assembled drug aggregates, or molecularly free drug. Additionally, all INX-315 ASD formulations sustain supersaturation up to 3 hours in intestinal conditions.

### Differential Scanning Calorimetry

The glass-transition temperature (T_{g}) and melting temperature (Tₘ) of ASD formulations were determined using a Thermal Analysis DSC 250 differential scanning calorimeter. Briefly, the samples were scanned from 0 to 278° C. at a ramp rate of 3.0° C./min, with a 1.0° C./min modulation. The midpoint of the glass-transition event was reported as the T_{g}.

A single Tₘ observed at 274° C. in the first scan (FIG. 9A). A single T_{g} was observed at 242° C. which was unusually high (FIG. 9B). The ratio of Tₘ/T_{g} was 1.06. The degradation onset (Tdeg) was measured at approximately 280° C.

Based on the high T_{g}, it was concluded that INX-315 had a low risk of physical de-stabilization as an ASD. Also, the low Tₘ/T_{g} ratio indicates a low tendency of INX-315 to crystallize from the amorphous form.

### Scanning Electron Microscopy (SEM) of INX-315 ASD Formulations

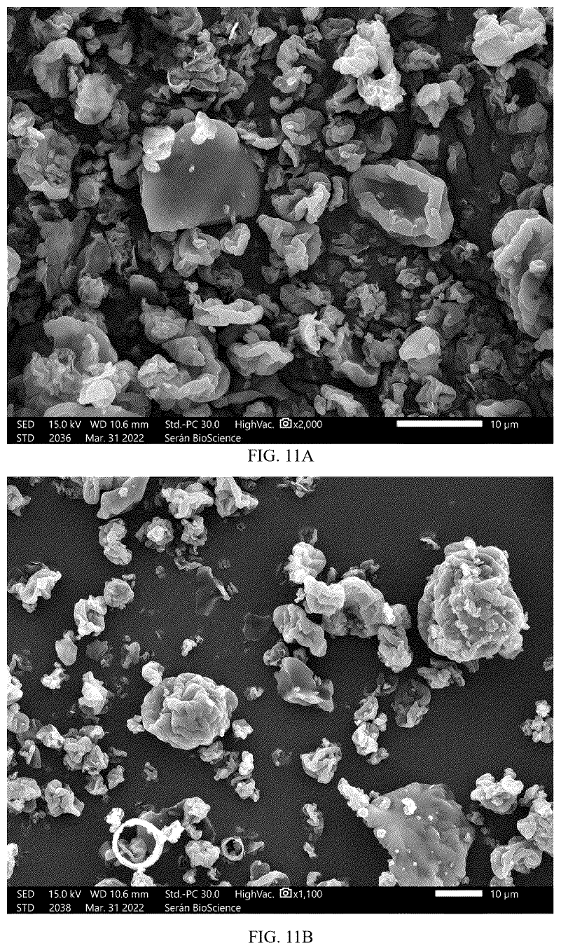

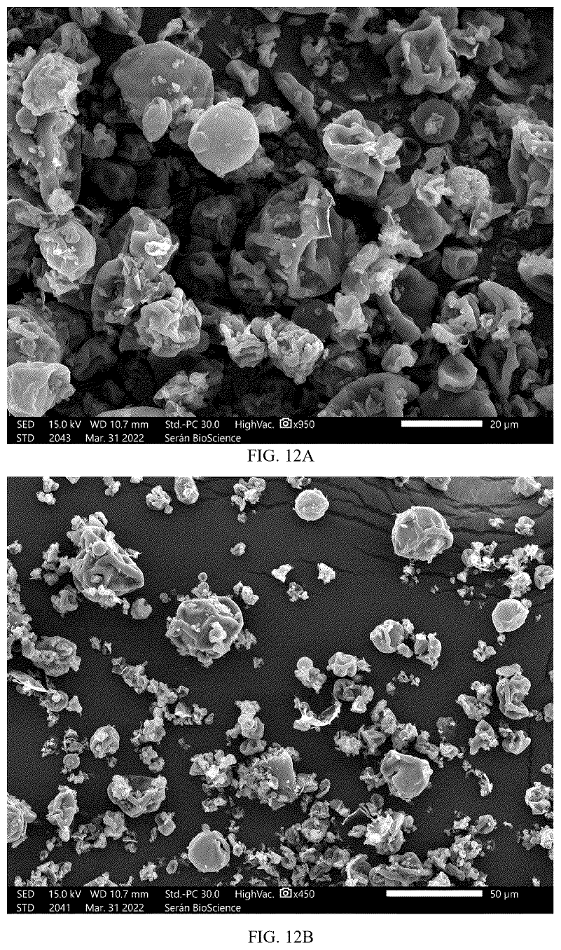

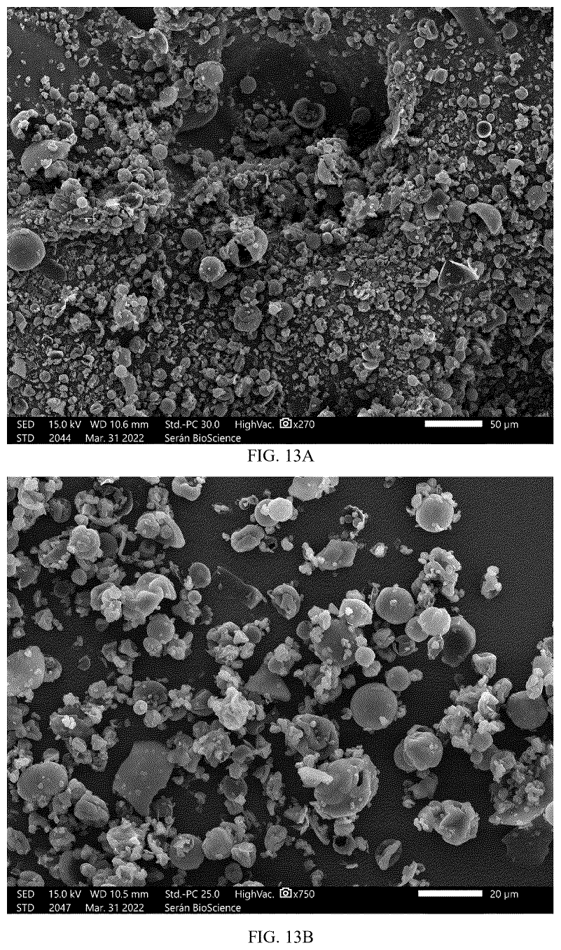

Various amorphous spray-dried dispersion (ASD) formulations were developed to enhance the solubility of INX-315 in solution. Four different INX-315-polymer formulations were tested, including i) 25% INX-315:HPMCAS-H (see FIG. 10A-10B); ii) 40% INX-315:HPMCAS-H (see FIG. 11A-11B); iii) 25% INX-315:HPMCAS-M (see FIG. 12A-12B); and iv) 40% INX-315:HPMC-E3 (see FIG. 13A-13B).

Spray Dry Equipment included the Spray dryer (custom spray dryer (SD-45) with 1.5° cyclone; open loop mode), Solution vessel (250 mL Erlenmeyer flask with magnetic stirring), Atomization (2-fluid atomization; solution fed with a peristaltic pump to a SS ¼ J 2850 nozzle with a SS ¼-J 64 air cap), and Secondary drying (Binder vacuum tray dryer).

Scanning Electron Microscopy (SEM) was used to obtain particle size and morphology information about the ASD formulations. Surprisingly, there was no crystallinity formed with any INX-315 ASD formulation tested (FIG. 10A-13B). All formulations had particle size distributions characteristic of a 2-fluid nozzle. Particle morphology in each formulation was wrinkled/collapsed spheres, which are commonly seen with HPMCAS ASDs. The morphologies also indicated the drying conditions were in an ideal range, although some particle shattering (FIG. 11A-11B, 13A-13B) and “blow holes” (FIG. 12A-12B) were observed.

### Powder X-Ray Diffraction (PXRD) Analysis

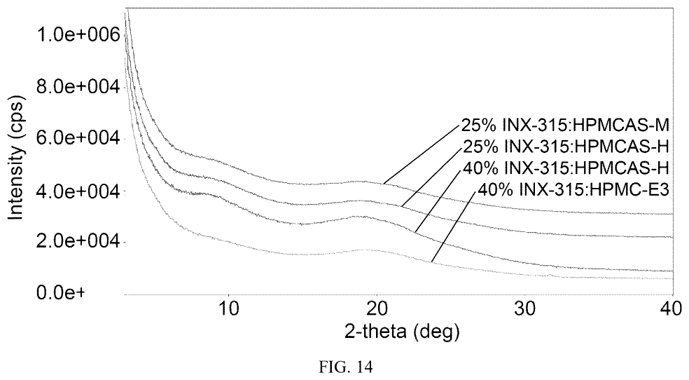

PXRD analysis of the INX-315 ASD formulations was carried out on a Rigaku Miniflex 600 detector, scanning the samples between 3°-40° 2θ using the following settings: Step size: 0.02°; Step rate: 2°/min; Voltage: 40 kV; Current: 15 mA; Sample disk: Zero background. The above settings were used to generate FIG. 14.

The diffraction spectrum showed halo consistent of amorphous material and no crystallinity detected for the ASD formulations with 25% INX-315 loading (FIG. 14). A single small peak was detected at 32° 2θ for the ASD formulations with 400% INX-315 loading, which may be due to residual salt of TINX-315 and not a sign of crystallinity.

Four different formulations were chosen for further spray dry-polymer parameter optimization, including i) 25% INX-315:HPMCAS-H; ii) 40% INX-315:HPMCAS-H; iii) 40% INX-315:HPMCAS-M; and iv) 40% INX-315:PVP-VA. HPMCAS-H is the most hydrophilic of the HPMCAS grades, having more acidic functional groups which can promote supersaturation and inhibit precipitation. HPMCAS-M, meanwhile, balances the hydrophilicity of HPMCAS-H and the hydrophobicity of HPMCAS-L, which could enable promotion of colloids and supersaturation sustainment. PVP-VA is a neutral polymer known to promote formation of colloids that was chosen in the event that INX-315 exhibited chemical sensitivity to the acidic functional groups in HPMCAS formulations.

TABLE 15
Spray Dry Manufacturing Parameters

| | 25% INX- | | 25% INX- | |
|---|---|---|---|---|
| | 315:HPMCAS-H | 40% INX- | 315:HPMCAS-M | 40% INX- |
| Formulation | rep. 1/rep. 2 | 315:HPMCAS-H | rep. 1/rep. 2 | 315:PVP-VA |
| Solvent system | 80:20 THF:H₂O | | | |
| (w/w) | | | | |
| Solids loading | 2.0/2.0 | 2.0 | 2.0/2.0 | 2.0 |
| (API wt %) | | | | |
| Solids loading | 6.0/6.0 | 3.0 | 6.0/6.0 | 3.0 |
| (Polymer wt %) | | | | |
| Batch size (g | 1.91/3.83 | 2.40 | 1.91/3.82 | 2.40 |
| solids) | | | | |
| Drying gas feed | 642/643 | 641 | 642/643 | 632 |
| rate (g/min) | | | | |
| Liquid feed rate | 31.3/30.6 | 29.0 | 31.6/30.1 | 29.9 |
| (g/min) | | | | |
| Atomization | 15.1/14.9 | 14.8 | 14.7/14.7 | 15.4 |
| pressure (psig) | | | | |
| Drying gas inlet | 106/109 | 110 | 112/108 | 112 |
| temperature (° C.) | | | | |
| Spray dryer | 44.9/45.9 | 44.1 | 44.9/45.8 | 45.0 |
| outlet | | | | |
| temperature (° C.) | | | | |
| Secondary | >12 hr at 40° C. and 180 mbar | | | |
| drying | | | | |
| conditions | | | | |
| Wet Yield (g) | 0.84/2.09 | 1.59 | 0.71/2.16 | 1.60 |
| Dry Yield (g) | 0.80/1.99 | 1.52 | 0.68/2.06 | 1.52 |
| % Dry Yield | 42.1/52.1 | 63.3 | 35.7/53.9 | 63.3 |

rep. 1, replicate 1; rep. 2, replicate 2.

Unexpectedly low yields were observed for the 25% INX-315:HPMCAS-H and 25% INX-315:HPMCAS-M formulations due to short spray times in replicate 1. When larger batch sizes and longer spray times were used in replicate 2, yield was increased but still lower than the 40% loading ASD formulations.

The 40% INX-315:HPMCAS-H and 40% INX-315:HPMC-E3 formulations were chosen as ASD formulation leads and re-tested in Table 16.

TABLE 16
Exemplary Spray Dry Formulations Manufacturing Parameters

| | 40% INX- | 40% INX- |
|---|---|---|
| Formulation | 315:HPMCAS-H | 315:HPMC-E3 |
| Solvent system (w/w) | 80:20 THF:H₂O | |
| Solids Loading (wt %) | 5 | |
| Batch Size (g solids) | 25.1 | 25.1 |
| Drying Gas Flowrate (g/min) | 726 | 727 |
| Solution Flowrate (g/min) | 34 | 32 |
| Atomization Pressure (psig) | 200 | 200 |
| Inlet Temperate (° C.) | 105 | 108 |
| Outlet Temperature (° C.) | 45 | 46 |
| Secondary Drying Conditions | 15 hr, 40° C., ~180 mbar | |
| Dry Yield (g) | 17.1 | 21.2 |
| % Dry Yield | 68 | 84 |

Yields were about 70-90% for both spray formulations. The primary yield losses were attributable to build-up on drying chamber walls which is typical for the processing conditions used.

The 40% INX-315:HPMCAS-H ASD formulation was advanced. Spray dry equipment parameters were optimized as follows: Spray dryer (Custom spray dryer (SD-90) with 4″ cyclone; open loop mode), Solution vessel (20 L stainless steel vessel with ⅓ hp overhead compressed air powered mixer), Atomization (Pressure swirl; solution fed with a SPX high pressure pump to SK 78-16 and 79-16 nozzles), and Secondary drying (Binder convection tray dryer). Optimization led to increased yield in Table 17.

TABLE 17
Exemplary 40% INX-315:60% HPMCAS-H Spray
Dry Formulation Manufacturing Parameters

| Solvent system (w:w) | 90:10 THF:H₂O |
|---|---|
| Solids loading (wt %) | 7.5 |
| Batch size (g solids) | 1266.5 |
| Drying gas feed rate (g/min) | 1496 |
| Liquid feed rate (g/min) | 196 |
| Atomization pressure (psig) | 569 |
| Drying gas inlet temperature (° C.) | 128 |
| Spray dryer outlet temperature (° C.) | 51 |
| Secondary drying conditions | 40° C., 2 hr then 50° C., 46 hr |
| Wet Yield (g) | 1216.3 |
| Dry Yield (g) | 980 |
| % Dry Yield | 77.4 |

### Conclusion

INX-315 is highly insoluble in water and the INX-315 ASD formulation disperses INX-315 across the polymer matrix which prevents crystallization of INX-315 in the ASD formulation. Furthermore, the INX-315 ASD formulations exhibited similar total drug and dissolved drug concentrations in the Non-Sink Dissolution method for prolonged periods, which implies that a majority of INX-315 exists in micelles, small colloids, small self-assembled drug aggregates, or molecularly free drug. While not wishing to be bound to one theory, high levels of INX-315 in micelles promote absorption by shuttling drug across the intestinal mucus boundary layer which is rapidly replaced by free drug as it is absorbed while the remaining INX-315 in drug/polymer colloids are rapidly dissolved to replace free drug as it is absorbed. The formation of a variety of INX-315 drug/polymer and micelle structures lead to sustained INX-315 dissolution and absorption of INX-315, creating a “depot effect” phenomenon. Also, high T_{g} and low Tₘ/T_{g} ratios indicate that INX-315 has a low tendency to crystallize from the amorphous form.

### Example 4. Drug Product Formulation

A dosage form including the INX-315 amorphous spray-dried dispersion (ASD) as described herein was developed wherein the free base was maintained in an amorphous form which would dissolve quickly.

### Tablet Manufacturing Process

INX-315 tablets (100 mg) are a modified oval biconvex design with a functional score on one side and comprised of common dosage form excipients. An exemplary drug product is manufactured in four parts, comprising:
(1) Spray-drying a 90:10 THF (tetrahydrofuran) solution containing 40% INX-315 API and 60% hydroxy propyl methylcellulose acetate succinate-high substitution (HPMCAS-HG);
(2) Dry granulating the 40% INX-315/60% HPMCAS-HG amorphous spray-dried dispersion (ASD) with microcrystalline cellulose, mannitol, croscarmellose sodium, colloidal silicon dioxide and sodium stearyl fumarate (Intragranular tablet component);
(3) Dry blending the intragranular tablet component with additional excipients including, for example croscarmellose sodium, colloidal silicon dioxide, and sodium stearyl fumarate (Extragranular tablet component); and
(4) Compressing into tablets with a nominal tablet strength of 100 mg.

A more detailed Manufacturing Process is provided below:
(i) Spray-drying
   (a) Add tetrahydrofuran (THF), purified water, HPMCAS-HG, and INX-315 sequentially into a jacketed 100 L stainless steel tank;
   (b) Mix until a solution is obtained which is essentially free of particles;
   (c) Connect a circulating batch to the jacketed 100 L tank set to maintain a 2-8° C. temperature;
   (d) Spray dry with an inlet temperature of about 105° C. and a feed rate of about 200 g/min;
   (e) Collect the solids in cyclone containers until all wet solids are collected;
   (f) Transfer the wet solids from the cyclones to tray dryer trays;
   (g) Dry the solids at 40-70° C. until THF amount is 3000 ppm or less;
   (h) Remove the trays containing dried product from the tray dryer and package in double-lined low-density polyethylene bags with dessicant between bags.
(ii) Dry granulating
   (a) Add about ⅓ of microcrystalline cellulose (MCC) to a 40 L blender and blend to pre-condition the blender;
   (b) Add an intragranular pre-lubricated blend comprising 40% INX-315 and 60% HPMCAS-HG ASD, mannitol, croscarmellose sodium, MCC/Colloidal silicon dioxide (about 2:1 w/w ratio), and microcrystalline cellulose (remaining) sequentially to the 40 L blender and blend;
   (c) Discharge the intragranular pre-lubricated blend into low-density polyethylene (LDPE) bags;
   (d) de-lump the intragranular pre-lubricated blend by passing through a conical mill and collect in LDPE bags;
   (e) Add pre-weighed sodium stearyl fumarate to the blender containing the de-lumped intragranular pre-lubricated blend;
   (f) Blend and collect the lubricated intragranular blend in LDPE bags.
(iii) Dry blending
   (a) weigh the lubricated intragranular blend to determine amounts of extra-granular excipients required;
   (b) weigh out extra-granular excipients and add the croscarmellose sodium and colloidal silicon dioxide to the blender and blend;
   (c) Add weighted extra-granular sodium stearyl fumarate;
   (d) Blend and discharge final blend into LDPE bags.
(iv) Tablet Compressing
   (a) Compress final blend into 8 mm×16.5 mm modified oval tablets containing a bisect on the upper punch with a target compression weight of 500 mg and a target hardness of 21 kp.

### Description and Composition of the INX-315 Manufacturing Process

### In Process Manufacturing Control Monitoring

The following in-process controls are performed on the product during the ASD and Tablet manufacturing processes as provided in Table 18.

TABLE 18
In Process Testing Performed During ASD and Tablet Manufacture

| Process | Control Test | Frequency | Specification | |
|---|---|---|---|---|
| ASD Drying | Residual THF level | As needed until | Not more than 3000 ppm | |
| | | specification is | | |
| | | met | | |
| Tableting | Individual Tablet Weight | Approximately | 475-525 | mg |
| | Individual Tablet Thickness | every 10 minutes | 5.11-5.41 | mm |
| | Individual Tablet Hardness | | 17.5-25.5 | kp |
| | Average Tablet Hardness | | 18.0-25.0 | kp |

| | | |
|---|---|---|
| Friability | Start-up only | Not more than 0.5% loss |

As stated above, the 100 mg INX-315 tablet has a functional score designed to be split using a pharmaceutical guillotine. The current clinical protocol specifies dose escalations in multiples of 100 mg so there may never be a need utilize the functional score. However, if a 50 mg dosing increment is desired the functional score can provide that dose. As part of development, tablets were split using a guillotine and evaluated physically and for dissolution rate. Content uniformity was not performed but can be inferred from the dissolution data. Table 19 provides a summary of the bisect data. The theoretical tablet weight is 500 mg.

TABLE 19
Disintegration and Dissolution Results for Bisected Tablets

| Test | Tablet 1 | Tablet 2 | Tablet 3 | Tablet 4 | Tablet 5 | Tablet 6 | % RSD |
|---|---|---|---|---|---|---|---|
| Disintegration - Whole | 45 seconds | | | | | | |
| Tablets (0.1N HCl) 18.0 | | | | | | | |
| average kp hardness | | | | | | | |
| Disintegration - Bisect | 40 seconds | | | | | | |
| Tablet Halves (0.1N | 41 seconds | | | | | | |
| HCl) 18.0 average kp | | | | | | | |
| hardness | | | | | | | |
| Dissolution % label | | | | | | | |
| strength - Paddles, 75 | | | | | | | |
| RPM. 900 mL 5 wt. % | | | | | | | |
| SLS in 50 mM sodium | | | | | | | |
| phosphate buffer, pH 6.8 | | | | | | | |
| (Whole Tablets) | Tablet 1 | Tablet 2 | Tablet 3 | Tablet 4 | Tablet 5 | Tablet 6 | % RSD |
| 30 minutes | 96.1 | 95.2 | 96.6 | 97.2 | 96.4 | 96.4 | — |
| 60 minutes | 97.0 | 97.0 | 98.1 | 98.3 | 97.0 | 97.9 | — |
| 90 minutes | 97.1 | 97.3 | 98.7 | 99.0 | 98.1 | 98.3 | 0.8% |
| Dissolution % label | | | | | | | |
| strength - Paddles, 75 | | | | | | | |
| RPM. 900 mL 5 wt. % | | | | | | | |
| SLS in 50 mM sodium | | | | | | | |
| phosphate buffer, pH 6.8 | | | | | | | |
| (Bisected Tablets) | Tablet 1 | Tablet 2 | Tablet 3 | Tablet 4 | Tablet 5 | Tablet 6 | |
| 30 minutes | 98.5 | 96.5 | 89.3 | 99.1 | 95.6 | 97.1 | — |
| 60 minutes | 99.5 | 98.3 | 91.1 | 100.7 | 97.0 | 98.3 | — |
| 90 minutes | 100.0 | 98.8 | 90.9 | 101.0 | 97.9 | 98.7 | 3.6% |

These data indicate that if bisecting the tablet is needed the resulting half tablet will provide a satisfactorily adequate dose which will dissolve in a similar manner to the whole tablets.

The ASD composition is provided in Table 20.

TABLE 20
Composition of INX-315 ASD Formulation
(40% INX-315, 60% HPMCAS-HG)

| Component | Quality Standard | Function | % (w/w) |
|---|---|---|---|
| INX-315 | In-house | Active | 40.0* |
| HPMCAS-HG | NF | Precipitation | 60.0 |
| | | Preventer | |
| Total Solids | | | 100.0 |
| Total Solids | NA | | 7.5% |
| THF | ACS Grade | Solvent (removed | 90% |
| | | during processing) | |
| Water | Purified Water, | Solvent (removed | 10% |
| | USP | during processing) | |
| Total Solvent | | | 92.5% |

| | |
|---|---|
| Total Solids + Total Solvent | 100% |

Abbreviations:
HPMCAS-HG—hydroxy propyl methylcellulose acetate succinate - high substitution;
NF—National Formulary;
USP—United States Pharmacopeia
*The amount of drug substance dispensed into a lot of drug product may be adjusted to obtain the theoretical concentration of INX-315. Therefore, the amount of a particular lot added to a batch may be factored for purity, moisture, volatiles and residue on ignition based on the certificate of analysis for the active ingredient used during manufacture. Adjustment is optional for factors of less than 1%.

### Description and Composition of the INX-315 Drug Product

INX-315 tablets for oral administration were manufactured in 100 mg active ingredient dose strengths. The composition of the tablet is provided in Table 21.

TABLE 21
Composition of INX-315 Tablets, 100 mg

| | | | | Amount |
|---|---|---|---|---|
| | | | | Per |
| | Quality | | % | Tablet |
| Component | Standard | Function | (w/w) | (mg) |
| 40% INX-315:60% | In-house | Active | 50.0 | 250.0 |
| HPMCAS-H ASD | | | | |
| Microcrystalline Cellulose | NF | Insoluble | 20.5 | 102.5 |
| (Avicel PH-101) | | diluent | | |
| Mannitol (Parteck M 100) | USP | Soluble | 20.5 | 102.5 |
| | | diluent | | |
| Croscarmellose Sodium | NF | Disintegrant | 3.67 | 18.3 |
| Colloidal Silicon Dioxide | NF | Glidant | 1.0 | 5.0 |
| Sodium Stearyl Fumarate | NF | Lubricant | 1.0 | 5.0 |
| Intragranular Portion | | | 96.67 | 483.3 |
| Croscarmellose Sodium | NF | Disintegrant | 2.33 | 11.7 |
| Colloidal Silicon Dioxide | NF | Glidant | 0.5 | 2.5 |
| Sodium Stearyl Fumarate | NF | Lubricant | 0.5 | 2.5 |

| | | |
|---|---|---|
| Extragranular Portion | 3.33 | 16.7 |
| Total | 100.0 | 500.0 |

Abbreviations:
HPMCAS-HG—hydroxy propyl methylcellulose acetate succinate - high substitution;
NF—National Formulary;
USP—United States Pharmacopeia

### Description and Composition of the INX-315 Batch Formula

Batch sizes at this stage of development will vary based on API availability and the clinical need. Presented below is the formula for a typical batch size of 5,290.8 g of 40% INX-315, 60% HPMCAS-H ASD. The ASD composition is provided in Table 22.

TABLE 22
Typical Batch of INX-315 ASD (40%
INX-315, 60% HPMCAS-HASD)

| Component | Quality Standard | Function | Weight | |
|---|---|---|---|---|
| INX-315* | In-house | Active | 2,116.3 | g |
| HPMCAS-HG | NF | Diluent/Dispersant | 3,174.5 | g |
| THF | ACS Grade | Solvent (removed | 58.8 | kg |
| Water | Purified Water, USP | during processing) | 6.5 | kg |

| | | |
|---|---|---|
| Total Solids | 5,290.8 | g |

Abbreviations:
HPMCAS-HG—hydroxy propyl methylcellulose acetate succinate - high substitution;
NF—National Formulary;
USP—United States Pharmacopeia
*The amount of drug substance dispensed into a lot of drug product may be adjusted to obtain the theoretical concentration of INX-315. Therefore, the amount of a particular lot added to a batch may be factored for purity, moisture, volatiles and residue on ignition based on the certificate of analysis for the active ingredient used during manufacture. A corresponding amount of HPMCAS-HG is removed such that the total solids amount is unchanged. Adjustment is optional for factors of less than 1%.

Atypical batch of 4.0 kg is presented in Table 23 below.

TABLE 23
Typical Batch of INX-315 Tablets, 100 mg

| | Quality | | | Amount Per |
|---|---|---|---|---|
| Component | Standard | Function | % (w/w) | Batch (g) |
| 40% INX-315, | In-house | Active | 50.0 | 2,000.0 |
| 60% HPMCAS-H | | | | |
| ASD | | | | |
| Microcrystalline | NF | Insoluble | 20.5 | 820.0 |
| Cellulose | | diluent | | |
| (Avicel PH-101) | | | | |
| Mannitol (Parteck | USP | Soluble diluent | 20.5 | 820.0 |
| M 100) | | | | |
| Croscarmellose | NF | Disintegrant | 3.67 | 146.8 |
| Sodium | | | | |
| Colloidal Silicon | NF | Glidant | 1.0 | 40.0 |
| Dioxide | | | | |
| Sodium Stearyl | NF | Lubricant | 1.0 | 40.0 |
| Fumarate | | | | |
| Intragranular Portion | | | 96.67 | 3866.8 |
| Croscarmellose | NF | Disintegrant | 2.33 | 93.2 |
| Sodium | | | | |
| Colloidal Silicon | NF | Glidant | 0.5 | 20.0 |
| Dioxide | | | | |
| Sodium Stearyl | NF | Lubricant | 0.5 | 20.0 |
| Fumarate | | | | |

| | | |
|---|---|---|
| Extragranular Portion | 3.33 | 133.2 |
| Total | 100.0 | 4000.0 |

Abbreviations:
HPMCAS-HG—hydroxy propyl methylcellulose acetate succinate - high substitution;
NF—National Formulary;
USP—United States Pharmacopeia

### Example 5. INX-315 Inhibits Tumor Growth in Rb-Negative Cancers when Administered as a Monotherapy or in Combination with Chemotherapy

The purpose of this study was to examine the efficacy of INX-315 administered alone or in combination with chemotherapy in an LU5202 [small cell lung cancer (SCLC)] patient-derived xenograft (PDX) model.

8-week-old NOD/SCID female mice (n=59) were split into two cohorts. A total of 29 mice were enrolled in cohort 1 of the study. A total of 30 mice were enrolled in cohort 3 of the study. When the average tumor volume reached 150-250 mm³, all animals were randomly allocated to the 6 different study groups using Study Log software (Table 24). Animals were monitored weekly for palpable tumors, or any changes in appearance or behavior. Once tumors were palpable, tumors were measured at least once weekly using calipers. Body weight was measured at least 2 times per week following randomization and initiation of treatment.

TABLE 24
Treatment Groups

| | | | | Dosing | | Dose |
|---|---|---|---|---|---|---|
| | | | Dose | Frequency & | Dose Level | Volume |
| Group | N | Treatment | Route | Duration | (mg/kg) | (mL/kg) |
| 1 | 10 | Vehicle | PO | BID, 35 days | — | 5 |
| | | (100% PEG-400) | | | | |
| 2 | 10 | INX-315 ASD | PO | BID, 28 days | 100 | 10 |
| 3 | 10 | INX-315 | PO | BID, 35 days | 100 | 5 |
| 4 | 10 | INX-315 | PO | BID, 35 days | 150 | 5 |
| 5 | 5* | 100% PEG-400* | PO* | BID, 35 days | — | 5 |
| | 5 | Cisplatin | IP | QD, 1 day | 4 | 10 |
| | | | | (optional dose on | | |
| | | | | Day 21) | | |
| | | Etoposide | IP | QD × 3 doses | 8 | 10 |
| | | | | (optional dose on | | |
| | | | | day 21) | | |
| 6 | 10 | INX-315 | PO | BID, 35 days | 100 | 5 |
| | | Cisplatin | IP | QD, 1 day | 4 | 10 |
| | | | | (optional dose on | | |
| | | | | Day 21) | | |
| | | Etoposide | IP | QD × 3 doses | 8 | 10 |
| | | | | (optional dose on | | |
| | | | | day 21) | | |

In cohort 1, INX-315 monotherapy at 100 mg/kg BID and 150 mg/kg BID inhibited tumor growth similarly (62% and 66%, respectively). Addition of INX-315 (100 mg/kg) to chemotherapy resulted in greater tumor growth inhibition (70% compared to 62 or −12%) (FIG. 15C). Mice treated with chemotherapy and INX-315 lost weight and did not tolerate the treatment well (FIG. 15A). To test if this was toxicity was due to test article or vehicle (PEG-400) related, cohort 2 mice receiving only chemotherapy were also given vehicle PG. All mice in cohort 2 receiving chemotherapy were given hydrogel prophylactically.

In cohort 2, INX-315 at 100 mg/kg BID and 150 mg/kg BID inhibited tumor growth at 70% and 87%, respectively (FIG. 15D). In addition to the above-mentioned changes to the chemotherapy groups, cohort 2 chemotherapy treatment at day 21 was changed from only receiving a second cisplatin dose, to receiving a second round of both cisplatin and etoposide. Chemotherapy treatment in cohort 2 resulted in 72% tumor growth inhibition, while the addition of INX-315 (100 mg/kg) improved tumor growth inhibition to 94% (FIG. 15D). Both chemotherapy only and chemotherapy plus INX-315 resulted in weight loss and two dead mice in the chemotherapy only group, suggesting that vehicle (PEG-400) is contributing to increased toxicity (FIG. 15B).

While not wishing to be bound to any one theory, the CDK2 inhibitor INX-315 is capable of acting through Rb-independent mechanisms and demonstrates anti-cancer efficacy in Rb-independent models of cancer.

### Example 6. INX-315 Administered in Combination with a CDK4/6 Inhibitor Inhibits Tumor Growth in CDK4/6 Inhibitor-Resistant Cancer

The purpose of this study was to examine the efficacy of INX-315 administered alone or in combination with CDK4/6 inhibitor ribociclib in a patient-derived xenograft (PDX) model representing human ER+, CDK4/6 inhibitor-resistant breast cancer in immune-deficient mice.

Athymic nude, outbread homozygous (Crl:NU(NCr)-Foxnl^{nu}) 6-12-week-old ST4316B [HX] mice were analyzed. Tumor fragments from ST4316B were harvested from host animals and implanted into immune-deficient mice and the study initiated at a mean tumor volume of approximately 150-300 mm³. INX-315 was run as a monotherapy and in combination with a CDK4/6 inhibitor (ribociclib). Data collected from this study included animal weights, observations, and tumor dimensions. This information was used to determine agent tolerability based on weight change and gross physiologic changes; and anticancer activity based on tumor growth inhibition or regression. The data analysis endpoint for this study was Day 31.

TABLE 25
Detailed Study Outline

| | | | Dose | | | Data |
|---|---|---|---|---|---|---|
| | | | (mg/ | ROA/ | Total | End- |
| Group | N | Treatment | kg) | Schedule | Dosed | point |
| 1 | 8 | Vehicle | — | PO/BID, 28 days | 90 | 45 |
| 2 | 8 | Ribociclib | 50 | PO/QD, 28 days | 50 | 49 |
| 3 | 8 | INX-315 | 25 | PO/BID, 28 days | 62 | 31 |
| 4 | 8 | INX-315 | 50 | PO/BID, 28 days | 62 | 31 |
| 5 | 8 | INX-315 | 100 | PO/BID, 28 days | 98 | 49 |
| 6 | 8 | Ribociclib | 50 | PO/QD, 28 days | 32 | 31 |
| | | INX-315 | 25 | PO/BID, 28 days | 62 | |
| 7 | 8 | Ribociclib | 50 | PO/QD, 28 days | 32 | 31 |
| | | INX-315 | 50 | PO/BID, 28 days | 62 | |
| 8 | 8 | Ribociclib | 50 | PO/QD, 28 days | 50 | 49 |
| | | INX-315 | 100 | PO/BID, 28 days | 98 | |

No notable model-related weight loss was reported in the tested model based on lack of weight loss in control animals (FIG. 16A). In this study, single agent and combination therapies were tolerated with a maximum weight loss of 10% in any group and no drug-related deaths; one technical death occurred on Day 16 (Group 6) and two deaths of unknown cause occurred, one on Day 21 (Group 4) and one on Day 24 (Group 5), unrelated to drug tolerability.

At Day 31, statistically significant tumor growth inhibition versus control was reported in the INX-315 (100 mg/kg)+ribociclib (50 mg/kg) group (p=0.0033) (FIG. 16B). The 100 mg/kg BID INX-315 dose was selected to continue dosing as monotherapy and in combination with ribociclib out to Day 49. On Day 45 (the final day for control mice) the tumor growth inhibition percentage (TGI %) for the INX-315 (100 mg/kg)+ribociclib (50 mg/kg) group (Group 8) was 65% (p<0.0002) (FIG. 16B).

Overall, single agent and combination therapies were tolerated and one combination group active towards the ST4316B ER+, CDK4/6 inhibitor-resistant breast PDX model.

### Example 7. Preliminary Clinical Data

Cohort 1 enrollment of 6 subjects dosed at 100 mg oral QD was completed in October of 2023. Patient characteristics are shown in Table 26.

TABLE 26
Patient Characteristics

| Subject | | | CCNE1 | |
|---|---|---|---|---|
| ID | Age/Sex | Tumor Type | Status | Prior Treatment |
| 101-001 | F/66 | ER+/HER2− Breast | N/A | Adjuvant: |
| | | Cancer | | anastrozole |
| | | | | Metastatic: |
| | | | | palbociclib/fulvestrant; alpelisib |
| 201-001 | F/58 | High-Grade Serous | CCNE1 | Adjuvant: |
| | | Ovarian Cancer | Amplified | carboplatin/paclitaxel; Metastatic: |
| | | | | olaparib + durvalumab (SOLACE trial); |
| | | | | carboplatin + liposomal doxorubicin; |
| | | | | AMT-151 (anti-folate receptor alpha |
| | | | | ADC) |
| 101-003 | M/42 | Esophageal | CCNE1 | Metastatic: |
| | | Adenocarcinoma | Amplified | Fluorouracil + leucavorin + oxaliplatin |
| | | | | (FOLFOX) + nivolumab; 5-FU; |
| | | | | irinotecan; ramucirumab + paclitaxel |
| 201-002 | F/60 | High-Grade Serous | CCNE1 | Adjuvant: |
| | | Ovarian Cancer | Amplified | carboplatin/paclitaxel |
| | | | | Metastatic: |
| | | | | tamoxifen; paclitaxel + bevacizumab; |
| | | | | bevacizumab (maintenance) |
| 103-001 | F/39 | ER+/HER2− Breast | N/A | Neoadjuvant: |
| | | Cancer | | ddAC-T |
| | | | | Adjuvant: |
| | | | | post mastectomy RT |
| | | | | Metastatic: |
| | | | | Lupron; anastrazole; xentuzumab, |
| | | | | abemaciclib, fulvestrant, xgeva, |
| | | | | Lupron; fulvestrant, Ibrance; |
| | | | | fulvestrant, Trelstar, Xgeva, |
| | | | | abemaciclib; fulvestrant, everolimus; |
| | | | | Xeloda; Xeloda; Enhertu |
| 102-001 | F/49 | TNBC | CCNE1 | Adjuvant: |
| | | | Amplified | doxorubicin/cyclophosphamide/ |
| | | | | paclitaxel; capecitabine; |
| | | | | gemcitabine/carboplatin; trastuzumab |
| | | | | deruxtecan |
| | | | | Metastatic: |
| | | | | Sacituzumab govitecan-HZIY |

No DLTs were reported and treatment was well tolerated at the 100 mg QD dose level. No significant changes in complete blood counts or clinical chemistries were observed.

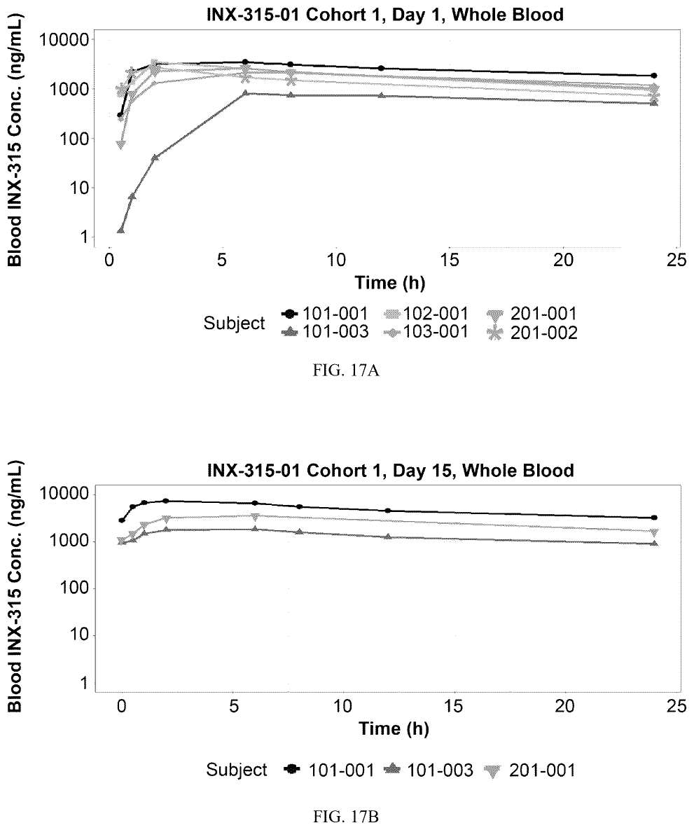

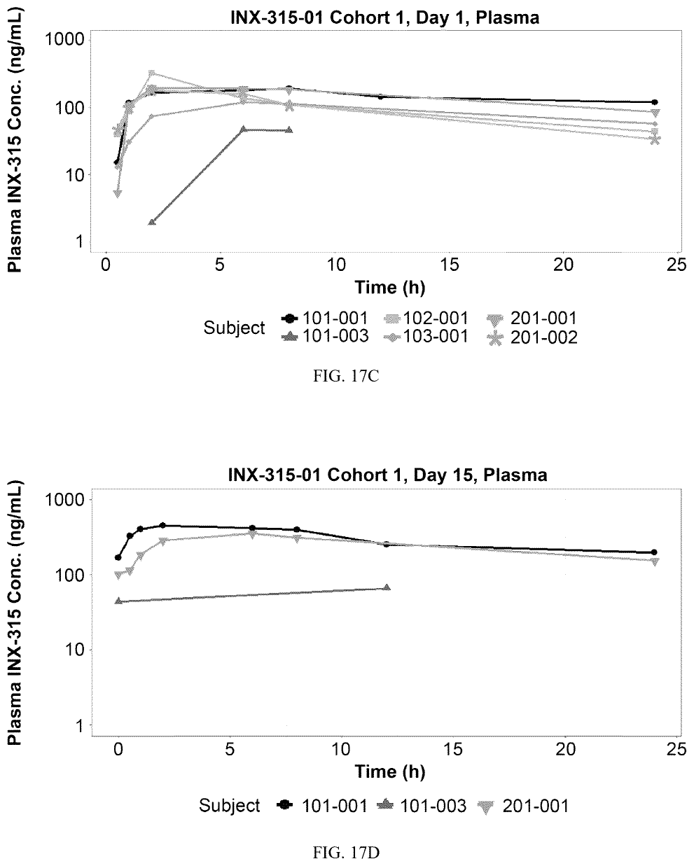

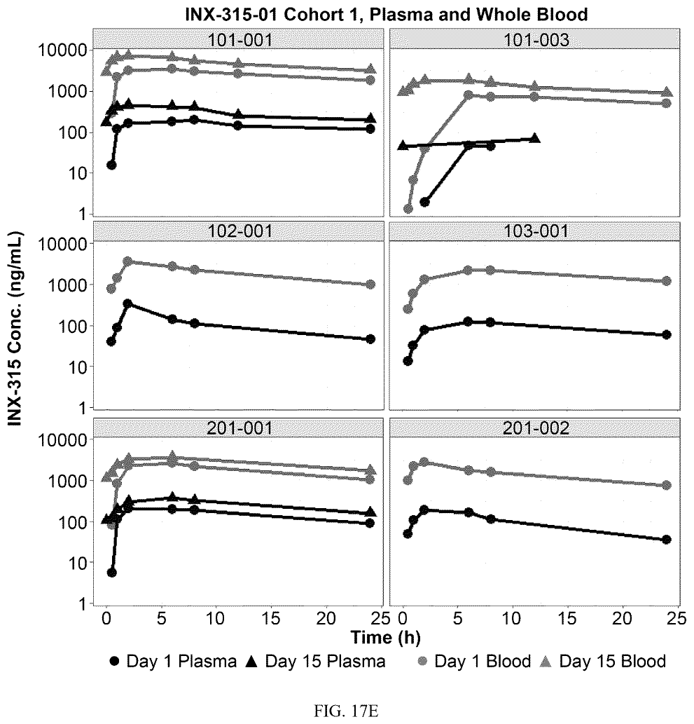

The PK parameters of INX-315 were tested in whole blood and plasma in six individual patients in Cohort 1 on days 1 and 15 (FIG. 17A-17E; Tables 27-28).

TABLE 27
Preliminary Plasma INX-315 PK Parameters

| | | | | | |
|---|---|---|---|---|---|
| | Cₘₐₓ | Tₘₐₓ | Cₗₐₛₜ | AUCₗₐₛₜ | t_{1/2} |
| Subject ID | (ng/ml) | (h) | (ng/ml) | (ng*h/ml) | (h) |

Day 1

| | | | | | |
|---|---|---|---|---|---|
| 101-001 | 194 | 8 | 119 | 3490 | NC |
| 102-001 | 326 | 2 | 43.9 | 2510 | 11.5 |
| 103-001 | 119 | 6 | 57.0 | 1990 | 16.7 |
| 201-001 | 197 | 2 | 86.3 | 3410 | 15.1 |
| 201-002 | 179 | 2 | 33.9 | 2160 | 8.88 |
| Geometric | 193 | 2 | 61.4 | 2640 | 12.6 |
| Mean | | | | | |
| CV % | 37.1 | (8, 2) | 54.0 | 26.1 | 28.9 |

Day 15

| | | | | | |
|---|---|---|---|---|---|
| 101-001 | 457 | 2 | 199 | 7240 | 17.0 |
| 201-001 | 358 | 6 | 155 | 5920 | 15.2 |

NC = not calculated due to insufficient data;
Tₘₐₓ reported as median (range)

TABLE 28
Preliminary Blood INX-315 PK Parameters

| | | | | | |
|---|---|---|---|---|---|
| Subject | Cₘₐₓ | Tₘₐₓ | Cₗₐₛₜ | AUCₗₐₛₜ | t_{1/2} |
| ID | (ng/ml) | (h) | (ng/ml) | (ng*h/ml) | (h) |

Day 1

| | | | | | |
|---|---|---|---|---|---|
| 101-001 | 3430 | 6 | 1840 | 60500 | 22.4 |
| 101-003 | 805 | 6 | 502 | 13400 | 27.4 |
| 102-001 | 3470 | 2 | 954 | 43600 | 12.8 |
| 103-001 | 2110 | 6 | 1180 | 37800 | 20.5 |
| 201-001 | 2570 | 6 | 1020 | 40600 | 13.9 |
| 201-002 | 2710 | 2 | 719 | 32500 | 14.5 |
| Geometric | 2280 | 6 | 956 | 34700 | 17.9 |
| Mean | | | | | |
| CV % | 58.6 | (2, 6) | 46.4 | 54.7 | 31.4 |

Day 15

| | | | | | |
|---|---|---|---|---|---|
| 101-001 | 7290 | 2 | 3220 | 117000 | 21.6 |
| 101-003 | 1820 | 6 | 891 | 31400 | 18.3 |
| 201-001 | 3580 | 6 | 1670 | 62700 | NC |
| Median | 3580 | 6 | 1670 | 62700 | NR |
| Range | (1820, 7290) | (2, 6) | (891, 3220) | (31400, | (18.3, 21.6) |
| | | | | 117000) | |

NC = not calculated due to insufficient data;
Tₘₐₓ reported as median (range)

Cohort 1 efficacy included 1 partial response (PR) subject, 3 stable disease (SD) subjects, and 2 progressive disease (PD) subjects.

Subject 101-001 exhibited a confirmed PR. Subject 201-001 exhibited SD at end of Cycle 2 (C2) with a 10% decrease in TL+reduction in CA-125, with subsequent clinical PD at C4 with no change observed on CT. Subject 101-003, a male with esophageal cancer, developed brain metastases and died within weeks of coming off the study. This particular subject's ability to absorb INX-315 was compromised due to these unforeseen circumstances.

The preliminary data of the expanded cohort suggest the t_{1/2} of INX-315 is at least 12 hours (Tables 27-28). The concentration versus time profile at 100 mg QD in this cohort supports the finding that INX-315 has a low Cₘₐₓ to Cₘᵢₙ ratio (Tables 27-28). The relatively flat concentration versus time profile of INX-315 indicates a lower exposure required to maintain concentrations above therapeutic threshold (FIG. 17A-17E).

### Example 8. Preliminary Clinical Data at 200 mg Once Daily (QD)

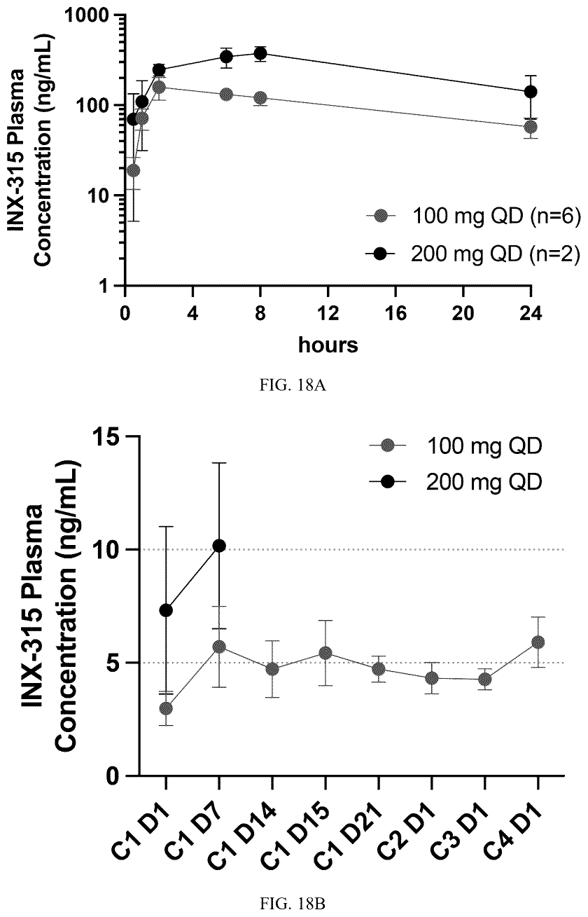

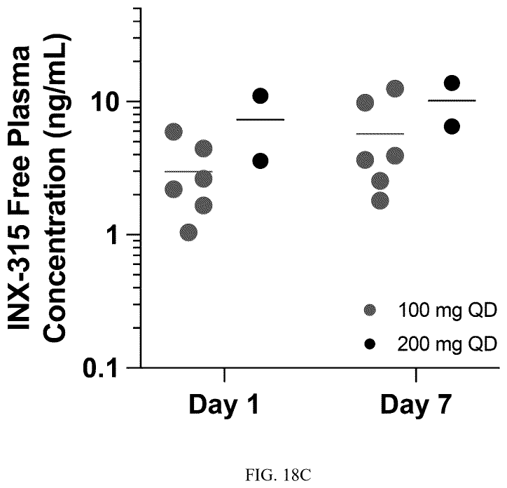

Patients were upgraded to the 200 mg QD dose level and PK/PD plasma parameters were measured (FIG. 18A).

TABLE 29
INX-315 Area Under the Curve (AUC) Comparison
between 100 mg QD and 200 mg QD

| | 100 mg QD (n = 6) | 200 mg QD (n = 2) |
|---|---|---|
| Total Area | 2398 (2743*) | 6259 |

*Adjusted AUC when removing esophageal cancer patient (101-103) with low exposure.

AUC values of patients dosed at 100 mg QD versus 200 mg QD were compared (Tables 29-30), in which a dose-proportional increase in exposure (or greater) was observed.

TABLE 30
INX-315 Plasma Concentrations by Timepoint

| | 100 mg QD (n = 6) | | 200 mg QD (n = 2) | | |
|---|---|---|---|---|---|
| Time | Mean | SEM | Mean | SEM | Ratio |
| (hr) | (ng/mL) | (ng/mL) | (ng/mL) | (ng/mL) | (200 mg/100 mg) |
| 0.5 | 18.9 | 7.3 | 69.6 | 64.4 | 3.68 |
| 1.0 | 71.4 | 18.9 | 109.2 | 77.9 | 1.53 |
| 2.0 | 158.1 | 44.9 | 246.5 | 35.5 | 1.56 |
| 6.0 | 131.8 | 20.5 | 343 | 85 | 2.60 |
| 8.0 | 120.6 | 22.1 | 374.5 | 69.5 | 3.10 |
| 24.0 | 57.4 | 14.5 | 140.9 | 71.2 | 2.46 |

Based on cohort 1 data, steady state trough concentrations appear to be reached by day 7 (FIG. 18B).

TABLE 31
INX-315 Mean Free Plasma Trough Concentration (ng/mL)

| | 100 mg QD (n) | 200 mg QD (n) |
|---|---|---|
| Day 1 | 2.98 (6) | 7.3 (2) |
| Day 7 | 5.7 (6) | 10.2 (2) |

Cohort 2 exhibits increased INX-315 free plasma concentrations at Day 1 and Day 7 (Tables 31-32; FIG. 18C).

TABLE 32
INX-315 Free Plasma Trough Concentrations by Timepoint

| | 100 mg QD | | | 200 mg QD | | |
|---|---|---|---|---|---|---|
| | Mean | SEM | | Mean | SEM | |
| Cycle/Day | (ng/mL) | (ng/mL) | N | (ng/mL) | (ng/mL) | N |
| C 1 D 1 | 2.98 | 0.75 | 6 | 7.31 | 3.71 | 2 |
| C 1 D 7 | 5.71 | 1.79 | 6 | 10.16 | 3.67 | 2 |
| C 1 D 14 | 4.72 | 1.25 | 4 | | | |
| C 1 D 15 | 5.43 | 1.45 | 5 | | | |
| C 1 D 21 | 4.72 | 0.57 | 5 | | | |
| C 2 D 1 | 4.32 | 0.69 | 4 | | | |
| C 3 D 1 | 4.27 | 0.47 | 3 | | | |
| C 4 D 1 | 5.91 | 1.11 | 2 | | | |

Despite the increase in exposure, no new safety issues have been reported to date.

### Example 9. Additional Spray Dried Dispersion Batch Formulation

An additional INX-315 ASD composition is provided in Table 33 below with a modified THF:H₂O Solvent Ratio from 90%:10% to 92%:8%.

TABLE 33
Batch of INX-315 ASD Formulation
(40% INX-315, 60% HPMCAS-HG)

| | | | Spray | | |
|---|---|---|---|---|---|
| | | | Dried | | Spray |
| | | | Dispersion | | Solution |
| | Quality | | Material | Weight | Material |
| Component | Standard | Function | (%) | (g) | (%) |
| INX-315* | | Active | 40.0 | 2050.0 | 3.8 |
| HPMCAS- | NF | Precipitation | 60.0 | 2824.0 | |
| HG | | Preventer | | | |

| | | | Spray | |
|---|---|---|---|---|
| | Quality | | Solvent | |
| Component | Standard | Function | (w/w %) | Weight (g) |
| THF Stabilized | ACS Grade | Solvent | 92% | 113.5 |
| with BHT | | (removed | | |
| | | during | | |
| | | processing) | | |
| Purified Water | USP | Solvent | 8% | 9.9 |
| | | (removed | | |
| | | during | | |
| | | processing) | | |

Abbreviations:
HPMCAS-HG—hydroxy propyl methylcellulose acetate succinate - high substitution;
NF—National Formulary;
USP—United States Pharmacopeia;
BHT—butylated hydroxytoluene.

The solids loading spray solution material was modified to 3.8%, whereas in a previous batch this solids loading spray solution material was 7.5% (see, e.g., Table 20). Additionally, the SDD drying temperature was modified to include 90° C.

### Example 10. Summary Preliminary Clinical Data at 100 mg, 200 mg, and 300 mg Once Daily (QD)

Patients were administered the 200 mg QD and 300 mg QD dose levels and PK/PD plasma parameters were measured.

The PK parameters of INX-315 were tested in whole blood and plasma in individual patients in Cohorts 1, 2, and 3 on days 1 and 15 (Tables 34-36).

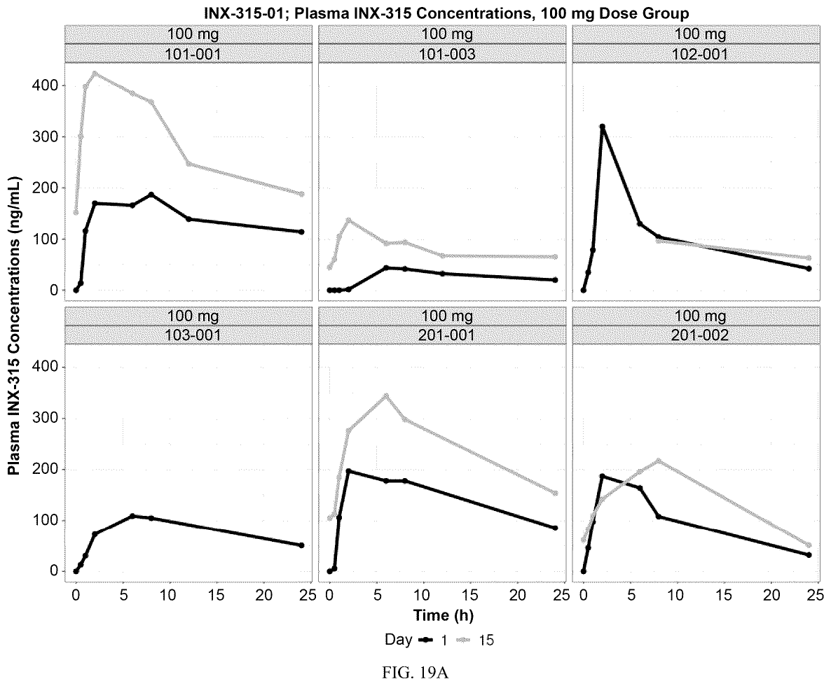

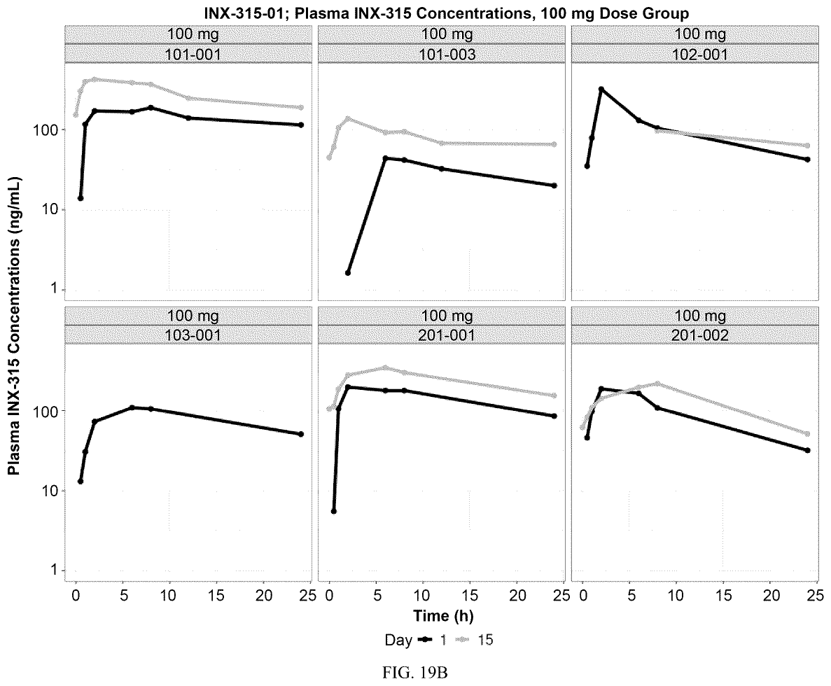

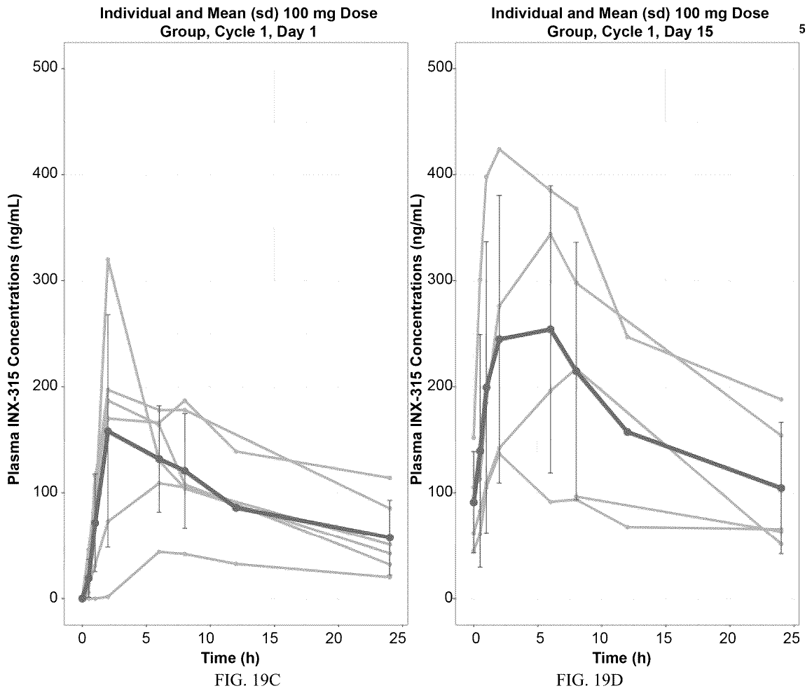

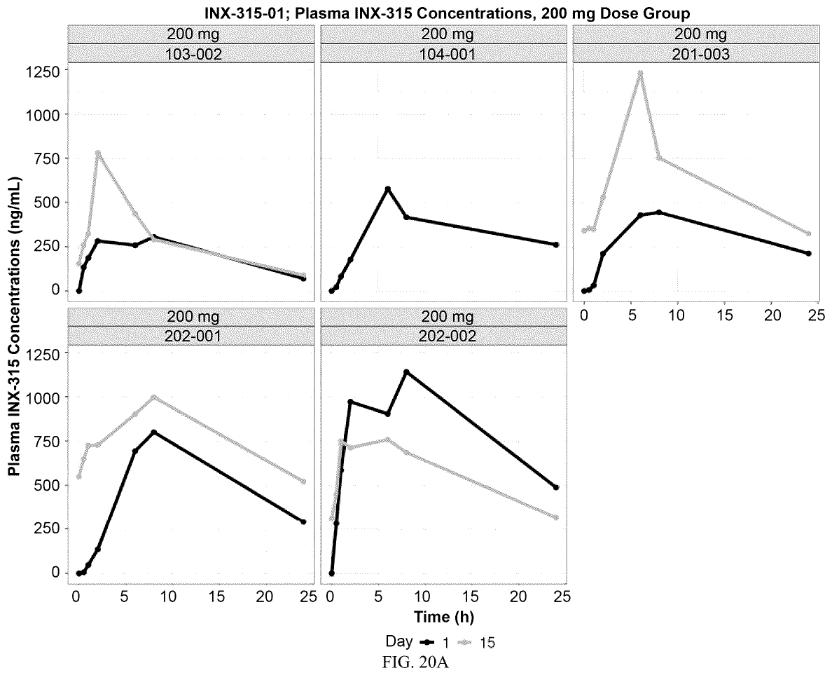

INX-315 plasma concentration values for individual subjects in Dose Cohort 1 (100 mg QD) on Cycle 1 Day 1 and Cycle 1 Day 15 are illustrated in FIG. 19A-19B. Individual and mean (solid black line) plasma INX-315 concentrations on Cycle 1 Day 1 and Cycle 1 Day 15 are illustrated in FIG. 19C-19D, respectively. These data are summarized in Table 34 below.

TABLE 34
Preliminary Plasma INX-315 Pharmacokinetic
Parameters 100 mg Dose Cohort

| | Cₘₐₓ | Tₘₐₓ | Tₗₐₛₜ | Cₗₐₛₜ | AUCₗₐₛₜ | t_{1/2} |
|---|---|---|---|---|---|---|
| Subject | (ng/mL) | (h) | (h) | (ng/mL) | (ng*h/mL) | (h) |
| | Day 1 | | | | | |
| 101-001 | 187 | 8 | 24 | 114 | 3360 | NR |
| 101-003 | 43.8 | 6 | 24 | 20 | 633 | 15.6 |
| 102-001 | 320 | 2 | 24 | 42.3 | 2410 | 11.5 |
| 103-001 | 109 | 6 | 24 | 50.7 | 1840 | 15.9 |
| 201-001 | 197 | 2 | 24 | 85.2 | 3300 | 16.2 |
| 201-002 | 187 | 2 | 24 | 31.9 | 2160 | 8.39 |
| Geometric | 148 | NR | NR | 48.7 | 2020 | 13.1 |
| Mean | (77.7) | | | (70.7) | (67.8) | (29.3) |
| (CV %) | | | | | | |
| Median | 187 | 4 | 24 | 46.5 | 2280 | 15.6 |
| (range) | (43.8, | (2, 8) | (24, 24) | (20, | (633, | (8.39, |
| | 320) | | | 114) | 3360) | 16.2) |
| | Day 15 | | | | | |
| 101-001 | 424 | 2 | 24 | 188 | 6840 | 17.6 |
| 101-003 | 137 | 2 | 24 | 65.4 | 1930 | NR |
| 102-001 | 96.4 | 8 | 24 | 63.1 | 1640 | NR |
| 201-001 | 344 | 6 | 24 | 154 | 5700 | 16.0 |
| 201-002 | 217 | 8 | 24 | 51.2 | 3120 | NR |
| Geometric | 211 | NR | NR | 90.6 | 3290 | NC |
| Mean | (68.2) | | | (64.1) | (70.3) | |
| (CV %) | | | | | | |
| Median | 217 | 6 | 24 | 65.4 | 3120 | NC |
| (range) | (96.4, | (2, 8) | (24, 24) | (51.2, | (1640, | |
| | 424) | | | 188) | 6840) | |

NC = not calculated, insufficient data;
NR = not reported

INX-315 PK values in plasma greatly exceeded predicted values estimated by a model based upon preclinical animal studies (Table 1). In Cohort 1, AUC and Cₘₐₓ values in plasma exceeded the model's prediction for each parameter on Day 1 by greater than a factor of 2.7 and 3.7, respectively, and more than a factor of 4.4 and 5.3, respectively, on day 15.

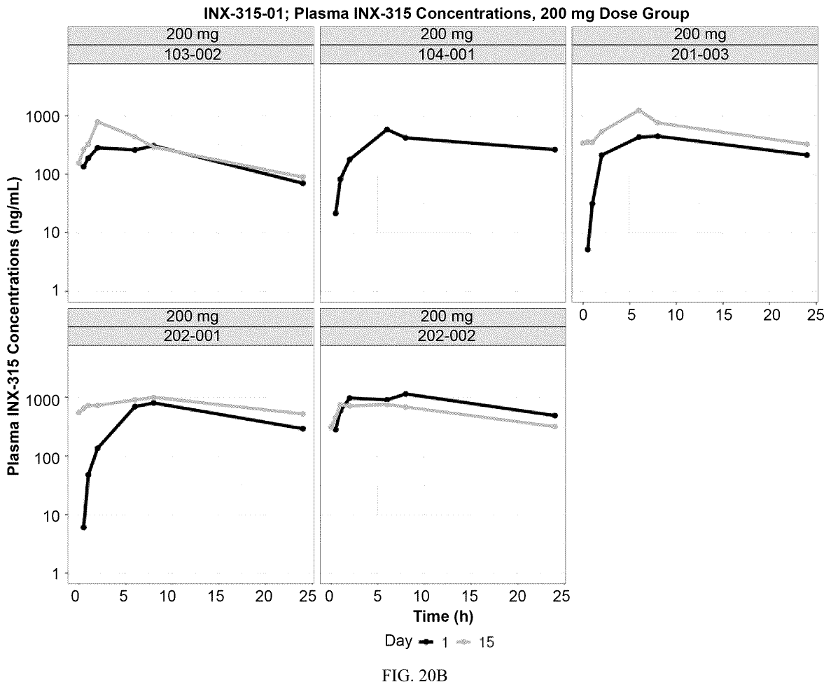

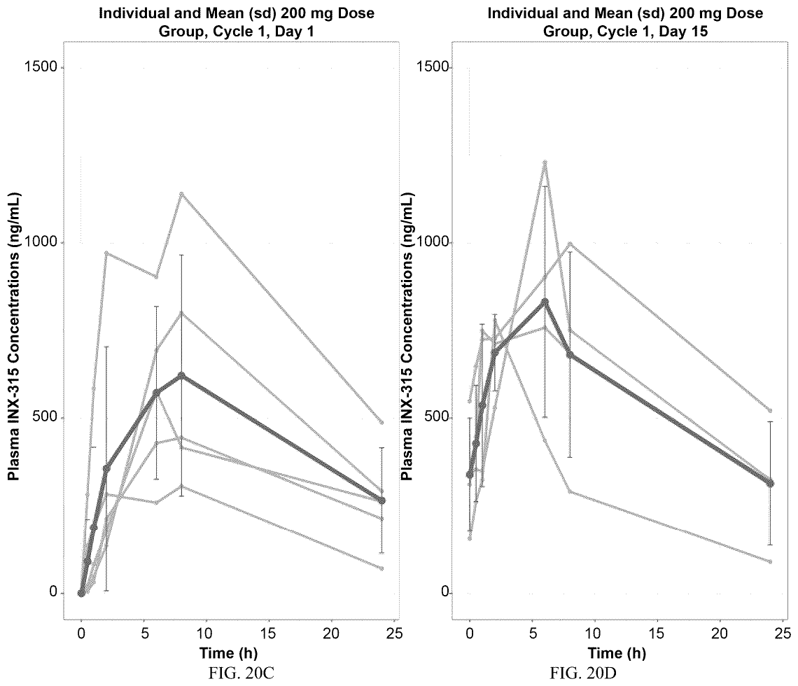

INX-315 plasma concentration values for individual subjects in Dose Cohort 2 (200 mg QD) on Cycle 1 Day 1 and Cycle 1 Day 15 are illustrated in FIG. 20A-20B. Individual and mean (solid black line) plasma INX-315 concentrations on Cycle 1 Day 1 and Cycle 1 Day 15 are illustrated in FIG. 20C-20D, respectively. These data are summarized in Table 35 below.

TABLE 35
Preliminary Plasma INX-315 Pharmacokinetic
Parameters 200 mg Dose Cohort

| | Cₘₐₓ | Tₘₐₓ | Tₗₐₛₜ | Cₗₐₛₜ | AUCₗₐₛₜ | t_{1/2} |
|---|---|---|---|---|---|---|
| Subject | (ng/mL) | (h) | (h) | (ng/mL) | (ng*h/mL) | (h) |
| | Day 1 | | | | | |
| 103-002 | 305 | 8 | 24 | 69.7 | 4540 | NR |
| 104-001 | 577 | 6 | 24 | 261 | 7970 | 18.1 |
| 201-003 | 444 | 8 | 24 | 212 | 7300 | NR |
| 202-001 | 800 | 8 | 24 | 291 | 11300 | NR |
| 202-002 | 1140 | 8 | 24 | 487 | 19100 | NR |
| Geometric | 590 | NR | NR | 223 | 8940 | NC |
| Mean | (54.6) | | | (82.1) | (57.6) | |
| (CV %) | | | | | | |
| Median | 577 | 8 | 24 | 261 | 7970 | NC |
| (range) | (305, | (6, 8) | (24, 24) | (69.7, | (4540, | |
| | 1140) | | | 487) | 19100) | |
| | Day 15 | | | | | |
| 103-002 | 779 | 2 | 24 | 88.8 | 6560 | 8.36 |
| 201-003 | 1230 | 6 | 24 | 324 | 14300 | 10.5 |
| 202-001 | 997 | 8 | 24 | 521 | 18100 | NR |
| 202-002 | 758 | 6 | 24 | 317 | 13200 | 14.3 |
| Geometric | 922 | NR | NR | 263 | 12200 | 10.8 |
| Mean | (23.1) | | | (88.1) | (45.8) | (27.6) |
| (CV %) | | | | | | |
| Median | 888 | 6 | 24 | 320 | 13700 | 10.5 |
| (range) | (758, | (2, 8) | (24, 24) | (88.8, | (6560, | (8.36, |
| | 1230) | | | 521) | 18100) | 14.3) |

NC = not calculated, insufficient data;
NR = not reported

INX-315 PK values in plasma again greatly exceeded predicted values estimated by a model based upon preclinical animal studies (Table 1). In Cohort 2, AUC and Cₘₐₓ values in plasma exceeded the model's prediction for each parameter on Day 1 by greater than a factor of 6.4 and 8. 1, respectively, and more than a factor of 8.8 and 12.7, respectively, on day 15.

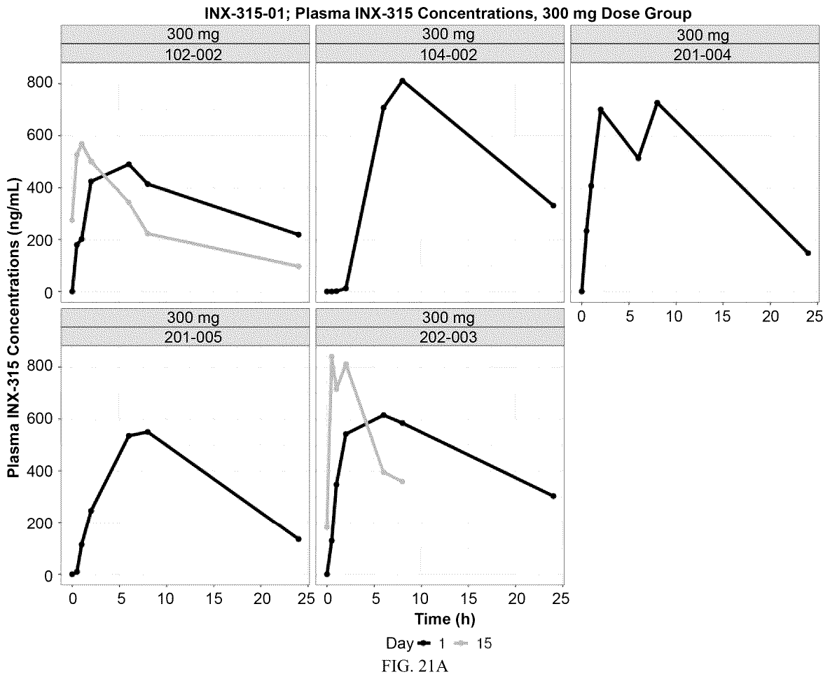

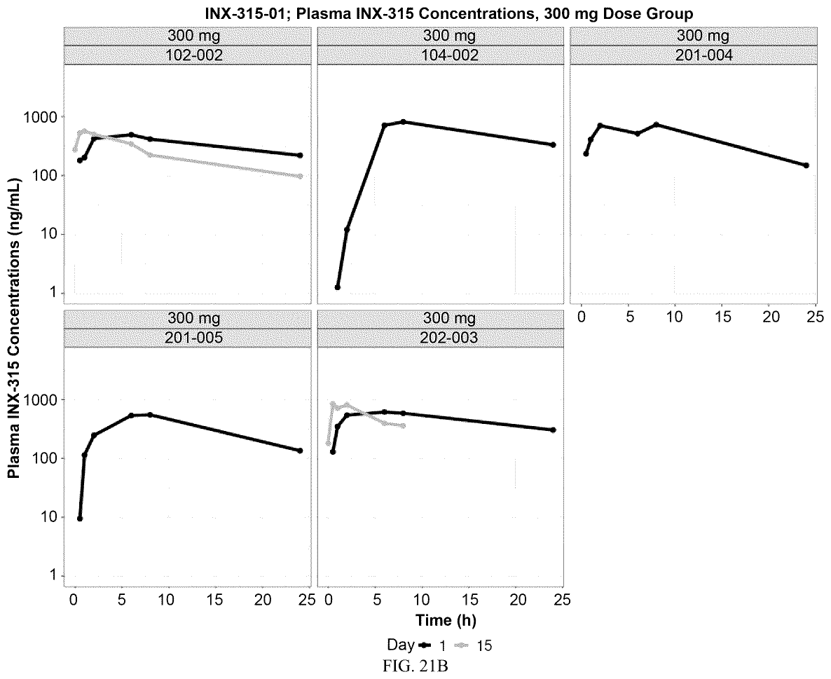

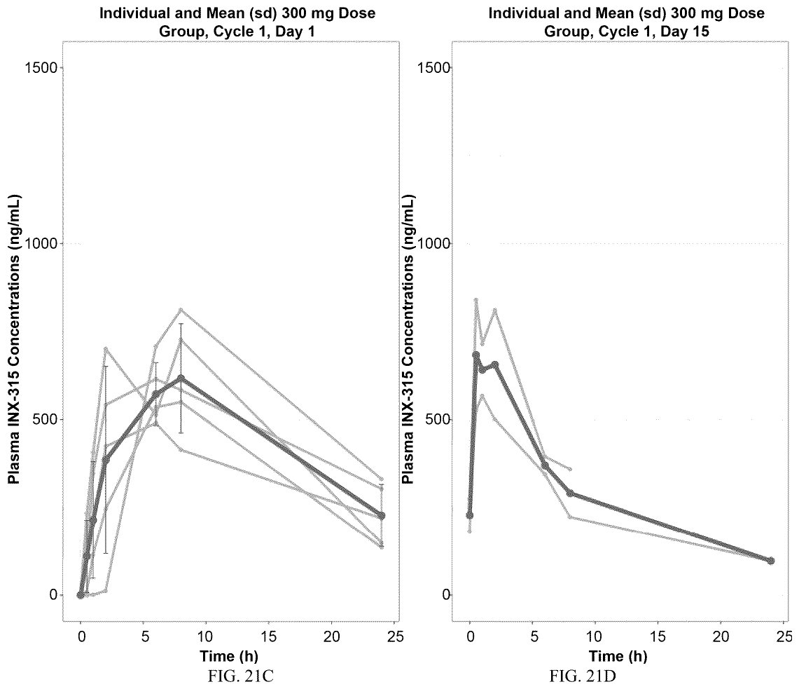

INX-315 plasma concentration values for individual subjects in Dose Cohort 3 (300 mg QD) on Cycle 1 Day 1 and Cycle 1 Day 15 are illustrated in FIG. 21A-21B. Individual and mean (solid black line) plasma INX-315 concentrations on Cycle 1 Day 1 and Cycle 1 Day 15 are illustrated in FIG. 21C-21D, respectively. These data are summarized in Table 36 below.

TABLE 16
Preliminary Plasma INX-315 Pharmacokinetic
Parameters 300 mg Dose Cohort

| | Cₘₐₓ | Tₘₐₓ | Tₗₐₛₜ | Cₗₐₛₜ | AUCₗₐₛₜ | t_{1/2} |
|---|---|---|---|---|---|---|
| Subject | (ng/mL) | (h) | (h) | (ng/mL) | (ng*h/mL) | (h) |
| | Day 1 | | | | | |
| 102-002 | 490 | 6 | 24 | 219 | 8080 | 16.2 |
| 104-002 | 812 | 8 | 24 | 331 | 11500 | NR |
| 201-004 | 727 | 8 | 24 | 148 | 10200 | NR |
| 201-005 | 550 | 8 | 24 | 135 | 7590 | NR |
| 202-003 | 615 | 6 | 24 | 303 | 11000 | 17.3 |
| Geometric | 628 | NR | NR | 213 | 9550 | NC |
| Mean | (20.7) | | | (42.3) | (18.9) | |
| (CV %) | | | | | | |
| Median | 615 | 8 | 24 | 219 | 10200 | NC |
| (range) | (490, 812) | (6, 8) | (24, 24) | (135, | (7590, | |
| | | | | 331) | 11500) | |
| | Day 15 | | | | | |
| 102-002 | 568 | 1 | 24 | 96.8 | 5590 | 9.23 |
| 202-003 | 840 | 0.5 | 8 | 359 | 4430 | 5.80 |

NC = not calculated, insufficient data;
NR = not reported

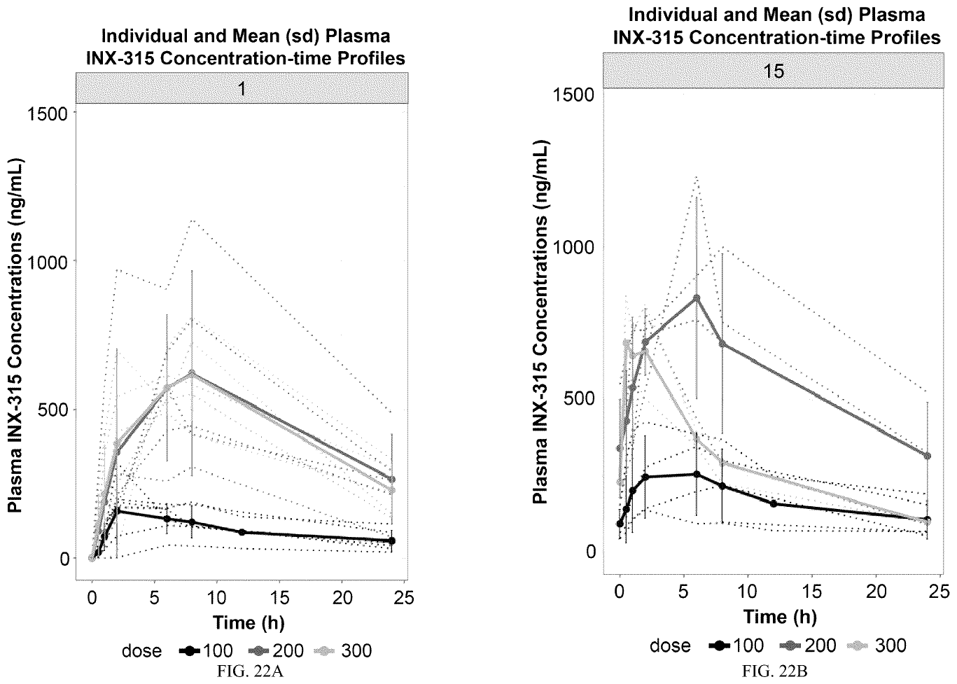

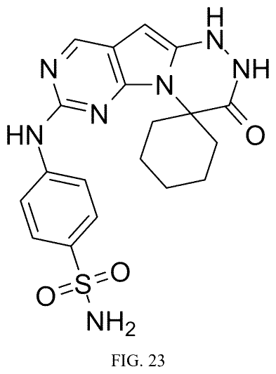

Pharmacokinetic parameter values of patients dosed at 100 mg QD versus 200 mg QD and 300 mg QD were compared, in which a dose-proportional increase in exposure (or greater) again was generally observed (FIG. 22A-22B).

These data for all Cohorts are summarized in Table 37.

TABLE 37
Preliminary INX-315 AUC Parameters

| | | | | | Plasma/Whole Blood | |
|---|---|---|---|---|---|---|
| Subject | Plasma | | Whole Blood | | Ratio | |
| ID | Day 1 | Day 15 | Day 1 | Day 15 | Day 1 | Day 15 |

Cohort 1 (100 mg QD)

| | | | | | | |
|---|---|---|---|---|---|---|
| 101-001 | 3370 | 6910 | 60740 | 118285 | 18.02 | 17.12 |
| 101-003 | 640.8 | 1951 | 13440 | 31754 | 20.97 | 16.28 |
| 102-001 | 2532 | 1276 | 44857 | ND | 17.72 | NA |
| 103-001 | 1886 | ND | 38499 | ND | 20.41 | ND |
| 201-001 | 3391 | 5858 | 41732 | 37642 | 12.31 | 6.43 |
| 201-002 | 2271 | 3444 | 33139 | 44316 | 14.59 | 12.87 |

Cohort 2 (200 mg QD)

| | | | | | | |
|---|---|---|---|---|---|---|
| 103-002 | 4955 | 6987 | 96650 | 94895 | 19.51 | 13.58 |
| 201-003 | 7528 | 6286 | 132257 | 236613 | 17.57 | 37.64 |
| 202-001 | 11982 | 18669 | 94340 | 183345 | 7.87 | 9.82 |
| 202-002 | 19801 | 13618 | 131625 | 112733 | 6.65 | 8.28 |

Cohort 3 (300 mg QD)

| | | | | | | |
|---|---|---|---|---|---|---|
| 102-002 | 8205 | 5824 | 134290 | 86383 | 16.37 | 14.83 |
| 104-002 | 12111 | ND | 149733 | ND | 12.36 | ND |
| 201-004 | 11382 | ND | 88350 | ND | 7.76 | ND |
| 201-005 | 8338 | ND | 116734 | ND | 14.00 | ND |
| 202-003 | 11173 | *4573 | 106858 | *43933 | 9.56 | ND |

*Tₗₐₛₜ = 6 hours; ND = not determined; NA = not applicable.

This specification has been described with reference to embodiments of the invention. One of ordinary skill in the art, however, appreciates that various modifications and changes can be made without departing from the scope of the invention as set forth in the claims below. Accordingly, the specification is to be regarded in an illustrative rather than a restrictive sense, and all such modifications are intended to be included within the scope of the invention.

What is claimed is:

1. A pharmaceutical composition comprising one or more pharmaceutically acceptable excipients and an amorphous spray-dried dispersion (ASD) comprising INX-315;
wherein INX-315 is a compound of structure: 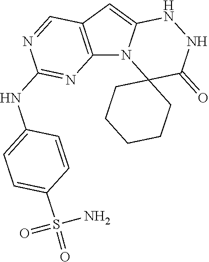 [SMILES: NS(=O)(=O)c1ccc(Nc2ncc3cc4n(c3n2)C2(CCCCC2)C(=O)NN4)cc1]
or a pharmaceutically acceptable salt thereof, and wherein the ASD further comprises one or more precipitation preventers;
and INX-315 in the ASD is between 20% and 60% by weight of the ASD and the precipitation preventers in the ASD are between 40% and 80% by weight of the ASD;
and wherein there is between 40% and 60% of the ASD by total weight in the pharmaceutical composition.

2. The pharmaceutical composition of claim 1, wherein INX-315 in the ASD is between 30% and 50% by weight of the ASD.

3. The pharmaceutical composition of claim 1, wherein INX-315 in the ASD is about 40% by weight of the ASD.

4. The pharmaceutical composition of claim 1, wherein the pharmaceutical composition comprises between 100 mg and 600 mg of INX-315.

5. The pharmaceutical composition of claim 1, wherein the pharmaceutical composition comprises about 100 mg of INX-315.

6. The pharmaceutical composition of claim 1, wherein the pharmaceutical composition comprises about 200 mg of INX-315.

7. The pharmaceutical composition of claim 1, wherein the pharmaceutical composition comprises about 300 mg of INX-315.

8. The pharmaceutical composition of claim 1, wherein the one or more precipitation preventers are selected from polyvinylpyrrolidone (PVP), PVP/VA (vinyl acetate), polymethacrylate, hypromellose (HPMC), hydroxyethyl cellulose (HEC), hydroxypropyl methylcellulose acetate succinate (HPMCAS), hydroxypropyl methylcellulose phthalate (HPMCP), cellulose acetate phthalate (CAP), sodium carboxymethyl cellulose (Na-CMC), polyacrylic acid, and polyethylene glycol.

9. The pharmaceutical composition of claim 8, wherein the one or more precipitation preventers are selected from HPMCAS-L, HPMCAS-LF, HPMCAS-LG, HPMCAS-M, HPMCAS-MF, HPMCAS-MG, HPMCAS-H, HPMCAS-HF, HPMCAS-HG, HPMC-E3, and HPMC 2910.

10. The pharmaceutical composition of claim 8, wherein the one or more precipitation preventers are selected from PEG 4000, PEG 6000, PEG 8000, and PEG 20000.

11. The pharmaceutical composition of claim 8, wherein the one or more precipitation preventers are selected from PVP K 30, PVP K 25, PVP VA64, and PVP VA37.

12. The pharmaceutical composition of claim 1, wherein the ASD comprises one precipitation preventer.

13. The pharmaceutical composition of claim 12, wherein the precipitation preventer is HPMCAS-HG.

14. The pharmaceutical composition of claim 1, wherein the ASD is about 50% by total weight in the pharmaceutical composition.

15. The pharmaceutical composition of claim 1, wherein the pharmaceutical composition comprises between 15% and 25% of an insoluble diluent by weight of the pharmaceutical composition.

16. The pharmaceutical composition of claim 15, wherein the insoluble diluent is microcrystalline cellulose.

17. The pharmaceutical composition of claim 1, wherein the pharmaceutical composition comprises between 15% and 25% of a soluble diluent by weight of the pharmaceutical composition.

18. The pharmaceutical composition of claim 17, wherein the soluble diluent is mannitol.

19. The pharmaceutical composition of claim 1, wherein the pharmaceutical composition comprises between 1% and 10% of a disintegrant by weight of the pharmaceutical composition.

20. The pharmaceutical composition of claim 19, wherein the disintegrant is croscarmellose sodium.

21. The pharmaceutical composition of claim 1, wherein the pharmaceutical composition comprises between 0.1% and 5% of a glidant by weight of the pharmaceutical composition.

22. The pharmaceutical composition of claim 21, wherein the glidant is colloidal silicon dioxide.

23. The pharmaceutical composition of claim 1, wherein the pharmaceutical composition comprises between 0.1% and 5% of a lubricant by weight of the pharmaceutical composition.

24. The pharmaceutical composition of claim 23, wherein the lubricant is sodium stearyl fumarate.

25. The pharmaceutical composition of claim 1, wherein the pharmaceutical composition comprises:
between 40% and 60% of the ASD by total weight in the pharmaceutical composition; wherein INX-315 in the ASD is between 30% and 50% by weight of the ASD and the precipitation preventers in the ASD are between 50% and 70% by weight of the ASD,
wherein the one or more pharmaceutically acceptable excipients comprise:
between 15% and 25% of an insoluble diluent by weight of the pharmaceutical composition,
between 15% and 25% of a soluble diluent by weight of the pharmaceutical composition,
between 1% and 10% of a disintegrant by weight of the pharmaceutical composition,
between 0.1% and 5% of a glidant by weight of the pharmaceutical composition, and
between 0.1% and 5% of a lubricant by weight of the pharmaceutical composition.

26. The pharmaceutical composition of claim 25, wherein INX-315 in the ASD is about 40% by weight of the ASD.

27. The pharmaceutical composition of claim 25, wherein the ASD is about 50% by total weight in the pharmaceutical composition.

28. The pharmaceutical composition of claim 25, wherein the ASD comprises one precipitation preventer.

29. The pharmaceutical composition of claim 28, wherein the precipitation preventer is HPMCAS-HG.

30. The pharmaceutical composition of claim 25, wherein the insoluble diluent is microcrystalline cellulose.

31. The pharmaceutical composition of claim 25, wherein the soluble diluent is mannitol.

32. The pharmaceutical composition of claim 25, wherein the disintegrant is croscarmellose sodium.

33. The pharmaceutical composition of claim 25, wherein the glidant is colloidal silicon dioxide.

34. The pharmaceutical composition of claim 25, wherein the lubricant is sodium stearyl fumarate.
